(19) United States
(12) Patent Application Publication
(10) Pub. No.: US 2025/0352549 A1
(43) Pub. Date: Nov. 20, 2025

(54) SUBSTITUTED HETEROCYCLIC COMPOUND DERIVATIVES AND THEIR PHARMACEUTICAL USE

(71) Applicant: AVELOS THERAPEUTICS INC., Seoul (KR)

(72) Inventors: Ki Seon BAEK, Seoul (KR); Ja Heouk KHOO, Gunpo-si Gyeonggi-do (KR); Soongyu CHOI, Seoul (KR); Young Whan PARK, Goyang-si Gyeonggi-do (KR); Darren LE GRAND, Sussex (GB); Wan-ling YAO, Seongnam-si Gyeonggi-do (KR); Jung Hwan KIM, Cheongju-si Chungcheongbuk-do (KR)

(73) Assignee: AVELOS THERAPEUTICS INC., Seoul (KR)

(21) Appl. No.: 19/234,431

(22) Filed: Jun. 11, 2025

Related U.S. Application Data
(63) Continuation of application No. 19/136,963, filed on Jan. 1, 0001.

(30) Foreign Application Priority Data
Dec. 12, 2022 (KR) 10-2022-0172653

(51) Int. Cl. A61K 31/53; A61K 31/506; A61K 45/06; C07B 59/00; C07D 401/14; C07D 403/04; C07D 403/14; C07D 417/14; C07D 471/04; C07D 487/04

(52) U.S. Cl. CPC A61K 31/53; A61K 31/506; A61K 45/06; C07B 59/002; C07D 401/14; C07D 403/04; C07D 403/14; C07D 417/14; C07D 471/04; C07D 487/04

(57) ABSTRACT

This invention relates to compounds which are microtubule associated serine/threonine-like kinase (MASTL) inhibitors and the use of the compounds in the treatment of diseases and medical conditions mediated by MASTL, for example in the treatment of cancer and other target related diseases.

### CROSS-REFERENCE TO RELATED APPLICATIONS

This application is a continuation of U.S. patent application Ser. No. 19/136,963 filed on Jun. 9, 2025, which is a national phase of International Application No. PCT/KR2023/020382 filed on Dec. 12, 2023, which claims priority to Korean Patent Application No. 10-2022-0172653 filed on Dec. 12, 2022, the entire contents of which are herein incorporated by reference.

### TECHNICAL FIELD

This invention relates to compounds which are microtubule associated serine/threonine-like kinase (MASTL) inhibitors and the use of the compounds in the treatment of diseases and medical conditions mediated by MASTL, for example in the treatment of cancer and other target related diseases.

### BACKGROUND ART

Microtubule-associated serine/threonine kinase-like (MASTL), also known as Greatwall kinase (GWL), is a member of the AGC kinase family that regulates the mitotic phosphatase complex PP2A/B55. MASTL is located on human chromosome 10p12.1 and encodes a protein of 850 amino acids. It is unique amongst kinases as it contains an approximately 500 amino acid insertion between kinase subdomains VII and VIII that corresponds to the activation loop. The protein modulates mitotic entry and exit through its ability to inactivate the phosphatase PP2A/B55 (Castilho et al., (2009). The M phase kinase Greatwall (Gwl) promotes inactivation of PP2A/B55delta, a phosphatase directed against CDK phosphosites. (Mol. Biol. Cell. 20 (22): 4777-89). MASTL inhibits the phosphatase indirectly through phosphorylation of ENSA and ARPP19 at S67 and S62 (pENSA/pARPP19), respectively (Gharbi-Ayachi et al., (2010). The substrate of Greatwall kinase, Arpp19, controls mitosis by inhibiting protein phosphatase 2A. (Science 330 1673-1677). PENSA and pARPP19 are substrates of PP2A/B55 and inhibit the complex by binding tightly to it and undergoing de-phosphorylation at a very slow rate, thus inhibiting the catalytic activity of PP2A/B55 by ‘unfair competition’(Williams et al., (2014). Greatwall-phosphorylated Endosulfine is both an inhibitor and a substrate of PP2A-B55 heterotrimers. (eLife 3: e01695.). Entry into cellular mitosis is governed by a rapid increase in the phosphorylation of numerous substrates by CDK1/CCNB1, which is accompanied by a reduction in the activity of PP2A/B55. MASTL is a substrate of CDK1/CCNB1 and a combination of their activities ensure MASTL activity peaks at mitosis. MASTL activity is essential to coordinate exit from mitosis by delaying the increase of PP2A/B55 activity until chromosomal segregation is complete. APC/C dependent ubiquitination of CCNB1, followed by its subsequent degradation by the proteasome, initiates anaphase entry. This attenuates CDK1 activity leading to the eventual deactivation of MASTL and an increase in the PP2A/B55 phosphatase activity that is required for timely exit from mitosis. Temporal control of PP2A/B55 reactivation by the PP2A-B55-ENSA/ARPP19-MASTL pathway is essential for orderly cytokinesis following chromosomal segregation (Cundell et al., (2013). The BEG (PP2A-B55/ENSA/Greatwall) pathway ensures cytokinesis follows chromosome separation. (Mol. Cell 52 393-405). Inhibiting the kinase activity of MASTL will result in premature cytokinesis, causing chromosome segregation defects and aneuploidy.

MASTL has been shown to be essential for cell-cycle progression during embryogenesis in a number of organisms, including mouse, frog and fruit-fly. In mouse it remains essential for up to one year after birth after which, its loss (total deletion) is tolerated (Belen Sanz Castillo: Role of MASTL in mammals: Molecular functions and physiological relevance, 2017). Furthermore a siRNA screen identified MASTL as a gene that can specifically inhibit the proliferation of transformed (thyroid cancer) cells but not non-transformed cells (Anania et al., (2015) Identification of thyroid tumor cell vulnerabilities through a siRNA-based functional screening. (Oncotarget 6, 34629-34648). These studies show that MASTL essentiality is not universal and that it is confined to embryonic and early development stages of organisms. Moreover, the studies show that the cell cycle control mechanisms in some cancer cells have reverted back to a state similar to that of embryonic cell cycles (where MASTL activity is essential) to render them sensitive to MASTL loss. Therefore inhibitors of MASTL kinase will have broad applicability across a multitude of cancers while also having a good therapeutic window, and as such is an ideal target for cancer therapy.

A number of studies have demonstrated that MASTL plays a critical role in cancer development. Overexpression of MASTL has been identified in a range of other human tumours, including breast (Alvarez-Fernández et al. (2017), oral (Wang et al., (2014). Mastl kinase, a promising therapeutic target, promotes cancer recurrence. (Oncotarget 5 11479-11489.) and gastric (Sun et al., (2017). Mastl overexpression is associated with epithelial to mesenchymal transition and predicts a poor clinical outcome in gastric cancer. (Oncol. Lett. 14 7283-7287.). Therapeutic relevance of the PP2A-B55 inhibitory kinase MASTL/Greatwall in breast cancer. (Cell Death Differ. 25, 828-840; Zhuge et al., (2017)). MASTL is a potential poor prognostic indicator in ER+breast cancer. (Eur. Rev. Med. Pharmacol. Sci. 21 2413-2420.), and colon (Vera et al., (2015). Greatwall promotes cell transformation by hyperactivating AKT in human malignancies. (eLife 4, e10115.). Mouse xenograft studies using doxycycline inducible knock out of MASTL by CRISPR/Cas9 in MDA-MB-231 cells showed a significant reduction in the tumour size when MASTL was depleted relative to control animals. Expression levels of MASTL protein correlated with aggressiveness in ER+breast cancer and were prognostic for poor patient survival (Álvarez-Fernández et al., (2018). Therapeutic relevance of the PP2A-B55 inhibitory kinase MASTL/Greatwall in breast cancer. (Cell Death Differ. 25 828-840). Upregulation of MASTL is correlated with cancer progression in head and neck tumours, and it is frequently associated with more aggressive forms of the disease (Wang et al., (2014). Mastl kinase, a promising therapeutic target, promotes cancer recurrence. (Oncotarget 5, 11479-11489). A high throughput siRNA screen in BCPAP thyroid cancer identified vulnerabilities to the loss of MASTL, which resulted in a significant reduction in cell proliferation (Anania et al. (2015)). In colorectal cancer, upregulation of MASTL is correlated with poor patient survival and can act as a prognostic biomarker for latent disease aggressiveness (Uppada et al., (2018). MASTL induces colon cancer progression and chemoresistance by promoting Wnt/Yâ-catenin signaling. (Mol. Cancer 17:111). In support of a therapeutic window, normal colonocytes do not express MASTL, or do so only at very low levels. Depletion of MASTL in HCT-116 cells resulted in G2/M arrest, induction of apoptosis through regulation of the anti-apoptotic proteins (Survivin and Bcl-xL, probably via Gsk3Yâ activation) and importantly reduced growth in vivo. In addition to having a direct effect on HCT-116 cell proliferation, the MASTL derived regulation of anti-apoptotic proteins resulted in increased sensitivity to 5-FU treatment. MASTL has been highlighted as a potential new therapeutic target for several cancers, such as acute myeloid leukemia (Tzelepis et al. (2016). A CRISPR dropout screen identifies genetic vulnerabilities and therapeutic targets in acute myeloid leukemia. (Cell Rep. 17, 1193-1205.), head and neck squamous cell carcinoma (Wang et al., 2014) and thyroid carcinoma (Anania et al., 2015).

In addition to its role as a regulator of the G2/M checkpoint, MASTL can inactivate checkpoint signalling and help recovery from DNA damage, supporting a role in potentiating effects of DNA damaging agents (Peng et al., (2010). A novel role for greatwall kinase in recovery from DNA damage. (Cell Cycle 9 4364-4369). An unbiased genome-wide siRNA loss of function screen in NSCLC cells identified MASTL as the primary hit for sensitising the cells to irradiation. The effect was not observed in primary human fibroblast, indicating the potential for selective sensitization of tumour cells over untransformed cells (Nagel et al., (2015). Genome-wide siRNA Screen identifies the radiosensitizing effect of downregulation of MASTL and FOXM1 in NSCLC. (Mol. Cancer Ther. 14 1434-1444). A similar effect was observed in a xenograft tumour model of UM-SSC-11-B cells derived from head and necks squamous cell carcinomas refractory to cisplatin (Wang et al., 2014). MASTL depletion re-sensitised the cells to cisplatin treatment. Additional flow cytometry studies in UM-SSC-11-B cells showed an increase sub G1 population and induction of apoptosis, while normal oral keratinocyte OKF4 cells depleted of MASTL were resistant to cell death with or without cisplatin treatment.

In addition to the role of MASTL in cancer through regulation of DNA damage repair pathways and mitosis, it also has a role in modulating PP2A activity in interphase (Belén Sanz Castillo, 2017). A point mutation in the MASTL gene was found to lead to an autosomal dominant inherited thrombocytopenia (Drachman et al., Autosomal dominant thrombocytopenia: incomplete megakaryocyte differentiation and linkage to human chromosome 10. (Blood. 2000; 96:118-125.), providing evidence of the role of MASTL in megakaryocytopoeisis. More recently it was discovered that this point mutation in MASTL does not result in reduced activity, as originally thought, but rather is accompanied by increased phosphorylation of the Cdk and PP2A substrates, indicating a gain-of-function alteration that results in decreased PP2A activity (Hurtado et al., (2018) Thrombocytopenia-associated mutations in Ser/Thr kinase MASTL deregulate actin cytoskeletal dynamics in platelets. (J Clin Invest. 128 (12): 5351-5367). A MASTL inhibitor may therefore have therapeutic potential in the treatment of metabolic diseases (such as diabetes and obesity) and platelet disorders, including the rare genetic disease MASTL-linked thrombocytopenia, through its effects on the regulation of the PI3K/AKT pathway and the cytoskeleton, respectively.

There is therefore a need for MASTL inhibitors which are expected to provide a beneficial therapeutic effect, for example in the treatment of cancer.

### SUMMARY OF INVENTION

In accordance with the present inventions there is provided a compound of the formula (I), or a pharmaceutically acceptable salt thereof:

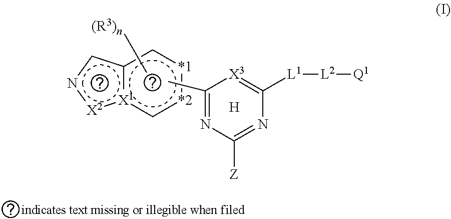

wherein the H ring in formula (I) is bonded to the carbon atom *1 or *2;
Z is —NR¹R² or —CN;
R¹ and R² is independently selected from: H, D, and C₁₋₆ alkyl,
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-;
p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from: C₃₋₁₂ cycloalkyl, C₃-12 cycloalkenyl, 3- to 12-membered heterocyclyl, C₆₋₁₀ aryl, and 5- to 10-membered heteroaryl,
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰)₂, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰)₂, —OC(O)N(R¹⁰)₂, —NR¹⁰SO₂R¹⁰, —SO₂N(R¹⁰)₂ and —NR¹⁰C(O)N(R¹⁰)₂;
wherein said C₁₋₆ alkyl, C₂₋₆ alkenyl and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{X1}R^{X2};wherein R^{X1} and R^{X2} are independently selected from: H, C₁₋₄alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when R⁴ or R⁵ is H or C₁₋₆ alkyl which is not deuterated, then Z is —CN, or
—NR¹R² wherein at least one of R¹ and R² is D or partially or fully deuterated C₁₋₆ alkyl.

In addition, in accordance with the present inventions there is provided a compound of the formula (II), or a pharmaceutically acceptable salt thereof:

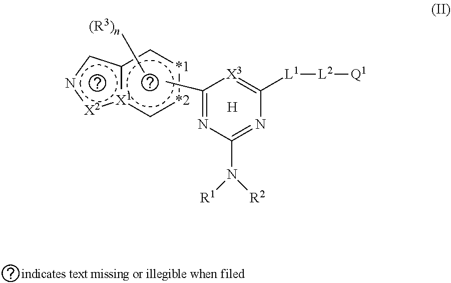

wherein the H ring in formula (II) is bonded to the carbon atom *1
R¹ and R² is independently selected from: H, C₁₋₆ alkyl, C₂₋₆ alkenyl, C₂₋₆ alkynyl, and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-;
p is an integer from 1 to 4;
R⁷ and R⁸ are each independently selected from: H, C₁₋₄ alkyl, C₆₋₁₀ aryl, COO—C₁₋₆ alkyl, NR^{X1}R^{X2}, C(O)NR^{x1}R^{X3}, and 5- to 10-membered heteroaryl;
wherein at least one of R⁷ and R⁸ in L² is not H, and said C₁₋₄ alkyl is substituted by 3- to 6-membered cycloalkyl, C₄₋₈ alkyl, or NR^{X1}R^{X2};
Q¹ is selected from: C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl, C₂₋₆ alkynyl and C₁₋₆ haloalkyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, NR^{x1}R^{X2} and C(O)NR^{x1}R^{X2}, wherein R^{X1} and R^{X2} are independently selected from: H, C₁₋₄alkyl optionally substituted by OH or 3- to 6-membered heterocyclyl, OH, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
wherein R^{X3} is selected from: OH and O—C₁₋₆ alkyl;
n is an integer from 0 to 4; and
x is an integer from 0 to 3.

In addition, in accordance with the present inventions there is provided a compound of the formula (III), or a pharmaceutically acceptable salt thereof:

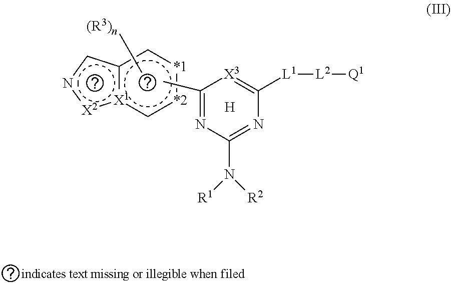

wherein the H ring in formula (III) is bonded to the carbon atom *1 or *2;
R¹ and R² is independently selected from: H, C₁₋₆ alkyl, C₂₋₆ alkenyl, C₂₋₆ alkynyl and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₁₋₄ alkyl is optionally partially or fully deuterated;
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-;
p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁₋₆ haloalkyl;
Q¹ is selected from C₆₋₁₀ aryl and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, C₃-C₆ cycloalkyl, 5- to 10-membered heterocyclyl, 5- to 10-membered heteroaryl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl, C₂₋₆ alkynyl, C₃-C₆ cycloalkyl, 5- to 10-membered heterocyclyl, and 5- to 10-membered heteroaryl is optionally substituted by one or more R¹¹,
wherein said C₁₋₆ alkyl is optionally partially or fully deuterated;
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when Q¹ is not substituted by one or more R⁹, or
when any one of one or more R⁹ on Q¹ is not C₃-C₆ cycloalkyl, 5- to 10-membered heterocyclyl, 5- to 10-membered heteroaryl, or C₁₋₆ alkyl which is partially or fully deuterated, then
R⁶ is C₁-4 alkyl which is partially or fully deuterated.

In addition, in accordance with the present inventions there is provided a compound of the formula (IV), or a pharmaceutically acceptable salt thereof:

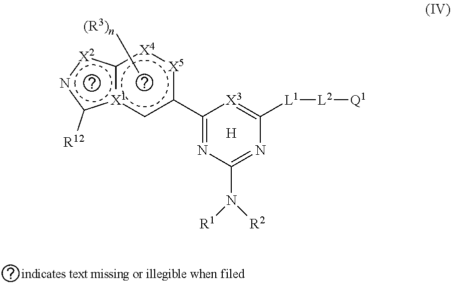

wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH;
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR 10C(O)N(R¹⁰) 2,
wherein said C₁₋₆ alkyl, C₂₋₆ alkenyl, and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3.

In addition, in accordance with the present inventions there are provided Compound Nos. 1-239 listed in Table 1 of the present specification, or a pharmaceutically acceptable salt thereof.

Also provided is a pharmaceutical composition comprising a compound of the invention, or a pharmaceutically acceptable salt thereof, and a pharmaceutically acceptable excipient.

Also provided is a compound of the invention, or a pharmaceutically acceptable salt thereof, for use as a medicament. In some embodiments the compound of the invention, or a pharmaceutically acceptable salt thereof, is for use in the treatment of a disease or medical condition mediated by microtubule associated serine/threonine-like kinase (MASTL).

Also provided is a compound of the invention, or a pharmaceutically acceptable salt thereof for use in the treatment of a disease in which PD-L1 expression is dependent on interferon.

Also provided is a method of treating a disease or medical condition mediated by MASTL in a subject in need thereof, the method comprising administering to the subject an effective amount of a compound of the invention, or a pharmaceutically acceptable salt thereof.

In certain embodiments the compounds of the invention are for use in the treatment of proliferative diseases, for example cancer. In certain embodiments a compound of the invention is for use in the prevention or inhibition of cancer progression, for example by preventing or inhibiting cancer cell migration, cancer cell invasion and/or preventing or inhibiting cancer metastasis.

In certain embodiments the compounds of the invention are for use in the treatment of a cancer.

In certain embodiments the compounds of the invention are for use in the treatment of a cancer that overexpresses MASTL. In certain embodiments the compounds of the invention are for use in the treatment of a cancer selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and haematological cancer (e.g. leukaemia, such as AML, lympoma, myeloma and multiple myeloma).

In certain embodiments, the compounds of the invention are for use in the treatment or prevention of a metabolic disorder, or symptoms or conditions associated with a metabolic disease.

In certain embodiments, the metabolic disorder may be insulin resistance, diabetes or obesity. Symptoms and conditions associated with a metabolic disorder may include one or more of: increased blood sugar, increased cholesterol, increased triglyceride levels, heart disease, stroke, high blood pressure, and an increased risk of blood clots (e.g. deep vein thrombosis).

In certain embodiments, the compounds of the invention are for use in the treatment of a platelet disorder, such as thrombocytopenia.

The compounds of the invention may be used alone or in combination with one or more anticancer agents and/or radiotherapy as described herein.

### DISCLOSURE

### Technical Solution

### Detailed Description

### Definitions

Unless otherwise stated, the following terms used in the specification and claims have the following meanings set out below.

The terms “treating” or “treatment” refers to any indicia of success in the treatment or amelioration of a disease, pathology or condition, including any objective or subjective parameter such as abatement; remission; diminishing of symptoms or making the pathology or condition more tolerable to the patient; slowing in the rate of degeneration or decline; making the final point of degeneration less debilitating; improving a patient's physical or mental well-being. For example, certain methods herein treat cancer by decreasing a symptom of cancer. Symptoms of cancer would be known or may be determined by a person of ordinary skill in the art. The term “treating” and conjugations thereof, include prevention of a pathology, condition, or disease (e.g. preventing the development of one or more symptoms of a cancer associated with MASTL.

The term “associated” or “associated with” in the context of a substance or substance activity or function associated with a disease (e.g. cancer) means that the disease (e.g. cancer) is caused by (in whole or in part), or a symptom of the disease is caused by (in whole or in part) the substance or substance activity or function. For example, a symptom of a disease or condition associated with MASTL pathway activity may be a symptom that results (entirely or partially) from an increase in the level of activity of MASTL protein pathway. As used herein, what is described as being associated with a disease, if a causative agent, could be a target for treatment of the disease. For example, a disease associated with an increase in the level of activity of MASTL, may be treated with an agent (e.g. compound as described herein) effective for decreasing the level of activity of MASTL.

As defined herein, the term “inhibition”, “inhibit”, “inhibiting” and the like in reference to a protein-inhibitor (e.g. antagonist) interaction means negatively affecting (e.g. decreasing) the level of activity or function of the protein (e.g. a component of the MASTL) protein pathway relative to the level of activity or function of the protein pathway in the absence of the inhibitor). In some embodiments inhibition refers to reduction of a disease or symptoms of disease (e.g. cancer associated with an increased level of activity of MASTL. In some embodiments, inhibition refers to a reduction in the level of activity of a signal transduction pathway or signalling pathway associated with MASTL. Thus, inhibition may include, at least in part, partially or totally blocking stimulation, decreasing, preventing, or delaying activation, or inactivating, desensitizing, or down-regulating signal transduction or enzymatic activity or the amount of a protein (e.g. the MASTL). Inhibition may include, at least in part, partially or totally decreasing stimulation, decreasing activation, or deactivating, desensitizing, or down-regulating signal transduction or enzymatic activity or the amount of a protein (e.g. a component of a MASTL protein pathway) that may modulate the level of another protein or modulate cell survival, cell proliferation or cell motility relative to a non-disease control.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

The term “halo” or “halogen” refers to one of the halogens, group 17 of the periodic table. In particular the term refers to fluorine, chlorine, bromine and iodine. Preferably, the term refers to fluorine or chlorine.

The term Cₘ₋ₙ refers to a group with m to n carbon atoms.

The term “C₁₋₆ alkyl” refers to a linear or branched hydrocarbon chain containing 1, 2, 3, 4, 5 or 6 carbon atoms, for example methyl, ethyl, n-propyl, iso-propyl, n-butyl, iso-butyl, sec-butyl, tert-butyl, n-pentyl and n-hexyl. “C₁₋₄ alkyl” similarly refers to such groups containing up to 4 carbon atoms. Alkylene groups are divalent alkyl groups and may likewise be linear or branched and have two points of attachment to the remainder of the molecule. Furthermore, an alkylene group may, for example, correspond to one of those alkyl groups listed in this paragraph. For example, C₁₋₆ alkylene may be —CH₂—, —CH₂CH₂—, —CH₂CH(CH₃)—, —CH₂CH₂CH₂— or —CH₂CH(CH₃) CH₂—. The alkyl and alkylene groups may be unsubstituted or substituted by one or more substituents. Possible substituents are described herein. For example, substituents for an alkyl or alkylene group may be halogen, e.g. fluorine, chlorine, bromine and iodine, OH, C₁-C₄ alkoxy, —NR′R″ amino, wherein R′ and R″ are independently H or alkyl. Other substituents for the alkyl group may alternatively be used.

The term “C₁₋₆ haloalkyl”, e.g. “C₁₋₄ haloalkyl” refers to a hydrocarbon chain substituted with at least one halogen atom independently chosen at each occurrence, for example fluorine, chlorine, bromine and iodine. The halogen atom may be present at any position on the hydrocarbon chain. For example, C₁₋₆ haloalkyl may refer to chloromethyl, fluoromethyl, trifluoromethyl, chloroethyl e.g. 1-chloromethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl. A haloalkyl group may be, for example, —CX₃, —CHX₂, —CH₂CX₃, —CH₂CHX₂ or —CX(CH₃) CH₃ wherein X is a halo(e.g. F, Cl, Br or I). A fluoroalkyl group, i.e. a hydrocarbon chain substituted with at least one fluorine atom (e.g. —CF₃, —CHF₂, —CH₂CF₃ or —CH₂CHF₂).

The term “C₂₋₆ alkenyl” includes a branched or linear hydrocarbon chain containing at least one double bond and having 2, 3, 4, 5 or 6 carbon atoms. The double bond(s) may be present as the E or Z isomer. The double bond may be at any possible position of the hydrocarbon chain. For example, the “C₂₋₆ alkenyl” may be ethenyl, propenyl, butenyl, butadienyl, pentenyl, pentadienyl, hexenyl and hexadienyl. Alkenylene groups are divalent alkenyl groups and may likewise be linear or branched and have two points of attachment to the remainder of the molecule. Furthermore, an alkenylene group may, for example, correspond to one of those alkenyl groups listed in this paragraph. For example alkenylene may be —CH—CH—, —CH₂CH═CH—, —CH(CH₃) CH═CH— or —CH₂CH═CH—. Alkenyl and alkenylene groups may unsubstituted or substituted by one or more substituents. Possible substituents are described herein. For example, substituents may be those described above as substituents for alkyl groups.

The term “C₂₋₆ alkynyl” includes a branched or linear hydrocarbon chain containing at least one triple bond and having 2, 3, 4, 5 or 6 carbon atoms. The triple bond may be at any possible position of the hydrocarbon chain. For example, the “C₂₋₆ alkynyl” may be ethynyl, propynyl, butynyl, pentynyl and hexynyl. Alkynylene groups are divalent alkynyl groups and may likewise be linear or branched and have two points of attachment to the remainder of the molecule. Furthermore, an alkynylene group may, for example, correspond to one of those alkynyl groups listed in this paragraph. For example alkynylene may be —CᵢǑC—, —CH₂CᵢǑC—, —CH₂CᵢǑCCH₂—, —CH(CH₃)CHᵢǑC— or —CH₂CᵢǑCCH₃. Alkynyl and alkynylene groups may unsubstituted or substituted by one or more substituents. Possible substituents are described herein. For example, substituents may be those described above as substituents for alkyl groups.

The term “C₃₋₁₂ cycloalkyl” includes a saturated hydrocarbon ring system containing 3 to 12 carbon atoms. The cycloalkyl group may be monocyclic or a fused, bridged or spiro saturated hydrocarbon ring system. The term “C₃₋₆ cycloalkyl” includes a saturated hydrocarbon ring system containing 3, 4, 5 or 6 carbon atoms. For example, the C₃-C₁₂ cycloalkyl may be cyclopropyl, cyclobutyl, cyclopentyl, cyclohexyl, bicyclo[1.1.1]pentane, bicyclo[2.1.1]hexane, bicyclo[2.2.1]heptane (norbornane), bicyclo[2.2.2]octane or tricyclo[3.3.1.1]decane (adamantyl). For example, the “C₃-C₆ cycloalkyl” may be cyclopropyl, cyclobutyl, cyclopentyl, cyclohexyl, bicyclo[2.1.1]hexane or bicyclo[1.1.1]pentane. Suitably the “C₃-C₆ cycloalkyl” may be cyclopropyl, cyclobutyl, cyclopentyl or cyclohexyl.

The term “C₃₋₁₂ cycloalkenyl” includes a hydrocarbon ring system containing 3 to 12 carbon atoms and at least one double bond (e.g. 1 or 2 double bonds). The cycloalkenyl group may be monocyclic or a fused, bridged or spiro hydrocarbon ring system. For example, C₃₋₁₂ cycloalkenyl may be cyclobutenyl, cyclopentenyl, cyclohexenyl,

The term “heterocyclyl”, “heterocyclic” or “heterocycle” includes a non-aromatic saturated or partially saturated monocyclic or fused, bridged, or spiro bicyclic heterocyclic ring system. Monocyclic heterocyclic rings may contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles may contain from 7 to 12-member atoms in the ring. Bicyclic heterocyclic(s) rings may be fused, spiro, or bridged ring systems. The heterocyclyl group may be a 3-12, for example, a 3- to 9-(e.g. a 3- to 7-) membered non-aromatic monocyclic or bicyclic saturated or partially saturated group comprising 1, 2 or 3 heteroatoms independently selected from O, S and N in the ring system (in other words 1, 2 or 3 of the atoms forming the ring system are selected from O, S and N). By partially saturated it is meant that the ring may comprise one or two double bonds. This applies particularly to monocyclic rings with from 5 to 7 members. The double bond will typically be between two carbon atoms but may be between a carbon atom and a nitrogen atom. Bicyclic systems may be spiro-fused, i.e. where the rings are linked to each other through a single carbon atom; vicinally fused, i.e. where the rings are linked to each other through two adjacent carbon or nitrogen atoms; or they may be share a bridgehead, i.e. the rings are linked to each other through two non-adjacent carbon or nitrogen atoms (a bridged ring system). Examples of heterocyclic groups include cyclic ethers such as oxiranyl, oxetanyl, tetrahydrofuranyl, dioxanyl, and substituted cyclic ethers. Heterocycles comprising at least one nitrogen in a ring position include, for example, azetidinyl, pyrrolidinyl, piperidinyl, piperazinyl, morpholinyl, thiomorpholinyl, tetrahydrotriazinyl, tetrahydropyrazolyl, tetrahydropyridinyl, homopiperidinyl, homopiperazinyl, 2,5-diaza-bicyclo[2.2.1]heptanyl and the like. Typical sulfur containing heterocycles include tetrahydrothienyl, dihydro-1,3-dithiol, tetrahydro-2H-thiopyran, and hexahydrothiepine. Other heterocycles include dihydro oxathiolyl, tetrahydro oxazolyl, tetrahydro-oxadiazolyl, tetrahydrodioxazolyl, tetrahydrooxathiazolyl, hexahydrotriazinyl, tetrahydro oxazinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo(═O), for example, 2 oxopyrrolidinyl, 2-oxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. For example, the term “piperidino” or “morpholino” refers to a piperidin-1-yl or morpholin-4-yl ring that is linked via the ring nitrogen. Reference to “heterocyclylene”, for example as may be represented by L¹ refers to a divalent “heterocyclyl”, for example 3,2-morpholinylene.

The term “bridged ring systems” includes ring systems in which two rings share more than two atoms, see for example Advanced Organic Chemistry, by Jerry March 4th Edition, Wiley Interscience, pages 131-133, 1992. Suitably the bridge is formed between two non-adjacent carbon or nitrogen atoms in the ring system. The bridge connecting the bridgehead atoms may be a bond or comprise one or more atoms. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane, and quinuclidine.

The term “spiro bi-cyclic ring systems” includes ring systems in which two ring systems share one common spiro carbon atom, i.e. the heterocyclic ring is linked to a further carbocyclic or heterocyclic ring through a single common spiro carbon atom. Examples of spiro ring systems include 3,8-diaza-bicyclo[3.2.1]octane, 2,5-diaza-bicyclo[2.2.1]heptane, 6-azaspiro[3.4]octane, 2-oxa-6-azaspiro[3.4]octane, 2-azaspiro[3.3]heptane, 2-oxa-6-azaspiro[3.3]heptane, 6-oxa-2-azaspiro[3.4]octane, 2,7-diaza-spiro[4.4]nonane, 2-azaspiro[3.5]nonane, 2-oxa-7-azaspiro[3.5]nonane and 2-oxa-6-azaspiro[3.5]nonane.

“Heterocyclyl-Cₘ₋ₙ alkyl” includes a heterocyclyl group covalently attached to a Cₘ₋ₙ alkylene group, both of which are defined herein; and wherein the Heterocyclyl-Cₘ₋ₙ alkyl group is linked to the remainder of the molecule via a carbon atom in the alkylene group. The groups “aryl-Cₘ₋ₙ alkyl”, “heteroaryl-Cₘ₋ₙ alkyl” and “cycloalkyl-Cₘ₋ₙ alkyl” are defined in the same way. “—Cₘ₋ₙ alkyl” substituted by —NRR″ and “Cₘ₋ₙ alkyl” substituted by —OR″ similarly refer to an —NRR″ or —OR″ group covalently attached to a Cₘ₋ₙ alkylene group and wherein the group is linked to the remainder of the molecule via a carbon atom in the alkylene group.

The term “aromatic” when applied to a substituent as a whole includes a single ring or polycyclic ring system with 4n+2 electrons in a conjugated ©£ system within the ring or ring system where all atoms contributing to the conjugated ©£ system are in the same plane.

The term “aryl” includes an aromatic hydrocarbon ring system. The ring system has 4n+2 electrons in a conjugated Of system within a ring where all atoms contributing to the conjugated Cf system are in the same plane. For example, the “aryl” may be phenyl and naphthyl. The aryl system itself may be substituted with other groups.

The term “heteroaryl” includes an aromatic mono- or bicyclic ring incorporating one or more (for example 1-4, particularly 1, 2 or 3) heteroatoms selected from nitrogen, oxygen or sulfur. The ring or ring system has 4n+2 electrons in a conjugated ©£ system where all atoms contributing to the conjugated ©£ system are in the same plane.

Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 1H-pyrazolo[4,3-d]-oxazolyl, 4H-imidazo[4,5-d]thiazolyl, pyrazino[2,3-d]pyridazinyl, imidazo[2,1-b]thiazolyl and imidazo[1,2-b][1,2,4]triazinyl. Examples of heteroaryl groups comprising at least one nitrogen in a ring position include pyrrolyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, indolyl, isoindolyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl and pteridinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl.

Examples of five-membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six-membered heteroaryl groups include but are not limited to pyridyl, pyrazinyl, pyridazinyl, pyrimidinyl and triazinyl.

Particular examples of bicyclic heteroaryl groups containing a six-membered ring fused to a five-membered ring include but are not limited to benzofuranyl, benzothiophenyl, benzimidazolyl, benzoxazolyl, benzisoxazolyl, benzothiazolyl, benzisothiazolyl, isobenzofuranyl, indolyl, isoindolyl, indolizinyl, indolinyl, isoindolinyl, purinyl(e.g., adeninyl, guaninyl), indazolyl, benzodioxolyl, pyrrolopyridine, and pyrazolopyridinyl groups.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term “oxo,” or “═O” as used herein, means an oxygen that is double bonded to a carbon atom.

The term “optionally substituted” includes either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Reference to a —NRR′ group forming a 4 to 6 membered heterocyclyl refers to R and R′ together with the nitrogen atom to which they are attached forming a 4 to 6 membered heterocyclyl group. For example, an —NRR′ such as a —NR^{X1}R^{X2} group may form:

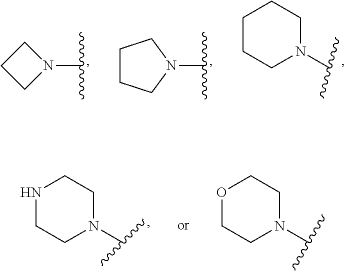

Similarly an —NRR′ group within a substituent may form a carbonyl-linked 4 to 6 membered heterocyclyl, for example a —C(O)NRR′ group may form:

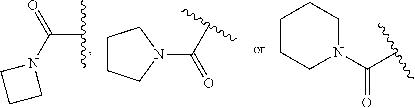

—NRR′ groups within substituents such as —OC(O)NRR′, —SO₂NRR′ and —NRC(O)NRR′, -, may similarly form a 4 to 6 membered heterocyclyl within such substituents.

*190 The phrase “compound of the invention” means those compounds which are disclosed herein, both generically and specifically. Accordingly compounds of the invention include compounds of the formulae (I), (II), (III), or (IV) and the compounds in the Examples.

A bond terminating in a “ [symbol] ” or “*” represents that the bond is connected to another atom that is not shown in the structure. A bond terminating inside a cyclic structure and not terminating at an atom of the ring structure represents that the bond may be connected to any of the atoms in the ring structure where allowed by valency.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The various functional groups and substituents making up the compounds of the present invention are typically chosen such that the molecular weight of the compound does not exceed 1000. More usually, the molecular weight of the compound will be less than 750, for example less than 700, or less than 650, or less than 600, or more preferably less than 550.

Suitable or preferred features of any compounds of the present invention may also be suitable features of any other aspect.

The invention contemplates pharmaceutically acceptable salts of the compounds of the invention. These may include the acid addition and base salts of the compounds. These may be acid addition and base salts of the compounds.

Suitable acid addition salts are formed from acids which form non-toxic salts. Examples include the acetate, aspartate, benzoate, besylate, bicarbonate/carbonate, bisulfate/sulfate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulfate, naphthylate, 1,5-naphthalenedisulfonate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts. For a review on suitable salts, see “Handbook of Pharmaceutical Salts: Properties, Selection, and Use” by Stahl and Wermuth (Wiley-VCH, Weinheim, Germany, 2002).

Pharmaceutically acceptable salts of compounds of the invention may be prepared by for example, one or more of the following methods:
(i) by reacting the compound of the invention with the desired acid or base;
(ii) by removing an acid- or base-labile protecting group from a suitable precursor of the compound of the invention or by ring-opening a suitable cyclic precursor, for example, a lactone or lactam, using the desired acid or base; or
(iii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterized by the absolute configuration of its asymmetric centre and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereo centres any combination of (R) and(S) stereoisomers is contemplated. The combination of (R) and(S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%, for example at least 90%, at least 95% or at least 99%.

The compounds of this invention may possess one or more asymmetric centres; such compounds can therefore be produced as individual (R)- or(S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess MASTL inhibitory activity.

Z/E (e.g. cis/trans) isomers may be separated by conventional techniques well known to those skilled in the art, for example, chromatography and fractional crystallisation.

Conventional techniques for the preparation/isolation of individual enantiomers when necessary include chiral synthesis from a suitable optically pure precursor or resolution of the racemate (or the racemate of a salt or derivative) using, for example, chiral high-pressure liquid chromatography (HPLC). Thus, chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and for specific examples, 0 to 5% by volume of an alkylamine e.g. 0.1% diethylamine. Concentration of the eluate affords the enriched mixture.

Alternatively, the racemate (or a racemic precursor) may be reacted with a suitable optically active compound, for example, an alcohol, or, in the case where the compound of the invention contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art-see, for example, “Stereochemistry of Organic Compounds” by E. L. Eliel and S. H. Wilen (Wiley, 1994).

Compounds and salts described in this specification may be isotopically-labelled (or “radio-labelled”). Accordingly, one or more atoms are replaced by an atom having an atomic mass or mass number different from the atomic mass or mass number typically found in nature. Examples of radionuclides that may be incorporated include ²H (also written as “D” for deuterium), 3H (also written as “T” for tritium), ¹¹C, ¹³C, ¹⁴C, ¹⁵O, ¹⁷O, ¹⁸O, ¹³N, ¹⁵N, ¹⁸F, ³⁶Cl, ¹²³I, ²⁵I, ³²P, ³⁵S and the like. The radionuclide that is used will depend on the specific application of that radio-labelled derivative. For example, for in-vitro competition assays, ³H or ¹⁴C are often useful. For radio-imaging applications, ¹¹C or ¹⁸F are often useful. In some embodiments, the radionuclide is ³H. In some embodiments, the radionuclide is ¹⁴C. In some embodiments, the radionuclide is ¹¹C. And in some embodiments, the radionuclide is ¹⁸F.

Isotopically-labelled compounds can generally be prepared by conventional techniques known to those skilled in the art or by processes analogous to those described using an appropriate isotopically-labelled reagent in place of the non-labelled reagent previously employed.

The selective replacement of hydrogen with deuterium in a compound may modulate the metabolism of the compound, the PK/PD properties of the compound and/or the toxicity of the compound. For example, deuteration may increase the half-life or reduce the clearance of the compound in-vivo. Deuteration may also inhibit the formation of toxic metabolites, thereby improving safety and tolerability. It is to be understood that the invention encompasses deuterated derivatives of compounds of formula (I). As used herein, the term deuterated derivative refers to compounds of the invention where in a particular position at least one hydrogen atom is replaced by deuterium. For example, one or more hydrogen atoms in a C₁₋₄-alkyl group may be replaced by deuterium to form a deuterated C₁₋₄-alkyl group.

Certain compounds of the invention may exist in solvated as well as unsolvated forms such as, for example, hydrated forms. It is to be understood that the invention encompasses all such solvated forms that possess MASTL inhibitory activity.

It is also to be understood that certain compounds of the invention may exhibit polymorphism, and that the invention encompasses all such forms that possess MASTL inhibitory activity.

Compounds of the invention may exist in a number of different tautomeric forms and references to compounds of the invention include all such forms. For the avoidance of doubt, where a compound can exist in one of several tautomeric forms, and only one is specifically described or shown, all others are nevertheless embraced by compounds of the invention. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

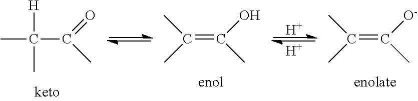

Amino substituted triazines can exhibit hindered rotation about the SP2 carbon-N bond giving rise to diastereomers (blocked rotamers)(Amm et al. (1998), Mag. Reson. Chem. 36 587-596). Reference to a compound of the invention encompasses all such blocked-rotamer forms of the compound.

The in-vivo effects of a compound of the invention may be exerted in part by one or more metabolites that are formed within the human or animal body after administration of a compound of the invention.

It is further to be understood that a suitable pharmaceutically-acceptable pro-drug of a compound of the formula (I) also forms an aspect of the present invention. Accordingly, the compounds of the invention encompass pro-drug forms of the compounds and the compounds of the invention may be administered in the form of a pro-drug (i.e. a compound that is broken down in the human or animal body to release a compound of the invention). A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in-vivo-cleavable ester derivatives that may be formed at a carboxy group or a hydroxy group in a compound of the invention and in-vivo-cleavable amide derivatives that may be formed at a carboxy group or an amino group in a compound of the invention.

Accordingly, the present invention includes those compounds of the invention as defined herein when made available by organic synthesis and when made available within the human or animal body by way of cleavage of a pro-drug thereof. Accordingly, the present invention includes those compounds of the formula (I) that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically-acceptable pro-drug of a compound of the invention is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically-acceptable pro-drug of a compound of the formula I that possesses a carboxy group is, for example, an in-vivo-cleavable ester thereof. An in-vivo-cleavable ester of a compound of the invention containing a carboxy group is, for example, a pharmaceutically-acceptable ester which is cleaved in the human or animal body to produce the parent acid. Suitable pharmaceutically-acceptable esters for carboxy include C₁₋₆ alkyl esters such as methyl, ethyl and tert-butyl, C₁₋₆ alkoxymethyl esters such as methoxymethyl esters, C₁₋₆ alkanoyloxymethyl esters such as pivaloyloxymethyl esters, 3-phthalidyl esters, C₃₋₈ cycloalkylcarbonyloxy-C₁₋₆ alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C₁₋₆ alkoxycarbonyloxy-C₁₋₆ alkyl esters such as methoxycarbonyloxymethyl and 1-methoxycarbonyloxyethyl esters. A suitable pharmaceutically-acceptable pro-drug of a compound of the invention that possesses a hydroxy group is, for example, an in-vivo-cleavable ester or ether thereof. An in-vivo-cleavable ester or ether of a compound of the invention containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀ alkanoyl groups such as acetyl, benzoyl, phenylacetyl and substituted benzoyl and phenylacetyl groups, C₁₋₁₀ alkoxycarbonyl groups such as ethoxycarbonyl, N,N-(C₁₋₆ alkyl) 2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄ alkyl) piperazin-1-ylmethyl. Suitable pharmaceutically-acceptable ether forming groups for a hydroxy group include ¥á-acyloxyalkyl groups such as acetoxymethyl and pivaloyloxymethyl groups.

A suitable pharmaceutically-acceptable pro-drug of a compound of the invention that possesses a carboxy group is, for example, an in-vivo-cleavable amide thereof, for example an amide formed with an amine such as ammonia, a C₁₋₄ alkylamine such as methylamine, a (C₁₋₄ alkyl) 2amine such as dimethylamine, N-ethyl-N-methylamine or diethylamine, a C₁₋₄ alkoxy-C₂₋₄ alkylamine such as 2-methoxyethylamine, a phenyl-C₁₋₄ alkylamine such as benzylamine and amino acids such as glycine or an ester thereof.

A suitable pharmaceutically-acceptable pro-drug of a compound of the invention that possesses an amino group is, for example, an in-vivo-cleavable amide or carbamate derivative thereof. Suitable pharmaceutically-acceptable amides from an amino group include, for example an amide formed with C₁₋₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and

4-(C₁₋₄ alkyl) piperazin-1-ylmethyl. Suitable pharmaceutically-acceptable carbamates from an amino group include, for example acyloxyalkoxycarbonyl and benzyloxycarbonyl groups.

### Compounds

In some embodiments there is provided the compound of formula (I) or a pharmaceutically acceptable salt thereof:

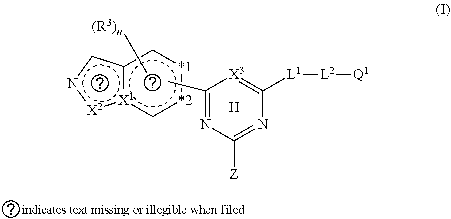

wherein the H ring in formula (I) is bonded to the carbon atom *1 or *2.
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from: C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{X1}R^{X2}; wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

In some embodiments there is provided the compound of formula (I) or a pharmaceutically acceptable salt thereof:
wherein the H ring in formula (I) is bonded to the carbon atom *1 or *2.
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from: C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{x1}R^{X2};wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

In some embodiments there is provided the compound of formula (I) or a pharmaceutically acceptable salt thereof:
wherein the H ring in formula (I) is bonded to the carbon atom *1 or *2.
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{X1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from: C₃₋₁₂ cycloalkyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{x1}R^{X2}; wherein R^{X1} and R^{x2} are independently selected from: H, C₁₋₄ alkyl optionally substituted by OH or 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

In some embodiments there is provided the compound of formula (II) or a pharmaceutically acceptable salt thereof:

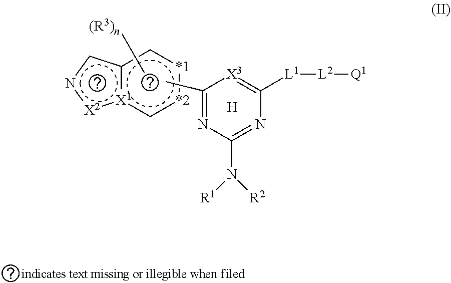

wherein the H ring in formula (II) is bonded to the carbon atom *1 or *2.
R¹ and R² is independently selected from: H, C₁₋₆ alkyl, C₂₋₆ alkenyl, C₂₋₆ alkynyl, and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-;
p is an integer from 1 to 4;
R⁷ and R⁸ are each independently selected from: H, C₁₋₄ alkyl, C₆₋₁₀ aryl, COO—C₁₋₆ alkyl, NR^{X1}R^{X2}, C(O)NR^{x1}R^{X3} and 5- to 10-membered heteroaryl;
wherein at least one of R⁷ and R⁸ in L² is not H, and
said C₁₋₄ alkyl is substituted by 3- to 6-membered cycloalkyl, C₄₋₈ alkyl, or NR^{X1}R^{X2};
Q¹ is selected from: C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl, C₂₋₆ alkynyl and C₁₋₆ haloalkyl is optionally substituted by one or more R¹¹, and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, NR^{X1}R^{X2} and C(O)NR^{X1}R^{X2}, wherein R^{X1} and R^{X2} are independently selected from: H, C₁₋₄ alkyl, optionally substituted by OH or 3- to 6-membered heterocyclyl, OH, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
wherein R^{X3} is selected from: OH and O—C₁₋₆ alkyl;
n is an integer from 0 to 4; and
x is integer from 0 to 3.

In some embodiments there is provided the compound of formula (II) or a pharmaceutically acceptable salt thereof:
wherein the H ring in formula (II) is bonded to the carbon atom *1 or *2.
R¹ and R² is independently selected from: H, C₁₋₆ alkyl, and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-;
wherein p is an integer from 1 to 4;
R⁷ and R⁸ are each independently selected from: H, C₁₋₄ alkyl, C₆₋₁₀ aryl, COO—C₁₋₆ alkyl, NR^{X1}R^{X2}, C(O)NR^{x1}R^{X3} and 5- to 10-membered heteroaryl;
wherein at least one of R⁷ and R⁸ in L² is not H; and
said C₁₋₄ alkyl is substituted by 3- to 6-membered cycloalkyl, C₄₋₈ alkyl, or NR^{X1}R^{X2};
Q¹ is selected from: C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl, C₂₋₆ alkynyl and C₁₋₆ haloalkyl is optionally substituted by one or more R¹¹, and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, NR^{X1}R^{X2} and C(O)NR^{x1}R^{X2}; wherein R^{X1} and R^{X2} are independently selected from: H, C₁₋₄ alkyl, optionally substituted by OH or 3- to 6-membered heterocyclyl, OH, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
wherein R^{X3} is selected from: OH and O—C₁₋₆ alkyl;
n is an integer from 0 to 4; and
x is integer from 0 to 3.

In some embodiments there is provided the compound of formula (II) or a pharmaceutically acceptable salt thereof:
wherein the H ring in formula (II) is bonded to the carbon atom *1 or *2;
R¹ and R² is independently selected from: H, C₁₋₆ alkyl, and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-;
wherein p is an integer from 1 to 4;
R⁷ and R⁸ are each independently selected from: H, C₁₋₄ alkyl, C₆₋₁₀ aryl, COO—C₁₋₆ alkyl, NR^{X1}R^{X2}, C(O)NR^{x1}R^{X3} and 5- to 10-membered heteroaryl;
wherein at least one of R⁷ and R⁸ in L² is not H; and
said C₁₋₄ alkyl is substituted by 3- to 6-membered cycloalkyl, C₄₋₈ alkyl, or NR^{X1}R^{X2};
Q¹ is selected from: C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R° is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl, C₂₋₆ alkynyl and C₁₋₆ haloalkyl is optionally substituted by one or more R¹¹, and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, NR^{X1}R^{X2} and C(O)NR^{x1}R^{X2}; wherein R^{X1} and R^{X2} are independently selected from: H, C₁₋₄ alkyl, optionally substituted by OH or 3- to 6-membered heterocyclyl, OH, —OC₁₋₆ alkyl and —C(O)—C₁-6 alkyl;
wherein R^{X3} is selected from: OH and O—C₁₋₆ alkyl;
n is an integer from 0 to 4; and
x is integer from 0 to 3.

In some preferred embodiments, the compound of formula (II) may not comprise

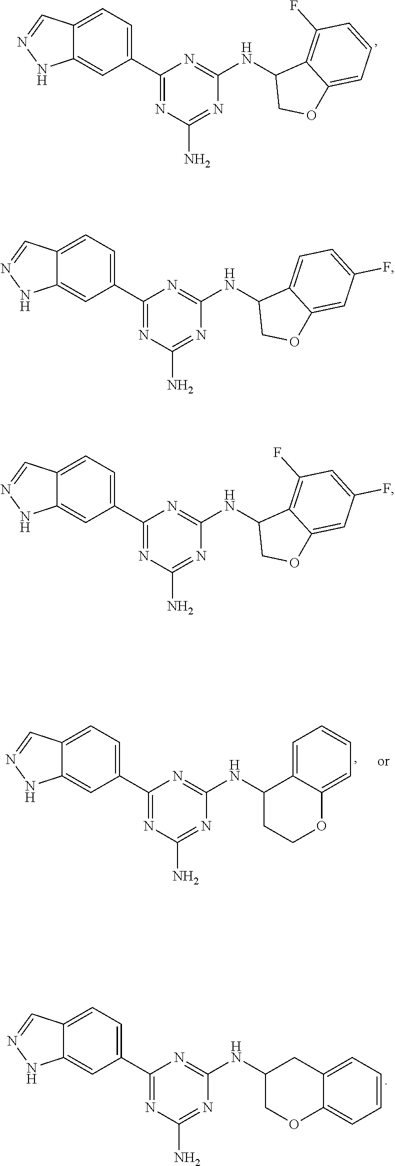 [SMILES: Nc1nc(NC2COc3cccc(F)c32)nc(-c2ccc3cn[nH]c3c2)n1 ; Nc1nc(NC2COc3cc(F)ccc32)nc(-c2ccc3cn[nH]c3c2)n1 ; Nc1nc(NC2COc3cc(F)cc(F)c32)nc(-c2ccc3cn[nH]c3c2)n1 ; Nc1nc(NC2CCOc3ccccc32)nc(-c2ccc3cn[nH]c3c2)n1 ; Nc1nc(NC2COc3ccccc3C2)nc(-c2ccc3cn[nH]c3c2)n1]

In some embodiments there is provided the compound of formula (III), or a pharmaceutically acceptable salt thereof:

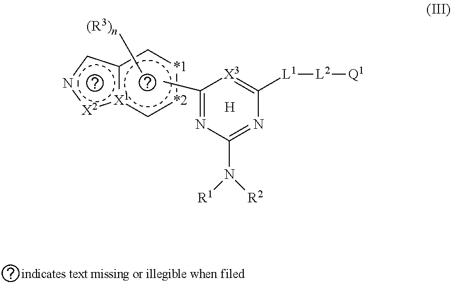

wherein the H ring in formula (III) is bonded to the carbon atom *1 or *2;
R¹ and R² is independently selected from: H, C₁₋₆ alkyl, C₂₋₆ alkenyl, C₂₋₆ alkynyl and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₁₋₄ alkyl is optionally partially or fully deuterated;
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from C₆₋₁₀ aryl and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, C₃-C₆ cycloalkyl, 5- to 10-membered heterocyclyl, 5- to 10-membered heteroaryl, —OR¹⁰, —S(O),R¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl, C₂₋₆ alkynyl, C₃-C₆ cycloalkyl, 5- to 10-membered heterocyclyl, and 5- to 10-membered heteroaryl is optionally substituted by one or more R¹¹,
wherein said C₁₋₆ alkyl is optionally partially or fully deuterated;
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl; and
n is an integer from 0 to 4; and
x is an integer from 0 to 3;
wherein when Q¹ is not substituted by one or more R⁹, or
when any one of one or more R⁹ on Q¹ is not C₃-C₆ cycloalkyl, 5- to 10-membered heterocyclyl, 5- to 10-membered heteroaryl, or C₁₋₆ alkyl which is partially or fully deuterated, then
R⁶ is C₁-4 alkyl which is partially or fully deuterated.

In some embodiments there is provided the compound of formula (III), or a pharmaceutically acceptable salt thereof:
wherein the H ring in formula (III) is bonded to the carbon atom *1 or *2;
R¹ and R² is independently selected from: H, C₁₋₆ alkyl, C₂₋₆ alkenyl, C₂₋₆ alkynyl and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or NR⁶;
R⁶ is selected from H, C₁₋₄ alkyl, and C₁₋₄ haloalkyl,
wherein said C₁₋₄ alkyl is optionally partially or fully deuterated;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₆₋₁₀ aryl and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, C₃-C₆ cycloalkyl, 5- to 10-membered heterocyclyl, 5- to 10-membered heteroaryl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl, C₂₋₆ alkynyl, C₃-C₆ cycloalkyl, 5- to 10-membered heterocyclyl, and 5- to 10-membered heteroaryl is optionally substituted by one or more R¹¹,
wherein said C₁₋₆ alkyl is optionally partially or fully deuterated;
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{x1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl; and
n is an integer from 0 to 4; and
x is an integer from 0 to 3;
wherein when Q¹ is not substituted by one or more R⁹, or
when any one of one or more R⁹ on Q¹ is not C₃-C₆ cycloalkyl, 5- to 10-membered heterocyclyl, 5- to 10-membered heteroaryl, or C₁₋₆ alkyl which is partially or fully deuterated, then
R⁶ is C₁-4 alkyl which is partially or fully deuterated.

In some embodiments there is provided the compound of formula (III), or a pharmaceutically acceptable salt thereof:
wherein the H ring in formula (III) is bonded to the carbon atom *1 or *2;
R¹ and R² is independently selected from: H, C₁₋₆ alkyl, C₂₋₆ alkenyl, C₂₋₆ alkynyl and C₁-6 haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴ and R⁵ are independently selected from: H, halo, CN, C₁₋₆ alkyl and C₁₋₆ haloalkyl;
L¹ is a bond or NR⁶;
R⁶ is selected from H, C₁₋₄ alkyl, and C₁₋₄ haloalkyl,
wherein said C₁₋₄ alkyl is optionally partially or fully deuterated;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₆₋₁₀ aryl and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, C₃-C₆ cycloalkyl, 5- to 10-membered heterocyclyl, 5- to 10-membered heteroaryl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl, C₂₋₆ alkynyl, C₃-C₆ cycloalkyl, 5- to 10-membered heterocyclyl, and 5- to 10-membered heteroaryl is optionally substituted by one or more R¹¹,
wherein said C₁₋₆ alkyl is optionally partially or fully deuterated;
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl; and
n is an integer from 0 to 4; and
x is an integer from 0 to 3;
wherein when Q¹ is not substituted by one or more R⁹, or
when any one of one or more R⁹ on Q¹ is not C₃-C₆ cycloalkyl, 5- to 10-membered heterocyclyl, 5- to 10-membered heteroaryl, or C₁₋₆ alkyl which is partially or fully deuterated, then
R⁶ is C₁-4 alkyl which is partially or fully deuterated.

In some embodiments there is provided the compound of formula (IV), or a pharmaceutically acceptable salt thereof:

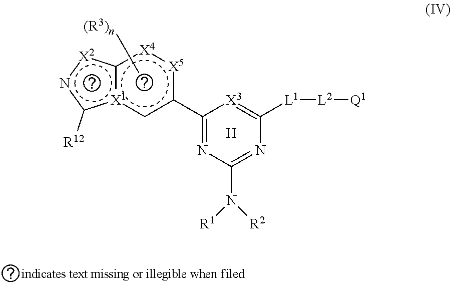

wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or is selected from: NR⁶, O, and S,
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{X1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁.

6 haloalkyl;
Q¹ is selected from C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3.

In some embodiments there is provided the compound of formula (IV), or a pharmaceutically acceptable salt thereof:
wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or NR⁶,
R⁶ is selected from H, C₁₋₄ alkyl, and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl and C₂₋₆ alkynyl is optionally substituted by one or more R¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3.

In some embodiments there is provided the compound of formula (IV), or a pharmaceutically acceptable salt thereof:
wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or NR⁶,
R⁶ is selected from H, C₁₋₄ alkyl, and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₃₋₁₂ cycloalkyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2, wherein said C₁₋₆ alkyl, C₂₋₆ alkenyl and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl;
n is an integer from 0 to 4; and
x is an integer from 0 to 3.

In some preferred embodiments, the compound of formula (IV) may not comprise

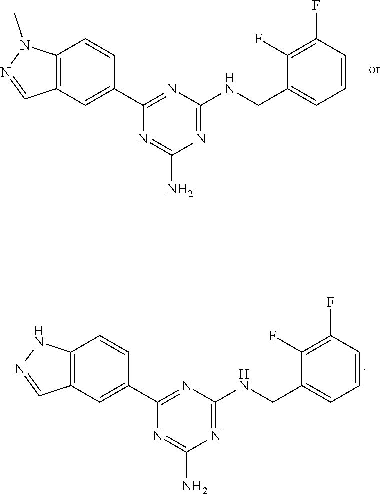 [SMILES: Cn1ncc2cc(-c3nc(N)nc(NCc4cccc(F)c4F)n3)ccc21 ; Nc1nc(NCc2cccc(F)c2F)nc(-c2ccc3[nH]ncc3c2)n1]

In some preferred embodiments, the group

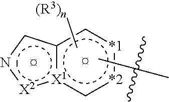

in the compounds of formulas (¥⁰), (¥¹) or (¥²) may be selected from any of the following structures:

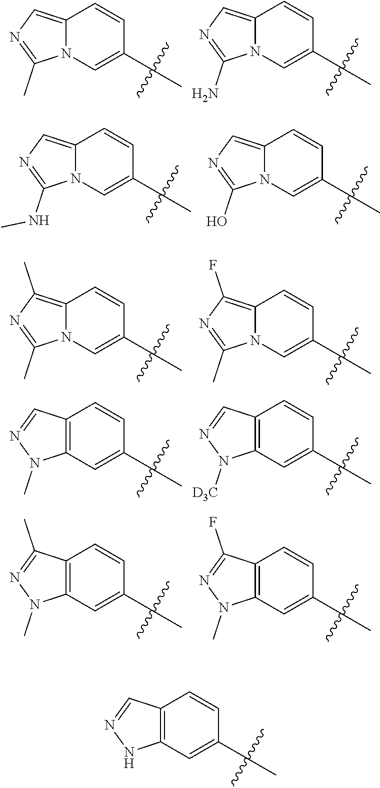

In some preferred embodiments, the group

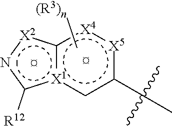

in the compounds of formula (¥³) may be selected from any of the following structures:

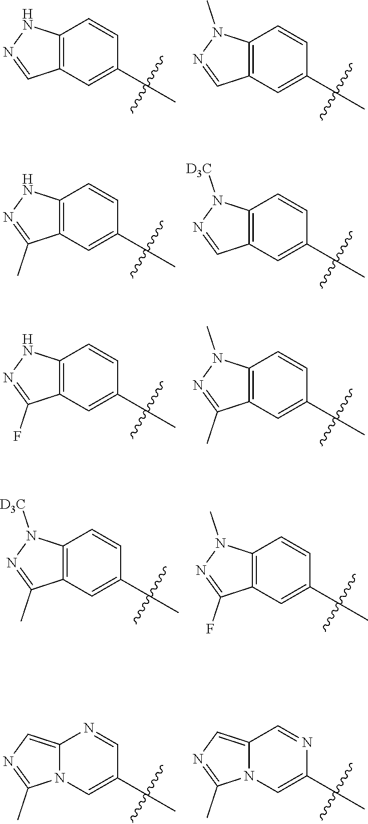

In some preferred embodiments, the group

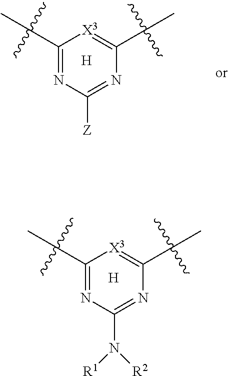

in the compounds of formulas (¥⁰), (¥¹), (¥²) or (¥³) may be selected from any of the following structures:

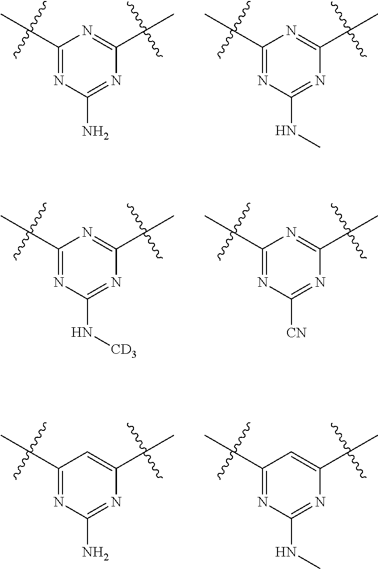

In some preferred embodiments, the group-L¹-L²-Q¹ in the compounds of formulas (¥⁰), (¥¹), (¥²) or (¥³) may be selected from any of the following structures:

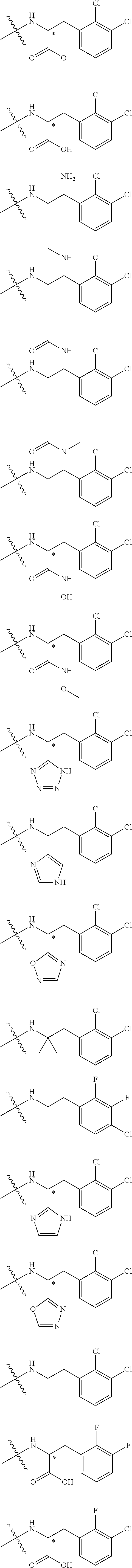

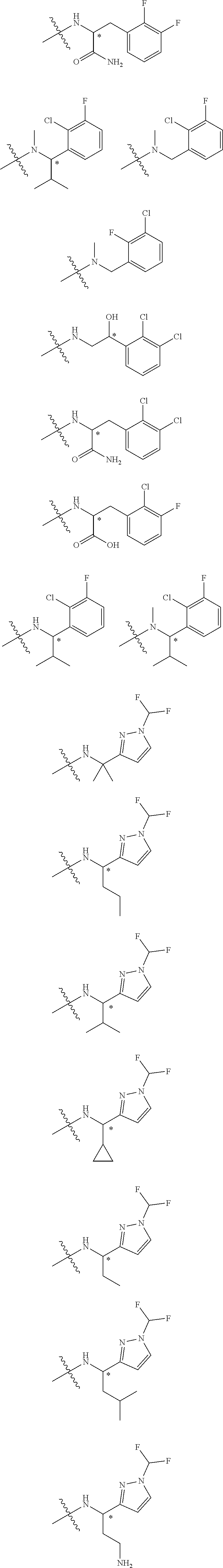

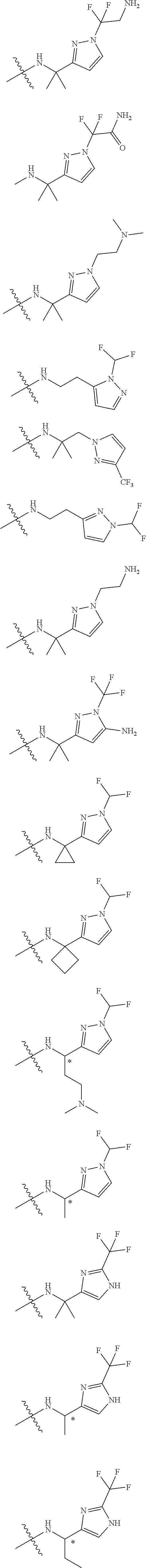

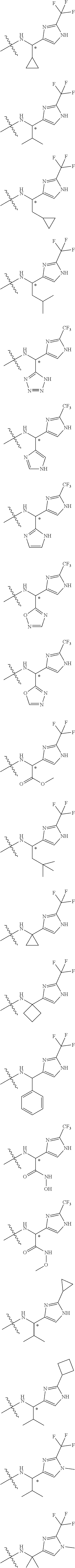

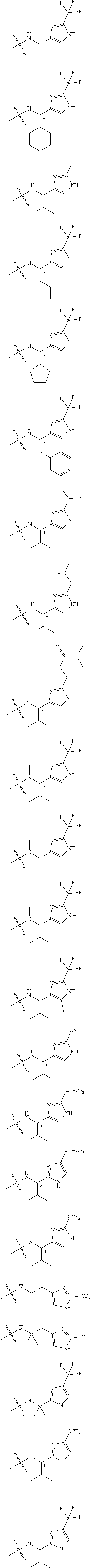

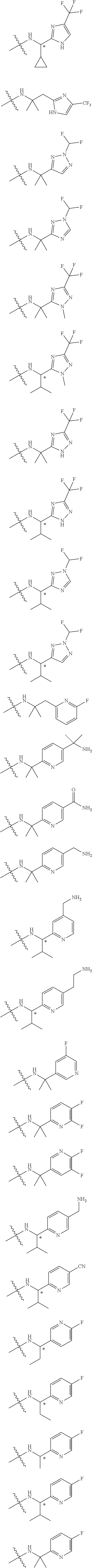

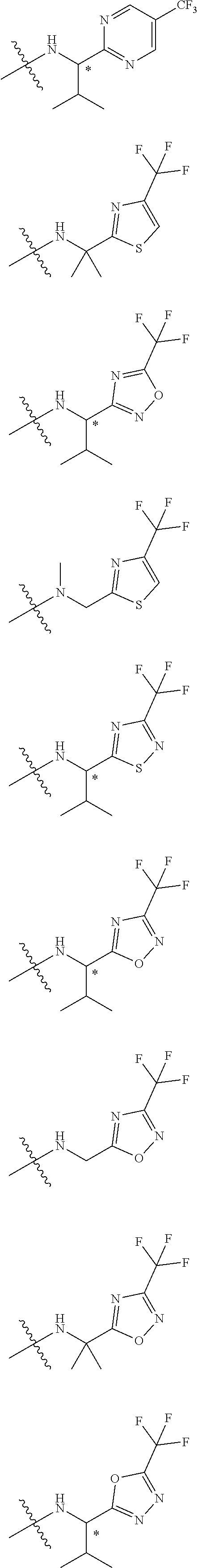

In another embodiments there is provided a compound selected from any one of Compound Nos. 1-239 listed in Table 1 herein, or a pharmaceutically acceptable salt, or prodrug thereof.

TABLE 1

| No. | Structure |
|---|---|
| 1 | 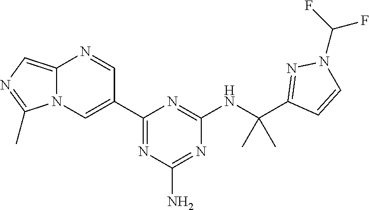 [SMILES: Cc1ncc2ncc(-c3nc(N)nc(NC(C)(C)c4ccn(C(F)F)n4)n3)cn12] |
| 2 | 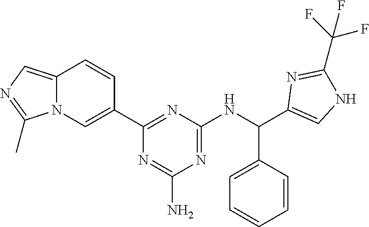 [SMILES: Cc1ncc2ccc(-c3nc(N)nc(NC(c4ccccc4)c4c[nH]c(C(F)(F)F)n4)n3)cn12] |
| 3 | 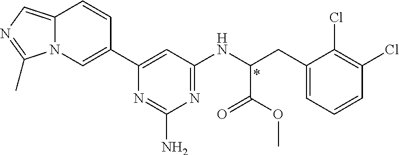 [SMILES: COC(=O)C(Cc1cccc(Cl)c1Cl)Nc1cc(-c2ccc3cnc(C)n3c2)nc(N)n1] |
| 4 | 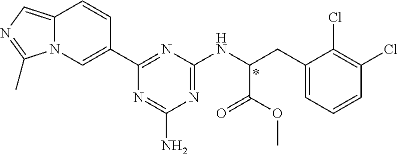 [SMILES: COC(=O)C(Cc1cccc(Cl)c1Cl)Nc1nc(N)nc(-c2ccc3cnc(C)n3c2)n1] |
| 5 | 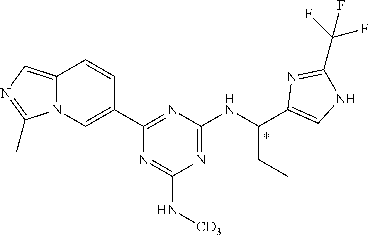 [SMILES: [2H]C([2H])([2H])Nc1nc(NC(CC)c2c[nH]c(C(F)(F)F)n2)nc(-c2ccc3cnc(C)n3c2)n1] |
| 6 | 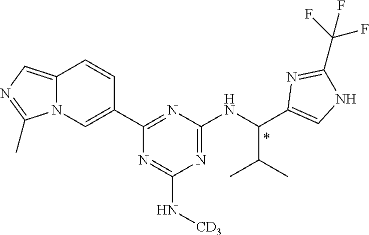 [SMILES: [2H]C([2H])([2H])Nc1nc(NC(c2c[nH]c(C(F)(F)F)n2)C(C)C)nc(-c2ccc3cnc(C)n3c2)n1] |
| 7 | 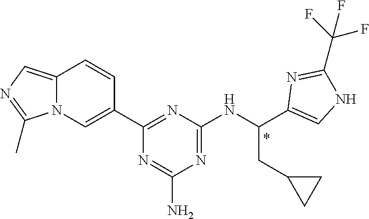 [SMILES: Cc1ncc2ccc(-c3nc(N)nc(NC(CC4CC4)c4c[nH]c(C(F)(F)F)n4)n3)cn12] |
| 8 | 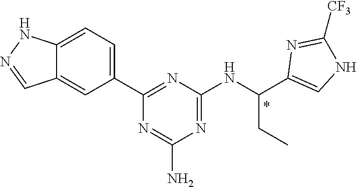 [SMILES: CCC(Nc1nc(N)nc(-c2ccc3[nH]ncc3c2)n1)c1c[nH]c(C(F)(F)F)n1] |
| 9 | 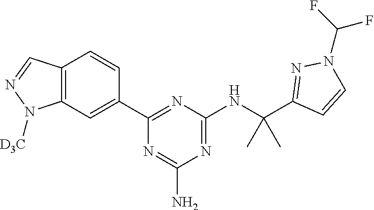 [SMILES: [2H]C([2H])([2H])n1ncc2ccc(-c3nc(N)nc(NC(C)(C)c4ccn(C(F)F)n4)n3)cc21] |
| 10 | 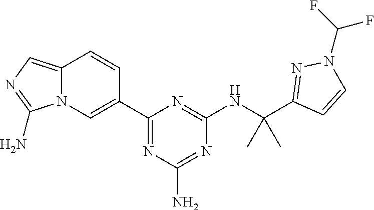 [SMILES: CC(C)(Nc1nc(N)nc(-c2ccc3cnc(N)n3c2)n1)c1ccn(C(F)F)n1] |
| 11 | 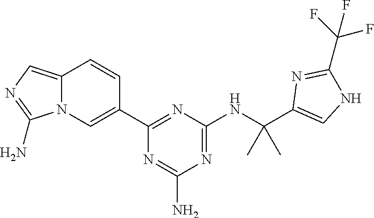 [SMILES: CC(C)(Nc1nc(N)nc(-c2ccc3cnc(N)n3c2)n1)c1c[nH]c(C(F)(F)F)n1] |
| 12 | 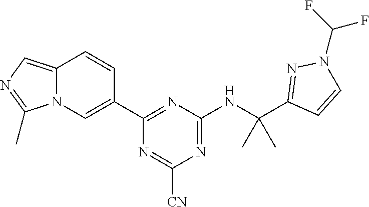 [SMILES: Cc1ncc2ccc(-c3nc(C#N)nc(NC(C)(C)c4ccn(C(F)F)n4)n3)cn12] |
| 13 | 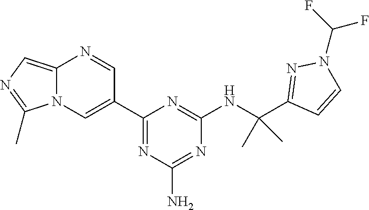 [SMILES: Cc1ncc2ncc(-c3nc(N)nc(NC(C)(C)c4ccn(C(F)F)n4)n3)cn12] |
| 14 | 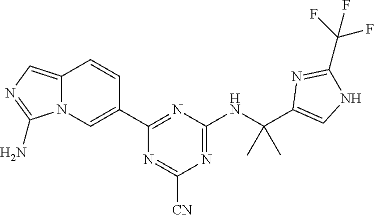 [SMILES: CC(C)(Nc1nc(C#N)nc(-c2ccc3cnc(N)n3c2)n1)c1c[nH]c(C(F)(F)F)n1] |
| 15 | 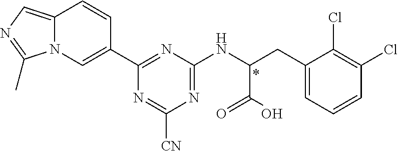 [SMILES: Cc1ncc2ccc(-c3nc(C#N)nc(NC(Cc4cccc(Cl)c4Cl)C(=O)O)n3)cn12] |
| 16 | 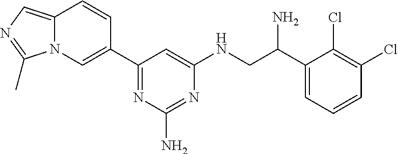 [SMILES: Cc1ncc2ccc(-c3cc(NCC(N)c4cccc(Cl)c4Cl)nc(N)n3)cn12] |
| 17 | 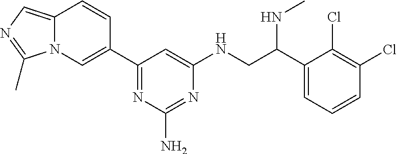 [SMILES: CNC(CNc1cc(-c2ccc3cnc(C)n3c2)nc(N)n1)c1cccc(Cl)c1Cl] |
| 18 | 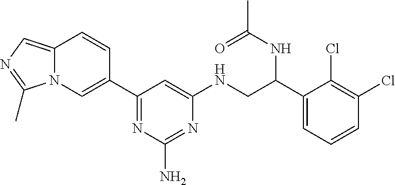 [SMILES: CC(=O)NC(CNc1cc(-c2ccc3cnc(C)n3c2)nc(N)n1)c1cccc(Cl)c1Cl] |
| 19 | 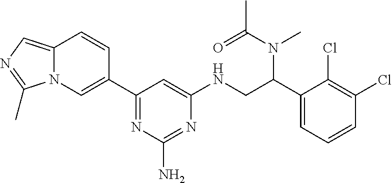 [SMILES: CC(=O)N(C)C(CNc1cc(-c2ccc3cnc(C)n3c2)nc(N)n1)c1cccc(Cl)c1Cl] |
| 20 | 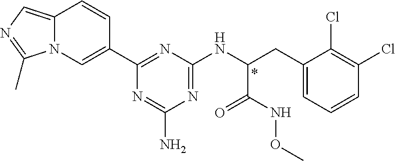 [SMILES: CONC(=O)C(Cc1cccc(Cl)c1Cl)Nc1nc(N)nc(-c2ccc3cnc(C)n3c2)n1] |
| 21 | 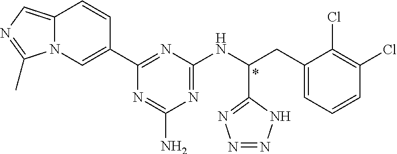 [SMILES: Cc1ncc2ccc(-c3nc(N)nc(NC(Cc4cccc(Cl)c4Cl)c4nnn[nH]4)n3)cn12] |
| 22 | 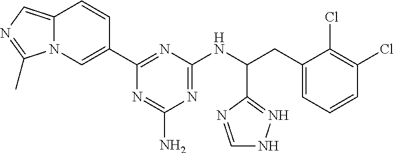 [SMILES: Cc1ncc2ccc(-c3nc(N)nc(NC(Cc4cccc(Cl)c4Cl)c4nc[nH]n4)n3)cn12] |
| 23 | 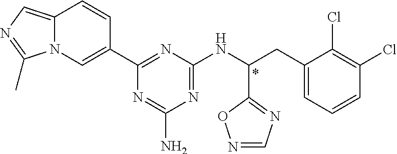 [SMILES: Cc1ncc2ccc(-c3nc(N)nc(NC(Cc4cccc(Cl)c4Cl)c4ncno4)n3)cn12] |
| 24 | 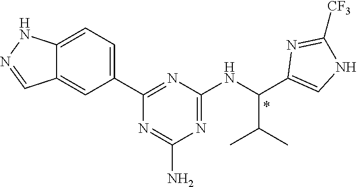 [SMILES: CC(C)C(Nc1nc(N)nc(-c2ccc3[nH]ncc3c2)n1)c1c[nH]c(C(F)(F)F)n1] |
| 25 | 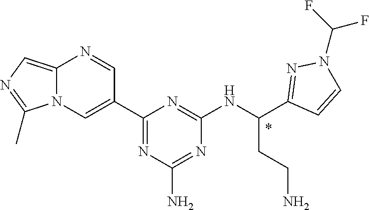 [SMILES: Cc1ncc2ncc(-c3nc(N)nc(NC(CCN)c4ccn(C(F)F)n4)n3)cn12] |
| 26 | 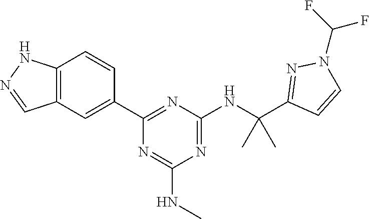 [SMILES: CNc1nc(NC(C)(C)c2ccn(C(F)F)n2)nc(-c2ccc3[nH]ncc3c2)n1] |
| 27 | 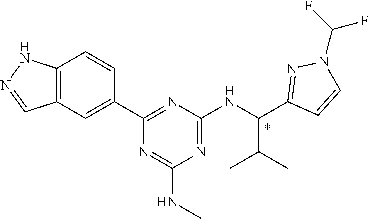 [SMILES: CNc1nc(NC(c2ccn(C(F)F)n2)C(C)C)nc(-c2ccc3[nH]ncc3c2)n1] |
| 28 | 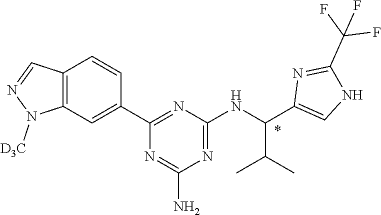 [SMILES: [2H]C([2H])([2H])n1ncc2ccc(-c3nc(N)nc(NC(c4c[nH]c(C(F)(F)F)n4)C(C)C)n3)cc21] |
| 29 | 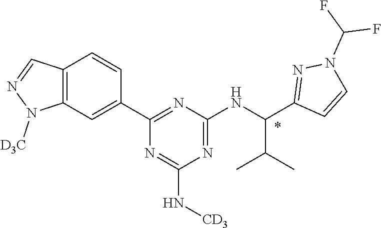 [SMILES: [2H]C([2H])([2H])Nc1nc(NC(c2ccn(C(F)F)n2)C(C)C)nc(-c2ccc3cnn(C([2H])([2H])[2H])c3c2)n1] |
| 30 | 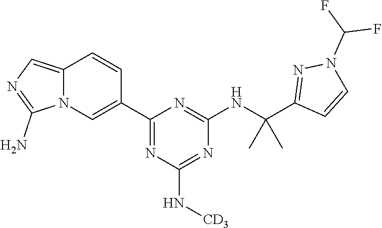 [SMILES: [2H]C([2H])([2H])Nc1nc(NC(C)(C)c2ccn(C(F)F)n2)nc(-c2ccc3cnc(N)n3c2)n1] |
| 31 | 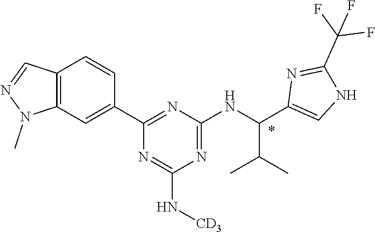 [SMILES: [2H]C([2H])([2H])Nc1nc(NC(c2c[nH]c(C(F)(F)F)n2)C(C)C)nc(-c2ccc3cnn(C)c3c2)n1] |
| 32 | 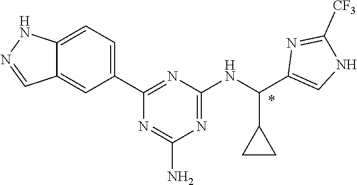 [SMILES: Nc1nc(NC(c2c[nH]c(C(F)(F)F)n2)C2CC2)nc(-c2ccc3[nH]ncc3c2)n1] |
| 33 | 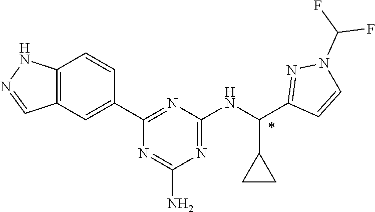 [SMILES: Nc1nc(NC(c2ccn(C(F)F)n2)C2CC2)nc(-c2ccc3[nH]ncc3c2)n1] |
| 34 | 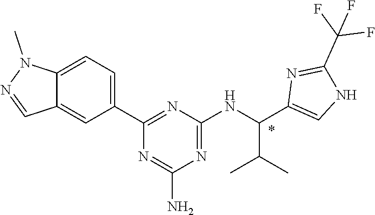 [SMILES: CC(C)C(Nc1nc(N)nc(-c2ccc3c(cnn3C)c2)n1)c1c[nH]c(C(F)(F)F)n1] |
| 35 | 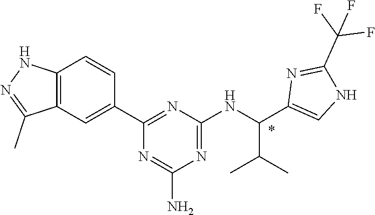 [SMILES: Cc1n[nH]c2ccc(-c3nc(N)nc(NC(c4c[nH]c(C(F)(F)F)n4)C(C)C)n3)cc12] |
| 36 | 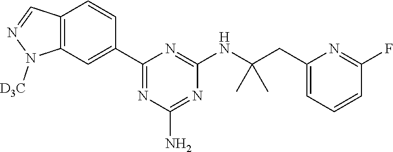 [SMILES: [2H]C([2H])([2H])n1ncc2ccc(-c3nc(N)nc(NC(C)(C)Cc4cccc(F)n4)n3)cc21] |
| 37 | 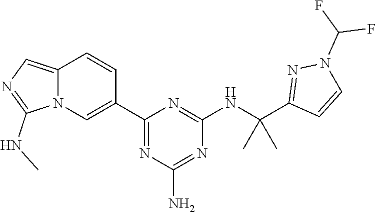 [SMILES: CNc1ncc2ccc(-c3nc(N)nc(NC(C)(C)c4ccn(C(F)F)n4)n3)cn12] |
| 38 | 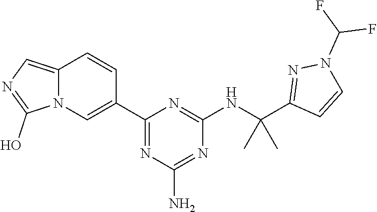 [SMILES: CC(C)(Nc1nc(N)nc(-c2ccc3cnc(O)n3c2)n1)c1ccn(C(F)F)n1] |
| 39 | 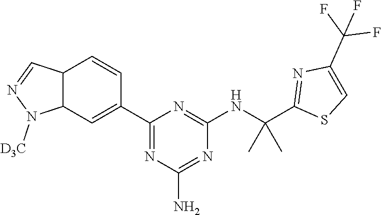 [SMILES: [2H]C([2H])([2H])N1N=CC2C=CC(c3nc(N)nc(NC(C)(C)c4nc(C(F)(F)F)cs4)n3)=CC21] |
| 40 | 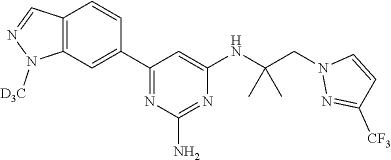 [SMILES: [2H]C([2H])([2H])n1ncc2ccc(-c3cc(NC(C)(C)Cn4ccc(C(F)(F)F)n4)nc(N)n3)cc21] |
| 41 | 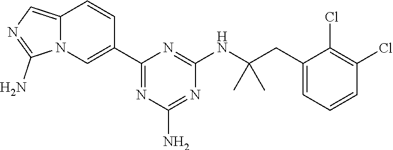 [SMILES: CC(C)(Cc1cccc(Cl)c1Cl)Nc1nc(N)nc(-c2ccc3cnc(N)n3c2)n1] |
| 42 | 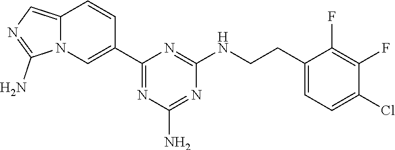 [SMILES: Nc1nc(NCCc2ccc(Cl)c(F)c2F)nc(-c2ccc3cnc(N)n3c2)n1] |
| 43 | 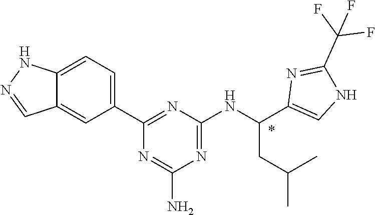 [SMILES: CC(C)CC(Nc1nc(N)nc(-c2ccc3[nH]ncc3c2)n1)c1c[nH]c(C(F)(F)F)n1] |
| 44 | 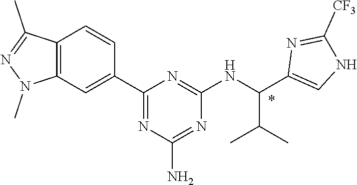 [SMILES: Cc1nn(C)c2cc(-c3nc(N)nc(NC(c4c[nH]c(C(F)(F)F)n4)C(C)C)n3)ccc12] |
| 45 | 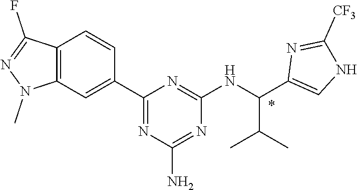 [SMILES: CC(C)C(Nc1nc(N)nc(-c2ccc3c(F)nn(C)c3c2)n1)c1c[nH]c(C(F)(F)F)n1] |
| 46 | 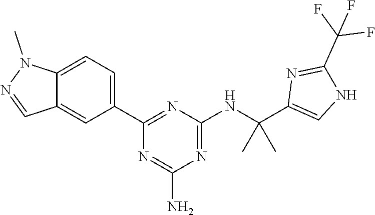 [SMILES: Cn1ncc2cc(-c3nc(N)nc(NC(C)(C)c4c[nH]c(C(F)(F)F)n4)n3)ccc21] |
| 47 | 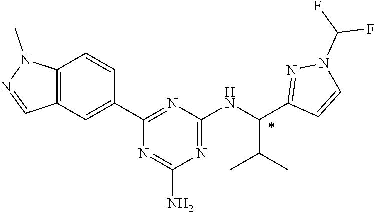 [SMILES: CC(C)C(Nc1nc(N)nc(-c2ccc3c(cnn3C)c2)n1)c1ccn(C(F)F)n1] |
| 48 | 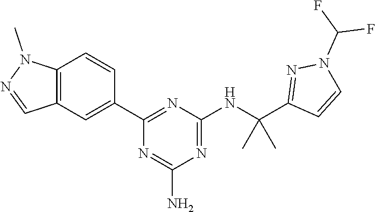 [SMILES: Cn1ncc2cc(-c3nc(N)nc(NC(C)(C)c4ccn(C(F)F)n4)n3)ccc21] |
| 49 | 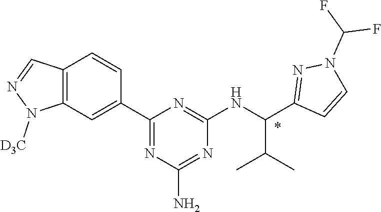 [SMILES: [2H]C([2H])([2H])n1ncc2ccc(-c3nc(N)nc(NC(c4ccn(C(F)F)n4)C(C)C)n3)cc21] |
| 50 | 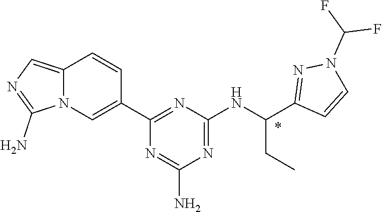 [SMILES: CCC(Nc1nc(N)nc(-c2ccc3cnc(N)n3c2)n1)c1ccn(C(F)F)n1] |
| 51 | 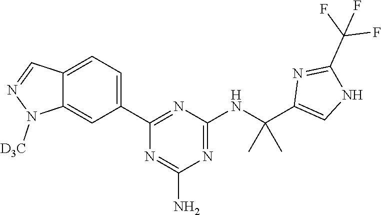 [SMILES: [2H]C([2H])([2H])n1ncc2ccc(-c3nc(N)nc(NC(C)(C)c4c[nH]c(C(F)(F)F)n4)n3)cc21] |
| 52 | 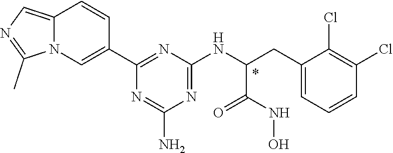 [SMILES: Cc1ncc2ccc(-c3nc(N)nc(NC(Cc4cccc(Cl)c4Cl)C(=O)NO)n3)cn12] |
| 53 | 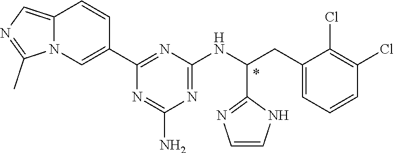 [SMILES: Cc1ncc2ccc(-c3nc(N)nc(NC(Cc4cccc(Cl)c4Cl)c4ncc[nH]4)n3)cn12] |
| 54 | 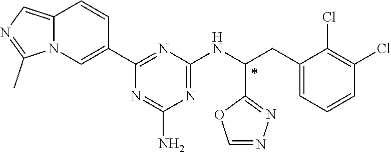 [SMILES: Cc1ncc2ccc(-c3nc(N)nc(NC(Cc4cccc(Cl)c4Cl)c4nnco4)n3)cn12] |
| 55 | 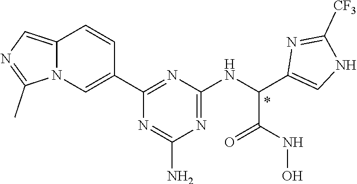 [SMILES: Cc1ncc2ccc(-c3nc(N)nc(NC(C(=O)NO)c4c[nH]c(C(F)(F)F)n4)n3)cn12] |
| 56 | 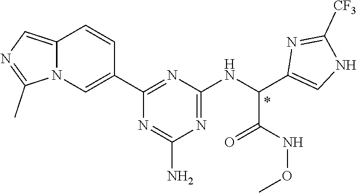 [SMILES: CONC(=O)C(Nc1nc(N)nc(-c2ccc3cnc(C)n3c2)n1)c1c[nH]c(C(F)(F)F)n1] |
| 57 | 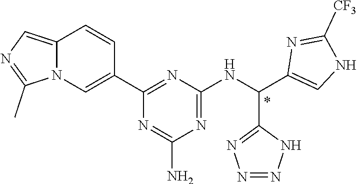 [SMILES: Cc1ncc2ccc(-c3nc(N)nc(NC(c4c[nH]c(C(F)(F)F)n4)c4nnn[nH]4)n3)cn12] |
| 58 | 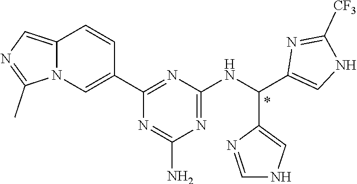 [SMILES: Cc1ncc2ccc(-c3nc(N)nc(NC(c4c[nH]cn4)c4c[nH]c(C(F)(F)F)n4)n3)cn12] |
| 59 | 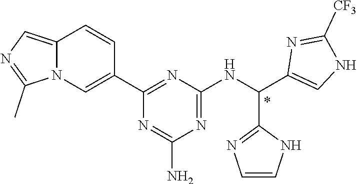 [SMILES: Cc1ncc2ccc(-c3nc(N)nc(NC(c4c[nH]c(C(F)(F)F)n4)c4ncc[nH]4)n3)cn12] |
| 60 | 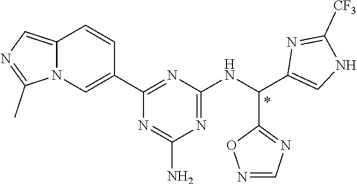 [SMILES: Cc1ncc2ccc(-c3nc(N)nc(NC(c4c[nH]c(C(F)(F)F)n4)c4ncno4)n3)cn12] |
| 61 | 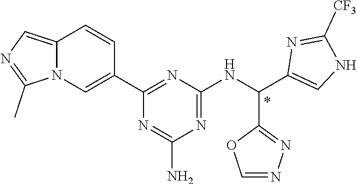 [SMILES: Cc1ncc2ccc(-c3nc(N)nc(NC(c4c[nH]c(C(F)(F)F)n4)c4nnco4)n3)cn12] |
| 62 | 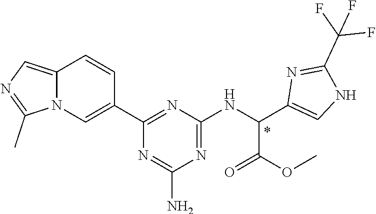 [SMILES: COC(=O)C(Nc1nc(N)nc(-c2ccc3cnc(C)n3c2)n1)c1c[nH]c(C(F)(F)F)n1] |
| 63 | 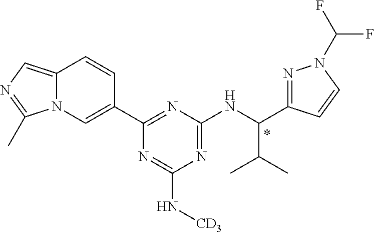 [SMILES: [2H]C([2H])([2H])Nc1nc(NC(c2ccn(C(F)F)n2)C(C)C)nc(-c2ccc3cnc(C)n3c2)n1] |
| 64 | 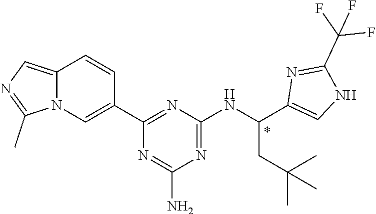 [SMILES: Cc1ncc2ccc(-c3nc(N)nc(NC(CC(C)(C)C)c4c[nH]c(C(F)(F)F)n4)n3)cn12] |
| 65 | 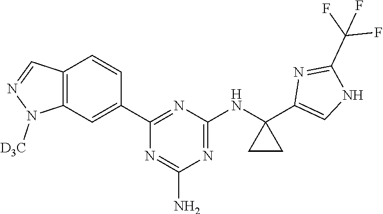 [SMILES: [2H]C([2H])([2H])n1ncc2ccc(-c3nc(N)nc(NC4(c5c[nH]c(C(F)(F)F)n5)CC4)n3)cc21] |
| 66 | 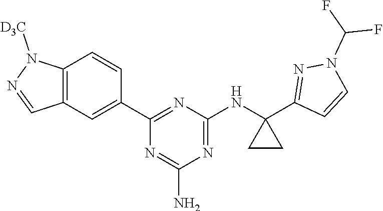 [SMILES: [2H]C([2H])([2H])n1ncc2cc(-c3nc(N)nc(NC4(c5ccn(C(F)F)n5)CC4)n3)ccc21] |
| 67 | 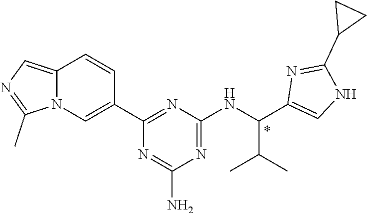 [SMILES: Cc1ncc2ccc(-c3nc(N)nc(NC(c4c[nH]c(C5CC5)n4)C(C)C)n3)cn12] |
| 68 | 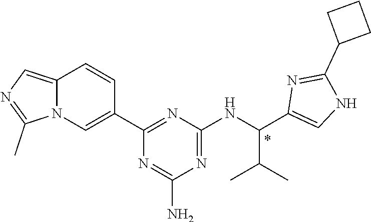 [SMILES: Cc1ncc2ccc(-c3nc(N)nc(NC(c4c[nH]c(C5CCC5)n4)C(C)C)n3)cn12] |
| 69 | 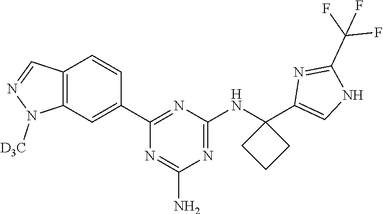 [SMILES: [2H]C([2H])([2H])n1ncc2ccc(-c3nc(N)nc(NC4(c5c[nH]c(C(F)(F)F)n5)CCC4)n3)cc21] |
| 70 | 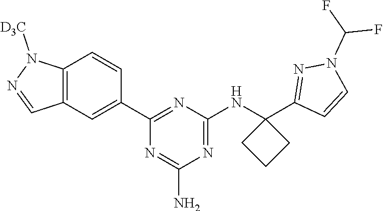 [SMILES: [2H]C([2H])([2H])n1ncc2cc(-c3nc(N)nc(NC4(c5ccn(C(F)F)n5)CCC4)n3)ccc21] |
| 71 | 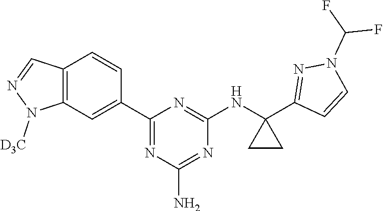 [SMILES: [2H]C([2H])([2H])n1ncc2ccc(-c3nc(N)nc(NC4(c5ccn(C(F)F)n5)CC4)n3)cc21] |
| 72 | 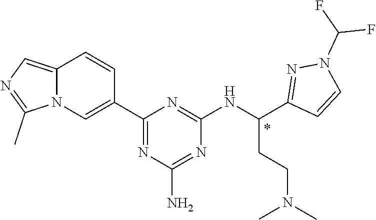 [SMILES: Cc1ncc2ccc(-c3nc(N)nc(NC(CCN(C)C)c4ccn(C(F)F)n4)n3)cn12] |
| 73 | 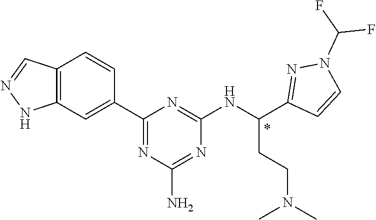 [SMILES: CN(C)CCC(Nc1nc(N)nc(-c2ccc3cn[nH]c3c2)n1)c1ccn(C(F)F)n1] |
| 74 | 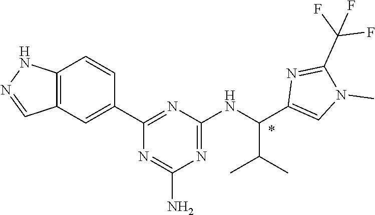 [SMILES: CC(C)C(Nc1nc(N)nc(-c2ccc3[nH]ncc3c2)n1)c1cn(C)c(C(F)(F)F)n1] |
| 75 | 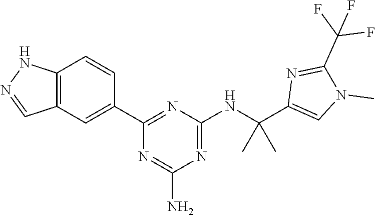 [SMILES: Cn1cc(C(C)(C)Nc2nc(N)nc(-c3ccc4[nH]ncc4c3)n2)nc1C(F)(F)F] |
| 76 | 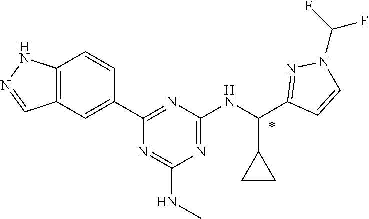 [SMILES: CNc1nc(NC(c2ccn(C(F)F)n2)C2CC2)nc(-c2ccc3[nH]ncc3c2)n1] |
| 77 | 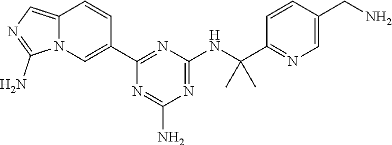 [SMILES: CC(C)(Nc1nc(N)nc(-c2ccc3cnc(N)n3c2)n1)c1ccc(CN)cn1] |
| 78 | 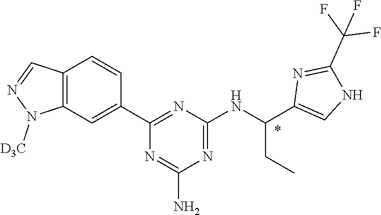 [SMILES: [2H]C([2H])([2H])n1ncc2ccc(-c3nc(N)nc(NC(CC)c4c[nH]c(C(F)(F)F)n4)n3)cc21] |
| 79 | 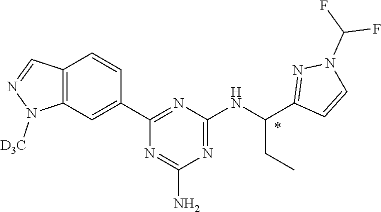 [SMILES: [2H]C([2H])([2H])n1ncc2ccc(-c3nc(N)nc(NC(CC)c4ccn(C(F)F)n4)n3)cc21] |
| 80 | 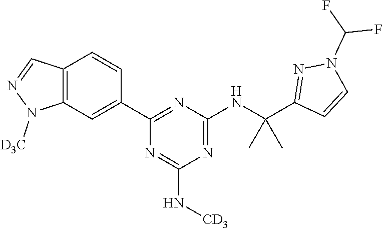 [SMILES: [2H]C([2H])([2H])Nc1nc(NC(C)(C)c2ccn(C(F)F)n2)nc(-c2ccc3cnn(C([2H])([2H])[2H])c3c2)n1] |
| 81 | 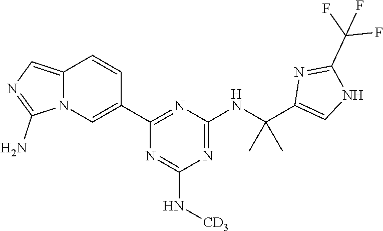 [SMILES: [2H]C([2H])([2H])Nc1nc(NC(C)(C)c2c[nH]c(C(F)(F)F)n2)nc(-c2ccc3cnc(N)n3c2)n1] |
| 82 | 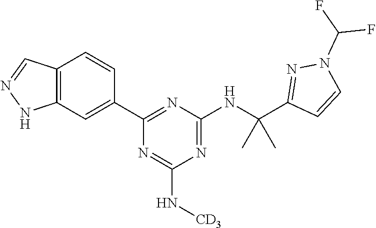 [SMILES: [2H]C([2H])([2H])Nc1nc(NC(C)(C)c2ccn(C(F)F)n2)nc(-c2ccc3cn[nH]c3c2)n1] |
| 83 | 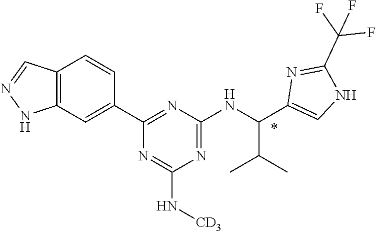 [SMILES: [2H]C([2H])([2H])Nc1nc(NC(c2c[nH]c(C(F)(F)F)n2)C(C)C)nc(-c2ccc3cn[nH]c3c2)n1] |
| 84 | 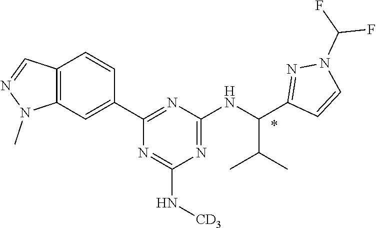 [SMILES: [2H]C([2H])([2H])Nc1nc(NC(c2ccn(C(F)F)n2)C(C)C)nc(-c2ccc3cnn(C)c3c2)n1] |
| 85 | 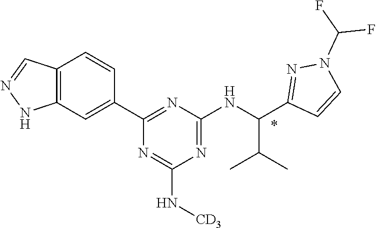 [SMILES: [2H]C([2H])([2H])Nc1nc(NC(c2ccn(C(F)F)n2)C(C)C)nc(-c2ccc3cn[nH]c3c2)n1] |
| 86 | 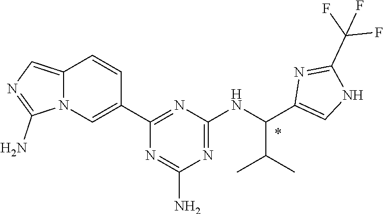 [SMILES: CC(C)C(Nc1nc(N)nc(-c2ccc3cnc(N)n3c2)n1)c1c[nH]c(C(F)(F)F)n1] |
| 87 | 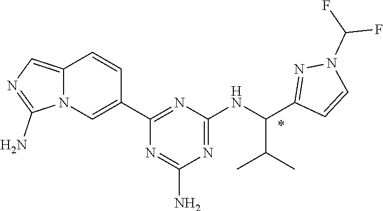 [SMILES: CC(C)C(Nc1nc(N)nc(-c2ccc3cnc(N)n3c2)n1)c1ccn(C(F)F)n1] |
| 88 | 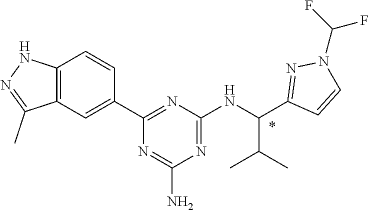 [SMILES: Cc1n[nH]c2ccc(-c3nc(N)nc(NC(c4ccn(C(F)F)n4)C(C)C)n3)cc12] |
| 89 | 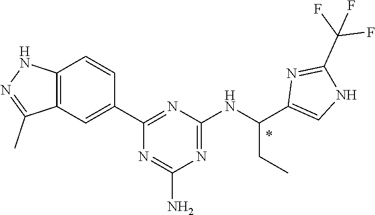 [SMILES: CCC(Nc1nc(N)nc(-c2ccc3[nH]nc(C)c3c2)n1)c1c[nH]c(C(F)(F)F)n1] |
| 90 | 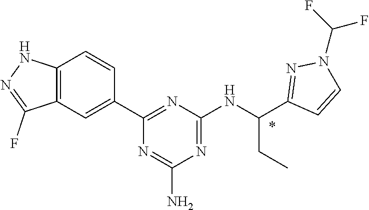 [SMILES: CCC(Nc1nc(N)nc(-c2ccc3[nH]nc(F)c3c2)n1)c1ccn(C(F)F)n1] |
| 91 | 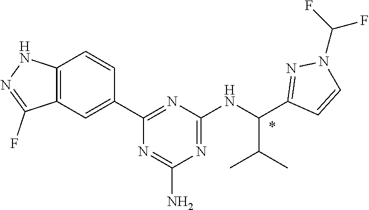 [SMILES: CC(C)C(Nc1nc(N)nc(-c2ccc3[nH]nc(F)c3c2)n1)c1ccn(C(F)F)n1] |
| 92 | 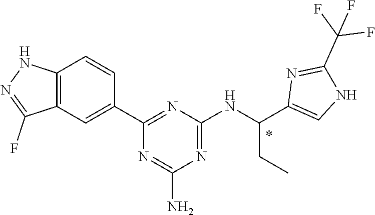 [SMILES: CCC(Nc1nc(N)nc(-c2ccc3[nH]nc(F)c3c2)n1)c1c[nH]c(C(F)(F)F)n1] |
| 93 | 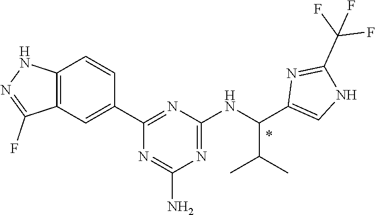 [SMILES: CC(C)C(Nc1nc(N)nc(-c2ccc3[nH]nc(F)c3c2)n1)c1c[nH]c(C(F)(F)F)n1] |
| 94 | 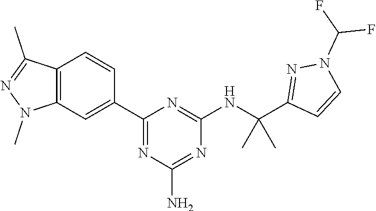 [SMILES: Cc1nn(C)c2cc(-c3nc(N)nc(NC(C)(C)c4ccn(C(F)F)n4)n3)ccc12] |
| 95 | 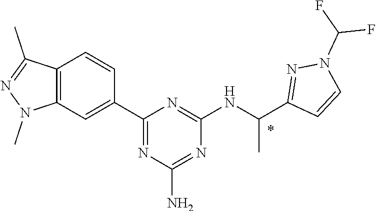 [SMILES: Cc1nn(C)c2cc(-c3nc(N)nc(NC(C)c4ccn(C(F)F)n4)n3)ccc12] |
| 96 | 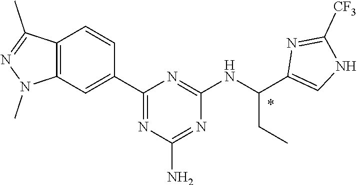 [SMILES: CCC(Nc1nc(N)nc(-c2ccc3c(C)nn(C)c3c2)n1)c1c[nH]c(C(F)(F)F)n1] |
| 97 | 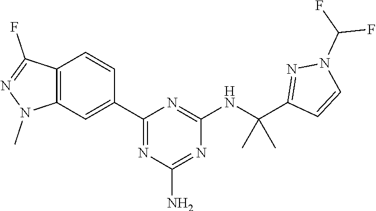 [SMILES: Cn1nc(F)c2ccc(-c3nc(N)nc(NC(C)(C)c4ccn(C(F)F)n4)n3)cc21] |
| 98 | 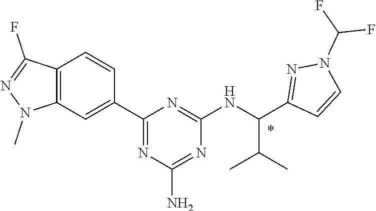 [SMILES: CC(C)C(Nc1nc(N)nc(-c2ccc3c(F)nn(C)c3c2)n1)c1ccn(C(F)F)n1] |
| 99 | 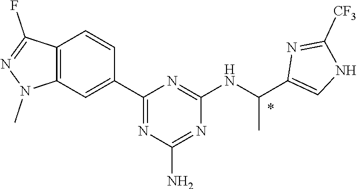 [SMILES: CC(Nc1nc(N)nc(-c2ccc3c(F)nn(C)c3c2)n1)c1c[nH]c(C(F)(F)F)n1] |
| 100 | 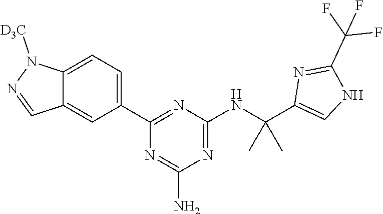 [SMILES: [2H]C([2H])([2H])n1ncc2cc(-c3nc(N)nc(NC(C)(C)c4c[nH]c(C(F)(F)F)n4)n3)ccc21] |
| 101 | 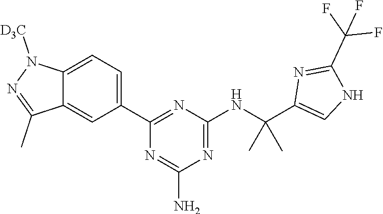 [SMILES: [2H]C([2H])([2H])n1nc(C)c2cc(-c3nc(N)nc(NC(C)(C)c4c[nH]c(C(F)(F)F)n4)n3)ccc21] |
| 102 | 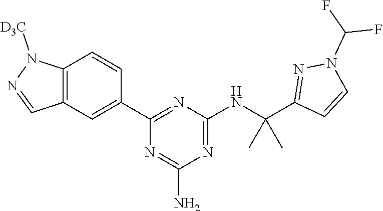 [SMILES: [2H]C([2H])([2H])n1ncc2cc(-c3nc(N)nc(NC(C)(C)c4ccn(C(F)F)n4)n3)ccc21] |
| 103 | 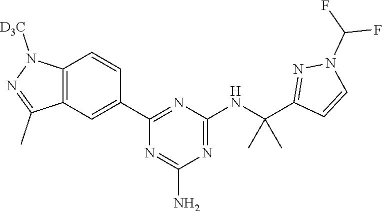 [SMILES: [2H]C([2H])([2H])n1nc(C)c2cc(-c3nc(N)nc(NC(C)(C)c4ccn(C(F)F)n4)n3)ccc21] |
| 104 | 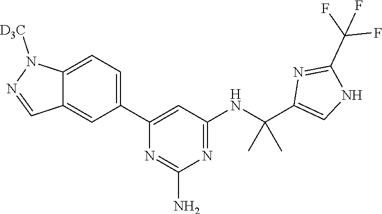 [SMILES: [2H]C([2H])([2H])n1ncc2cc(-c3cc(NC(C)(C)c4c[nH]c(C(F)(F)F)n4)nc(N)n3)ccc21] |
| 105 | 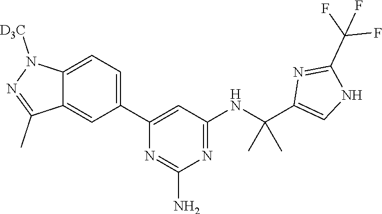 [SMILES: [2H]C([2H])([2H])n1nc(C)c2cc(-c3cc(NC(C)(C)c4c[nH]c(C(F)(F)F)n4)nc(N)n3)ccc21] |
| 106 | 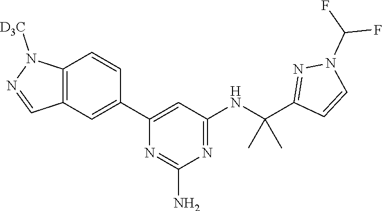 [SMILES: [2H]C([2H])([2H])n1ncc2cc(-c3cc(NC(C)(C)c4ccn(C(F)F)n4)nc(N)n3)ccc21] |
| 107 | 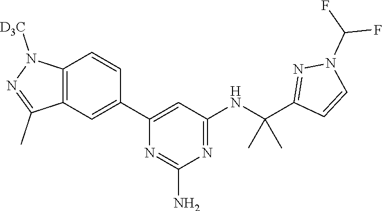 [SMILES: [2H]C([2H])([2H])n1nc(C)c2cc(-c3cc(NC(C)(C)c4ccn(C(F)F)n4)nc(N)n3)ccc21] |
| 108 | 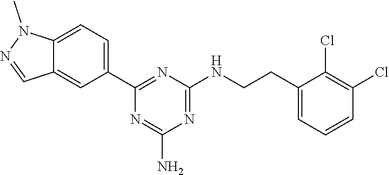 [SMILES: Cn1ncc2cc(-c3nc(N)nc(NCCc4cccc(Cl)c4Cl)n3)ccc21] |
| 109 | 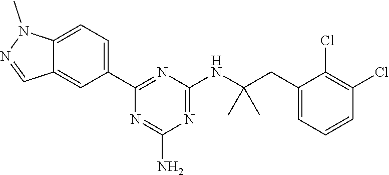 [SMILES: Cn1ncc2cc(-c3nc(N)nc(NC(C)(C)Cc4cccc(Cl)c4Cl)n3)ccc21] |
| 110 | 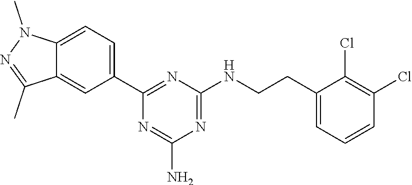 [SMILES: Cc1nn(C)c2ccc(-c3nc(N)nc(NCCc4cccc(Cl)c4Cl)n3)cc12] |
| 111 | 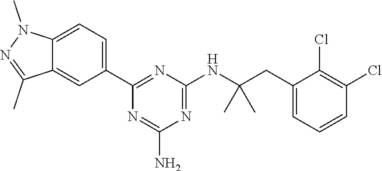 [SMILES: Cc1nn(C)c2ccc(-c3nc(N)nc(NC(C)(C)Cc4cccc(Cl)c4Cl)n3)cc12] |
| 112 | 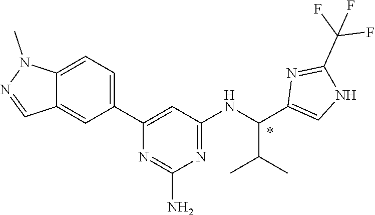 [SMILES: CC(C)C(Nc1cc(-c2ccc3c(cnn3C)c2)nc(N)n1)c1c[nH]c(C(F)(F)F)n1] |
| 113 | 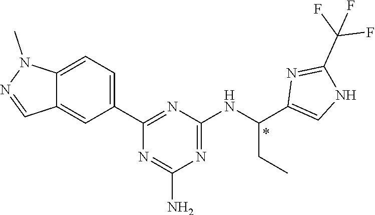 [SMILES: CCC(Nc1nc(N)nc(-c2ccc3c(cnn3C)c2)n1)c1c[nH]c(C(F)(F)F)n1] |
| 114 | 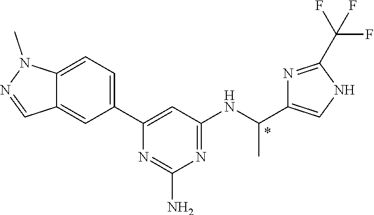 [SMILES: CC(Nc1cc(-c2ccc3c(cnn3C)c2)nc(N)n1)c1c[nH]c(C(F)(F)F)n1] |
| 115 | 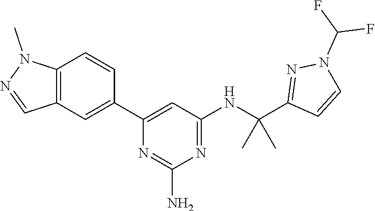 [SMILES: Cn1ncc2cc(-c3cc(NC(C)(C)c4ccn(C(F)F)n4)nc(N)n3)ccc21] |
| 116 | 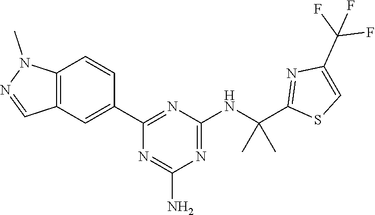 [SMILES: Cn1ncc2cc(-c3nc(N)nc(NC(C)(C)c4nc(C(F)(F)F)cs4)n3)ccc21] |
| 117 | 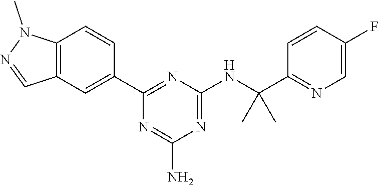 [SMILES: Cn1ncc2cc(-c3nc(N)nc(NC(C)(C)c4ccc(F)cn4)n3)ccc21] |
| 118 | 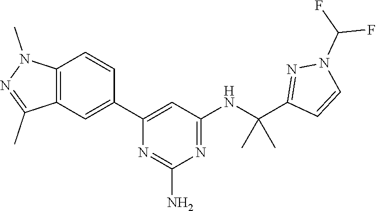 [SMILES: Cc1nn(C)c2ccc(-c3cc(NC(C)(C)c4ccn(C(F)F)n4)nc(N)n3)cc12] |
| 119 | 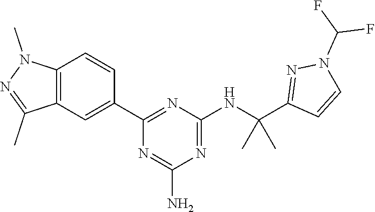 [SMILES: Cc1nn(C)c2ccc(-c3nc(N)nc(NC(C)(C)c4ccn(C(F)F)n4)n3)cc12] |
| 120 | 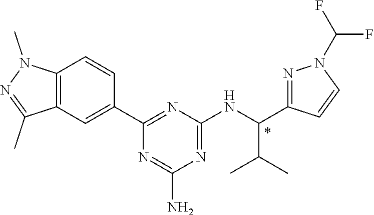 [SMILES: Cc1nn(C)c2ccc(-c3nc(N)nc(NC(c4ccn(C(F)F)n4)C(C)C)n3)cc12] |
| 121 | 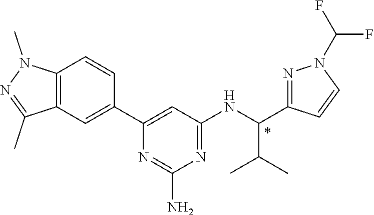 [SMILES: Cc1nn(C)c2ccc(-c3cc(NC(c4ccn(C(F)F)n4)C(C)C)nc(N)n3)cc12] |
| 122 | 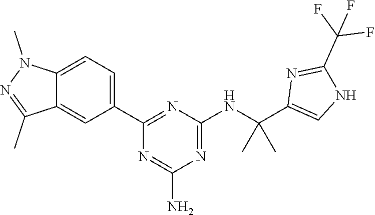 [SMILES: Cc1nn(C)c2ccc(-c3nc(N)nc(NC(C)(C)c4c[nH]c(C(F)(F)F)n4)n3)cc12] |
| 123 | 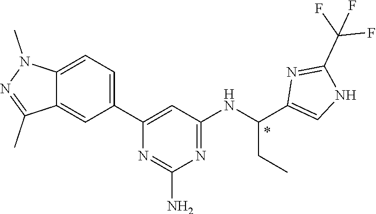 [SMILES: CCC(Nc1cc(-c2ccc3c(c2)c(C)nn3C)nc(N)n1)c1c[nH]c(C(F)(F)F)n1] |
| 124 | 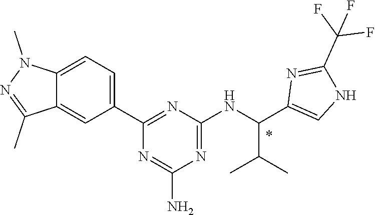 [SMILES: Cc1nn(C)c2ccc(-c3nc(N)nc(NC(c4c[nH]c(C(F)(F)F)n4)C(C)C)n3)cc12] |
| 125 | 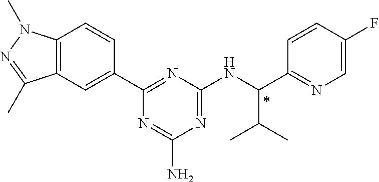 [SMILES: Cc1nn(C)c2ccc(-c3nc(N)nc(NC(c4ccc(F)cn4)C(C)C)n3)cc12] |
| 126 | 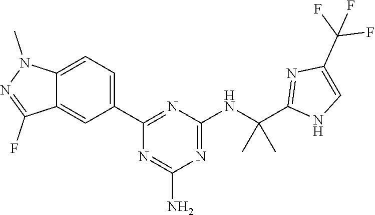 [SMILES: Cn1nc(F)c2cc(-c3nc(N)nc(NC(C)(C)c4nc(C(F)(F)F)c[nH]4)n3)ccc21] |
| 127 | 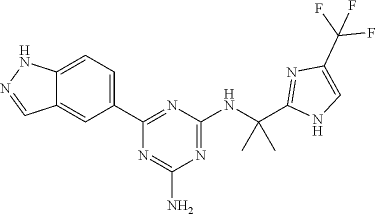 [SMILES: CC(C)(Nc1nc(N)nc(-c2ccc3[nH]ncc3c2)n1)c1nc(C(F)(F)F)c[nH]1] |
| 128 | 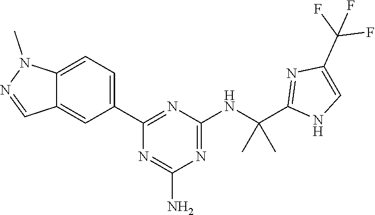 [SMILES: Cn1ncc2cc(-c3nc(N)nc(NC(C)(C)c4nc(C(F)(F)F)c[nH]4)n3)ccc21] |
| 129 | 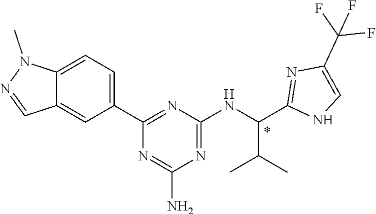 [SMILES: CC(C)C(Nc1nc(N)nc(-c2ccc3c(cnn3C)c2)n1)c1nc(C(F)(F)F)c[nH]1] |
| 130 | 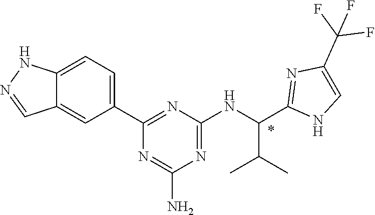 [SMILES: CC(C)C(Nc1nc(N)nc(-c2ccc3[nH]ncc3c2)n1)c1nc(C(F)(F)F)c[nH]1] |
| 131 | 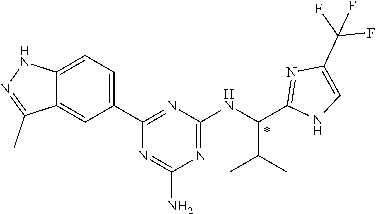 [SMILES: Cc1n[nH]c2ccc(-c3nc(N)nc(NC(c4nc(C(F)(F)F)c[nH]4)C(C)C)n3)cc12] |
| 132 | 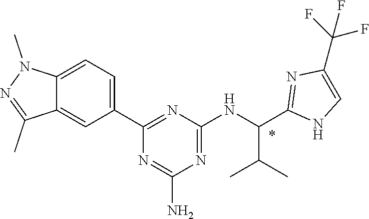 [SMILES: Cc1nn(C)c2ccc(-c3nc(N)nc(NC(c4nc(C(F)(F)F)c[nH]4)C(C)C)n3)cc12] |
| 133 | 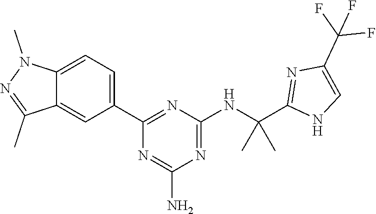 [SMILES: Cc1nn(C)c2ccc(-c3nc(N)nc(NC(C)(C)c4nc(C(F)(F)F)c[nH]4)n3)cc12] |
| 134 | 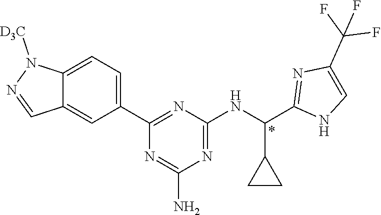 [SMILES: [2H]C([2H])([2H])n1ncc2cc(-c3nc(N)nc(NC(c4nc(C(F)(F)F)c[nH]4)C4CC4)n3)ccc21] |
| 135 | 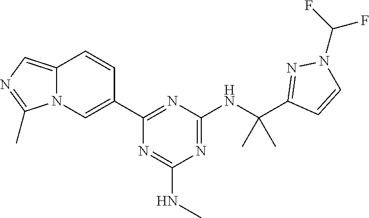 [SMILES: CNc1nc(NC(C)(C)c2ccn(C(F)F)n2)nc(-c2ccc3cnc(C)n3c2)n1] |
| 136 | 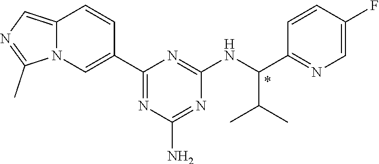 [SMILES: Cc1ncc2ccc(-c3nc(N)nc(NC(c4ccc(F)cn4)C(C)C)n3)cn12] |
| 137 | 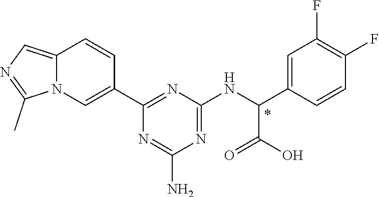 [SMILES: Cc1ncc2ccc(-c3nc(N)nc(NC(C(=O)O)c4ccc(F)c(F)c4)n3)cn12] |
| 138 | 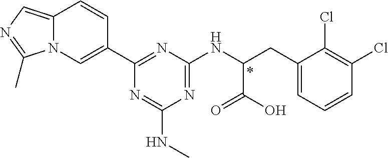 [SMILES: CNc1nc(NC(Cc2cccc(Cl)c2Cl)C(=O)O)nc(-c2ccc3cnc(C)n3c2)n1] |
| 139 | 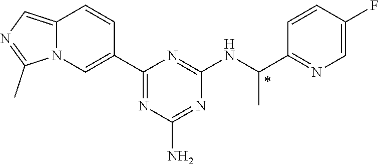 [SMILES: Cc1ncc2ccc(-c3nc(N)nc(NC(C)c4ccc(F)cn4)n3)cn12] |
| 140 | 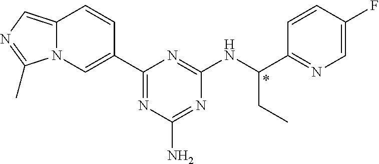 [SMILES: CCC(Nc1nc(N)nc(-c2ccc3cnc(C)n3c2)n1)c1ccc(F)cn1] |
| 141 | 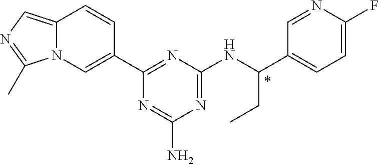 [SMILES: CCC(Nc1nc(N)nc(-c2ccc3cnc(C)n3c2)n1)c1ccc(F)nc1] |
| 142 | 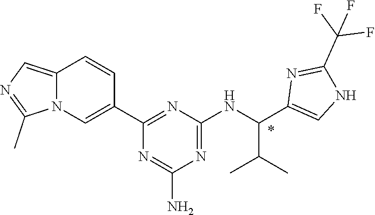 [SMILES: Cc1ncc2ccc(-c3nc(N)nc(NC(c4c[nH]c(C(F)(F)F)n4)C(C)C)n3)cn12] |
| 143 | 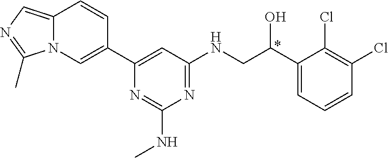 [SMILES: CNc1nc(NCC(O)c2cccc(Cl)c2Cl)cc(-c2ccc3cnc(C)n3c2)n1] |
| 144 | 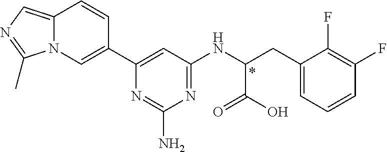 [SMILES: Cc1ncc2ccc(-c3cc(NC(Cc4cccc(F)c4F)C(=O)O)nc(N)n3)cn12] |
| 145 | 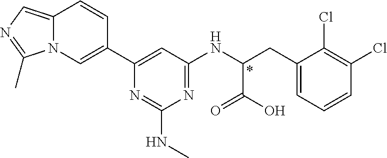 [SMILES: CNc1nc(NC(Cc2cccc(Cl)c2Cl)C(=O)O)cc(-c2ccc3cnc(C)n3c2)n1] |
| 146 | 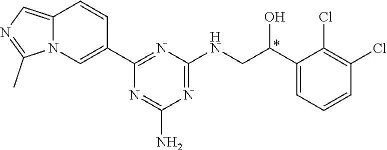 [SMILES: Cc1ncc2ccc(-c3nc(N)nc(NCC(O)c4cccc(Cl)c4Cl)n3)cn12] |
| 147 | 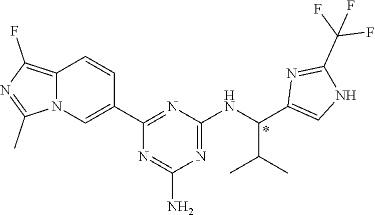 [SMILES: Cc1nc(F)c2ccc(-c3nc(N)nc(NC(c4c[nH]c(C(F)(F)F)n4)C(C)C)n3)cn12] |
| 148 | 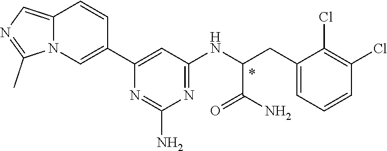 [SMILES: Cc1ncc2ccc(-c3cc(NC(Cc4cccc(Cl)c4Cl)C(N)=O)nc(N)n3)cn12] |
| 149 | 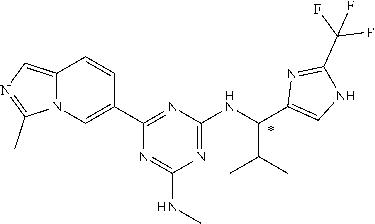 [SMILES: CNc1nc(NC(c2c[nH]c(C(F)(F)F)n2)C(C)C)nc(-c2ccc3cnc(C)n3c2)n1] |
| 150 | 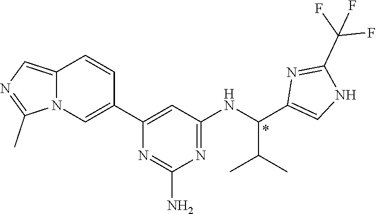 [SMILES: Cc1ncc2ccc(-c3cc(NC(c4c[nH]c(C(F)(F)F)n4)C(C)C)nc(N)n3)cn12] |
| 151 | 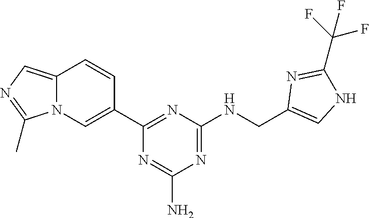 [SMILES: Cc1ncc2ccc(-c3nc(N)nc(NCc4c[nH]c(C(F)(F)F)n4)n3)cn12] |
| 152 | 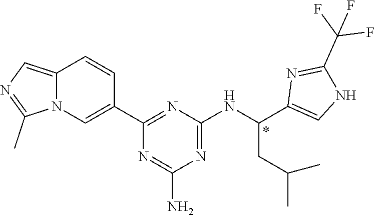 [SMILES: Cc1ncc2ccc(-c3nc(N)nc(NC(CC(C)C)c4c[nH]c(C(F)(F)F)n4)n3)cn12] |
| 153 | 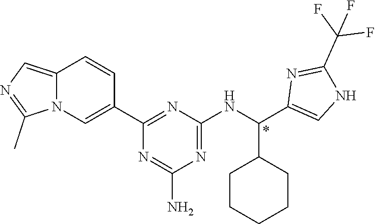 [SMILES: Cc1ncc2ccc(-c3nc(N)nc(NC(c4c[nH]c(C(F)(F)F)n4)C4CCCCC4)n3)cn12] |
| 154 | 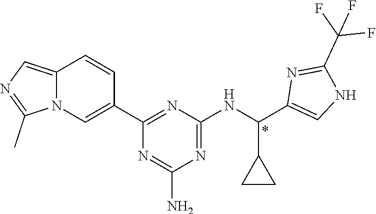 [SMILES: Cc1ncc2ccc(-c3nc(N)nc(NC(c4c[nH]c(C(F)(F)F)n4)C4CC4)n3)cn12] |
| 155 | 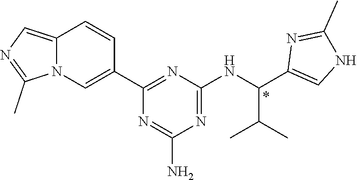 [SMILES: Cc1nc(C(Nc2nc(N)nc(-c3ccc4cnc(C)n4c3)n2)C(C)C)c[nH]1] |
| 156 | 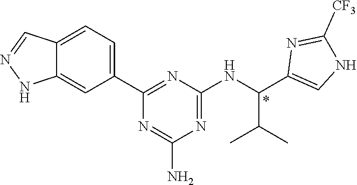 [SMILES: CC(C)C(Nc1nc(N)nc(-c2ccc3cn[nH]c3c2)n1)c1c[nH]c(C(F)(F)F)n1] |
| 157 | 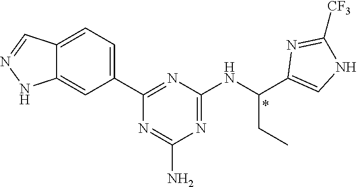 [SMILES: CCC(Nc1nc(N)nc(-c2ccc3cn[nH]c3c2)n1)c1c[nH]c(C(F)(F)F)n1] |
| 158 | 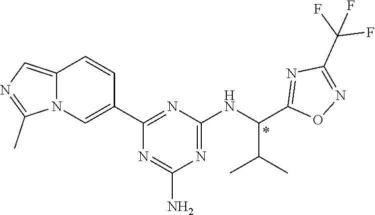 [SMILES: Cc1ncc2ccc(-c3nc(N)nc(NC(c4nc(C(F)(F)F)no4)C(C)C)n3)cn12] |
| 159 | 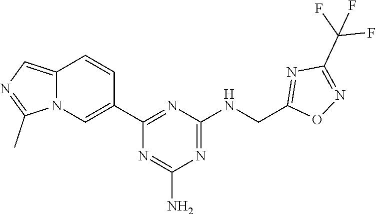 [SMILES: Cc1ncc2ccc(-c3nc(N)nc(NCc4nc(C(F)(F)F)no4)n3)cn12] |
| 160 | 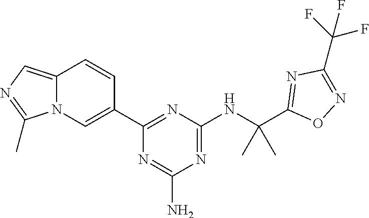 [SMILES: Cc1ncc2ccc(-c3nc(N)nc(NC(C)(C)c4nc(C(F)(F)F)no4)n3)cn12] |
| 161 | 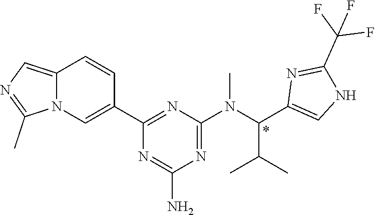 [SMILES: Cc1ncc2ccc(-c3nc(N)nc(N(C)C(c4c[nH]c(C(F)(F)F)n4)C(C)C)n3)cn12] |
| 162 | 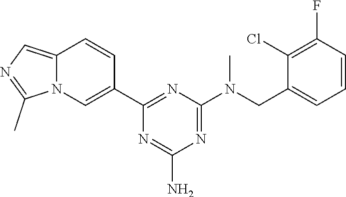 [SMILES: Cc1ncc2ccc(-c3nc(N)nc(N(C)Cc4cccc(F)c4Cl)n3)cn12] |
| 163 | 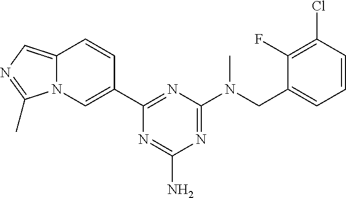 [SMILES: Cc1ncc2ccc(-c3nc(N)nc(N(C)Cc4cccc(Cl)c4F)n3)cn12] |
| 164 | 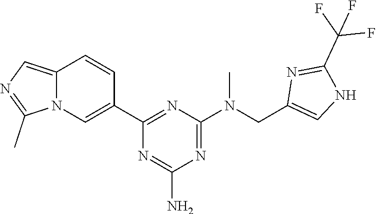 [SMILES: Cc1ncc2ccc(-c3nc(N)nc(N(C)Cc4c[nH]c(C(F)(F)F)n4)n3)cn12] |
| 165 | 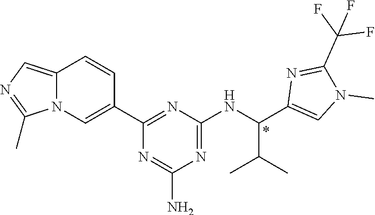 [SMILES: Cc1ncc2ccc(-c3nc(N)nc(NC(c4cn(C)c(C(F)(F)F)n4)C(C)C)n3)cn12] |
| 166 | 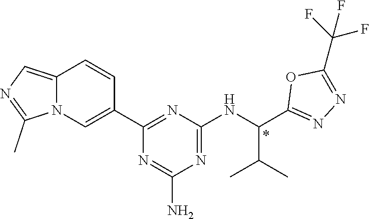 [SMILES: Cc1ncc2ccc(-c3nc(N)nc(NC(c4nnc(C(F)(F)F)o4)C(C)C)n3)cn12] |
| 167 | 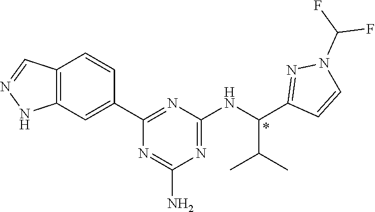 [SMILES: CC(C)C(Nc1nc(N)nc(-c2ccc3cn[nH]c3c2)n1)c1ccn(C(F)F)n1] |
| 168 | 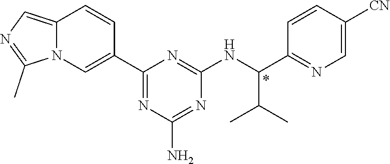 [SMILES: Cc1ncc2ccc(-c3nc(N)nc(NC(c4ccc(C#N)cn4)C(C)C)n3)cn12] |
| 169 | 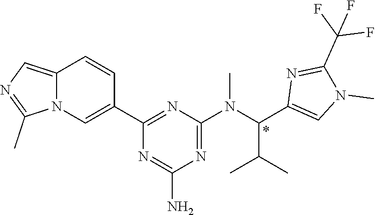 [SMILES: Cc1ncc2ccc(-c3nc(N)nc(N(C)C(c4cn(C)c(C(F)(F)F)n4)C(C)C)n3)cn12] |
| 170 | 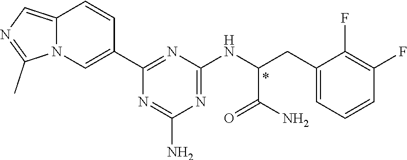 [SMILES: Cc1ncc2ccc(-c3nc(N)nc(NC(Cc4cccc(F)c4F)C(N)=O)n3)cn12] |
| 171 | 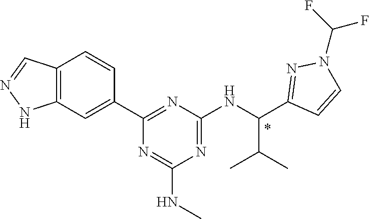 [SMILES: CNc1nc(NC(c2ccn(C(F)F)n2)C(C)C)nc(-c2ccc3cn[nH]c3c2)n1] |
| 172 | 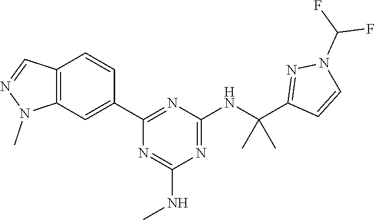 [SMILES: CNc1nc(NC(C)(C)c2ccn(C(F)F)n2)nc(-c2ccc3cnn(C)c3c2)n1] |
| 173 | 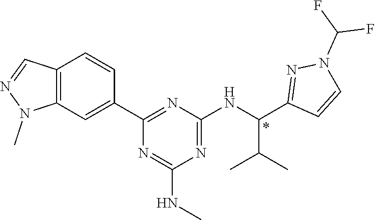 [SMILES: CNc1nc(NC(c2ccn(C(F)F)n2)C(C)C)nc(-c2ccc3cnn(C)c3c2)n1] |
| 174 | 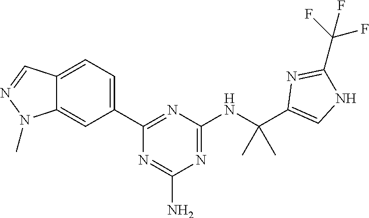 [SMILES: Cn1ncc2ccc(-c3nc(N)nc(NC(C)(C)c4c[nH]c(C(F)(F)F)n4)n3)cc21] |
| 175 | 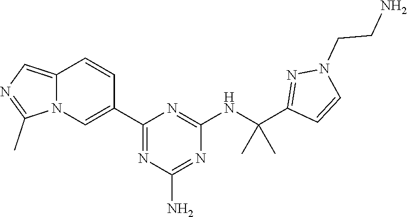 [SMILES: Cc1ncc2ccc(-c3nc(N)nc(NC(C)(C)c4ccn(CCN)n4)n3)cn12] |
| 176 | 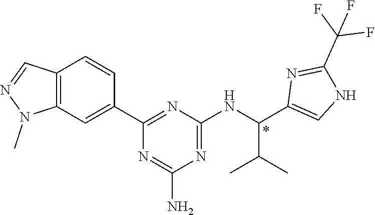 [SMILES: CC(C)C(Nc1nc(N)nc(-c2ccc3cnn(C)c3c2)n1)c1c[nH]c(C(F)(F)F)n1] |
| 177 | 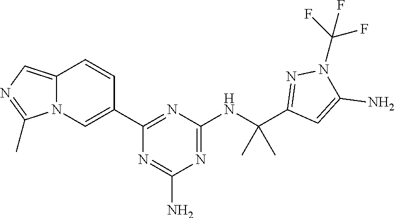 [SMILES: Cc1ncc2ccc(-c3nc(N)nc(NC(C)(C)c4cc(N)n(C(F)(F)F)n4)n3)cn12] |
| 178 | 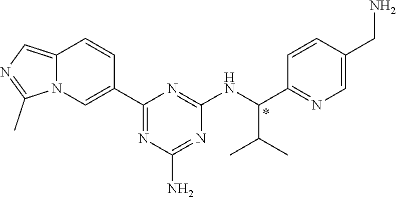 [SMILES: Cc1ncc2ccc(-c3nc(N)nc(NC(c4ccc(CN)cn4)C(C)C)n3)cn12] |
| 179 | 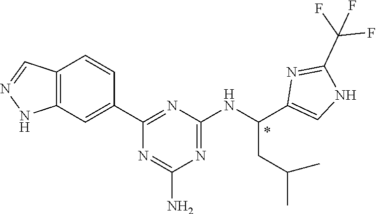 [SMILES: CC(C)CC(Nc1nc(N)nc(-c2ccc3cn[nH]c3c2)n1)c1c[nH]c(C(F)(F)F)n1] |
| 180 | 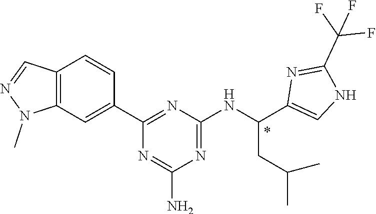 [SMILES: CC(C)CC(Nc1nc(N)nc(-c2ccc3cnn(C)c3c2)n1)c1c[nH]c(C(F)(F)F)n1] |
| 181 | 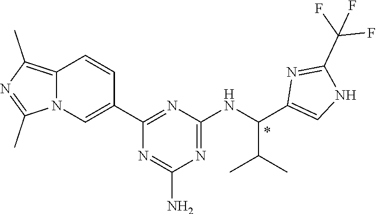 [SMILES: Cc1nc(C)n2cc(-c3nc(N)nc(NC(c4c[nH]c(C(F)(F)F)n4)C(C)C)n3)ccc12] |
| 182 | 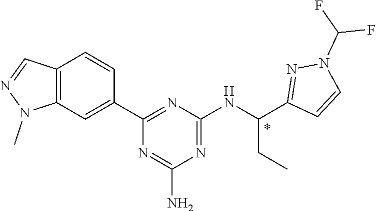 [SMILES: CCC(Nc1nc(N)nc(-c2ccc3cnn(C)c3c2)n1)c1ccn(C(F)F)n1] |
| 183 | 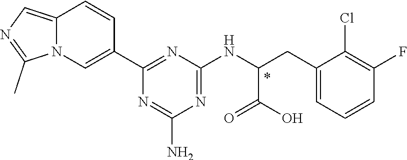 [SMILES: Cc1ncc2ccc(-c3nc(N)nc(NC(Cc4cccc(F)c4Cl)C(=O)O)n3)cn12] |
| 184 | 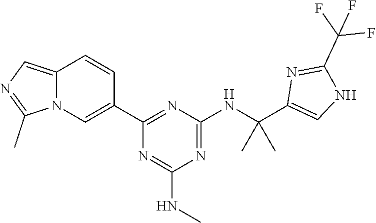 [SMILES: CNc1nc(NC(C)(C)c2c[nH]c(C(F)(F)F)n2)nc(-c2ccc3cnc(C)n3c2)n1] |
| 185 | 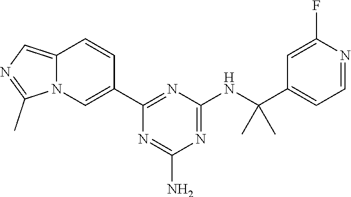 [SMILES: Cc1ncc2ccc(-c3nc(N)nc(NC(C)(C)c4ccnc(F)c4)n3)cn12] |
| 186 | 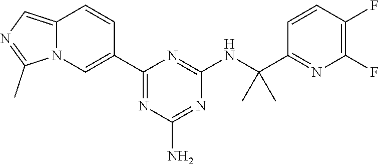 [SMILES: Cc1ncc2ccc(-c3nc(N)nc(NC(C)(C)c4ccc(F)c(F)n4)n3)cn12] |
| 187 | 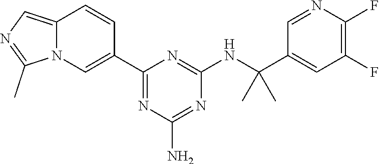 [SMILES: Cc1ncc2ccc(-c3nc(N)nc(NC(C)(C)c4cnc(F)c(F)c4)n3)cn12] |
| 188 | 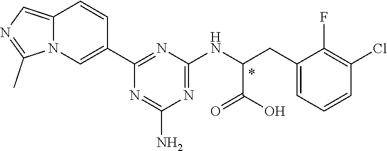 [SMILES: Cc1ncc2ccc(-c3nc(N)nc(NC(Cc4cccc(Cl)c4F)C(=O)O)n3)cn12] |
| 189 | 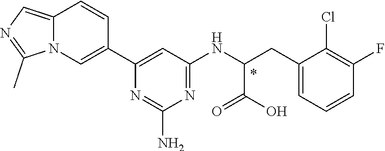 [SMILES: Cc1ncc2ccc(-c3cc(NC(Cc4cccc(F)c4Cl)C(=O)O)nc(N)n3)cn12] |
| 190 | 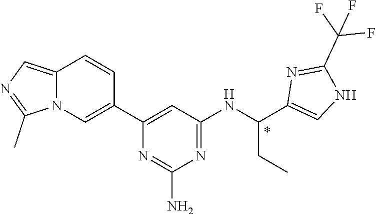 [SMILES: CCC(Nc1cc(-c2ccc3cnc(C)n3c2)nc(N)n1)c1c[nH]c(C(F)(F)F)n1] |
| 191 | 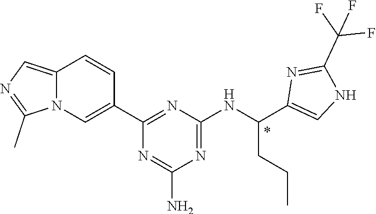 [SMILES: CCCC(Nc1nc(N)nc(-c2ccc3cnc(C)n3c2)n1)c1c[nH]c(C(F)(F)F)n1] |
| 192 | 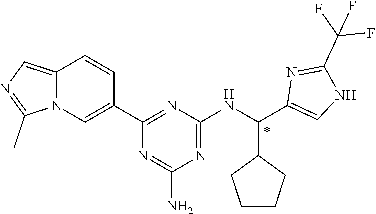 [SMILES: Cc1ncc2ccc(-c3nc(N)nc(NC(c4c[nH]c(C(F)(F)F)n4)C4CCCC4)n3)cn12] |
| 193 | 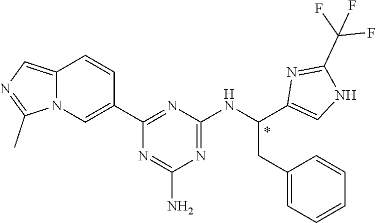 [SMILES: Cc1ncc2ccc(-c3nc(N)nc(NC(Cc4ccccc4)c4c[nH]c(C(F)(F)F)n4)n3)cn12] |
| 194 | 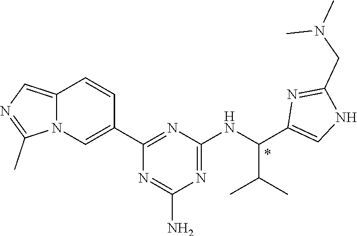 [SMILES: Cc1ncc2ccc(-c3nc(N)nc(NC(c4c[nH]c(CN(C)C)n4)C(C)C)n3)cn12] |
| 195 | 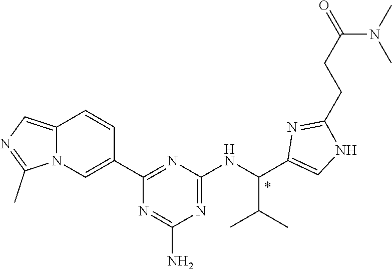 [SMILES: Cc1ncc2ccc(-c3nc(N)nc(NC(c4c[nH]c(CCC(=O)N(C)C)n4)C(C)C)n3)cn12] |
| 196 | 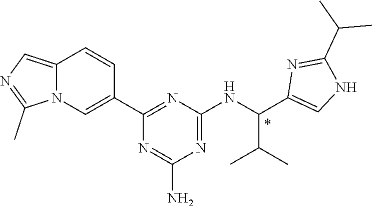 [SMILES: Cc1ncc2ccc(-c3nc(N)nc(NC(c4c[nH]c(C(C)C)n4)C(C)C)n3)cn12] |
| 197 | 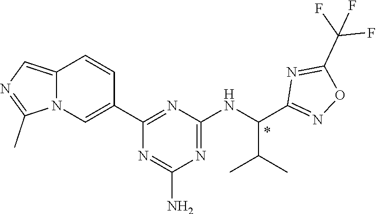 [SMILES: Cc1ncc2ccc(-c3nc(N)nc(NC(c4noc(C(F)(F)F)n4)C(C)C)n3)cn12] |
| 198 | 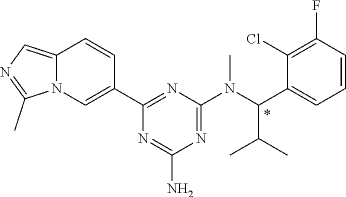 [SMILES: Cc1ncc2ccc(-c3nc(N)nc(N(C)C(c4cccc(F)c4Cl)C(C)C)n3)cn12] |
| 199 | 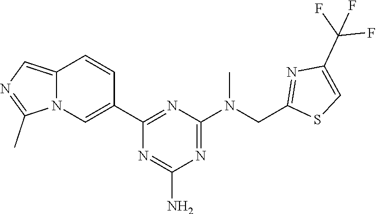 [SMILES: Cc1ncc2ccc(-c3nc(N)nc(N(C)Cc4nc(C(F)(F)F)cs4)n3)cn12] |
| 200 | 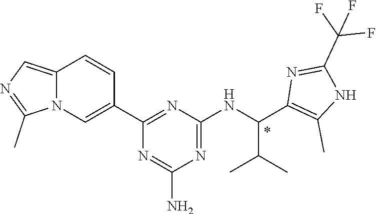 [SMILES: Cc1[nH]c(C(F)(F)F)nc1C(Nc1nc(N)nc(-c2ccc3cnc(C)n3c2)n1)C(C)C] |
| 201 | 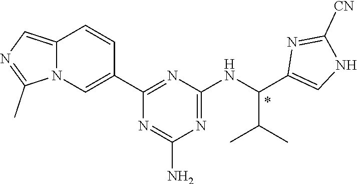 [SMILES: Cc1ncc2ccc(-c3nc(N)nc(NC(c4c[nH]c(C#N)n4)C(C)C)n3)cn12] |
| 202 | 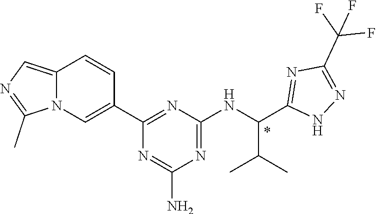 [SMILES: Cc1ncc2ccc(-c3nc(N)nc(NC(c4nc(C(F)(F)F)n[nH]4)C(C)C)n3)cn12] |
| 203 | 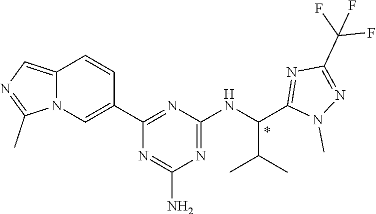 [SMILES: Cc1ncc2ccc(-c3nc(N)nc(NC(c4nc(C(F)(F)F)nn4C)C(C)C)n3)cn12] |
| 204 | 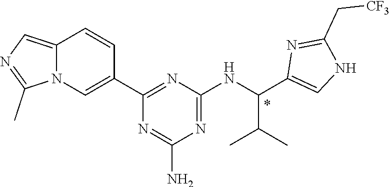 [SMILES: Cc1ncc2ccc(-c3nc(N)nc(NC(c4c[nH]c(CC(F)(F)F)n4)C(C)C)n3)cn12] |
| 205 | 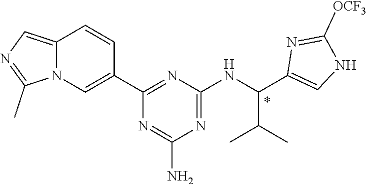 [SMILES: Cc1ncc2ccc(-c3nc(N)nc(NC(c4c[nH]c(OC(F)(F)F)n4)C(C)C)n3)cn12] |
| 206 | 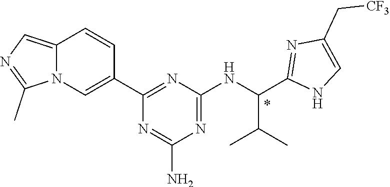 [SMILES: Cc1ncc2ccc(-c3nc(N)nc(NC(c4nc(CC(F)(F)F)c[nH]4)C(C)C)n3)cn12] |
| 207 | 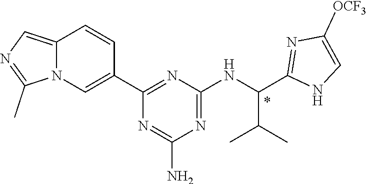 [SMILES: Cc1ncc2ccc(-c3nc(N)nc(NC(c4nc(OC(F)(F)F)c[nH]4)C(C)C)n3)cn12] |
| 208 | 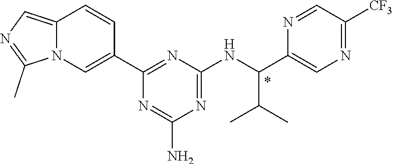 [SMILES: Cc1ncc2ccc(-c3nc(N)nc(NC(c4cnc(C(F)(F)F)cn4)C(C)C)n3)cn12] |
| 209 | 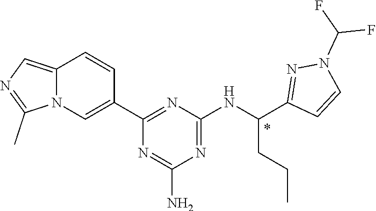 [SMILES: CCCC(Nc1nc(N)nc(-c2ccc3cnc(C)n3c2)n1)c1ccn(C(F)F)n1] |
| 210 | 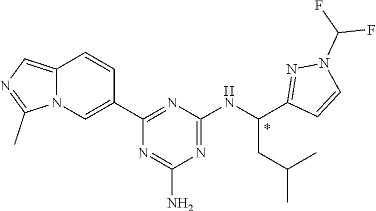 [SMILES: Cc1ncc2ccc(-c3nc(N)nc(NC(CC(C)C)c4ccn(C(F)F)n4)n3)cn12] |
| 211 | 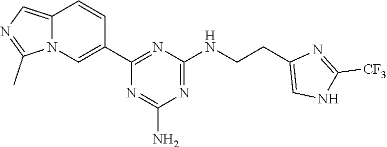 [SMILES: Cc1ncc2ccc(-c3nc(N)nc(NCCc4c[nH]c(C(F)(F)F)n4)n3)cn12] |
| 212 | 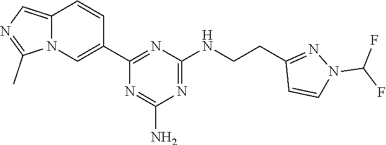 [SMILES: Cc1ncc2ccc(-c3nc(N)nc(NCCc4ccn(C(F)F)n4)n3)cn12] |
| 213 | 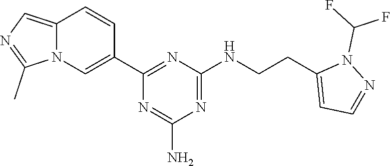 [SMILES: Cc1ncc2ccc(-c3nc(N)nc(NCCc4ccnn4C(F)F)n3)cn12] |
| 214 | 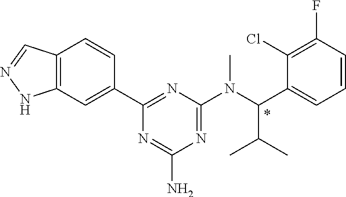 [SMILES: CC(C)C(c1cccc(F)c1Cl)N(C)c1nc(N)nc(-c2ccc3cn[nH]c3c2)n1] |
| 215 | 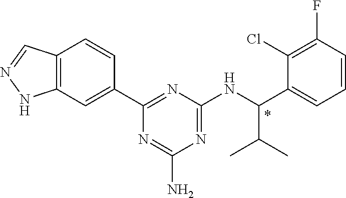 [SMILES: CC(C)C(Nc1nc(N)nc(-c2ccc3cn[nH]c3c2)n1)c1cccc(F)c1Cl] |
| 216 | 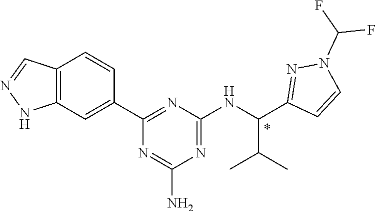 [SMILES: CC(C)C(Nc1nc(N)nc(-c2ccc3cn[nH]c3c2)n1)c1ccn(C(F)F)n1] |
| 217 | 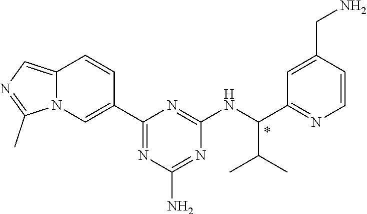 [SMILES: Cc1ncc2ccc(-c3nc(N)nc(NC(c4cc(CN)ccn4)C(C)C)n3)cn12] |
| 218 | 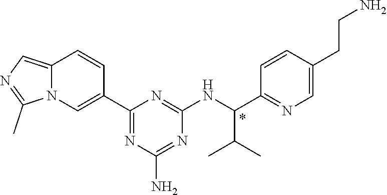 [SMILES: Cc1ncc2ccc(-c3nc(N)nc(NC(c4ccc(CCN)cn4)C(C)C)n3)cn12] |
| 219 | 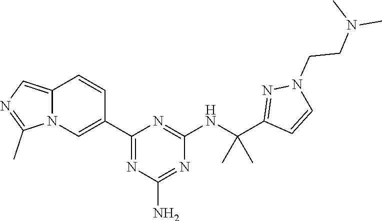 [SMILES: Cc1ncc2ccc(-c3nc(N)nc(NC(C)(C)c4ccn(CCN(C)C)n4)n3)cn12] |
| 220 | 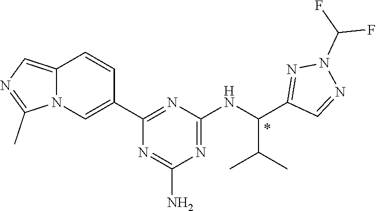 [SMILES: Cc1ncc2ccc(-c3nc(N)nc(NC(c4cnn(C(F)F)n4)C(C)C)n3)cn12] |
| 221 | 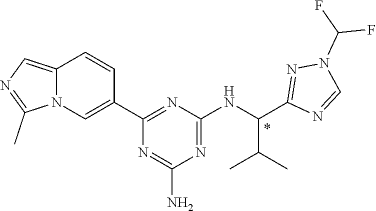 [SMILES: Cc1ncc2ccc(-c3nc(N)nc(NC(c4ncn(C(F)F)n4)C(C)C)n3)cn12] |
| 222 | 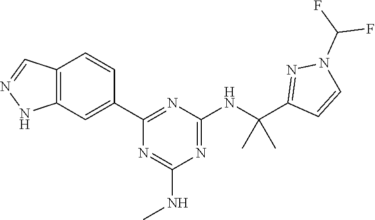 [SMILES: CNc1nc(NC(C)(C)c2ccn(C(F)F)n2)nc(-c2ccc3cn[nH]c3c2)n1] |
| 223 | 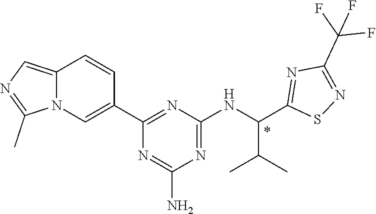 [SMILES: Cc1ncc2ccc(-c3nc(N)nc(NC(c4nc(C(F)(F)F)ns4)C(C)C)n3)cn12] |
| 224 | 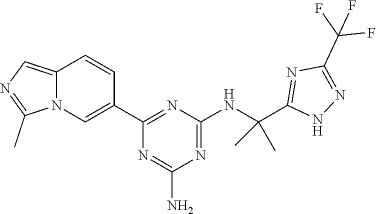 [SMILES: Cc1ncc2ccc(-c3nc(N)nc(NC(C)(C)c4nc(C(F)(F)F)n[nH]4)n3)cn12] |
| 225 | 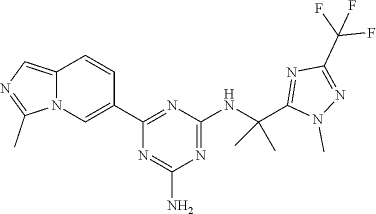 [SMILES: Cc1ncc2ccc(-c3nc(N)nc(NC(C)(C)c4nc(C(F)(F)F)nn4C)n3)cn12] |
| 226 | 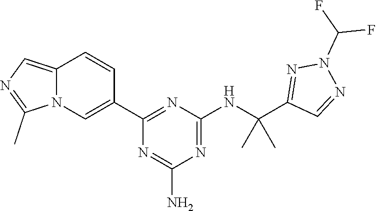 [SMILES: Cc1ncc2ccc(-c3nc(N)nc(NC(C)(C)c4cnn(C(F)F)n4)n3)cn12] |
| 227 | 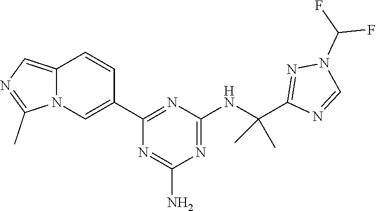 [SMILES: Cc1ncc2ccc(-c3nc(N)nc(NC(C)(C)c4ncn(C(F)F)n4)n3)cn12] |
| 228 | 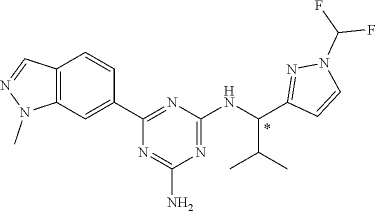 [SMILES: CC(C)C(Nc1nc(N)nc(-c2ccc3cnn(C)c3c2)n1)c1ccn(C(F)F)n1] |
| 229 | 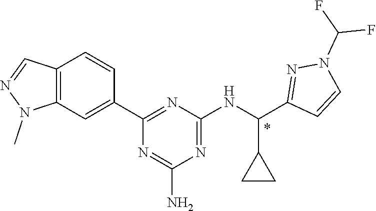 [SMILES: Cn1ncc2ccc(-c3nc(N)nc(NC(c4ccn(C(F)F)n4)C4CC4)n3)cc21] |
| 230 | 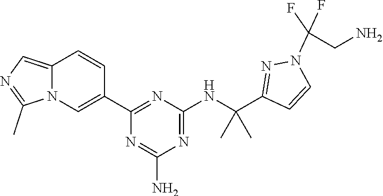 [SMILES: Cc1ncc2ccc(-c3nc(N)nc(NC(C)(C)c4ccn(C(F)(F)CN)n4)n3)cn12] |
| 231 | 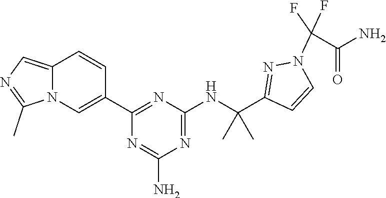 [SMILES: Cc1ncc2ccc(-c3nc(N)nc(NC(C)(C)c4ccn(C(F)(F)C(N)=O)n4)n3)cn12] |
| 232 | 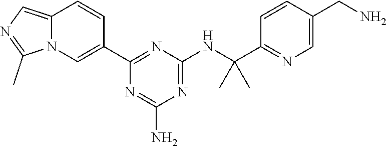 [SMILES: Cc1ncc2ccc(-c3nc(N)nc(NC(C)(C)c4ccc(CN)cn4)n3)cn12] |
| 233 | 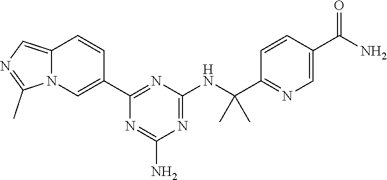 [SMILES: Cc1ncc2ccc(-c3nc(N)nc(NC(C)(C)c4ccc(C(N)=O)cn4)n3)cn12] |
| 234 | 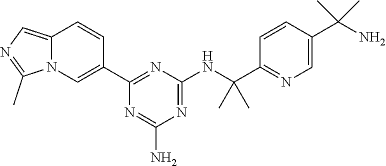 [SMILES: Cc1ncc2ccc(-c3nc(N)nc(NC(C)(C)c4ccc(C(C)(C)N)cn4)n3)cn12] |
| 235 | 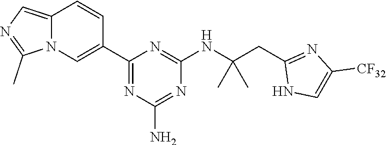 |
| 236 | 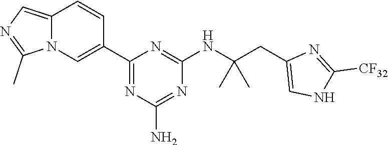 |
| 237 | 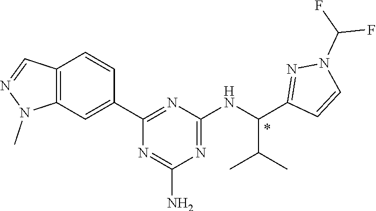 [SMILES: CC(C)C(Nc1nc(N)nc(-c2ccc3cnn(C)c3c2)n1)c1ccn(C(F)F)n1] |
| 238 | 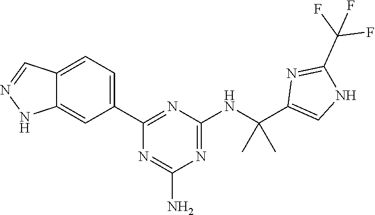 [SMILES: CC(C)(Nc1nc(N)nc(-c2ccc3cn[nH]c3c2)n1)c1c[nH]c(C(F)(F)F)n1] |
| 239 | 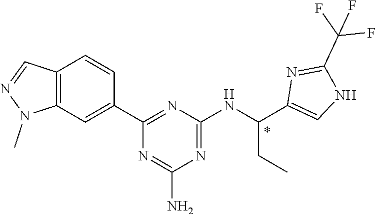 [SMILES: CCC(Nc1nc(N)nc(-c2ccc3cnn(C)c3c2)n1)c1c[nH]c(C(F)(F)F)n1] |

### Pharmaceutical Compositions

In accordance with another aspect, the present invention provides a pharmaceutical composition comprising a compound of the invention, or a pharmaceutically acceptable salt thereof, and a pharmaceutically acceptable excipient.

Conventional procedures for the selection and preparation of suitable pharmaceutical compositions are described in, for example, “Pharmaceuticals—The Science of Dosage Form Designs”, M. E. Aulton, Churchill Livingstone, 1988.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy of a condition is an amount sufficient to symptomatically relieve in a warm-blooded animal, particularly a human the symptoms of the condition or to slow the progression of the condition.

The amount of active ingredient that is combined with one or more excipients to produce a single dosage form will necessarily vary depending upon the host treated and the particular route of administration. For example, a formulation intended for oral administration to humans will generally contain, for example, from 0.1 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the invention will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, a daily dose selected from 0.1 mg/kg to 100 mg/kg, 1 mg/kg to 750 mg/kg, 1 mg/kg to 600 mg/kg, 1 mg/kg to 550 mg/kg, 1 mg/kg to 75 mg/kg, 1 mg/kg to 50 mg/kg, 1 mg/kg to 20 mg/kg or 5 mg/kg to 10 mg/kg body weight is received, given if required in divided doses. In general, lower doses will be administered when a parenteral route is employed. Thus, for example, for intravenous, subcutaneous, intramuscular or intraperitoneal administration, a dose in the range, for example, 0.1 mg/kg to 30 mg/kg body weight will generally be used. In certain embodiments the compound of the invention is administered intravenously, for example in a daily dose of from 1 mg/kg to 750 mg/kg, 1 mg/kg to 600 mg/kg, 1 mg/kg to 550 mg/kg, or 5 mg/kg to 550 mg/kg, for example at about 10, 20, 30, 40, 50, 60, 70, 80, 90, 100, 125, 150, 175, 180, 200, 225, 250, 275, 300, 350, 400, 450, 500, 540, 550 or 575 mg/kg. Similarly, for administration by inhalation, a dose in the range, for example, 0.05 mg/kg to 25 mg/kg body weight will be used. Suitably the compound of the invention is administered orally, for example in the form of a tablet, or capsule dosage form. The daily dose administered orally may be, for example a total daily dose selected from 1 mg to 1000 mg, 5 mg to 1000 mg, 10 mg to 750 mg or 25 mg to 500 mg. Typically, unit dosage forms will contain about 0.5 mg to 0.5 g of a compound of this invention. In a particular embodiment the compound of the invention is administered parenterally, for example by intravenous administration. In another particular embodiment the compound of the invention is administered orally.

### Therapeutic Uses and Applications

In accordance with another aspect, the present invention provides a compound of the invention, or a pharmaceutically acceptable salt thereof, for use as a medicament.

A further aspect of the invention provides the compound of the invention, or a pharmaceutically acceptable salt thereof, is for use in the treatment of a disease or medical condition mediated by microtubule associated serine/threonine-like kinase (MASTL).

A further aspect of the invention provides the compound of the invention, or a pharmaceutically acceptable salt thereof, is for use in the treatment of a disease in which PD-L1 expression is dependent on interferon.

Disclosed in CN 116942819 is the application of MASTL inhibitors in preparing medicaments for treating tumors in which PD-L1 expression depends on interferon. The MASTL kinase inhibitor-1 (MASTL Kinase Inhibitor-1, MKI-1) had a remarkable inhibition effect (breast cancer MDA-MB-468 cells) on the up-regulation of the interferon-induced PD-L1 expression, and could effectively enhance the anti-tumor function of T cells; whereas it was not possible to effect on the expression of tumor cells which expressed PD-L1 in a high level and which were not affected by interferon (breast cancer MDA-MB-231 cells).

In some embodiments, a disease in which PD-L1 expression is dependent on interferon is the proliferative disease. In some embodiments, the proliferative disease is a cancer, optionally wherein the cancer is selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and haematological cancer (e.g. leukaemia, such as AML, lympoma, myeloma and multiple myeloma).

Also provided is the use of a compound of the invention, or a pharmaceutically acceptable salt thereof, in the manufacture of a medicament for the treatment of a disease or medical condition mediated by MASTL.

Also provided is a method of treating a disease or medical condition mediated by MASTL in a subject in need thereof, the method comprising administering to the subject an effective amount of a compound of the invention, or a pharmaceutically acceptable salt thereof.

In the following sections of the application reference is made to a compound of the invention, or a pharmaceutically acceptable salt thereof for use in the treatment of certain diseases or conditions. It is to be understood that any reference herein to a compound for a particular use is also intended to be a reference to (i) the use of the compound of the invention, or pharmaceutically acceptable salt thereof, in the manufacture of a medicament for the treatment of that disease or condition; and (ii) a method of treating the disease or condition in a subject, the method comprising administering to the subject a therapeutically effective amount of the compound of the invention, or pharmaceutically acceptable salt thereof.

The disease of medical condition mediated by MASTL may be any of the diseases or medical conditions listed in this application, for example a proliferative disease, particularly cancer.

The subject to which the compound of the invention is administered may be a warm-blooded mammal, for example human or animal. In particular embodiments the subject or patient is a human. In other embodiments the subject is an animal, for example a rat, mouse, dog, cat, a primate or a horse.

The association of MASTL with diseases in humans and animals is set out in the Background of the Invention. This disclosure and the associated references provide further support for the therapeutic uses of the compounds of the invention. As such the supporting references linking MASTL with diseases and conditions also form part of the disclosure of the utility of the compounds of the invention in the treatment and prevention of the medical conditions described herein.

### Proliferative Diseases

MASTL has been shown to play a role in a number of diseases, including various cancers, and there is growing interest in the use of MASTL inhibitors as a therapeutic strategy (Marzec and Burgess, The Oncogenic Functions of MASTL Kinase, Front Cell Dev. Biol. (2018);6:162). This is supported by the observation that MASTL inhibition is able to reduce tumour growth in vitro and in vivo (Wang et al., (2014), Vera et al., (2015), Anania et al., (2015), Alvarez-Fernandez et al., (2018)). MASTL depletion has been shown to increase the radiosensitivity of breast cancer cells and reduce the formation of radioresistant breast cancer cells suggesting the therapeutic combination of MASTL inhibitors with radiotherapy (Yoon et al., MASTL inhibition promotes mitotic catastrophe through PP2A activation to inhibit cancer growth and radioresistance in breast cancer cells, BMC Cancer (2018) 18, 716). Knockdown of MASTL was also found to reduce the viability of thyroid cancer cells without significantly affecting normal cell proliferation (Anania et al., 2015), suggesting that MASTL inhibitors may be relatively non-toxic. ⱼᵢ

In certain embodiments the compounds of the invention are for use in the treatment of proliferative diseases, including cancer and benign proliferative disease.

### Cancer

In certain embodiments a compound of the invention is for use in the prevention or inhibition of cancer progression, for example by preventing or inhibiting cancer cell migration, cancer cell invasion and/or preventing or inhibiting cancer metastasis.

In certain embodiments the compounds of the invention are for use in the treatment of a cancer.

In certain embodiments the compounds of the invention are for use in the treatment of a cancer that overexpresses MASTL. Compounds of the invention may useful in the treatment and/or prevention of, for example:

Carcinoma, including for example tumours derived from stratified squamous epithelia (squamous cell carcinomas) and tumours arising within organs or glands (adenocarcinomas). Examples include breast, colon, lung, prostate, ovary, esophageal carcinoma (including, but not limited to, esophageal adenocarcinoma and squamous cell carcinoma), basal-like breast carcinoma, basal cell carcinoma (a form of skin cancer), squamous cell carcinoma (various tissues), head and neck carcinoma (including, but not limited to, squamous cell carcinomas), stomach carcinoma (including, but not limited to, stomach adenocarcinoma, gastrointestinal stromal tumour), signet ring cell carcinoma, bladder carcinoma (including transitional cell carcinoma (a malignant neoplasm of the bladder)), bronchogenic carcinoma, colorectal carcinoma (including, but not limited to, colon carcinoma and rectal carcinoma), anal carcinoma, gastric carcinoma, lung carcinoma (including but not limited to small cell carcinoma and non-small cell carcinoma of the lung, lung adenocarcinoma, squamous cell carcinoma, large cell carcinoma, bronchioloalveolar carcinoma, and mesothelioma), neuroendocrine tumours (including but not limited to carcinoids of the gastrointestinal tract, breast, and other organs), adrenocortical carcinoma, thyroid carcinoma, pancreatic carcinoma, breast carcinoma (including, but not limited to, ductal carcinoma, lobular carcinoma, inflammatory breast cancer, clear cell carcinoma, mucinous carcinoma), ovarian carcinoma (including, but not limited to, ovarian epithelial carcinoma or surface epithelial-stromal tumour including serous tumour, endometrioid tumour and mucinous cystadenocarcinoma, sex-cord-stromal tumour), liver and bile duct carcinoma (including, but not limited to, hepatocellular carcinoma, cholangiocarcinoma and hemangioma), prostate carcinoma, adenocarcinoma, brain tumours (including, but not limited to glioma, glioblastoma and medulloblastoma), germ cell tumours, sweat gland carcinoma, sebaceous gland carcinoma, papillary carcinoma, papillary adenocarcinoma, cystadenocarcinoma, kidney carcinoma (including, but not limited to, renal cell carcinoma, clear cell carcinoma and Wilm's tumour), medullary carcinoma, ductal carcinoma in situ or bile duct carcinoma, choriocarcinoma, seminoma, embryonal carcinoma, cervical carcinoma, uterine carcinoma (including, but not limited to, endometrial adenocarcinoma, uterine papillary serous carcinoma, uterine clear-cell carcinoma, uterine sarcomas and leiomyosarcomas, mixed mullerian tumours), testicular carcinoma, osteogenic carcinoma, epithelial carcinoma, sarcomatoid carcinoma, nasopharyngeal carcinoma, laryngeal carcinoma; oral and oropharyngeal squamous carcinoma;

Sarcomas, including: osteosarcoma and osteogenic sarcoma (bone); chondrosarcoma (cartilage); leiomyosarcoma (smooth muscle); rhabdomyosarcoma (skeletal muscle); mesothelial sarcoma and mesothelioma (membranous lining of body cavities); fibrosarcoma (fibrous tissue); angiosarcoma and hemangioendothelioma (blood vessels); liposarcoma (adipose tissue); glioma and astrocytoma (neurogenic connective tissue found in the brain); myxosarcoma (primitive embryonic connective chordoma, tissue); endotheliosarcoma, lymphangiosarcoma, lymphangioendotheliosarcoma, synovioma, Ewing's sarcoma, mesenchymous and mixed mesodermal tumour (mixed connective tissue types) and other soft tissue sarcomas;

Solid tumours of the nervous system including medulloblastoma, craniopharyngioma, ependymoma, pinealoma, hemangioblastoma, acoustic neuroma, oligodendroglioma, meningioma, neuroblastoma and schwannoma;

Melanoma, uveal melanoma and retinoblastoma;

Myeloma and multiple myeloma, including light chain myeloma, non secretory myeloma, plasmacytoma, amyloidosis, smoldering multiple myeloma (SMM), immunoglobulin D myeloma, immunoglobulin E myeloma, and conditions related to myeloma including monoclonal gammopathy of undetermined significance (MGUS);

Hematopoietic tumours, including: myelogenous and granulocytic leukaemia (malignancy of the myeloid and granulocytic white blood cell series, e.g. acute myeloid leukemia (AML)); lymphatic, lymphocytic, and lymphoblastic leukaemia (malignancy of the lymphoid and lymphocytic blood cell series); polycythemia vera and erythremia (malignancy of various blood cell products, but with red cells predominating); myelofibrosis; and

Lymphomas, including: Hodgkin and Non-Hodgkin lymphomas.

In some embodiments, a compound of the invention, or a pharmaceutically acceptable salt thereof is for use in the treatment of a solid tumour, for example any of the solid tumours listed above.

In certain embodiments the compounds of the invention are for use in the treatment of a cancer selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and haematological cancer (e.g. leukaemia, such as AML, lympoma, myeloma and multiple myeloma).

In another embodiment the compound of the invention, or a pharmaceutically acceptable salt thereof, is for use in the treatment of a breast cancer selected from Luminal A breast cancer (hormone-receptor positive (estrogen-receptor and/or progesterone-receptor positive), HER2 negative and low levels of the protein Ki-67); Luminal B breast cancer (hormone-receptor positive (estrogen-receptor and/or progesterone-receptor positive), and either HER2 positive or HER2 negative with high levels of Ki-67); triple negative breast cancer (i.e. the tumour is estrogen receptor-negative, progesterone receptor-negative and HER2-negative); HER2 positive breast cancer or normal-like breast cancer (classifications as defined in Table 1 of Dai et al. Am. J. Cancer Research. 2015;5 (10): 2929-2943).

In an embodiment a compound of the invention, or a pharmaceutically acceptable salt thereof is for use in the treatment of a cancer selected from: pancreatic cancer, triple negative breast cancer (i.e. the tumour is estrogen receptor-negative, progesterone receptor-negative and HER2-negative), hormone refractory prostate cancer and non-small cell lung cancer.

In embodiments the compounds of the invention provide an anti-cancer effect on a cancer (for example any of the cancers disclosed herein) selected from one or more of an antiproliferative effect, a pro-apoptotic effect, an anti-mitotic effect an anti-angiogenic effect, inhibition of cell migration, inhibition or prevention of tumour invasion and/or preventing or inhibiting metastasis.

Compounds of the invention may be used to prevent or inhibit the progression of a cancer. A compound of the invention may be for use in slowing, delaying or stopping cancer progression. The progress of a cancer is typically determined by assigning a stage to the cancer. Staging is typically carried out by assigning a number from I to IV to the cancer, with I being an isolated cancer and IV being an advanced stage of the disease where the cancer that has spread to other organs. The stage generally takes into account the size of a tumour, whether it has invaded adjacent organs, the number of lymph nodes it has spread to, and whether the cancer has metastasised. Preventing or inhibiting progression of the cancer is particularly important for preventing the spread of the cancer, for example the progression from Stage I to Stage II where the cancer spreads locally, or the progression from Stage III to Stage IV where the cancer metastasises to other organs.

It may be that a compound of the invention is for use in the treatment of a cancer wherein the cancer is a primary cancer, which may be a second primary cancer.

It may be that a compound of the invention is for use in the prevention or inhibition of occurrence of a second primary cancer.

It may be that a compound of the invention is for use in the treatment of a cancer wherein the cancer is refractory (resistant) to an anti-cancer agent (e.g. chemotherapy) and/or radio therapy. The cancer may be resistant at the beginning of treatment or it may become resistant during treatment.

It may be that a compound of the invention is for use in the treatment of a cancer wherein the cancer is a recurrent cancer, which may be local, regional or distant. A recurrent cancer is a cancer which returns after initial treatment and after a period of time during which the cancer cannot be detected. The same cancer may return in the same tissue or in a different part of the body.

It may be that a compound of the invention is for use in the prevention or inhibition of recurrence of a cancer.

It may be that a compound of the invention is for use in the treatment of a cancer wherein the cancer is a metastatic or secondary cancer.

It may be that a compound of the invention is for use in the prevention or inhibition of cancer metastasis. The treatment of a metastatic cancer may be the same or different to the therapy previously used to treat the primary tumour. For example, in certain embodiments, a primary tumour may be surgically resected and a compound of the invention is for use in preventing the spread of cancer cells that may remain following surgery, or which may have already escaped the primary tumour. In other embodiments, the primary tumour may be treated using radiotherapy. In yet other embodiments, the primary tumour may be treated by chemotherapy. Combination therapies are commonly used to treat cancer to improve the treatment and, typically, maximise the length and depth of the remission. Any of the combination therapies disclosed herein may be used with a compound of the invention.

When the primary tumour has already metastasised and a secondary tumour has established, a compound of the invention may be used to treat the secondary tumour. This may involve both treatment of the secondary tumour and prevention of that secondary tumour metastasising. Reference to metastasis herein is intended to encompass metastasis of any of the tumours disclosed herein. Generally, the secondary tumour will be in a different tissue to that of the primary tumour. For example the secondary tumour may be a secondary tumour in bone. In a particular embodiment a compound of the invention is for use in the treatment of a secondary tumour in bone, for example for use in the treatment of a secondary bone tumour, wherein the primary tumour is a breast or prostate tumour.

### Benign Proliferative Disease

A compound of the invention, or a pharmaceutically acceptable salt thereof the invention may be for use in the treatment of a benign proliferative disease. The benign disease may be a benign tumour, for example hemangiomas, hepatocellular adenoma, cavernous haemangioma, focal nodular hyperplasia, acoustic neuromas, neurofibroma, bile duct adenoma, bile duct cystanoma, fibroma, lipomas, leiomyomas, mesotheliomas, teratomas, myxomas, nodular regenerative hyperplasia, trachomas, pyogenic granulomas, moles, uterine fibroids, thyroid adenomas, adrenocortical adenomas or pituitary adenomas.

In some embodiments, the benign proliferative disease is a hyperproliferative skin disorder. Benign hyperproliferative skin disorders include psoriasis, common warts, keratoacanthoma, seborrhea, ichthyosis, actinic keratosis, Bowen's Disease, papilloma, seborrhoeic keratosis, eczema, atopic dermatitis, keloids, and Epidermolysis Bullosa (EB).

### Other Diseases and Conditions

In certain embodiments, the compounds of the invention are for use in the treatment or prevention of a metabolic disorder, or symptoms or conditions associated with a metabolic disorder.

The metabolic disorder may be a glucose metabolism disorder, or a body weight disorder.

The term “glucose metabolism disorder” encompasses any disorder characterized by a clinical symptom or a combination of clinical symptoms that is associated with an elevated level of glucose and/or an elevated level of insulin in a subject relative to a healthy individual. Elevated levels of glucose and/or insulin may be manifested in the following diseases, disorders and conditions: hyperglycemia, type II diabetes, gestational diabetes, type I diabetes, insulin resistance, impaired glucose tolerance, hyperinsulinemia, impaired glucose metabolism, prediabetes, other metabolic disorders (such as metabolic syndrome), and obesity, among others.

The term “insulin resistance” as used herein refers to a condition wherein a normal amount of insulin is unable to produce a normal physiological or molecular response.

The term “hyperglycemia”, as used herein, refers to a condition in which an elevated amount of glucose circulates in the blood plasma of a subject relative to a healthy individual. Hyperglycemia can be diagnosed using methods known in the art, including measurement of fasting blood glucose levels.

The term “hyperinsulinemia”, as used herein, refers to a condition in which there are elevated levels of circulating insulin when, concomitantly, blood glucose levels are either elevated or normal. Hyperinsulinemia can be caused by insulin resistance which is associated with dyslipidemia, such as high triglycerides, high cholesterol, high low-density lipoprotein (LDL) and low high-density lipoprotein (HDL); high uric acids levels; polycystic ovary syndrome; type II diabetes and obesity. Hyperinsulinemia can be diagnosed as having a plasma insulin level higher than about 2 μl/ml.

The phrase “body weight disorder” refers to conditions associated with excessive body weight and/or enhanced appetite. Various parameters are used to determine whether a subject is overweight compared to a reference healthy individual, including the subject's age, height, sex and health status. For example, a subject may be considered overweight or obese by assessment of the subject's Body Mass Index (BMI), which is calculated by dividing a subject's weight in kilograms by the subject's height in meters. An adult having a BMI in the range of −18.5 to −24.9 kg/m is considered to have a normal weight; an adult having a BMI between −25 and −29.9 kg/m may be considered overweight (pre-obese); and an adult having a BMI of −30 kg/m or higher may be considered obese. Thus, in some embodiments the body weight disorder is obesity.

Symptoms and conditions associated with metabolic disorders may thus include, but are not limited to, increased blood sugar (hyperglycemia), decreased insulin production, metabolic syndrome, increased cholesterol, increased triglyceride levels, heart disease, stroke, high blood pressure, an increased risk of blood clots (e.g. deep vein thrombosis), glucosuria, metabolic acidosis, diabetic neuropathy, diabetic nephropathy, diabetic retinopathy, and diabetic cardiomyopathy.

The term “metabolic syndrome” refers to an associated cluster of traits that includes, but is not limited to, hyperinsulinemia, abnormal glucose tolerance, redistribution of fat to the abdominal or upper body compartment, hypertension, dysfibrinolysis, and dyslipidemia characterized by high triglycerides, low high density lipoprotein (HDL)-cholesterol, and high small dense low density lipoprotein (LDL) particles. Subjects having metabolic syndrome are at risk for development of type 2 diabetes and/or other disorders (e.g., atherosclerosis).

Compounds of the invention may be used to prevent or inhibit the progression or symptoms of the metabolic disorder or condition associated therewith. For example, compounds of the invention may lower blood glucose, insulin, triglyceride, or cholesterol levels to a range found in a healthy subject; reduce body weight; improve glucose tolerance, energy expenditure, or insulin sensitivity; delay the onset or progression of diabetes; reduce blood pressure; and/or reduce the risk of blood clots, heart disease or stroke.

In certain embodiments, the compounds of the invention are for use in the treatment of a platelet disorder, such as thrombocytopenia.

The compounds of the invention may be used alone or in combination with one or more anticancer agents and/or radiotherapy as described herein.

### Combination Therapies

The compounds of the invention may be used alone to provide a therapeutic effect. The compounds of the invention may also be used in combination with one or more additional therapies.

In some embodiments, the compounds of the invention are used in combination with one or more anti-cancer agents and/or radiotherapy.

The rationale for this is based on results showing that overexpression of MASTL is associated with resistance to cisplatin (Wang et al., 2014) by accelerating checkpoint recovery (Wong et al., 2016). Conversely, knockdown of MASTL has been observed to sensitize cancer cells to cisplatin, radiotherapy and 5-fluorouracil (5FU) in several cancer types (Wang et al., (2014). ᵢᵢ Mastl kinase, a promising therapeutic target, promotes cancer recurrence. ᵢᵢ Oncotarget ᵢᵢ 5 ᵢᵢ 11479-11489; ᵢᵢ Nagel et al., (2015). ᵢᵢ Genome-wide siRNA Screen identifies the radiosensitizing effect of downregulation of MASTL and FOXM1 in NSCLC. ⱼⱼ(Mol. Cancer Ther. ᵢᵢ 14 ᵢᵢ 1434-1444; ᵢᵢ Uppada et al (2018). ᵢᵢ MASTL induces colon cancer progression and chemoresistance by promoting Wnt/¥-catenin signaling. ᵢᵢ (Mol. Cancer ᵢᵢ 17: 111; ᵢᵢ Yoon et al., (2018). ᵢᵢ MASTL inhibition promotes mitotic catastrophe through PP2A activation to inhibit cancer growth and radioresistance in breast cancer cells. ᵢᵢ BMC Cancer ᵢᵢ 18:716).

Compounds of the invention may therefore be used to prevent or reduce resistance of cells to anti-cancer agents, including chemotherapeutic agents, radiotherapy.

*667 Such chemotherapy may include one or more of the following categories of anti-cancer agents:
(i) antiproliferative/antineoplastic drugs and combinations thereof, such as alkylating agents (for example cis-platin, oxaliplatin, carboplatin, cyclophosphamide, nitrogen mustard, uracil mustard, bendamustin, melphalan, chlorambucil, chlormethine, busulphan, temozolamide, nitrosoureas, ifosamide, melphalan, pipobroman, triethylene-melamine, triethylenethiophoporamine, carmustine, lomustine, stroptozocin and dacarbazine); antimetabolites (for example gemcitabine and antifolates such as fluoropyrimidines like 5-fluorouracil and tegafur, raltitrexed, methotrexate, pemetrexed, cytosine arabinoside, floxuridine, cytarabine, 6-mercaptopurine, 6-thioguanine, fludarabine phosphate, pentostatine, and gemcitabine and hydroxyurea); antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); proteasome inhibitors, for example carfilzomib and bortezomib; interferon therapy; and topoisomerase inhibitors (for example epipodophyllotoxins like etoposide and teniposide, amsacrine, topotecan, irinotecan, mitoxantrone and camptothecin); bleomcin, dactinomycin, daunorubicin, doxorubicin, epirubicin, idarubicin, ara —C, paclitaxel (Taxol™), nab paclitaxel (albumin-bound paclitaxel), docetaxel, mithramycin, deoxyco-formycin, mitomycin-C, L-asparaginase, interferons (especially IFN-alpha), etoposide, teniposide, DNA-demethylating agents, (for example, azacitidine or decitabine); and histone de-acetylase (HDAC) inhibitors (for example vorinostat, MS-275, panobinostat, romidepsin, valproic acid, mocetinostat (MGCD0103) and pracinostat SB939);
(ii) cytostatic agents such as antiestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5¥á-reductase such as finasteride; and navelbene, CPT-11, anastrazole, letrazole, capecitabine, reloxafme, cyclophosphamide, ifosamide, and droloxafine;
(iii) anti-invasion agents, for example dasatinib and bosutinib (SKI-606), and metalloproteinase inhibitors, inhibitors of urokinase plasminogen activator receptor function or antibodies to Heparanase;
(iv) inhibitors of growth factor function: for example such inhibitors include growth factor antibodies and growth factor receptor antibodies, for example the anti-erbB2 antibody trastuzumab [Herceptin™ ], the anti-EGFR antibody panitumumab, the anti-erbB1 antibody cetuximab, tyrosine kinase inhibitors, for example inhibitors of the epidermal growth factor family (for example EGFR family tyrosine kinase inhibitors such as gefitinib, erlotinib, 6-acrylamido-N-(3-chloro-4-fluorophenyl)-7-(3-morpholinopropoxy)-quinazolin-4-amine (CI 1033), afatinib, vandetanib, osimertinib and rociletinib) erbB2 tyrosine kinase inhibitors such as lapatinib) and antibodies to costimulatory molecules such as CTLA-4, 4-1BB and PD-1, or antibodies to cytokines IL-10, TGF-beta); inhibitors of the hepatocyte growth factor family; inhibitors of the insulin growth factor family; modulators of protein regulators of cell apoptosis (for example Bcl-2 inhibitors); inhibitors of the platelet-derived growth factor family such as imatinib and/or nilotinib (AMN107); inhibitors of serine/threonine kinases (for example Ras/Raf signalling inhibitors such as farnesyl transferase inhibitors, sorafenib, tipifarnib and lonafarnib), inhibitors of cell signalling through MEK and/or AKT kinases, c-kit inhibitors, abl kinase inhibitors, PI3 kinase inhibitors, Plt3 kinase inhibitors, CSF-1R kinase inhibitors, IGF receptor, kinase inhibitors, for example dalotuzumab; aurora kinase inhibitors and cyclin dependent kinase inhibitors such as CDK2 and/or CDK4 inhibitors; CCR2, CCR4 or CCR6 antagonists; RAF kinase inhibitors such as those described in WO2006043090, WO2009077766, WO2011092469 or WO2015075483; and Hedgehog inhibitors, for example vismodegib.
v) antiangiogenic agents such as those which inhibit the effects of vascular endothelial growth factor, [for example the anti-vascular endothelial cell growth factor antibody bevacizumab (Avastin™)]; thalidomide; lenalidomide; and for example, a VEGF receptor tyrosine kinase inhibitor such as vandetanib, vatalanib, sunitinib, axitinib, pazopanib and cabozantinib;
(vi) gene therapy approaches, including for example approaches to replace aberrant genes such as aberrant p53 or aberrant BRCA1 or BRCA2;
(vii) immunotherapy approaches, including for example antibody therapy such as alemtuzumab, rituximab, ibritumomab tiuxetan(Zevalinçç) and ofatumumab; interferons such as interferon ¥a; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); gp100;dendritic cell-based vaccines (such as Ad.p53 DC); toll-like receptor modulators for example TLR-7 or TLR-9 agonists; PD-1, PD-L1, PD-L2 and CTL4-A modulators (for example Nivolumab), antibodies and vaccines; other IDO inhibitors (such as indoximod); anti-PD-1 monoclonal antibodies (such as MK-3475 and nivolumab); anti-PDL1 monoclonal antibodies (such as MEDI-4736 and RG-7446); anti-PDL2 monoclonal antibodies; and anti-CTLA-4 antibodies (such as ipilumumab), CAR-T cell therapies; and
(viii) cytotoxic agents for example fludaribine (fludara), cladribine, pentostatin (Nipent™);
(ix) targeted therapies, for example PI3K inhibitors, for example idelalisib and perifosine; SMAC (second mitochondriaderived activator of caspases) mimetics, also known as Inhibitor of Apoptosis Proteins (IAP) antagonists (IAP antagonists). These agents act to supress IAPs, for example XIAP, cIAP1 and cIAP2, and thereby re-establish cellular apoptotic pathways. Particular SMAC mimetics include Birinapant (TL32711, TetraLogic Pharmaceuticals), LCL161 (Novartis), AEG40730 (Aegera Therapeutics), SM-164 (University of Michigan), LBW242 (Novartis), ML101 (Sanford-Burnham Medical Research Institute), AT-406 (Ascenta Therapeutics/University of Michigan), GDC-0917 (Genentech), AEG35156 (Aegera Therapeutic), and HGS1029 (Human Genome Sciences); and agents which target ubiquitin proteasome system (UPS), for example, bortezomib, carfilzomib, marizomib (NPI-0052) and MLN9708; a CXCR4 antagonist, for example plerixafor or BL-8040;
(x) PARP inhibitors, for example niraparib (MK-4827), talazoparib (BMN-673), veliparib ᵢᵢ (ABT-888); olaparib, CEP 9722, and BGB-290
(xi) chimeric antigen receptors, anticancer vaccines and arginase inhibitors;
(xii) agents which degrade hyaluronan, for example the hyaluronidase enzyme PEGPH₂O

The additional anti-cancer agent may be a single agent or one or more of the additional agents listed herein.

Particular anti-cancer agents which may be used together with a compound of the invention include, for example, paclitaxel (including nab paclitaxel), gemcitabine, oxaliplatin, irinotecan, leucovorin and 5-fluorouracil. In some embodiments the additional anti-cancer agent selected from capecitabine, gemcitabine and 5-fluorouracil (5FU).

In some embodiments, the compounds of the invention are used in combination with one or more therapies for treating or preventing a metabolic disorder, including therapeutic agents, LDL apheresis, dietary restrictions and/or surgery (e.g. bariatric surgery).

Therapeutic agents for treating or preventing a metabolic disorder may include one or more of the following categories of agents:
(i) diabetes treatments, for example metformin, sulfonylureas (e.g. glyburide, glipizide and glimepiride), meglitinides (e.g. repaglinide and nateglinide), thiazolidinediones (e.g. rosiglitazone and pioglitazone), DPP-4 inhibitors (e.g. sitagliptin, saxagliptin and linagliptin), GLP-1 receptor agonists (e.g. exenatide, liraglutide and semaglutide), SGLT2 inhibitors (e.g. canagliflozin, dapagliflozin and empagliflozin), insulin (e.g. long-acting insulin such as glargine or insulin detemir) and aspirin;
(ii) cholesterol-lowering agents, for example statins (e.g. Atorvastatin, Lovastatin, Pitavastatin, Pravastatin, Rosuvastatin, Simvastatin); cholesterol absorption inhibitors (e.g. ezetimibe); PCSK9 inhibitors (e.g. repatha and praluent);
(iii) triclyceride-lowering agents, for example statins, fibrates, nicotinic acid, and omega-3 fatty acids;
(iv) anti-clotting agent, for example anticoagulants (e.g. heparin, warfarin, rivaroxaban, ᵢᵢ dabigatran, apixaban, edoxaban, enoxaparin, fondaparinux);
(v) blood-pressure lowering agents, for example diuretics (e.g. thiazide diuretics such as chlorthalidone, chlorothiazide, hydrochlorothiazide, indapamide and metolazone; potassium-sparing diuretics such as amiloride, spironolactone and triamterene; loop diuretics such as bumetanide, furosemide, torsemide; combination diuretics such as amiloride hydrochloride/hydrochlorothiazide, spironolactone/hydrochlorothiazide, triamterene/hydrochlorothiazide), beta blockers (e.g. acebutolol, atenolol, ᵢᵢ betaxolol, bisoprolol, bisoprolol/hydrochlorothiazide, metoprolol tartrate, metoprolol succinate, nadolol, pindolol, propranolol, ᵢᵢ solotol, timolol), ACE inhibitors (e.g. benazepril, captopril, enalapril, fosinopril, lisinopril, moexipril, perindopril, quinapril, ramipril, trandolapril), angiotensis II receptor blockers (ARBs)(e.g. candesartan, eprosartan, irbesartan, losartan, ᵢᵢ telmisartan, valsartan), calcium channel blockers (e.g. amlodipine, ᵢᵢ diltiazem, felodipine, isradipine, nicardipine, nifedipine, nisoldipine, verapamil), alpha blockers (e.g. doxazosin, prazosin, terazosin), alpha-beta blockers (e.g. carvedilol, labetalol), central agonists (e.g. methyldopa, clonidine, guanfacine), vasodilators (e.g. hydralazine, minoxidil), aldosterone receptor antagonists (e.g. eplerenone, spironolactone), direct renin inhibitors (e.g. aliskiren).

Such combination treatment may be achieved by way of the simultaneous, sequential or separate dosing of the individual components of the treatment. Such combination products employ the compounds of this invention within a therapeutically effective dosage range described hereinbefore and the other pharmaceutically-active agent within its approved dosage range.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

In some embodiments in which a combination treatment is used, the amount of the compound of the invention and the amount of the other pharmaceutically active agent(s) are, when combined, therapeutically effective to treat a targeted disorder in the patient. In this context, the combined amounts are “therapeutically effective amount” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound of the invention and an approved or otherwise published dosage range(s) of the other pharmaceutically active compound(s).

According to a further aspect of the invention there is provided a compound of the invention as defined hereinbefore and an additional anti-cancer agent as defined hereinbefore, for use in the conjoint treatment of cancer.

According to a further aspect of the invention there is provided a pharmaceutical product comprising a compound of the invention as defined hereinbefore and an additional anti-cancer agent as defined hereinbefore for the conjoint treatment of cancer.

According to a further aspect of the invention there is provided a method of treatment of a human or animal subject suffering from a cancer comprising administering to the subject a therapeutically effective amount of a compound of the invention, or a pharmaceutically acceptable salt thereof simultaneously, sequentially or separately with an additional anti-cancer agent as defined hereinbefore.

According to a further aspect of the invention there is provided a compound of the invention, or a pharmaceutically acceptable salt thereof for use simultaneously, sequentially or separately with an additional anti-cancer agent as defined hereinbefore, in the treatment of a cancer.

The compound of the invention may also be used be used in combination with radiotherapy. Suitable radiotherapy treatments include, for example X-ray therapy, proton beam therapy or electron beam therapies. Radiotherapy may also encompass the use of radionuclide agents, for example ¹³¹I, ³²P, ⁹⁰Y, ⁸⁹Sr, ¹⁵³Sm or ²²³Ra. Such radionuclide therapies are well known and commercially available.

According to a further aspect of the invention there is provided a compound of the invention, or a pharmaceutically acceptable salt thereof as defined hereinbefore for use in the treatment of cancer conjointly with radiotherapy.

According to a further aspect of the invention there is provided a method of treatment of a human or animal subject suffering from a cancer comprising administering to the subject a therapeutically effective amount of a compound of the invention, or a pharmaceutically acceptable salt thereof simultaneously, sequentially or separately with radiotherapy.

### Biological Assays

The biological effects of the compounds may be assessed using one of more of the assays described herein in the Examples.

In certain embodiments the compounds have an pIC₅₀ of 7.0 or less in the MASTLwt activity assay described in the Examples.

### Synthesis

Compounds of the invention may be prepared using analogous methods to the General Synthetic Methods described in the Examples. In the description of the synthetic methods described below and in the referenced synthetic methods that are used to prepare the staring materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

Necessary starting materials may be obtained by standard procedures of organic chemistry. The preparation of such starting materials is described in conjunction with the following representative process variants and within the accompanying Examples. Alternatively, necessary starting materials are obtainable by analogous procedures to those illustrated which are within the ordinary skill of an organic chemist.

It will be appreciated that during the synthesis of the compounds of the invention in the processes defined below, or during the synthesis of certain starting materials, it may be desirable to protect certain substituent groups to prevent their undesired reaction. The skilled chemist will appreciate when such protection is required, and how such protecting groups may be put in place, and later removed.

For examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of example, a suitable protecting group for an amino or alkylamino group is, for example, an acyl group, for example an alkanoyl group such as acetyl or trifluoroacetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively, an acyl group such as a tert-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example BF₃. OEt₂. A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, or sodium hydroxide, or ammonia. Alternatively, an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

### EXAMPLES

Throughout this specification these abbreviations have the following meanings:

| | |
|---|---|
| Aq. = aqueous | DCM = dichloromethane |
| DMF = N,N-dimethylformamide | DMSO = dimethyl sulfoxide |
| Et = ethyl | EtOAc = ethyl acetate |
| h = hours | MeOH = methanol |
| Me = methyl | min = minutes |
| mol = mole | \|cPr = cyclopropyl |
| iPr = isopropyl | Rt = retention time |
| RT = Room temperature | Sat. = saturated |
| THF = tetrahydrofuran | T3P = propylphosphonic |
| | anhydride |
| DIEA = N,N-Diisopropylethylamine | Et3N = Triethylamine |
| HOBt = 1-Hydroxybenzotriazole hydrate | NH4Cl = Ammonium |
| EDCI•HCl = 1-Ethyl-3-(3- | chloride |
| dimethylaminopropyl)carbodiimide | |
| Hydrochloride | |
| EtOH = Ethanol | NaOAc = Sodium acetate |
| NaHCO3 = Sodium bicarbonate | NaOH = Sodium hydroxide |
| KF = Potassium fluoride | MeMgBr = Methylmagnesium |
| | bromide |
| NaBH3CN = Sodium cyanoborohydride | NH3 = Ammonia |
| HATU = 1- | |
| [Bis(dimethylamino)methylene]-1H-1,2,3- | |
| triazolo[4,5-b]pyridinium 3-oxid | |
| hexafluorophosphate | |
| NBS = N-Bromosuccinimide | NH2NH2•H2O = Hydrazine |
| | monohydrate |
| H3PO4 = Phosphoric acid | Na2SO4 = Sodium sulfate |
| MeCN = Acetonitrile | |

### Materials and Methods

Solvents, reagents and starting materials were purchased from commercial vendors and used as received unless otherwise described. All reactions were performed at RT unless otherwise stated. Flash column chromatography was carried out using pre-packed columns filled with Merck flash silica gel 60 (40-63 μm) or C18 flash silica on an ISCO Combiflash Nextgen or a Biotage Selekt.

### LCMS

LCMS data was recorded on a Waters 2695 HPLC using a Waters 2487 UV detector and a Thermo LCQ ESI-MS. Samples were eluted through a Phenomenex Luna 3 u C18 50 mm×4.6 mm column, using water and Acetonitrile acidified by 0.1% formic acid at 1.5 mL/min and detected at 254 nm.

The following methods were used:

Method 1:4 minute method

The gradient employed was:

| Time | % Water + | % Acetonitrile + |
|---|---|---|
| (minutes) | 0.1% formic acid | 0.1% formic acid |
| 0.0 | 65 | 35 |
| 3.5 | 10 | 90 |
| 3.9 | 10 | 90 |
| 4.0 | 65 | 35 |

Method 2:5 minute method

The gradient employed was:

| Time | % Water + | % Acetonitrile + |
|---|---|---|
| (minutes) | 0.1% formic acid | 0.1% formic acid |
| 0.0 | 90 | 10 |
| 0.5 | 90 | 10 |
| 4.0 | 10 | 90 |
| 4.7 | 10 | 90 |
| 4.8 | 65 | 35 |
| 5.0 | 65 | 35 |

Method 3:10 minute method

The gradient employed was:

| Time | % Water + | % Acetonitrile + |
|---|---|---|
| (minutes) | 0.1% formic acid | 0.1% formic acid |
| 0.0 | 95 | 5 |
| 8.0 | 5 | 95 |
| 8.5 | 5 | 95 |
| 9.0 | 95 | 5 |
| 9.5 | 95 | 5 |

### Chemical Synthesis

Microwave reactions were conducted using a Biotage Robot 60+microwave reactor.

### Intermediate A1

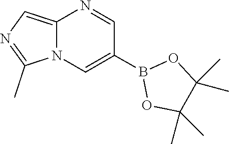 [SMILES: Cc1ncc2ncc(B3OC(C)(C)C(C)(C)O3)cn12]

### 6-methyl-3-(4,4,5,5-tetramethyl-1,3,2-dioxaborolan-2-yl) imidazo[1,5-a]pyrimidine

Step 1: N-[(5-bromopyrimidin-2-yl)methyl]acetamide

To a solution of (5-bromopyrimidin-2-yl) methanamine (3.23 g, 17.16 mmol) in DCM (30 mL) was added dropwise Et₃N (5.21 g, 51.48 mmol, 7.17 mL) and acetyl chloride (2.69 g, 34.32 mmol) at 0 ᵢÉ. After addition, the mixture was stirred at 25 ᵢÉ for 16 h. The reaction mixture was diluted with H₂O (50 mL) and extracted with DCM (100 mL×3). The combined organic layers were washed with brine (40 mL×3), dried over anhydrous Na₂SO₄, filtered and concentrated under reduced pressure to give N-[(5-bromopyrimidin-2-yl)methyl]acetamide (2.44 g, 53.84% yield) as a yellow solid.

MS(ESI) m/z=232.0 [M+H]⁺.

Step 2:3-bromo-6-methyl-imidazo[1,5-a]pyrimidine

A mixture of N-[(5-bromopyrimidin-2-yl)methyl]acetamide (2.64 g, 11.48 mmol), POCl₃ (5.28 g, 34.43 mmol, 3.20 mL) in toluene (25 mL) was degassed and purged with N₂ and then the mixture was stirred at 110 ᵢÉ for 12 h under N₂. The reaction mixture was diluted with H₂O (50 mL) and extracted with EA (100 mL×3). The combined organic layers were washed with brine (40 mL×3), dried over anhydrous Na₂SO₄, filtered and concentrated under reduced pressure to give 3-bromo-6-methyl-imidazo[1,5-a]pyrimidine (0.65 g, 26.02% yield) as a yellow solid.

MS(ESI) m/z=212.0 [M+H]⁺.

Step 3: 6-methyl-3-(4,4,5,5-tetramethyl-1,3,2-dioxaborolan-2-yl) imidazo[1,5-a]pyrimidine

A mixture of 3-bromo-6-methyl-imidazo[1,5-a]pyrimidine (200 mg, 943.19 μmol), 4,4,5,5-tetramethyl-2-(4,4,5,5-tetramethyl-1,3,2-dioxaborolan-2-yl)-1,3,2-dioxaborolane (2.40 g, 9.43 mmol), AcOK (277.70 mg, 2.83 mmol) and Pd(dppf) Cl₂ (34.51 mg, 47.16 μmol) in dioxane (3 mL) was degassed and purged with N₂ and then the mixture was stirred at 100 ᵢ É for 5 h under N₂. The reaction mixture was concentrated under reduced pressure to give 6-methyl-3-(4,4,5,5-tetramethyl-1,3,2-dioxaborolan-2-yl) imidazo[1,5-a]pyrimidine (2.67 g, crude) as a yellow solid.

MS(ESI) m/z=178.0 [boronic acid+H]⁺.

### Intermediate A2

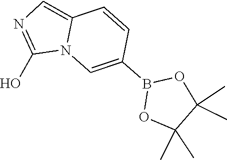 [SMILES: CC1(C)OB(c2ccc3cnc(O)n3c2)OC1(C)C]

### 6-(4,4,5,5-tetramethyl-1,3,2-dioxaborolan-2-yl) imidazo[1,5-a]pyridin-3-ol

Step 1:6-bromoimidazo[1,5-a]pyridin-3-ol

To a solution of (5-bromo-2-pyridyl) methanamine (3 g, 13.42 mmol) in DCM (60 mL) were dropwise added a solution of NaHCO₃ (3.38 g, 40.27 mmol) in H₂O (30 mL) and a solution of Triphosgene (3.98 g, 13.42 mmol) in DCM (60 mL) at 0° C., then the mixture was stirred at 25° C. under N₂ for 3 hr. Separate the aqueous layer and extract with DCM (50 mL×3). The organic layer was washed with brine (50 mL), dried over with Na₂SO₄ and concentrated under reduced pressure to give a residue. The residue was purified by flash silica gel chromatography to give the 6-bromoimidazo[1,5-a]pyridin-3-ol (640 mg, 22.38% yield) as a yellow solid.

MS(ESI) m/z=213.1 [M+H]⁺.

Step 2:6-(4,4,5,5-tetramethyl-1,3,2-dioxaborolan-2-yl) imidazo[1,5-a]pyridin-3-ol

To a stirred solution of 6-bromoimidazo[1,5-a]pyridin-3-ol (300 mg, 1.41 mmol) in 1,4-Dioxane (4 mL) were added 4,4,5,5-tetramethyl-2-(4,4,5,5-tetramethyl-1,3,2-dioxaborolan-2-yl)-1,3,2-dioxaborolane (429.13 mg, 1.69 mmol), AcOK (276.42 mg, 2.82 mmol) and Pd(dppf) C₁₂ (115.00 mg, 140.82 μmol) under N₂, and the reaction mixture was stirred at 110° C. for 2 h. The reaction mixture was concentrated under reduced pressure to give a residue. The residue was purified by flash silica gel chromatography to give 6-(4,4,5,5-tetramethyl-1,3,2-dioxaborolan-2-yl) imidazo[1,5-a]pyridin-3-ol (300 mg, crude) as a yellow solid.

MS(ESI) m/z=161.1 [boronic acid+H]⁺.

### Intermediate A3

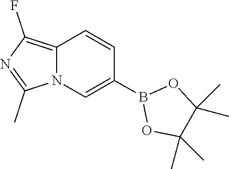 [SMILES: Cc1nc(F)c2ccc(B3OC(C)(C)C(C)(C)O3)cn12]

### 1-fluoro-3-methyl-6-(4,4,5,5-tetramethyl-1,3,2-dioxaborolan-2-yl) imidazo[1,5-a]peridine

Step 1:6-bromo-1-fluoro-3-methyl-imidazo[1,5-a]pyridine

To a solution of 6-bromo-3-methyl-imidazo[1,5-a]pyridine (250 mg, 1.18 mmol) in DMF (5 mL) was added 1-fluoro-2, 4, 6-trimethyl-pyridin-1-ium; tetrafluoroborate (537.72 mg, 2.37 mmol). After addition, the mixture was stirred at 60° C. for 1 hr, and then 1-fluoro-2,4,6-trimethyl-pyridin-1-ium; tetrafluoroborate (537.72 mg, 2.37 mmol) was added. After addition, the mixture was stirred at 60° C. for 1 hr, and then 1-fluoro-2,4,6-trimethyl-pyridin-1-ium; tetrafluoroborate (537.72 mg, 2.37 mmol) was added. The mixture was stirred at 60° C. for 10 hr under N₂. The reaction mixture was quenched by addition H₂O (5 mL). The residue was purified by column chromatography to give product 6-bromo-1-fluoro-3-methyl-imidazo[1,5-a]pyridine (43 mg, 187.73 μmol, 15.85% yield) as a yellow solid.

1H NMR (400 MHZ, DMSO-d6) δ=8.35 (s, 1H), 7.42 (d, 1H), 6.70 (d, 1H), 2.52 (s, 3H).

Step 2:1-fluoro-3-methyl-6-(4,4,5,5-tetramethyl-1,3,2-dioxaborolan-2-yl) imidazo[1,5-a]pyridine

To a solution of 6-bromo-1-fluoro-3-methyl-imidazo[1,5-a]pyridine (88 mg, 384.20 μmol) in dioxane (2 mL) were added 4,4,5,5-tetramethyl-2-(4,4,5,5-tetramethyl-1,3,2-dioxaborolan-2-yl)-1,3,2-dioxaborolane (117.08 mg, 461.04 μmol), KOAc (75.41 mg, 768.40 μmol) and Pd(dppf) C₁₂ (15.69 mg, 19.21 μmol). The mixture was stirred at 110° C. for 12 hr under N₂ atmosphere. The mixture was filtered and then the mixture was concentrated under reduced pressure to give a residue. The residue was added a little DCM (0.5 mL), and then the suspension was filtered through a pad of Celite and the filter cake was washed with Petroleum ether (5 mL). The combined filtrates were concentrated to dryness to give crude product 1-fluoro-3-methyl-6-(4,4,5,5-tetramethyl-1,3,2-dioxaborolan-2-yl) imidazo[1,5-a]pyridine (130 mg, crude) as a brown oil.

1H NMR (400 MHZ, DMSO-d6) δ=8.05 (s, 1H), 7.37 (dd, 1H), 6.69 (d, 1H), 2.56 (s, 3H), 1.31 (s, 12H).

### Intermediate A4

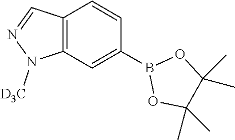 [SMILES: [2H]C([2H])([2H])n1ncc2ccc(B3OC(C)(C)C(C)(C)O3)cc21]

### 6-(4,4,5,5-tetramethyl-1,3,2-dioxaborolan-2-yl)-1-(trideuteriomethyl) indazole

Step 1:6-bromo-1-(trideuteriomethyl) indazole

To a stirred solution of 6-bromo-1H-indazole (20 g, 101.51 mmol) in THF (240 mL) were added 60% NaH (4.47 g, 111.66 mmol) at 0° C., Then trideuterio (iodo) methane (21.61 g, 152.26 mmol) was added, and the reaction mixture was stirred at 20° C. under N₂ for 2 h. The reaction mixture was quenched with H₂O (10 mL), and extracted with ethyl acetate (20 mL×3). The combined organic layers were washed with brine (20 mL), dried over anhydrous sodium sulfate, and concentrated. The residue was purified by flash silica gel chromatography to give 6-bromo-1-(trideuteriomethyl) indazole (11.6 g, 53.38% yield) as red oil.

MS(ESI) m/z=214.1 [M+H]⁺.

Step 2:6-(4,4,5,5-tetramethyl-1,3,2-dioxaborolan-2-yl)-1-(trideuteriomethyl) indazole

To a stirred solution of 6-bromo-1-(trideuteriomethyl) indazole (10.6 g, 49.51 mmol) in 1,4-Dioxane (206 mL) were added 4,4,5,5-tetramethyl-2-(4,4,5,5-tetramethyl-1,3,2-dioxaborolan-2-yl)-1,3,2-dioxaborolane (15.09 g, 59.42 mmol), AcOK (9.72 g, 99.03 mmol) and Pd(dppf) C₁₂ (4.04 g, 4.95 mmol) under N₂ and the reaction mixture was stirred at 110° C. for 12 h. The reaction mixture was concentrated under reduced pressure to give a residue. The residue was purified by flash silica gel chromatography to give 6-(4,4,5,5-tetramethyl-1,3,2-dioxaborolan-2-yl)-1-(trideuteriomethyl) indazole (11.4 g, 88.16% yield) as an off-white solid.

1H NMR (400 MHZ, DMSO-d6) δ=8.06 (d, 1H), 7.94 (d, 1H), 7.74 (dd, 1H), 7.40 (d, 1H), 1.32 (s, 12H).

### General Method for Intermediate B

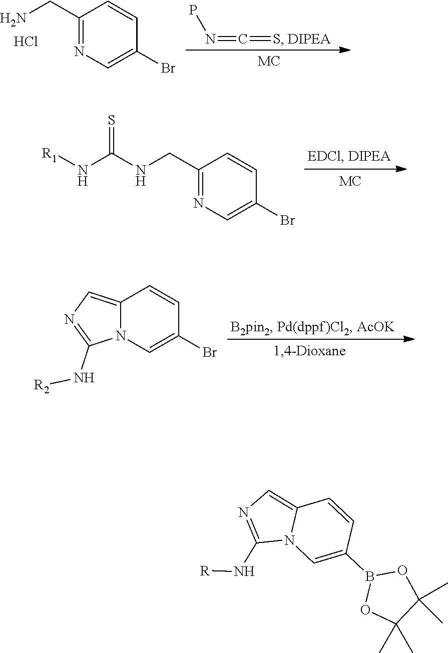

### Intermediate B1

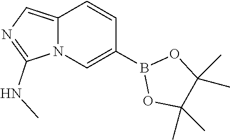 [SMILES: CNc1ncc2ccc(B3OC(C)(C)C(C)(C)O3)cn12]

### N-methyl-6-(4,4,5,5-tetramethyl-1,3,2-dioxaborolan-2-yl) imidazo[1,5-a]pyridin-3-amine

Step 1:1-[(5-bromo-2-pyridyl)methyl]-3-methyl-thiourea

To a stirred solution of (5-bromo-2-pyridyl) methanamine (2 g, 8.95 mmol) in DCM (20 mL) were added DIPEA (2.31 g, 17.90 mmol) and methylimino(thioxo) methane (654.30 mg, 8.95 mmol) and the reaction mixture was stirred at 15° C. under N₂ for 16 h. The reaction mixture was concentrated under reduced pressure to to give a residue. The residue was purified by flash silica gel chromatography to give 1-[(5-bromo-2-pyridyl)methyl]-3-methyl-thiourea (2.13 g, 91.49% yield) as a green solid.

MS(ESI) m/z=261.5 [M+H]⁺.

Step 2:6-bromo-N-methyl-imidazo[1,5-a]pyridin-3-amine

To a stirred solution of 1-[(5-bromo-2-pyridyl)methyl]-3-methyl-thiourea (2.13 g, 8.19 mmol) in DCM (18 mL) and DMF (6 mL) were added DIPEA (2.12 g, 16.37 mmol) and EDCI (2.35 g, 12.28 mmol), and the reaction mixture was stirred at 15° C. under N₂ for 16 h. The reaction mixture was concentrated under reduced pressure to to give a residue. The residue was purified by flash silica gel chromatography to give 6-bromo-N-methyl-imidazo[1,5-a]pyridin-3-amine (1.3 g, 70.23% yield) as a green solid.

MS(ESI) m/z=226.1 [M+H]⁺.

Step 3: N-methyl-6-(4,4,5,5-tetramethyl-1,3,2-dioxaborolan-2-yl) imidazo[1,5-a]pyridin-3-amine

To a stirred solution of 6-bromo-N-methyl-imidazo[1,5-a]pyridin-3-amine (1.3 g, 5.75 mmol) in dioxane (30 mL) were added 4,4,5,5-tetramethyl-2-(4,4,5,5-tetramethyl-1,3,2-dioxaborolan-2-yl)-1,3,2-dioxaborolane (1.75 g, 6.90 mmol), AcOK (1.13 g, 11.50 mmol) and Pd(dppf) Cl₂ (469.60 mg, 575.04 μmol) and the reaction mixture was stirred at 110° C. under N₂ for 12 h. The reaction mixture was concentrated under reduced pressure to give a residue. The residue was purified by flash silica gel chromatography to to give N-methyl-6-(4,4,5,5-tetramethyl-1,3,2-dioxaborolan-2-yl) imidazo[1,5-a]pyridin-3-amine (597 mg, 2.19 mmol, 38.01% yield) as a green solid.

1H NMR (400 MHz, DMSO-d6) δ=8.06 (s, 1H), 7.93 (s, 1H), 7.16 (d, 1H), 6.86 (s, 1H), 6.48-6.44 (m, 1H), 6.42 (d, 1H), 2.88 (d, 3H), 1.29 (s, 12H).

| Example | Structure | NMR |
|---|---|---|
| Intermediate-B2 | 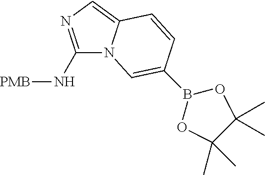 | 1H NMR (DMSO-d6) δ = 8:18 (s, 1H), 7.32 (d, 2H), 7.16 (d, 1H), 7.00 (t, 1H), 6.91-6.83 (m 3H), 6.43 (d, 1H), 4.44 (d, 2H), 3.71 (s, 3H), 1.28 (s, 12H). |

General Method for Intermediate C (Imidazole amine)

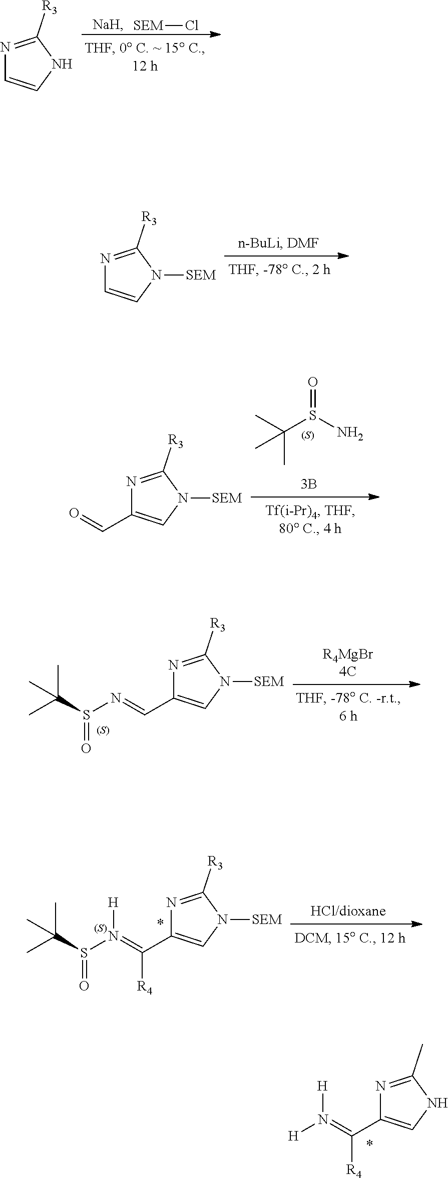

### Intermediate C1A

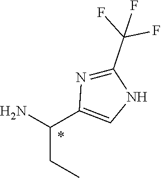

### (R or S)-1-(2-(trifluoromethyl)-1H-imidazol-4-yl) propan-1-amine

Step 1: trimethyl-[2-[[2-(trifluoromethyl) imidazol-1-yl]methoxy]ethyl]silane

A mixture of 2-(trifluoromethyl)-1H-imidazole (3.00 g, 22.05 mmol) in THF (60 mL) was degassed and purged with N₂ for 3 times, and then the mixture was added 60% NaH (3.53 g, 88.19 mmol) and stirred at 0° C. for 2 h under N₂. Then SEM-C₁ (4.41 g, 26.46 mmol) was added at 0° C. and stirred at 25° C. for 2 h. The reaction mixture was diluted with H₂O (80 mL) and extracted with EtOAc (60 mL×3). The combined organic layers were dried over anhydrous Na₂SO₄, filtered, and concentrated under reduced pressure to give a residue. The residue was purified by column chromatography give trimethyl-[2-[[2-(trifluoromethyl) imidazol-1-yl]methoxy]ethyl]silane(7 g, 95.37% yield) as a yellow oil.

1H NMR (400 MHZ, CDCl₃): δ=7.21 (d, 1H), 7.15 (d, 1H), 5.42 (s, 2H), 3.57-3.51 (m, 2H), 0.92-0.84 (m, 2H), 0.00 (s, 9H).

Step 2:2-(trifluoromethyl)-1-(2-trimethylsilylethoxymethyl) imidazole-4-carbaldehyde

A mixture of trimethyl-[2-[[2-(trifluoromethyl) imidazol-1-yl]methoxy]ethyl]silane(5.7 g, 21.40 mmol) in THF (60 mL) was added n-BuLi (2.5 M, 10.27 mL) and stirred at −78° C. for 0.5 hr under N₂. Then the reaction was added DMF (9.39 g, 128.41 mmol) and stirred at −78° C. for 1 h. The reaction mixture was quenched by addition aqueous NH₄Cl (100 mL) at 0° C., and extracted with EtOAc (80 mL×3). The combined organic layers were dried over anhydrous Na₂SO₄, filtered, and concentrated under reduced pressure to give a residue. The residue was purified by column chromatography to obtain 2-(trifluoromethyl)-1-(2-trimethylsilylethoxymethyl) imidazole-4-carbaldehyde (4.7 g, 63.42% yield) as a white oil.

1H NMR (400 MHZ, CDCl₃) δ=9.92 (s, 1H), 7.87 (s, 1H), 5.88 (s, 2H), 3.65-3.60 (m, 2H), 0.96-0.91 (m, 2H), 0.00 (s, 9H).

Step 3: (NE,R)-2-methyl-N-[[2-(trifluoromethyl)-1-(2-trimethylsilylethoxymethyl) imidazol-4-yl]methylene]propane-2-sulfinamide

To a solution of 2-(trifluoromethyl)-1-(2-trimethylsilylethoxymethyl) imidazole-4-carbaldehyde (2.3 g, 7.81 mmol) in THF (50 mL) was added Ti (OEt) 4 (8.91 g, 39.07 mmol) and (R)-2-methylpropane-2-sulfinamide (4.74 g, 39.07 mmol). The mixture was stirred at 80° C. for 3 h. The reaction mixture was quenched by H₂O (10 mL) at 25° C., and then filtered and concentrated under reduced pressure to give a residue. The residue was purified by column chromatography to obtain (NE,R)-2-methyl-N-[[2-(trifluoromethyl)-1-(2-trimethylsilylethoxymethyl) imidazol-4-yl]methylene]propane-2-sulfinamide (2.2 g, 67.28% yield) as a white oil.

1H NMR (400 MHZ, CD3OD) δ=8.61 (s, 1H), 7.83 (s, 1H), 5.96 (d, 1H), 5.87 (d, 1H), 3.62 (t, 2H), 1.28 (s, 9H), 0.95-0.85 (m, 2H),−0.03 (s, 9H).

Step 4: (R)-2-methyl-N-((S or R)-1-(2-(trifluoromethyl)-1-((2-(trimethylsilyl) ethoxy)methyl)-1H-imidazol-4-yl) propyl) propane-2-sulfinamide

To a solution of EtMgBr (3 M, 10 mL) was added (NE,R)-2-methyl-N-[[2-(trifluoromethyl)-1-(2-trimethylsilylethoxymethyl) imidazol-4-yl]methylene]propane-2-sulfinamide (500 mg, 1.26 mmol) in THF (5 mL) at 0° C. The mixture was stirred at 25° C. for 12 h. The reaction mixture was quenched by addition aqueous NH₄Cl (60 mL) at 25° C., and extracted with EtOAc (40 mL×3). The combined organic layers were dried over anhydrous Na₂SO₄, filtered, and concentrated under reduced pressure to give a residue. The residue was purified by column chromatography to give (R)-2-methyl-N-((S or R)-1-(2-(trifluoromethyl)-1-((2-(trimethylsilyl) ethoxy)methyl)-1H-imidazol-4-yl) propyl) propane-2-sulfinamide (200 mg, 33.47% yield) as an off-white oil.

1H NMR (400 MHZ, CD3OD) δ=7.19 (s, 1H), 5.64 (d, 1H), 5.48 (d, 1H), 4.49 (t, 1H), 3.65-3.55 (m, 2H), 2.03 (t, 2H), 1.19 (s, 9H), 1.02 (t, 3H), 0.96-0.88 (m, 2H), 0.00 (s, 9H)

Step 5: (S or R)-1-[2-(trifluoromethyl)-1H-imidazol-4-yl]propan-1-amine

To a solution of (R)-2-methyl-N-((S or R)-1-(2-(trifluoromethyl)-1-((2-(trimethylsilyl) ethoxy)methyl)-1H-imidazol-4-yl) propyl) propane-2-sulfinamide (160 mg, 374.18 μmol) in DCM (1.5 mL) was added HCl/dioxane (4 M, 0.5 mL). The mixture was stirred at 25° C. for 0.5 h. The reaction mixture was concentrated under reduced pressure to give a residue. The residue was no further purification and (S or R)-1-[2-(trifluoromethyl)-1H-imidazol-4-yl]propan-1-amine (85 mg, crude) was obtained as a colorless oil.

MS(ESI) m/z=192.2 [M−H]⁻.

| Example | Structure | LCMS |
|---|---|---|
| Intermediate C2A | 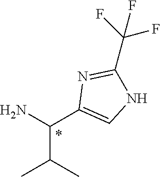 | MS (ESI) m/z = 208.1 [M + H]+. |
| Intermediate C2B | | MS (ESI) m/z = |
| | | 208.1 [M + H]+. |
| Intermediate C3A | 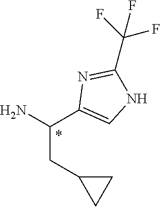 [SMILES: NC(CC1CC1)c1c[nH]c(C(F)(F)F)n1] | MS (ESI) m/z = 220.0 [M + H]+. |
| Intermediate C3B | | MS (ESI) m/z = |
| | | 220.1 [M + H]+. |
| Intermediate C4A | 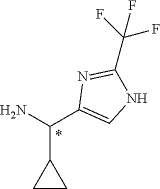 [SMILES: NC(c1c[nH]c(C(F)(F)F)n1)C1CC1] | MS (ESI) m/z = 206.1 [M + H]+. |
| Intermediate C4B | | MS (ESI) m/z = |
| | | 206.3 [M + H]+. |
| Intermediate C5A | 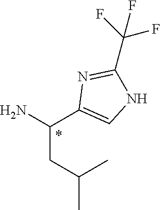 | MS (ESI) m/z = 221.2 [M + H]+. |
| Intermediate C5B | | MS (ESI) m/z = |
| | | 221.1 [M + H]+. |
| Intermediate C6A | 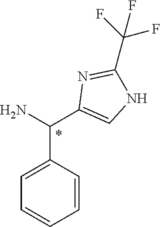 [SMILES: NC(c1ccccc1)c1c[nH]c(C(F)(F)F)n1] | MS (ESI) m/z = 2 42.1 [M + H]+. |
| Intermediate C7A | 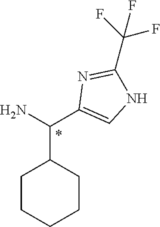 [SMILES: NC(c1c[nH]c(C(F)(F)F)n1)C1CCCCC1] | MS (ESI) m/z = 248.1 [M + H]+. |
| Intermediate C7B | | MS (ESI) m/z = |
| | | 248.1 [M + H]+. |
| Intermediate C8A | 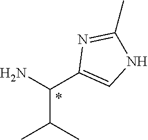 | MS (ESI) m/z = 154.1 [M + H]+. |
| Intermediate C8B | | MS (ESI) m/z = |
| | | 154.0 [M + H]+. |

General Method for Intermediate D (Indazole amine)

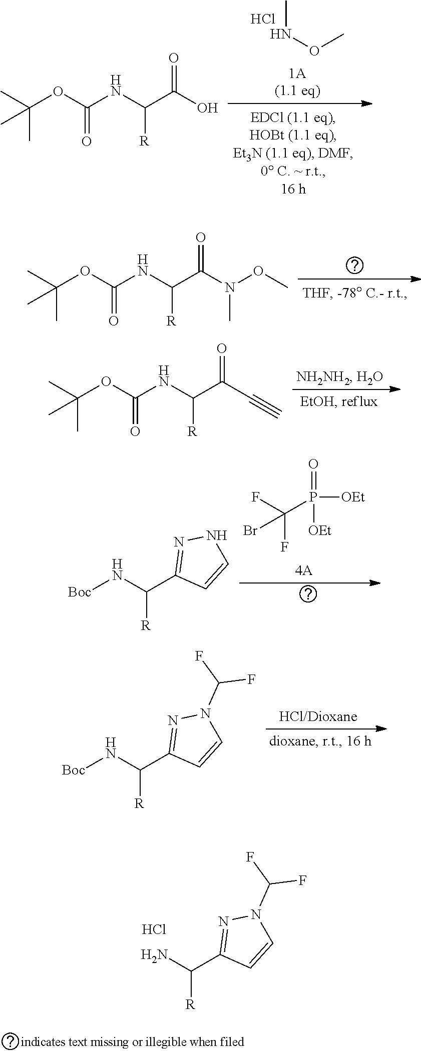

### Intermediate D1A

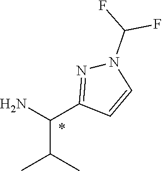

### (1S or 1R)-1-[1-(difluoromethyl) pyrazol-3-yl]-2-methyl-propan-1-amine

Step 1: tert-butyl N-[(1S or 1R)-1-[methoxy(methyl) carbamoyl]-2-methyl-propyl]carbamate

To a solution of (2S or 2R)-2-(tert-butoxycarbonylamino)-3-methyl-butanoic acid (5 g, 23.01 mmol) in DMF (50 mL) was added EDCI (4.85 g, 25.32 mmol) and HOBt (3.42 g, 25.32 mmol) at 0° C., then N-methoxymethanamine; hydrochloride (2.47 g, 25.32 mmol) and TEA (2.56 g, 25.32 mmol) was added at 15° C. and stirred at 15° C. for 16 h under N₂ atmosphere. It was concentrated under reduced pressure to give a crude product. The crude product was purified by flash silica gel chromatography y to give the tert-butyl N-[(1S or 1R)-1-[methoxy(methyl) carbamoyl]-2-methyl-propyl]carbamate (6.1 g, crude) as a yellow solid.

1H NMR (400 MHZ, CDCl₃) δ=5.13 (d, 1H), 4.57 (br s, 1H), 3.77 (s, 3H), 3.21 (s, 3H), 2.04-1.92 (m, 1H), 1.43 (s, 9H), 0.95 (d, 3H), 0.90 (d, 3H).

Step 2: tert-butyl(R)-(2-methyl-4-oxohex-5-yn-3-yl) carbamate

To a solution of tert-butyl N-[(1S or 1R)-1-[methoxy(methyl) carbamoyl]-2-methyl-propyl]carbamate (6 g, 23.05 mmol) in THF (10 mL) was added bromo (ethynyl) magnesium (0.5 M, 230.48 mL, in THF) at −78° C. under N₂ for 1 h and then stirred at 30° C. for 16 h under N₂ atmosphere. THF was evaporated in vacuo, and the aqueous residue was extracted with EtOAc (100 mL). The combined organic phase was washed with sat. NaHCO₃ aq. (15 mL), and brine (15 mL×3), dried over Na₂SO₄, filtered and the filtrate was concentrated under reduced pressure to give tert-butyl(R or S)-(2-methyl-4-oxohex-5-yn-3-yl) carbamate (5.46 g, crude) as a brown oil.

1H NMR (400 MHZ, CDCl₃) δ=5.04 (br d, 1H), 4.40 (dd, 1H), 3.37 (s, 1H), 2.46 (m, 1H), 1.45 (s, 9H), 1.05 (d, 3H), 0.84 (d, 3H).

Step 3: tert-butyl N-[(1S or 1R)-2-methyl-1-(1H-pyrazol-3-yl) propyl]carbamate

To a solution of tert-butyl(R or S)-(2-methyl-4-oxohex-5-yn-3-yl) carbamate (5.46 g, 24.24 mmol) in EtOH (100 mL) was added NH₂NH₂·H₂O (2.48 g, 48.47 mmol) at 80° C. and the mixture was stirred at 80° C. under N₂ for 0.5 h. The suspension was filtered, and the filtrate was concentrated under reduced pressure to afford a crude product. The crude product was purified by column chromatography to give tert-butyl N-[(1S or 1R)-2-methyl-1-(1H-pyrazol-3-yl) propyl]carbamate (2.2 g, 37.93% yield) as a yellow oil.

1H NMR (400 MHz, CDCl₃) δ=7.51 (d, J=1.6 Hz, 1H), 6.16 (br s, 1H), 5.35 (d, J=5.6 Hz, 1H), 4.64 (d, J=6.8 Hz, 1H), 2.14 (br d, J=6.0 Hz, 1H), 0.92 (dd, J=16.0, 6.8 Hz, 6H).

Step 4: tert-butyl N-[(1S or 1R)-1-[1-(difluoromethyl) pyrazol-3-yl]-2-methyl-propyl]carbamate

To a solution of tert-butyl N-[(1S or 1R)-2-methyl-1-(1H-pyrazol-3-yl) propyl]carbamate (2 g, 8.36 mmol) and KF (1.46 g, 25.07 mmol) in MeCN (30 mL) was added 1-[[bromo (difluoro) methyl]-ethoxy-phosphoryl]oxyethane (2.68 g, 10.03 mmol). The mixture was stirred at 30° C. for 12 h under N₂. The reaction mixture was diluted with water (50 mL), and the resulting mixture was extracted with EtOAc (30 mL×3). The combined organic layer was washed with brine (30 mL), dried over anhydrous Na₂SO₄ and concentrated under reduced pressure to give a residue. The residue was purified by column chromatography to give tert-butyl N-[(1S or 1R)-1-[1-(difluoromethyl) pyrazol-3-yl]-2-methyl-propyl]carbamate (440 mg, 18.20% yield) as a white solid.

1H NMR (400 MHZ, CDCl₃) δ=7.73 (d, J=2.4 Hz, 1H), 7.14 (t, J=61.2 Hz, 1H), 6.30 (d, J=2.4 Hz, 1H), 5.15 (br s, 1H), 4.67 (br s, 1H), 2.14-2.10 (m, 1H), 1.49-1.42 (s, 9H), 0.90 (d, J=6.8 Hz, 6H).

Step 5: (1S or 1R)-1-[1-(difluoromethyl) pyrazol-3-yl]-2-methyl-propan-1-amine

To a solution of give tert-butyl N-[(1S or 1R)-1-[1-(difluoromethyl) pyrazol-3-yl]-2-methyl-propyl]carbamate (440 mg, 1.52 mmol) in 1,4-Dioxane (8 mL) was added HCl/dioxane (8 mL, 4M) at 15° C. and stirred at 15° C. for 16 h under N₂. It was concentrated under reduced pressure to give (1S or 1R)-1-[1-(difluoromethyl) pyrazol-3-yl]-2-methyl-propan-1-amine (330.3 mg, crude) as a white solid.

1H NMR (400 MHZ, DMSO-d6) δ=8.68 (br s, 3H), 8.31 (d, J=2.8 Hz, 1H), 7.84 (t, J=60.0 Hz, 1H), 6.72 (d, J=2.8 Hz, 1H), 4.15 (br s, 1H), 2.29-2.17 (m, 1H), 0.96 (d, J=6.8 Hz, 3H), 0.80 (d, J=6.8 Hz, 3H).

| Example | Structure | LCMS |
|---|---|---|
| Intermediate D2 | 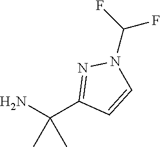 | MS (ESI) m/z = 176.1 [M + H]+. |
| Intermediate D3A | 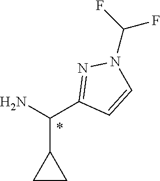 [SMILES: NC(c1ccn(C(F)F)n1)C1CC1] | MS (ESI) m/z = 188.0 [M + H]+. |

### Intermediate E

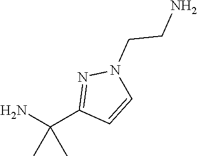

### tert-butyl(2-(1-(2-aminoethyl)-1H-pyrazol-3-yl) propan-2-yl) carbamate

Step 1: give tert-butyl N-[2-[methoxy(methyl)amino]-1, 1-dimethyl-2-oxo-ethyl]carbamate

To a stirred mixture of 2-(tert-butoxycarbonylamino)-2-methyl-propanoic acid (91 mg, 447.76 μmol), N-methoxymethanamine (52.41 mg, 537.31 μmol), DMAP (65.64 g, 537.31 μmol), DIPEA (69.44 mg, 537.31 μmol) in DCM (2 mL), then DCC (110.86 mg, 537.31 μmol) was added, the mixture was stirred at 20° C. for 16 hr. The mixture was filtered to remove precipitated N,N′-dicyclohexylurea and the filtrate was evaporated under reduced pressure. The residue was dissolved in ethyl acetate (20 mL), washed with 10% aqueous citric acid (20 mL×3), 10% aqueous NaHCO₃ (10 mL) and saturated aqueous sodium chloride (20 mL×3), and dried over anhydrous Na₂SO₄. The residue was purified by flash silica gel chromatography to give tert-butyl

N-[2-[methoxy(methyl)amino]-1, 1-dimethyl-2-oxo-ethyl]carbamate (105 mg, 76.17% yield) as a white solid.

MS(ESI) m/z=247.1 [M+H]⁺.

Step 2: give tert-butyl N-(1,1-dimethyl-2-oxo-but-3-ynyl) carbamate

To a solution of tert-butyl N-[2-[methoxy(methyl)amino]-1,1-dimethyl-2-oxo-ethyl]carbamate (32 mg, 129.92 μmol) in THF (5 mL) at −78° C. was dropwise added bromo (ethynyl) magnesium (0.5 M, 1.04 mL), the mixture was stirred 25° C. for 24 hr under N₂. The reaction mixture was quenched by addition H₂O (5 mL) at 30° C., and extracted with EtOAc (5 mL×3). The combined organic layers were washed with brine (5 mL), dried over anhydrous Na₂SO₄, filtered and concentrated under reduced pressure to give tert-butyl N-(1,1-dimethyl-2-oxo-but-3-ynyl) carbamate (45 mg, crude) as a yellow solid.

MS(ESI) m/z=156.1 [M-tBu+H]⁺.

Step 3: tert-butyl N-[1-methyl-1-(1H-pyrazol-3-yl)ethyl]carbamate

To a solution of tert-butyl N-(1,1-dimethyl-2-oxo-but-3-ynyl) carbamate (44 mg, 208.28 μmol) in EtOH (5 mL) was added NH2NH2ᵢo H₂O (24.53 mg, 416.55 μmol), the mixture was stirred at 80° C. for 0.5 hr. The reaction mixture was concentrated under reduced pressure to remove EtOH and residual N2H4ᵢo H2O at 30° C. The residue was diluted with H₂O (10 mL) and extracted with EtOAc (10 mL×3). The combined organic layers were washed with brine (10 mL), dried over anhydrous Na₂SO₄, filtered and concentrated under reduced pressure to give a residue. The residue was purified by flash silica gel chromatography to give tert-butyl N-[1-methyl-1-(1H-pyrazol-3-yl)ethyl]carbamate (17 mg, 20.65% yield) as an yellow oil.

MS(ESI) m/z=226.1 [M+H]⁺.

Step 4: tert-butyl N-[1-[1-[2-(benzyloxycarbonylamino) ethyl]pyrazol-3-yl]-1-methyl-ethyl]carbamate

To a solution of tert-butyl N-[1-methyl-1-(1H-pyrazol-3-yl)ethyl]carbamate (305 mg, 1.35 mmol) in THF (8 mL) was added NaH (108.30 mg, 2.71 mmol, 60% purity) at 0° C., and stirred at 20° C. for 0.5 h, then benzyl N-(2-bromoethyl) carbamate (419.33 mg, 1.62 mmol) was added at 20° C. for 12 h under N₂. The reaction mixture was quenched by addition water (10 mL) at 25° C., then the mixture was concentrated under reduced pressure to give a crude product. The crude was purified by column chromatography to give tert-butyl N-[1-[1-[2-(benzyloxycarbonylamino) ethyl]pyrazol-3-yl]-1-methyl-ethyl]carbamate (78 mg, 14.31% yield) as a white solid.

MS(ESI) m/z=402.9 [M+H]⁺.

Step 5: tert-butyl(2-(1-(2-aminoethyl)-1H-pyrazol-3-yl) propan-2-yl) carbamate

Con. HCl (1.27 g, 12.56 mmol, 1.25 mL, 36% purity) was added to tert-butyl N-[1-[1-[2-(benzyloxycarbonylamino) ethyl]pyrazol-3-yl]-1-methyl-ethyl]carbamate (15 mg, 28.49 μmol) and stirred at 30° C. for 12 h under N₂. The reaction mixture was concentrated under reduced pressure to give a crude product. The crude was purified by column chromatography to give tert-butyl(2-(1-(2-aminoethyl)-1H-pyrazol-3-yl) propan-2-yl) carbamate (2.2 mg, 19.68% yield) as a yellow solid.

MS(ESI) m/z=269.1 [M+H]⁺.

General Method for Intermediate F (Oxadiazole amine)

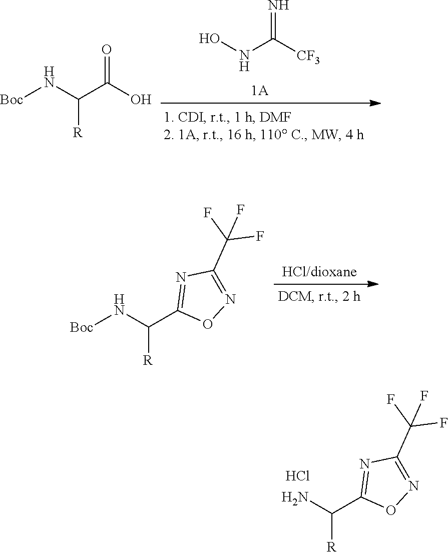

### Intermediate F1A

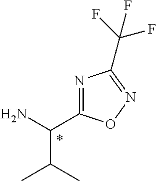

### (1S or 1R)-2-methyl-1-[3-(trifluoromethyl)-1,2,4-oxadiazol-5-yl]propan-1-amine

Step 1: tert-butyl N-[(1S or 1R)-2-methyl-1-[3-(trifluoromethyl)-1,2,4-oxadiazol-5-yl]propyl]carbamate

To a solution of (2S or 2R)-2-(tert-butoxycarbonylamino)-3-methyl-butanoic acid (300 mg, 1.38 mmol,) in DMF (5 mL), then CDI (335.85 mg, 2.07 mmol) was added, the mixture was stirred at 25° C. for 1 hr, then 2,2,2-trifluoro-N-hydroxy-acetamidine (265.23 mg, 2.07 mmol) was added and stirred at 25° C. for 16 hr, The mixture was stirred at 110° C. for 4 h under microwave. The reaction was concentrated under reduced pressure to give residue. The crude product was purified column chromatography to give tert-butyl N-[(1S or 1R)-2-methyl-1-[3-(trifluoromethyl)-1,2,4-oxadiazol-5-yl]propyl]carbamate (110 mg, 21.89% yield) as a yellow solid.

1H NMR (400 MHZ, DMSO-d6) δ=7.90 (d, J=7.6 Hz, 1H), 4.78 (t, J=7.6 Hz, 1H), 2.27-2.11 (m, 1H), 1.38 (s, 9H), 0.95 (d, J=6.8 Hz, 3H), 0.86 (d, J=6.8 Hz, 3H).

Step 2: (1S or 1R)-2-methyl-1-[3-(trifluoromethyl)-1,2,4-oxadiazol-5-yl]propan-1-amine

To a solution of tert-butyl N-[(1S or 1R)-2-methyl-1-[3-(trifluoromethyl)-1,2,4-oxadiazol-5-yl]propyl]carbamate (100 mg, 323.33 μmol) in DCM (5 mL), then HCl/dioxane (4 M, 2.5 mL) was added, the mixture was stirred at 25° C. for 2 hr. The mixture was concentrated under reduced pressure to give (1S or 1R)-2-methyl-1-[3-(trifluoromethyl)-1,2,4-oxadiazol-5-yl]propan-1-amine (210 mg, crude) as a yellow solid.

MS(ESI) m/z=210.1 [M+H]⁺.

| Example | Structure | LCMS |
|---|---|---|
| Intermediate F2 | 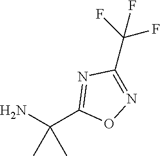 | MS (ESI) m/z = 210.0 |

General Method for Intermediate G (Pyridine amine)

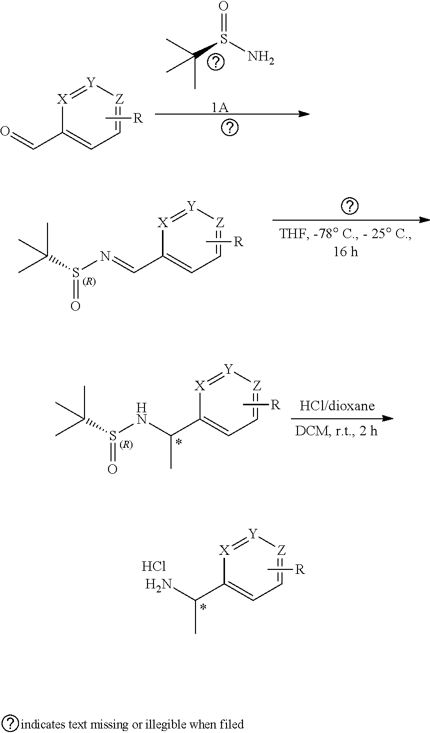

### Intermediate G1

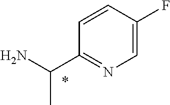

### (1R or 1S)-1-(5-fluoro-2-pyridyl) ethanamine

Step 1: (NE,R or S)—N-[(5-fluoro-2-pyridyl)methylene]-2-methyl-propane-2-sulfinamide

To a solution of 5-fluoropyridine-2-carbaldehyde (13.5 g, 107.91 mmol) in THF (150 mL) was added tetraisopropoxytitanium (122.68 g, 431.65 mmol, 127.39 mL) and (R)-2-methylpropane-2-sulfinamide (26.16 g, 215.83 mmol). The mixture was stirred at 50° C. for 3 h. The reaction was concentrated under reduced pressure to give a residue. The residue was purified by flash silica gel chromatography to give (NE,R or S)—N-[(5-fluoro-2-pyridyl)methylene]-2-methyl-propane-2-sulfinamide (20.2 g, 73.80% yield) as a yellow oil.

1H NMR (400 MHZ, DMSO-d6) δ=8.74 (d, J=2.4 Hz, 1H), 8.46 (s, 1H), 8.17 (dd, J=8.8, 4.8 Hz, 1H), 7.91 (dt, J=8.8, 2.8 Hz, 1H), 1.18 (s, 9H).

Step 2: (R)—N-[(1R or 1S)-1-(5-fluoro-2-pyridyl)ethyl]-2-methyl-propane-2-sulfinamide

To a solution of (NE,R or S)—N-[(5-fluoro-2-pyridyl)methylene]-2-methyl-propane-2-sulfinamide (1 g, 4.38 mmol) in THF (20 mL), then MeMgBr (1 M, 105.13 mL) was added at −78° C., the mixture was stirred at 25° C. for 16 hr. The reaction mixture was quenched by addition NH4Cl (100 mL) at 25° C., and then diluted with EtOAc (100 mL) and extracted with EtOAc (100 mL×3). The combined organic layers were washed with brine (100 mL), dried over anhydrous Na₂SO₄, filtered, and concentrated under reduced pressure to give a residue. The crude product was purified by column chromatography to give (R)—N-[(1R or 1S)-1-(5-fluoro-2-pyridyl)ethyl]-2-methyl-propane-2-sulfinamide (0.5 g, 39.71% yield) as a yellow oil.

MS(ESI) m/z=273.1 [M+H]⁺.

Step 3: (1R or 1S)-1-(5-fluoro-2-pyridyl) ethanamine

To a solution of (R)—N-[(1R or 1S)-1-(5-fluoro-2-pyridyl)ethyl]-2-methyl-propane-2-sulfinamide (0.5 g, 2.05 mmol) in DCM (5 mL) was added HCl/dioxane (4 M, 2.5 mL). The mixture was stirred at 25° C. for 3 hr. The reaction mixture was concentrated under reduced pressure to give (1R or 1S)-1-(5-fluoro-2-pyridyl) ethanamine (0.45 g, 100% Yield) as a white solid.

MS(ESI) m/z=141.0 [M+H]⁺.

| Example | Structure | LOS |
|---|---|---|
| Intermediate G2 | 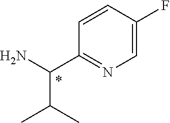 | MS (ESI) m/z = 169.0 [M + H]⁺. |
| Intermediate G3 | 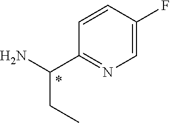 | MS (ESI) m/z = 155.2 [M + H]⁺. |
| Intermediate G4 | 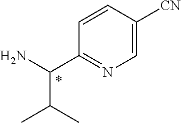 | MS (ESI) m/z = 176.1 [M + H]⁺. |

### Intermediate H

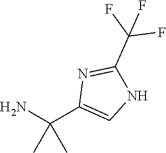

### 2-[2-(trifluoromethyl)-1H-imidazol-4-yl]propan-2-amine

Step 1:2-(trifluoromethyl)-1-((2-(trimethylsilyl) ethoxy)methyl)-1H-imidazole

To a solution of 60% NaH (646.64 mg, 16.17 mmol) in THF (40 mL) was added 2-(trifluoromethyl)-1H-imidazole (2 g, 14.70 mmol) at 0° C. and stirred for 30 min. The mixture was added dropwise SEM-C₁ (2.70 g, 16.17 mmol, 2.86 mL) at 0° C. The mixture was allowed to warm to 20° C. and stirred for 2 hr. The reaction mixture was quenched by addition H₂O (50 mL) at 0° C., and extracted with EtOAc (120 mL×3), dried over Na₂SO₄, filtered, and concentrated under reduced pressure to give a residue. The residue was purified by flash silica gel chromatography to give 2-(trifluoromethyl)-1-((2-(trimethylsilyl) ethoxy)methyl)-1H-imidazole (3.1 g, 77.61% yield) as a white liquid.

1H NMR (400 MHZ, CHLOROFORM-d) 8=7.15 (d, J=1.2 Hz, 1H), 7.08 (d, J=1.2 Hz, 1H), 5.36 (s, 2H), 3.48 (t, J=8.4 Hz, 2H), 0.87 (t, J=8.0 Hz, 2H), 0.07 (s, 9H).

Step 2:2-[[4-bromo-2-(trifluoromethyl) imidazol-1-yl]methoxy]ethyl-trimethyl-silane

To a solution of 2-(trifluoromethyl)-1-((2-(trimethylsilyl) ethoxy)methyl)-1H-imidazole (2 g, 7.51 mmol) in CHCl3 (20 mL) and DMF (20 mL) was added dropwise NBS (1.47 g, 8.26 mmol) at 25° C. for 16 hr. The reaction mixture was concentrated under reduced pressure at 20° C. and extracted with EtOAc (100 mL×3) and H₂O (30 mL). The combined organic layers were washed with brine (30 mL×3), dried over Na₂SO₄, filtered, and concentrated under reduced pressure to give a residue. The residue was purified by flash silica gel chromatography to give 2-[[4-bromo-2-(trifluoromethyl) imidazol-1-yl]methoxy]ethyl-trimethyl-silane(1.64 g, 59.46% yield) as a white oil.

1H NMR (400 MHZ, CHLOROFORM-d) 8=5.45 (s, 2H), 3.62 (t, J=8.8 Hz, 2H), 0.99 (t, J=8.4 Hz, 2H), 0.05 (s, 9H).

Step 3:2-[[4-(1-ethoxyvinyl)-2-(trifluoromethyl) imidazol-1-yl]methoxy]ethyl-trimethyl-silane

A mixture of 2-[[4-bromo-2-(trifluoromethyl) imidazol-1-yl]methoxy]ethyl-trimethyl-silane(1.3 g, 3.77 mmol), tributyl(1-ethoxyvinyl) stannane (2.04 g, 5.65 mmol, 1.91 mL) in 1,4-dioxane (30 mL) was degassed and purged with N2, and then the mixture was added Pd(PPh3) 4 (435.14 mg, 376.56ᵢo μmol) and stirred at 100° C. for 16 hr under N₂. The reaction mixture was cooled to 20° C., and then diluted with H₂O (30 mL) and extracted with EtOAc (100 mL×3). The combined organic layers were washed with brine (50 mL×3), dried over Na₂SO₄, filtered and concentrated under reduced pressure to give 2-[[4-(1-ethoxyvinyl)-2-(trifluoromethyl) imidazol-1-yl]methoxy]ethyl-trimethyl-silane(2.7 g, crude) as a black oil, which was directly used for next step without further purification.

LCMS m/z (ESI+) 337.1 [M+H]⁺.

Step 4:1-[2-(trifluoromethyl)-1-(2-trimethylsilylethoxymethyl) imidazol-4-yl]ethanone

A mixture of 2-[[4-(1-ethoxyvinyl)-2-(trifluoromethyl) imidazol-1-yl]methoxy]ethyl-trimethyl-silane(2.7 g, 8.03 mmol) in THF (10 mL) and HCl (2 M, 20.77 mL) was stirred at 20° C. for 3 hr. The reaction mixture was diluted with H₂O (10 mL) and extracted with EtOAc (50 mL×3). The combined organic layers were washed with brine (30 mL), dried over Na₂SO₄, filtered, and concentrated under reduced pressure to give a residue. The residue was purified by flash silica gel chromatography to give 1-[2-(trifluoromethyl)-1-(2-trimethylsilylethoxymethyl) imidazol-4-yl]ethanone (1 g, 38.79% yield) as a yellow oil.

1H NMR (400 MHZ, CHLOROFORM-d) 8=7.81 (s, 1H), 5.44 (s, 2H), 3.55 (t, J=8.4 Hz, 2H), 2.59 (s, 3H), 0.94 (t, J=8.0 Hz, 2H), 0.002 (s, 9H).

Step 5:2-[2-(trifluoromethyl)-1-(2-trimethylsilylethoxymethyl) imidazol-4-yl]propan-2-ol

To a solution of 1-[2-(trifluoromethyl)-1-(2-trimethylsilylethoxymethyl) imidazol-4-yl]ethanone (11 g, 35.67 mmol) in THF (250 mL), then MeMgBr (3 M, 35.67 mL, 3 eq) was added at −78° C., then the mixture was stirred at 25° C. for 16 hr under N₂. The mixture was poured into a cold (0° C.) sat. NH4Cl solution (500 mL) and stirred for 1 h. THF was evaporated in vacuo (35° C.), and the aqueous layer was extracted with EtOAc (500 mL). The combined organic phase was washed successively with sat. aq. NaHCO₃ (100 mL) and brine (100 mL×3), dried over anhydrous Na₂SO₄, filtered and the filtrate was evaporated in vacuo. The residue was purified by flash silica gel chromatography to give 2-[2-(trifluoromethyl)-1-(2-trimethylsilylethoxymethyl) imidazol-4-yl]propan-2-ol (9 g, 77.77% yield) was obtained as yellow oil.

MS(ESI) m/z 325.1 [M+H]⁺.

Step 6:2-chloro-N-[1-methyl-1-[2-(trifluoromethyl)-1H-imidazol-4-yl]ethyl]acetamide

A mixture of 2-[2-(trifluoromethyl)-1-(2-trimethylsilylethoxymethyl) imidazol-4-yl]propan-2-ol (4.2 g, 12.95 mmol, 1eq) and 2-chloroacetonitrile (10 mL) in AcOH (6.22 g, 103.57 mmol, 5.93 mL, 8 eq) was cooled 0° C. in an ice bath before H2SO₄ (11.43 g, 116.52 mmol, 6.21 mL, 9 eq) was added. The reaction was allowed to warm to 25° C. and was stirred for 18 hrs. The reaction mixture was quenched by addition NaOH (1 M) 500 mL at 30° C., extracted with EtOAc (500 mL*3). The combined organic layers were washed with brine 100 mL, dried over Na₂SO₄, filtered, and concentrated under reduced pressure to give a residue. The residue was purified by flash silica gel chromatography to give 2-chloro-N-[1-methyl-1-[2-(trifluoromethyl)-1H-imidazol-4-yl]ethyl]acetamide (6.5 g, 88.44% yield) was obtained as yellow solid.

MS(ESI) m/z 270.1 [M+H]⁺.

Step 7:2-[2-(trifluoromethyl)-1H-imidazol-4-yl]propan-2-amine

To a solution of 2-chloro-N-[1-methyl-1-[2-(trifluoromethyl)-1H-imidazol-4-yl]ethyl]acetamide (2 g, 7.42 mmol, 1eq) in EtOH (20 mL) was added thiourea (677.51 mg, 8.90 mmol) and acetic acid (8.02 g, 133.51 mmol, 7.64 mL, 18 eq) at 25° C. the mixture was stirred at 80° C. for 16 hr. The reaction mixture was quenched by addition NaOH (1 M) 100 mL at 30° C., extracted with EtOAc (200 mL*3). The combined organic layers were washed with brine (100 mL), dried over Na₂SO₄, filtered, and concentrated under reduced pressure to give a residue. 2-[2-(trifluoromethyl)-1H-imidazol-4-yl]propan-2-amine hydrochloride (1.1 g, 33.01% yield) was obtained as yellow solid.

LCMS m/z (ESI+) 194.1 [M+H]⁺.

### Intermediate I

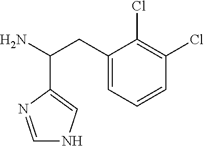 [SMILES: NC(Cc1cccc(Cl)c1Cl)c1c[nH]cn1]

### 2-(2,3-dichlorophenyl)-1-(1H-imidazol-4-yl) ethan-1-amine

Step 1:2-(2,3-dichlorophenyl)-N-methoxy-N-methyl-acetamide

To a solution of 2-(2,3-dichlorophenyl) acetic acid (5 g, 24.39 mmol) and N-methoxymethanamine; hydrochloride (2.85 g, 29.26 mmol) in DCM (50 mL) was added HATU (18.54 g, 48.77 mmol) and DIEA (15.76 g, 121.93 mmol) at 15° C. The mixture was stirred at 15° C. for 12 h under N₂. The mixture was concentrated under reduced pressure to give a crude product. The crude product was purified by flash silica gel chromatography to give 2-(2,3-dichlorophenyl)-N-methoxy-N-methyl-acetamide (5.02 g, 82.97% yield) as a purple solid.

MS(ESI) m/z=247.6 [M+H]⁺.

Step 2:2-(2, 3-dichlorophenyl)-1-(1-tritylimidazol-4-yl) ethanone

To a solution of 4-iodo-1-trityl-imidazole (9.5 g, 21.77 mmol) in DCM (160 mL) was added EtMgBr (3 M in THF, 7.26 mL) at −10° C. under N₂ atmosphere and allowed to stir at −10° C. for 1 h under N₂. A solution of 2-(2,3-dichlorophenyl)-N-methoxy-N-methyl-acetamide (4.59 g, 18.51 mmol) in DCM (60 mL) was added to the mixture at −10° C. under N₂ atmosphere and stirred at 15° C. for 12 h under N₂. The mixture was poured into a cold (0° C.) sat. NH4Cl aq. (100 mL) and stirred for 1 h. The combined organic phase was washed with sat. NaHCO₃ aq. (50 mL) and brine (30 mL×3), dried over Na₂SO₄, filtered and the filtrate was concentrated under reduced pressure to give a crude product. The crude product was purified by flash silica gel chromatography to give 2-(2, 3-dichlorophenyl)-1-(1-tritylimidazol-4-yl) ethanone (3.28 g, 30.28% yield) as a white solid.

1H NMR (400 MHZ, DMSO-d6) δ=7.66 (d, J=1.2 Hz, 1H), 7.58 (d, J=1.2 Hz, 1H), 7.54 (dd, J=7.6, 1.6 Hz, 1H), 7.46-7.40 (m, 9H), 7.37-7.34 (m, 1H), 7.33-7.29 (m, 1H), 7.24-7.15 (m, 6H), 4.50 (s, 2H).

Step 3:2-(2,3-dichlorophenyl)-1-(1-tritylimidazol-4-yl) ethanamine

To a solution of 2-(2,3-dichlorophenyl)-1-(1-tritylimidazol-4-yl) ethanone (1 g, 2.01 mmol) in MeOH (20 mL) was added NaBH₃CN (189.51 mg, 3.02 mmol) and NH₄OAc (1.55 g, 20.10 mmol) at 15° C., then the reaction mixture was heated to 70° C. and stirred for 12 h under N₂ atmosphere. The mixture was poured into a cold (0° C.) sat. NH4Cl aq. (50 mL) and stirred for 1 h. Then the mixture was extracted with EtOAc (50 mL), the combined organic phase was washed with brine (15 mL×3), dried over Na₂SO₄, filtered, and the filtrate was concentrated under pressure to give a crude product. The crude product was purified by column chromatography to give 2-(2,3-dichlorophenyl)-1-(1-tritylimidazol-4-yl) ethanamine (330 mg, 32.93% yield) as a yellow solid.

MS(ESI) m/z=499.7 [M+3H]⁺.

Step 4:2-(2,3-dichlorophenyl)-1-(1H-imidazol-4-yl) ethan-1-amine

To a solution of 2-(2,3-dichlorophenyl)-1-(1-tritylimidazol-4-yl) ethanamine (0.33 g, 179.89 μmol) in MeOH (2 mL) was added HCl (1.84 g, 2.52 mmol, 1.80 mL, 1M) at 20° C. Then the mixture was stirred at 50° C. for 2 h under N₂. The mixture was concentrated under reduced pressure to give a crude product. to give 2-(2,3-dichlorophenyl)-1-(1H-imidazol-4-yl) ethan-1-amine (22.15 mg, 36.8% yield) as a yellow solid.

MS(ESI) m/z=256.1 [M+H]⁺.

### Intermediate J

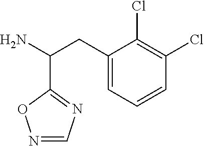 [SMILES: NC(Cc1cccc(Cl)c1Cl)c1ncno1]

### 2-(2,3-dichlorophenyl)-1-(1,2,4-oxadiazol-5-yl) ethan-1-amine

Step 1: tert-butyl(1-amino-3-(2,3-dichlorophenyl)-1-oxopropan-2-yl) carbamate

To a stirred solution of 2-((tert-butoxycarbonyl)amino)-3-(2,3-dichlorophenyl) propanoic acid (693 mg, 2.07 mmol) in DMF (10 mL) were added EDCI (596.28 mg, 3.11 mmol) and HOBt (420.30 mg, 3.11 mmol), and then DIEA (804.01 mg, 6.22 mmol) and NH₄Cl (166.38 mg, 3.11 mmol) was added. The reaction mixture was stirred at 15° C. under N₂ atmosphere for 12 h. The reaction mixture was concentrated under reduced pressure to remove DMF. The crude product was purified column chromatography to give tert-butyl(1-amino-3-(2,3-dichlorophenyl)-1-oxopropan-2-yl) carbamate (418 mg, 60.50% yield) as an off-white solid.

MS(ESI) m/z=233.0 [M-Boc+H]⁺.

Step 2: tert-butyl(Z)-(3-(2,3-dichlorophenyl)-1-(((dimethylamino)methylene)amino)-1-oxopropan-2-yl) carbamate

To a stirred solution of tert-butyl(1-amino-3-(2,3-dichlorophenyl)-1-oxopropan-2-yl) carbamate (318 mg, 954.35 μmol) in DCM (12 mL) were added 1,1-dimethoxy-N,N-dimethyl-methanamine (147.84 mg, 1.24 mmol). The reaction mixture was stirred at 45ᵢÉunder N₂ atmosphere for 2 h. The reaction mixture was concentrated under reduced pressure to obtain a residue. The product tert-butyl tert-butyl(Z)-(3-(2,3-dichlorophenyl)-1-(((dimethylamino)methylene)amino)-1-oxopropan-2-yl) carbamate (544 mg, 100%) as a colorless oil.

MS(ESI) m/z=388.0 [M+H]⁺.

Step 3: tert-butyl(2-(2,3-dichlorophenyl)-1-(1,2,4-oxadiazol-5-yl)ethyl) carbamate

To a stirred solution of tert-butyl(Z)-(3-(2,3-dichlorophenyl)-1-(((dimethylamino)methylene)amino)-1-oxopropan-2-yl) carbamate (370 mg, 952.90 μmol) in EtOH (15 mL) were added Hydroxylamine Hydrochloride (132.44 mg, 1.91 mmol). The reaction mixture was stirred at 75 ᵢÉunder N₂ atmosphere for 12 h. The reaction mixture was concentrated The crude product was purified by column under reduced pressure to give crude product. chromatography to give tert-butyl(2-(2,3-dichlorophenyl)-1-(1,2,4-oxadiazol-5-yl)ethyl) carbamate (202 mg, 59.18% yield) as an off-white solid.

MS(ESI) m/z=302.0 [M-tBu+H]⁺.

Step 4:2-(2,3-dichlorophenyl)-1-(1,2,4-oxadiazol-5-yl) ethan-1-amine

To a stirred solution of tert-butyl(2-(2,3-dichlorophenyl)-1-(1,2,4-oxadiazol-5-yl)ethyl) carbamate (110 mg, 307.07 μmol) in HFIP (10 mL) was added 4-methylbenzenesulfonic acid (211.52 mg, 1.23 mmol). The reaction mixture was concentrated under reduced pressure to give crude product. The crude product was purified by column chromatography to give 2-(2,3-dichlorophenyl)-1-(1,2,4-oxadiazol-5-yl) ethan-1-amine (114.2 mg) as an off-white solid.

MS(ESI) m/z=258.0 [M+H]⁺.

### Intermediate K

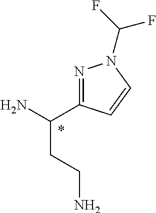 [SMILES: NCCC(N)c1ccn(C(F)F)n1]

### (R or S)-1-(1-(difluoromethyl)-1H-pyrazol-3-yl) propane-1,3-diamine

Step 1: (R or S)-4-(((benzyloxy) carbonyl)amino)-2-((tert-butoxycarbonyl)amino) butanoic acid

To a stirred solution of (2R or 2S)-4-amino-2-(tert-butoxycarbonylamino) butanoic acid (10 g, 45.82 mmol) in Acetone (125 mL) at 0° C. was added aq. NaHCO₃ (1.1 M, 125.00 mL) and the reaction mixture was stirred at 0° C. for 10 min under N₂ atmosphere. Then benzyl carbonochloridate (9.38 g, 55.00 mmol) in toluene (3 mL) was added, and the reaction mixture was stirred at 15° C. for 12 h under N₂ atmosphere. The reaction mixture was concentrated under reduced pressure to give (R or S)-4-(((benzyloxy) carbonyl)amino)-2-((tert-butoxycarbonyl)amino) butanoic acid (16.14 g, 100% Yield).

MS(ESI) m/z=375.0 [M+Na]⁺.

Step 2: tert-butyl N-[(1R or 1S)-3-(benzyloxycarbonylamino)-1-[methoxy(methyl) carbamoyl]propyl]carbamate

To a solution of (R or S)-4-(((benzyloxy) carbonyl)amino)-2-((tert-butoxycarbonyl)amino) butanoic acid (12 g, 34.05 mmol) in DMF (20 mL) was added EDCI (7.18 g, 37.46 mmol) and HOBt (5.06 g, 37.46 mmol) at 0° C. Then N-methoxymethanamine; hydrochloride (3.65 g, 37.46 mmol) and TEA (3.79 g, 37.46 mmol) was added to the mixture and stirred at 15° C. for 12 h under N₂. The reaction mixture was concentrated under reduced pressure to residue. The residue was purified by flash silica gel chromatography to give tert-butyl N-[(1R or 1S)-3-(benzyloxycarbonylamino)-1-[methoxy(methyl) carbamoyl]propyl]carbamate (8.7 g, 64.60% yield) as a yellow oil.

MS(ESI) m/z=395.8 [M+H]⁺.

Step 3: tert-butyl N-[(1R or 1S)-1-[2-(benzyloxycarbonylamino)ethyl]-2-oxo-propyl]carbamate

To a solution of tert-butyl N-[(1R or 1S)-3-(benzyloxycarbonylamino)-1-[methoxy(methyl) carbamoyl]propyl]carbamate (6 g, 15.17 mmol) in THF (60 mL) was added bromo (methyl) magnesium (3 M in diethyl ether, 15.17 mL) at −78° C. for 1 h under N₂ and then stirred at 30° C. for 16 h under N₂. The mixture was poured into sat. NH4Cl aq. (20 mL) at 0° C. and stirred for 1 h. Then the mixture was extracted with EtOAc (300 mL×2). The combined organic phase was washed with sat. aq. NaHCO₃ (15 mL) and brine (15 mL×3), dried over anhydrous Na₂SO₄, filtered, and the filtrate was concentrated under reduced pressure to give tert-butyl N-[(1R or 1S)-1-[2-(benzyloxycarbonylamino)ethyl]-2-oxo-propyl]carbamate (4.67 g, crude) as a yellow oil.

MS(ESI) m/z=no desired mass.

Step 4: tert-butyl N-[(E,1R or 1S)-1-[2-(benzyloxycarbonylamino)ethyl]-4-(dimethylamino)-2-oxo-but-3-enyl]carbamate

To a solution of tert-butyl N-[(1R or 1S)-1-[2-(benzyloxycarbonylamino)ethyl]-2-oxo-propyl]carbamate (4.67 g, 13.33 mmol) in MeCN (50 mL) was added DMF-DMA (3.18 g, 26.65 mmol). The reaction mixture was stirred at 85° C. for 12 h under N₂. The mixture was concentrated under reduced pressure to afford tert-butyl N-[(E,1R or 1S)-1-[2-(benzyloxycarbonylamino)ethyl]-4-(dimethylamino)-2-oxo-but-3-enyl]carbamate (5 g, crude) as a yellow oil.

MS(ESI) m/z=no desired mass.

Step 5: tert-butyl N-[(1R or 1S)-3-(benzyloxycarbonylamino)-1-(1H-pyrazol-3-yl) propyl]carbamate

To a solution of tert-butyl N-[(E,1R or 1S)-1-[2-(benzyloxycarbonylamino)ethyl]-4-(dimethylamino)-2-oxo-but-3-enyl]carbamate (5 g, 12.33 mmol) in EtOH (80 mL) was added Hydrazine Hydrate (1.26 g, 24.66 mmol) at 80° C. and stirred at 80° C. under N₂ for 0.5 h. The reaction mixture was quenched by addition H₂O (30 mL). The reaction mixture was concentrated under reduced pressure to remove EtOH, and then extracted with EtOAc (100 mL×3). The combined organic layers were dried over Na₂SO₄, filtered, and concentrated under reduced pressure to give a residue. The residue was purified by column chromatography to give tert-butyl N-[(1R or 1S)-3-(benzyloxycarbonylamino)-1-(1H-pyrazol-3-yl) propyl]carbamate (420 mg, 9.10% yield) as a yellow oil.

MS(ESI) m/z=375.2 [M+H]⁺.

Step 6: tert-butyl N-[(1R or 1S)-3-(benzyloxycarbonylamino)-1-[1-(difluoromethyl) pyrazol-3-yl]propyl]carbamate

To a solution of tert-butyl tert-butyl N-[(1R or 1S)-3-(benzyloxycarbonylamino)-1-(1H-pyrazol-3-yl) propyl]carbamate (150 mg, 400.61 μmol) and KF (69.82 mg, 1.20 mmol) in MeCN (3 mL) was added 1-[[bromo (difluoro) methyl]-ethoxy-phosphoryl]oxyethane (128.36 mg, 480.73 μmol). The mixture was stirred at 50° C. for 12 h under N₂. The reaction mixture was concentrated under reduced pressure to give a residue. The residue was purified by column chromatography to give tert-butyl N-[(1R or 1S)-3-(benzyloxycarbonylamino)-1-[1-(difluoromethyl) pyrazol-3-yl]propyl]carbamate (42 mg, 24.70% yield) as a white solid.

1H NMR (400 MHZ, METHANOL-d4) δ=8.04 (s, 1H), 7.59-7.34 (m, 5H), 7.33-7.20 (m, 1H), 7.18-7.00 (m, 1H), 6.53 (s, 1H), 5.17 (s, 2H), 4.96 (s, 2H), 2.23-2.00 (m, 2H), 1.54 (s, 9H).

Step 7: (R or S)-1-(1-(difluoromethyl)-1H-pyrazol-3-yl) propane-1,3-diamine

To a solution of tert-butyl N-[(1R or 1S)-3-(benzyloxycarbonylamino)-1-[1-(difluoromethyl) pyrazol-3-yl]propyl]carbamate (82 mg, 193.20 μmol) in 1,4-dioxane (3 mL) was added HCl (5 mL) at 15° C. and stirred at 15° C. for 16 h under N₂ atmosphere. It was concentrated under reduced pressure to give (R or S)-1-(1-(difluoromethyl)-1H-pyrazol-3-yl) propane-1,3-diamine (36.7 mg, crude) as a yellow oil.

MS(ESI) m/z=325.2 [M+H]⁺.

### Intermediate L

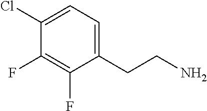 [SMILES: NCCc1ccc(Cl)c(F)c1F]

### 2-(4-chloro-2,3-difluoro-phenyl) ethanamine

Step 1: tert-butyl N-[2-(4-chloro-2, 3-difluoro-phenyl)ethyl]carbamate

To a solution of 1-bromo-4-chloro-2,3-difluoro-benzene (1 g, 4.40 mmol), potassium;2-(tert-butoxycarbonylamino)ethyl-trifluoro-boranuide (1.10 g, 4.40 mmol) in toluene (36 mL) and H₂O (6 mL) were added Cs₂CO₃ (4.30 g, 13.19 mmol), Pd(OAc) 2 (98.71 mg, 439.69 μmol) and dicyclohexyl-[2-(2,6-diisopropoxyphenyl)phenyl]phosphane (205.17 mg, 439.69 μmol), the mixture was stirred at 100° C. for 12 hr under N₂. The reaction mixture was diluted with water (20 mL) and extracted with EtOAc (100 mL×3). The combined organic layers were washed with brine (100 mL), dried over Na₂SO₄, filtered and concentrated under reduced pressure to give a residue. The residue was purified by flash silica gel chromatography to give tert-butyl N-[2-(4-chloro-2, 3-difluoro-phenyl)ethyl]carbamate (960 mg, 74.84% yield) as a yellow oil.

1H NMR (400 MHZ, CHCl3-d) δ=7.15-7.05 (m, 1H), 6.91 (t, J=7.2 Hz, 1H), 3.34 (d, J=6.4 Hz, 2H), 2.84 (t, J=6.4 Hz, 2H), 1.41 (s, 9H).

Step 2:2-(4-chloro-2,3-difluoro-phenyl) ethanamine

To a solution of tert-butyl N-[2-(4-chloro-2,3-difluoro-phenyl)ethyl]carbamate (960 mg, 3.29 mmol) in dioxane (12 mL) was added HCl/dioxane (4 M, 12 mL). The mixture was stirred at 25° C. for 4 hr under N₂. The reaction mixture was concentrated under reduced pressure to give crude product 2-(4-chloro-2,3-difluoro-phenyl) ethanamine (750 mg, crude) as a white solid.

1H NMR (400 MHZ, DMSO-d6) δ=8.21 (br s, 2H), 7.50-7.35 (m, 1H), 7.32-7.14 (m, 1H), 3.10-2.95 (m, 4H).

### Intermediate M

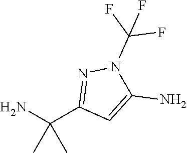

### 5-(1-amino-1-methyl-ethyl)-2-(difluoromethyl) pyrazol-3-amine

Step 1: tert-butyl N-(3-cyano-1,1-dimethyl-2-oxo-propyl) carbamate

To a solution of CH₃CN (5.10 g, 124.27 mmol) in THF (100 mL) was added dropwise NaHMDS (1 M, 124.27 mL) at −78° C. After addition, the mixture was stirred at −50° C. for 20 min, and then methyl 2-(tert-butoxycarbonylamino)-2-methyl-propanoate (9 g, 41.42 mmol) in THF (100 mL) was added dropwise at −78° C. The resulting mixture was stirred at −50° C. for 1 hr. CH₃COOH (7.96 g, 132.56 mmol) was added to the mixture at −78° C. The reaction mixture was concentrated under reduced pressure to give tert-butyl N-(3-cyano-1,1-dimethyl-2-oxo-propyl) carbamate (7 g, crude) as a black brown solid.

MS(ESI) m/z=227.1 [M+H]⁺.

Step 2: tert-butyl N-[1-(5-amino-1H-pyrazol-3-yl)-1-methyl-ethyl]carbamate

To a solution of tert-butyl N-(3-cyano-1,1-dimethyl-2-oxo-propyl) carbamate (7 g, 30.94 mmol) in EtOH (100 mL) was added CH₃COOH (5.57 g, 92.81 mmol,) and N₂H₄ⱼo H₂O (5.47 g, 92.81 mmol) at 0° C. The mixture was stirred at 25° C. for 24 hr. The reaction mixture was concentrated under reduced pressure to give a residue. The residue was purified by flash silica gel chromatography to give tert-butyl N-[1-(5-amino-1H-pyrazol-3-yl)-1-methyl-ethyl]carbamate (3.5 g, 37.66% yield) as a yellow oil.

MS(ESI) m/z=141.2

Step 3: tert-butyl N-[1-[5-amino-1-(difluoromethyl) pyrazol-3-yl]-1-methyl-ethyl]carbamate

To a solution of 1-[[bromo (difluoro) methyl]-ethoxy-phosphoryl]oxyethane (1.17 g, 4.37 mmol) and KF (483.56 mg, 8.32 mmol) in MeCN (15 mL) was added tert-butyl N-[1-(5-amino-1H-pyrazol-3-yl)-1-methyl-ethyl]carbamate (1 g, 4.16 mmol) at 20° C. under N₂. The mixture was stirred at 20° C. for 18 hr. The reaction mixture was diluted with H₂O (30 mL) and extracted with DCM (40 mL×3). The combined organic layers were washed with brine (20 mL×3), dried over Na₂SO₄, filtered and concentrated under reduced pressure to give a residue. The residue was purified by flash silica gel chromatography to give tert-butyl N-[1-[5-amino-1-(difluoromethyl) pyrazol-3-yl]-1-methyl-ethyl]carbamate (355 mg, 22.33% yield) was obtained as a white solid.

MS(ESI) m/z=291.2 [M+H]⁺.

Step 4:5-(1-amino-1-methyl-ethyl)-2-(difluoromethyl) pyrazol-3-amine

A mixture of tert-butyl N-[1-[5-amino-1-(difluoromethyl) pyrazol-3-yl]-1-methyl-ethyl]carbamate (355 mg, 1.22 mmol) and HCl/dioxane (4 M, 2.60 mL) in DCM (6 mL) was stirred at 25° C. for 18 hr. The reaction mixture was concentrated under reduced pressure to give 5-(1-amino-1-methyl-ethyl)-2-(difluoromethyl) pyrazol-3-amine (360 mg, crude) as a white solid.

MS(ESI) m/z=191.1 [M+H]⁺.

### Intermediate N

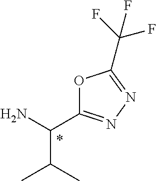

### (1S or 1R)-2-methyl-1-[5-(trifluoromethyl)-1,3,4-oxadiazol-2-yl]propan-1-amine

Step 1: tert-butyl N-[(1S or 1R)-1-(hydrazinecarbonyl)-2-methyl-propyl]carbamate

To a solution of methyl(2S or 2R)-2-(tert-butoxycarbonylamino)-3-methyl-butanoate (8 g, 34.59 mmol) in MeOH (80 mL) was added hydrazine hydrate (5.19 g, 103.77 mmol, 5.04 mL), then the reaction mixture was stirred at 85° C. for 12 h. The reaction mixture was concentrated under reduced pressure to obtain a residue. The residue was dissolved in DCM (100 mL), then extracted with DCM (100 mL×3) and water (100 mL), the combined organic phases were washed with brine (120 mL), dried over anhydrous Na₂SO₄, filtered, and concentrated under reduced pressure to obtain crude product (12.2 g, 100% Yield).

1H NMR (400 MHZ, DMSO-d6) δ=9.04 (s, 1H), 6.63 (d, J=8.8 Hz, 1H), 4.22 (s, 2H), 3.69 (t, J=8.4 Hz, 1H), 1.88-1.82 (m, 1H), 1.38 (s, 9H), 0.86-0.80 (m, 6H).

Step 2: tert-butyl N-[(1S or 1R)-2-methyl-1-[[(2,2,2-trifluoroacetyl)amino]carbamoyl]propyl]carbamate

To a solution of tert-butyl N-[(1S or 1R)-1-(hydrazinecarbonyl)-2-methyl-propyl]carbamate (2.0 g, 8.65 mmol) and DIEA (2.24 g, 17.29 mmol, 3.01 mL) in DCM (20 mL) was added dropwise (CF₃CO) 20 (2.18 g, 10.38 mmol, 1.44 mL) at 0° C., the reaction mixture was stirred at 25° C. for 12 hr. The reaction mixture was poured into water (30 mL) and extracted with DCM (50 mL×3), the combined organic phases were washed with brine (50 mL), dried over anhydrous Na₂SO₄, filtered and concentrated under reduced pressure to obtain tert-butyl N-[(1S or 1R)-2-methyl-1-[[(2,2,2-trifluoroacetyl)amino]carbamoyl]propyl]carbamate (2.47 g, crude) as a colorless oil.

LCMS m/z (ESI+) 271.1 [M+H]⁺.

Step 3: tert-butyl N-[(1S or 1R)-2-methyl-1-[5-(trifluoromethyl)-1,3,4-oxadiazol-2-yl]propyl]carbamate

To a solution of tert-butyl N-[(1S or 1R)-2-methyl-1-[[(2,2,2-trifluoroacetyl)amino]carbamoyl]propyl]carbamate (1.5 g, 4.58 mmol) in THF (30 mL) was added Burgess reagent (4.37 g, 18.33 mmol) at 25° C., the reaction mixture was stirred at 25° C. for 12 hr. The reaction mixture was poured to water (30 mL) and extracted with EtOAc (50 mL×3), the combined organic phases were washed with brine (50 mL), dried over Na₂SO₄, filtered, and concentrated under reduced pressure to obtain a residue. The residue was purified by flash silica gel chromatography to give tert-butyl N-[(1S or 1R)-2-methyl-1-[5-(trifluoromethyl)-1,3,4-oxadiazol-2-yl]propyl]carbamate (1.1 g, 72.95% yield) as a yellow oil.

LCMS m/z (ESI+) 254.0 [M+H]⁺.

Step 4: (1S or 1R)-2-methyl-1-[5-(trifluoromethyl)-1,3,4-oxadiazol-2-yl]propan-1-amine

To a solution of tert-butyl N-[(1S)-2-methyl-1-[5-(trifluoromethyl)-1,3,4-oxadiazol-2-yl]propyl]carbamate (100 mg, 323.33 μmol) in HFIP (1 mL). The mixture was stirred at 150° C. for 1 hr in microwave. The reaction was concentrated under reduced pressure to give residue to give (1S or 1R)-2-methyl-1-[5-(trifluoromethyl)-1,3,4-oxadiazol-2-yl]propan-1-amine (67 mg, crude) as a yellow oil.

1H NMR (400 MHZ, DMSO-d6) δ=3.92 (d, J=6.4 Hz, 1H), 2.05-1.91 (m, 1H), 0.92 (d, J=6.7 Hz, 3H), 0.84 (d, J=6.8 Hz, 3H).

### General Method for Target Compounds A

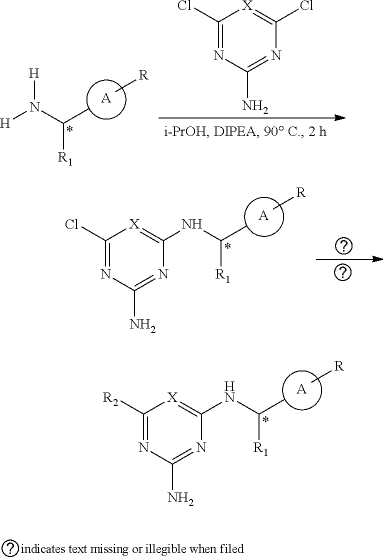

### General Method for Target Compounds B

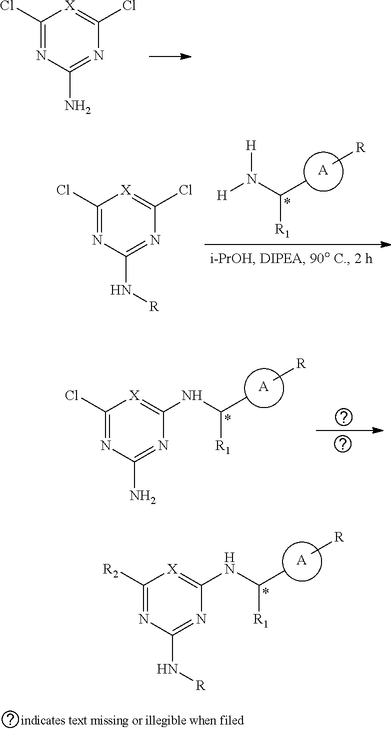

### General Method for Target Compounds C

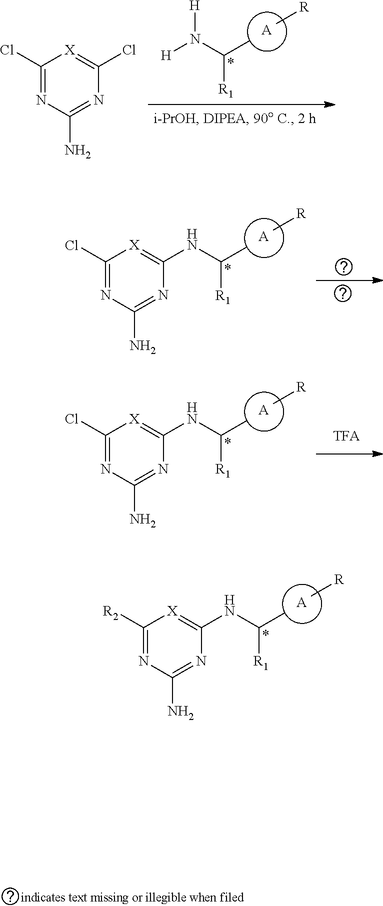

### General Method for Target Compounds D

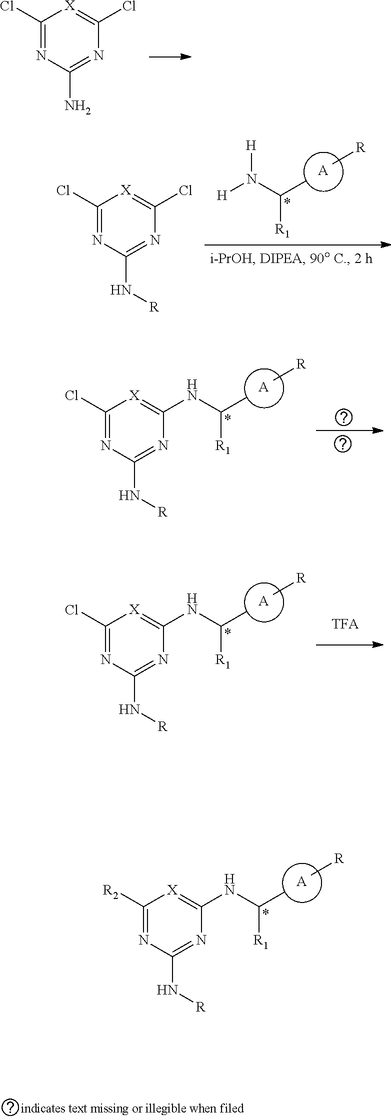

### General Method for Target Compounds E

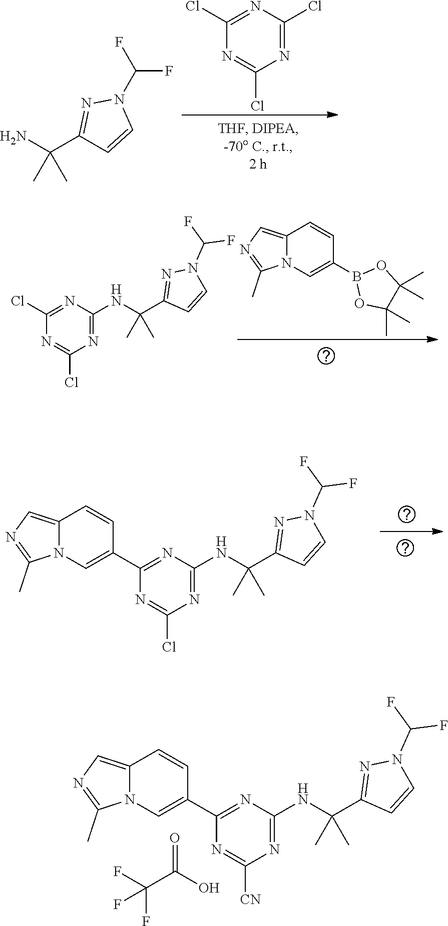

### Example 1

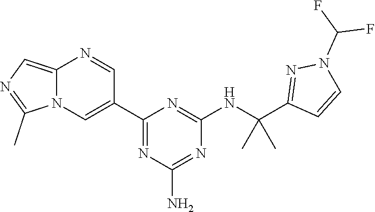 [SMILES: Cc1ncc2ncc(-c3nc(N)nc(NC(C)(C)c4ccn(C(F)F)n4)n3)cn12]

### N2-(2-(1-(difluoromethyl)-1H-pyrazol-3-yl) propan-2-yl)-6-(6-methylimidazo[1,5-a]pyrimidin-3-yl)-1,3,5-triazine-2,4-diamine (General Method for Target Compounds A)

Step 1:6-chloro-N2-[1-[1-(difluoromethyl) pyrazol-3-yl]-1-methyl-ethyl]-1,3,5-triazine-2,4-diamine

A mixture of 2-[1-(difluoromethyl) pyrazol-3-yl]propan-2-amine (Intermediate D2, 200.12 mg, 945.57 μmol), 4,6-dichloro-1,3,5-triazin-2-amine (130 mg, 787.97 μmol), DIPEA (408 mg, 3.15 mmol) in 1,4-Dioxane (8 mL) was degassed and purged with N2, and then the mixture was stirred at 90° C. for 12 h under N₂. The reaction mixture was concentrated under reduced pressure to obtain a residue. The residue was purified by flash silica gel chromatography to give 6-chloro-N2-[1-[1-(difluoromethyl) pyrazol-3-yl]-1-methyl-ethyl]-1,3,5-triazine-2,4-diamine (160 mg, 46.80% yield) as a white solid.

MS(ESI) m/z=304.1 [M+H]⁺.

Step 2: N2-[1-[1-(difluoromethyl) pyrazol-3-yl]-1-methyl-ethyl]-6-(6-methylimidazo[1,5-a]pyrimidin-3-yl)-1,3,5-triazine-2,4-diamine

A mixture of 6-chloro-N2-[1-[1-(difluoromethyl) pyrazol-3-yl]-1-methyl-ethyl]-1,3,5-triazine-2,4-diamine (50 mg, 164.64 μmol), 6-methyl-3-(4,4,5,5-tetramethyl-1,3,2-dioxaborolan-2-yl) imidazo[1,5-a]pyrimidine (Intermediate A1, 2.67 g, 10.30 mmol), K₃PO₄ (87.37 mg, 411.59 μmol) and ditert-butyl(cyclopentyl)phosphane; dichloropalladium; iron (10.73 mg, 16.46 μmol) in THF (10 mL) and H₂O (1 mL) was degassed and purged with N2, and then the mixture was stirred at 90° C. for 12 h under N₂. The reaction mixture was concentrated under reduced pressure to give Compound N2-[1-[1-(difluoromethyl) pyrazol-3-yl]-1-methyl-ethyl]-6-(6-methylimidazo[1,5-a]pyrimidin-3-yl)-1,3,5-triazine-2,4-diamine (6.0 mg, 4.55% yield) as a yellow solid.

MS(ESI) m/z=401.0 [M+H]⁺.

1H NMR (400 MHZ, Methanol-d4): δ=8.96 (t, 2H), 7.90 (d, J=2.8 Hz, 1H), 7.59-7.24 (m, 2H), 6.46 (d, J=2.8 Hz, 1H), 2.71 (br s, 3H), 1.82 (s, 6H).

### Example 2

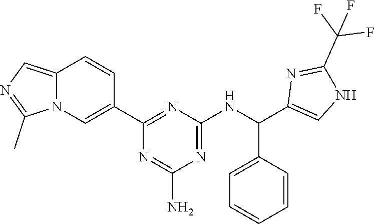 [SMILES: Cc1ncc2ccc(-c3nc(N)nc(NC(c4ccccc4)c4c[nH]c(C(F)(F)F)n4)n3)cn12]

### 6-(3-methylimidazo[1,5-a]pyridin-6-yl)-N2-[phenyl-[2-(trifluoromethyl)-1H-imidazol-4-yl]methyl]-1,3,5-triazine-2,4-diamine (General Method for Target Compounds A)

LCMS m/z (ESI+) 466.0 [M+H]⁺.

1H NMR (400 MHZ, DMSO-d6) δ=8.72 (s, 1H), 8.35 (br s, 1H), 7.41 (d, J=1.6 Hz, 1H), 7.39 (s, 2H), 7.36-7.31 (m, 1H), 7.27 (t, J=7.6 Hz, 2H), 7.21-7.16 (m, 1H), 6.98 (br s, 1H), 6.56 (br s, 3H), 5.74 (s, 1H), 2.63 (s, 3H).

### Example 3

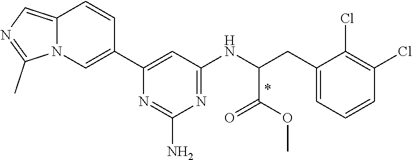 [SMILES: COC(=O)C(Cc1cccc(Cl)c1Cl)Nc1cc(-c2ccc3cnc(C)n3c2)nc(N)n1]

### methyl(S or R)-2-((2-amino-6-(3-methylimidazo[1,5-a]pyridin-6-yl)pyrimidin-4-yl)amino)-3-(2,3-dichlorophenyl) propanoate (General Method for Target Compounds A)

LCMS m/z (ESI+) 471.0 [M+H]⁺.

1H NMR (400 MHZ, Methanol-d4): δ=8.54 (s, 1H), 7.57 (d, J=9.6 Hz, 1H), 7.43 (dd, J=8.0, 1.6 Hz, 1H), 7.33 (s, 1H), 7.30-7.28 (m, 1H), 7.24-7.18 (m, 1H), 7.15 (d, J=10.4 Hz, 1H), 6.31 (br s, 1H), 5.13-5.12 (m, 1H), 3.74 (s, 3H), 3.49 (dd, J=14.0, 6.0 Hz, 1H), 3.26 (dd, J=14.0, 8.8 Hz, 1H), 2.71 (s, 3H).

### Example 4

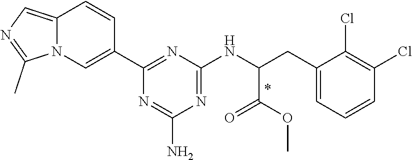 [SMILES: COC(=O)C(Cc1cccc(Cl)c1Cl)Nc1nc(N)nc(-c2ccc3cnc(C)n3c2)n1]

### methyl(2S or 2R)-2-[[4-amino-6-(3-methylimidazo[1,5-a]pyridin-6-yl)-1,3,5-triazin-2-yl]amino]-3-(2,3-dichlorophenyl) propanoate (General Method for Target Compounds A)

LCMS m/z (ESI+) 472.0 [M+H]⁺.

1H NMR (400 MHZ, Methanol-d4) δ=8.84 (br s, 1H), 8.25 (s, 1H), 7.62-7.40 (m, 2H), 7.33-7.25 (m, 2H), 7.23-7.16 (m, 1H), 7.15-7.04 (m, 1H), 5.27-5.14 (m, 1H), 3.80-3.67 (m, 3H), 3.64-3.45 (m, 1H), 3.27-3.10 (m, 1H), 2.81-2.63 (m, 3H).

### Example 5

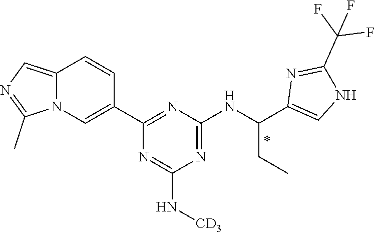 [SMILES: [2H]C([2H])([2H])Nc1nc(NC(CC)c2c[nH]c(C(F)(F)F)n2)nc(-c2ccc3cnc(C)n3c2)n1]

### 6-(3-methylimidazo[1,5-a]pyridin-6-yl)-N2-(trideuteriomethyl)-N4-[(1R or 1S)-1-[2-(trifluoromethyl)-1H-imidazol-4-yl]propyl]-1,3,5-triazine-2,4-diamine (General Method for Target Compounds B)

Step 1:4,6-dichloro-N-(trideuteriomethyl)-1,3,5-triazin-2-amine

2,4,6-trichloro-1,3,5-triazine (2 g, 10.85 mmol) was dissolved in THF (20 mL) and cooled to −70° C. DIPEA (2.80 g, 21.69 mmol) and trideuteriomethanamine; hydrochloride (764.99 mg, 10.85 mmol) were added to the reaction mixture, which was stirred at −70° C. for 1 h and allowed to warm to 25° C. for 12 h. The reaction mixture was concentrated under reduced pressure to give a residue. The residue was purified by flash silica gel chromatography to give 4,6-dichloro-N-(trideuteriomethyl)-1,3,5-triazin-2-amine (1.2 g, 46.20% yield) as a white solid.

MS(ESI) m/z=182.1 [M+H]⁺

Step 2:6-chloro-N2-(trideuteriomethyl)-N4-[(1R or 1S)-1-[2-(trifluoromethyl)-1H-imidazol-4-yl]propyl]-1,3,5-triazine-2,4-diamine

To a solution of 4,6-dichloro-N-(trideuteriomethyl)-1,3,5-triazin-2-amine (100 mg, 549.37 μmol) and (1R or 1S)-1-[2-(trifluoromethyl)-1H-imidazol-4-yl]propan-1-amine (106.12 mg, 462.14 μmol) in n-BuOH (5 mL) was added DIPEA (213.01 mg, 1.65 mmol). The mixture was stirred at 90° C. for 12 hr. The reaction mixture was concentrated under reduced pressure to give a residue. The residue was purified by flash silica gel chromatography to give 6-chloro-N2-(trideuteriomethyl)-N4-[(1R or 1S)-1-[2-(trifluoromethyl)-1H-imidazol-4-yl]propyl]-1,3,5-triazine-2,4-diamine (155 mg, 74.96% yield) as a white solid.

MS(ESI) m/z=339.2 [M+H]⁺

Step 3:6-(3-methylimidazo[1,5-a]pyridin-6-yl)-N2-(trideuteriomethyl)-N4-[(1R or 1S)-1-[2-(trifluoromethyl)-1H-imidazol-4-yl]propyl]-1,3,5-triazine-2,4-diamine

A mixture of 6-chloro-N2-(trideuteriomethyl)-N4-[(1R os 1S)-1-[2-(trifluoromethyl)-1H-imidazol-4-yl]propyl]-1,3,5-triazine-2,4-diamine (150 mg, 442.83 μmol), 3-methyl-6-(4,4,5,5-tetramethyl-1,3,2-dioxaborolan-2-yl) imidazo[1,5-a]pyridine (228.61 mg, 885.65 μmol), ditert-butyl(cyclopentyl)phosphane; dichloropalladium; iron (14.43 mg, 22.14 μmol), K₃PO₄ (187.99 mg, 885.65 μmol) in THF (5 mL) and H₂O (5 mL) was degassed and purged with N2, and then the mixture was stirred at 90° C. for 12 hr under N₂. The reaction was concentrated under reduced pressure to give residue. The crude product was purified by column chromatography to give 6-(3-methylimidazo[1,5-a]pyridin-6-yl)-N2-(trideuteriomethyl)-N4-[(1R or 1S)-1-[2-(trifluoromethyl)-1H-imidazol-4-yl]propyl]-1,3,5-triazine-2,4-diamine (12.2 mg, 6.34% yield) as a yellow solid.

MS(ESI) m/z 435.2 [M+H]⁺.

1H NMR (400 MHZ, DMSO-d6) δ=8.82 (s, 1H), 7.52 (s, 2H), 7.28 (s, 1H), 7.16 (s, 2H), 7.00 (br s, 2H), 5.15 (s, 1H), 2.65 (s, 3H), 1.96-1.85 (m, 2H), 0.92 (t, J=7.6 Hz, 3H).

### Example 6

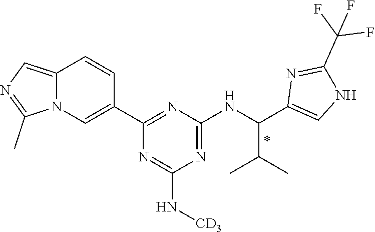 [SMILES: [2H]C([2H])([2H])Nc1nc(NC(c2c[nH]c(C(F)(F)F)n2)C(C)C)nc(-c2ccc3cnc(C)n3c2)n1]

### (S or R)—N2-(methyl-d3)-N4-(2-methyl-1-(2-(trifluoromethyl)-1H-imidazol-4-yl) propyl)-6-(3-methylimidazo[1,5-a]pyridin-6-yl)-1,3,5-triazine-2,4-diamine (General Method for Target Compounds B)

MS(ESI) m/z 449.2 [M+H]⁺

1H NMR (400 MHZ, Methanol-d4) δ=8.89 (s, 1H), 8.18 (s, 1H), 7.61 (s, 1H), 7.49 (d, J=9.6 Hz, 1H), 7.30 (s, 1H), 7.19 (br s, 1H), 5.25-5.08 (m, 1H), 2.71 (s, 3H), 2.24 (s, 1H), 0.99 (m, 6H).

### Example 7A and Example 7B

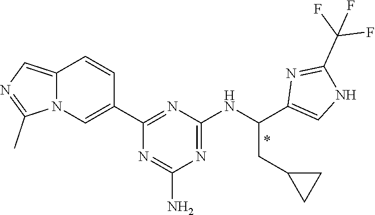 [SMILES: Cc1ncc2ccc(-c3nc(N)nc(NC(CC4CC4)c4c[nH]c(C(F)(F)F)n4)n3)cn12]

### N4-[(1S or 1R)-2-cyclopropyl-1-[2-(trifluoromethyl)-1H-imidazol-4-yl]ethyl]-6-(3-methylimidazo[1,5-a]pyridin-6-yl)-1,3,5-triazine-2,4-diamine (General Method for Target Compounds B)

### 7A

MS(ESI) m/z 444.2 [M+H]⁺.

1H NMR (400 MHZ, DMSO-d6) δ=13.06 (s, 1H), 8.90 (s, 1H), 7.54 (s, 2H), 7.38 (s, 2H), 7.23-7.14 (m, 1H), 6.81 (br s, 2H), 5.96-5.78 (m, 1H), 5.40-5.10 (m, 1H), 5.07-5.01 (m, 1H), 5.00-4.89 (m, 1H), 2.65 (s, 3H), 2.14-2.08 (m, 2H), 2.03-1.94 (m, 2H).

### 7B

LCMS m/z (ESI+) 444.2 [M+H]⁺.

1H NMR (400 MHZ, DMSO-d6) δ=11.60 (s, 1H), 8.81 (s, 1H), 7.56 (s, 2H), 7.28 (s, 2H), 7.18 (s, 1H), 6.70 (br s, 2H), 5.91-5.81 (m, 1H), 5.37-5.15 (m, 1H), 5.04 (d, J=1.6 Hz, 1H), 5.00-4.95 (m, 1H), 2.65 (s, 3H), 2.14-2.07 (m, 2H), 2.04-1.95 (m, 2H).

### Example 8

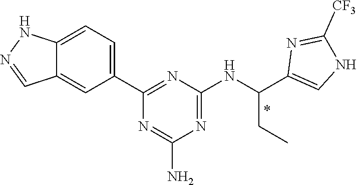 [SMILES: CCC(Nc1nc(N)nc(-c2ccc3[nH]ncc3c2)n1)c1c[nH]c(C(F)(F)F)n1]

### 6-(1H-indazol-5-yl)-N4-[(1R or 1S)-1-[2-(trifluoromethyl)-1H-imidazol-4-yl]propyl]-1, 3, 5-triazine-2, 4-diamine (General Method for Target Compounds C)

Step 1:6-chloro-N4-[(1R or 1S)-1-[2-(trifluoromethyl)-1H-imidazol-4-yl]propyl]-1,3,5-triazine-2,4-diamine

To a solution of (1R or 1S)-1-[2-(trifluoromethyl)-1H-imidazol-4-yl]propan-1-amine (248.45 mg, 1.08 mmol) and 4,6-dichloro-1,3,5-triazin-2-amine (170 mg, 1.03 mmol) in i-PrOH (6 mL) was added DIPEA (532.69 mg, 4.12 mmol). The mixture was stirred at 90° C. for 12 hr. The reaction mixture was concentrated under reduced pressure to give a residue. The residue was purified by flash silica gel chromatography to obtain 6-chloro-N4-[(1R or 1S)-1-[2-(trifluoromethyl)-1H-imidazol-4-yl]propyl]-1,3,5-triazine-2,4-diamine (589 mg, 100% Yield) as a yellow oil.

MS(ESI) m/z=322.0 [M+H]⁺.

Step 2:6-(1-tetrahydropyran-2-ylindazol-5-yl)-N4-[(1R or 1S)-1-[2-(trifluoromethyl)-1H-imidazol-4-yl]propyl]-1, 3, 5-triazine-2,4-diamine

To a solution of 6-chloro-N4-[(1R or 1S)-1-[2-(trifluoromethyl)-1H-imidazol-4-yl]propyl]-1,3,5-triazine-2,4-diamine (520 mg, 1.62 mmol) and 1-tetrahydropyran-2-yl-5-(4,4,5,5-tetramethyl-1,3,2-dioxaborolan-2-yl) indazole (1.06 g, 3.23 mmol) in THF (7 mL) and H₂O (0.7 mL) was added K₃PO₄ (1.03 g, 4.85 mmol) and ditert-butyl(cyclopentyl)phosphane; dichloropalladium; iron (105.35 mg, 161.65 μmol) was degassed and purged with N2, and then the mixture was stirred at 90° C. for 12 hr under N₂. The reaction mixture was diluted with H₂O (10 mL) and extracted with dichloromethane (10 mL×3). The combined organic layers were washed with brine (10 mL), dried over anhydrous Na₂SO₄, filtered, and concentrated under reduced pressure to give a residue. The residue was purified by flash silica gel chromatography to obtain 6-(1-tetrahydropyran-2-ylindazol-5-yl)-N4-[(1R or 1S)-1-[2-(trifluoromethyl)-1H-imidazol-4-yl]propyl]-1, 3, 5-triazine-2,4-diamine (173.2 mg, 21.32% yield) as a yellow oil.

MS(ESI) m/z=488.2 [M+H]⁺.

Step 3:6-(1H-indazol-5-yl)-N4-[(1R or 1S)-1-[2-(trifluoromethyl)-1H-imidazol-4-yl]propyl]-1, 3, 5-triazine-2, 4-diamine

To a solution of 6-(1-tetrahydropyran-2-ylindazol-5-yl)-N4-[(1R or 1S)-1-[2-(trifluoromethyl)-1H-imidazol-4-yl]propyl]-1, 3, 5-triazine-2,4-diamine (173 mg, 354.89 μmol) in DCM (3 mL) was added TFA (1.54 g, 13.51 mmol). The mixture was stirred at 25° C. for 4 hr. The reaction mixture was concentrated under reduced pressure to give a residue. The residue was purified by column chromatography to obtain 6-(1H-indazol-5-yl)-N4-[(1R or 1S)-1-[2-(trifluoromethyl)-1H-imidazol-4-yl]propyl]-1, 3, 5-triazine-2, 4-diamine (69.7 mg, 48.69% yield) as a white solid.

LCMS m/z (ESI+) 404.0 [M+H]⁺.

1H NMR (400 MHZ, Methanol-d4): δ=8.71 (br s, 1H), 8.32-8.22 (m, 2H), 7.75-7.68 (m, 1H), 7.29 (s, 1H), 5.49-5.43 (m, 1H), 2.15-1.99 (m, 2H), 1.12-1.02 (m, 3H).

### Example 9

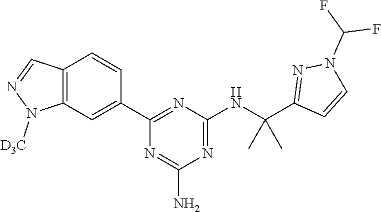 [SMILES: [2H]C([2H])([2H])n1ncc2ccc(-c3nc(N)nc(NC(C)(C)c4ccn(C(F)F)n4)n3)cc21]

### N2-(2-(1-(difluoromethyl)-1H-pyrazol-3-yl) propan-2-yl)-6-(1-(methyl-d3)-1H-indazol-6-yl)-1,3,5-triazine-2,4-diamine (General Method for Target Compounds A)

LCMS m/z (ESI+) 403.1 [M+H]⁺.

1H NMR (400 MHZ, DMSO-d6) δ=8.34 (br s, 1H), 8.07-8.02 (m, 1H), 8.02-7.97 (m, 1H), 7.93 (br s, 1H), 7.87-7.51 (m, 2H), 7.01 (br s, 1H), 6.62-6.36 (m, 3H), 1.78 (s, 6H)

### Example 10

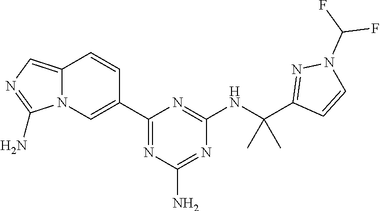 [SMILES: CC(C)(Nc1nc(N)nc(-c2ccc3cnc(N)n3c2)n1)c1ccn(C(F)F)n1]

### 6-(3-aminoimidazo[1,5-a]pyridin-6-yl)-N2-(2-(1-(difluoromethyl)-1H-pyrazol-3-yl) propan-2-yl)-1,3,5-triazine-2,4-diamine (General Method for Target Compounds C)

Step 1:6-chloro-N4-[1-[1-(difluoromethyl) pyrazol-3-yl]-1-methyl-ethyl]-1,3,5-triazine-2,4-diamine

To a solution of 2-[1-(difluoromethyl) pyrazol-3-yl]propan-2-amine (Intermediate D2, 2 g, 9.45 mmol, HCl) and 4,6-dichloro-1,3,5-triazin-2-amine (1.56 g, 9.45 mmol) in i-PrOH (20 mL) was added DIPEA (3.66 g, 28.35 mmol), the mixture was stirred at 90° C. for 16 hr. The reaction was concentrated under reduced pressure to give residue. The residue was purified by flash silica gel chromatography to give 6-chloro-N4-[1-[1-(difluoromethyl) pyrazol-3-yl]-1-methyl-ethyl]-1,3,5-triazine-2,4-diamine (1.8 g, 43.28% yield) as a light yellow solid.

MS(ESI) m/z=304.0 [M+H]⁺.

Step 2:6-(3-aminoimidazo[1,5-a]pyridin-6-yl)-N2-(2-(1-(difluoromethyl)-1H-pyrazol-3-yl) propan-2-yl)-1,3,5-triazine-2,4-diamine

To a stirred solution of 6-chloro-N4-[1-[1-(difluoromethyl) pyrazol-3-yl]-1-methyl-ethyl]-1,3,5-triazine-2,4-diamine (1.27 g, 4.17 mmol) in THF (67.5 mL) and H₂O (6.75 mL) were added N-[(4-methoxyphenyl)methyl]-6-(4,4,5,5-tetramethyl-1,3,2-dioxaborolan-2-yl) imidazo[1,5-a]pyridin-3-amine (1.9 g, 5.01 mmol), ditert-butyl(cyclopentyl)phosphane; dichloropalladium; iron (272.09 mg, 417.48 μmol) and K₂CO₃ (1.15 g, 8.35 mmol), and the reaction mixture was stirred at 75° C. under N₂ for 12 h. The reaction was filtered and concentrated under reduced pressure to give a residue. The residue was purified by flash silica gel chromatography to give N2-[1-[1-(difluoromethyl) pyrazol-3-yl]-1-methyl-ethyl]-6-[3-[(4-methoxyphenyl)methylamino]imidazo[1,5-a]pyridin-6-yl]-1,3,5-triazine-2,4-diamine (1.03 g, 47.40% yield) as a brown solid.

MS(ESI) m/z=521.2 [M+H]⁺.

Step 3:6-(3-aminoimidazo[1,5-a]pyridin-6-yl)-N2-[1-[1-(difluoromethyl) pyrazol-3-yl]-1-methyl-ethyl]-1,3,5-triazine-2,4-diamine

To a solution of N2-[1-[1-(difluoromethyl) pyrazol-3-yl]-1-methyl-ethyl]-6-[3-[(4-methoxyphenyl)methylamino]imidazo[1,5-a]pyridin-6-yl]-1, 3, 5-triazine-2,4-diamine (2 g, 3.84 mmol) in TFA (20 mL) at 15° C. The mixture was stirred at 15° C. for 6 h. The reaction was filtered and concentrated under reduced pressure to give a residue. The crude product was purified by column chromatography to give 6-(3-aminoimidazo[1,5-a]pyridin-6-yl)-N2-[1-[1-(difluoromethyl) pyrazol-3-yl]-1-methyl-ethyl]-1,3,5-triazine-2,4-diamine (410 mg, 26.65% yield) as a yellow solid.

MS(ESI) m/z=401.1 [M+H]⁺.

1H NMR (400 MHZ, Methanol-d4) δ=9.11-8.69 (m, 1H), 7.90 (br s, 1H), 7.60-7.13 (m, 4H), 6.46 (s, 1H), 1.81 (s, 6H).

### Example 11

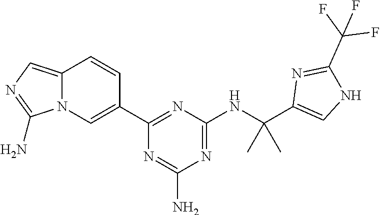 [SMILES: CC(C)(Nc1nc(N)nc(-c2ccc3cnc(N)n3c2)n1)c1c[nH]c(C(F)(F)F)n1]

### 6-(3-aminoimidazo[1,5-a]pyridin-6-yl)-N2-(2-(2-(trifluoromethyl)-1H-imidazol-4-yl) propan-2-yl)-1,3,5-triazine-2,4-diamine (General Method for Target Compounds C)

LCMS m/z (ESI+) 419.2 [M+H]⁺.

1H NMR (400 MHZ, METHANOL-d4) δ=9.03-8.44 (m, 1H), 8.25 (br s, 1H), 7.72-7.34 (m, 4H), 7.33-7.25 (m, 1H), 7.24-6.77 (m, 1H), 1.93-1.62 (m, 6H)

### Example 12

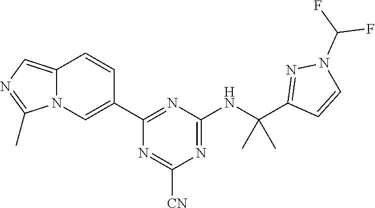 [SMILES: Cc1ncc2ccc(-c3nc(C#N)nc(NC(C)(C)c4ccn(C(F)F)n4)n3)cn12]

### 4-((2-(1-(difluoromethyl)-1H-pyrazol-3-yl) propan-2-yl)amino)-6-(3-methylimidazo[1,5-a]pyridin-6-yl)-1,3,5-triazine-2-carbonitrile (General Method for Target Compounds E)

Step 1. 4,6-dichloro-N-(2-(1-(difluoromethyl)-1H-pyrazol-3-yl) propan-2-yl)-1,3,5-triazin-2-amine

To a stirred solution of 2-[1-(difluoromethyl) pyrazol-3-yl]propan-2-amine (165 mg, 779.63 μmol) in THF (4 mL) were added DIPEA (201.52 mg, 1.56 mmol) and 2,4,6-trichloro-1,3,5-triazine (129.39 mg, 701.66 μmol) at −70° C. under N₂, and the reaction mixture was stirred at −70° C. for 1 h under N₂, then allowed to warm to 15° C. for 1 hr at N2. The reaction mixture was concentrated under reduced pressure to give a residue. The residue was purified by flash silica gel chromatography to give 4,6-dichloro-N-(2-(1-(difluoromethyl)-1H-pyrazol-3-yl) propan-2-yl)-1,3,5-triazin-2-amine (180 mg, 557.05 μmol) as an off-white solid.

MS(ESI) m/z=323.0 [M+H]⁺.

Step 2 4-chloro-N-(2-(1-(difluoromethyl)-1H-pyrazol-3-yl) propan-2-yl)-6-(3-methylimidazo[1,5-a]pyridin-6-yl)-1,3,5-triazin-2-amine

To a solution of 4,6-dichloro-N-(2-(1-(difluoromethyl)-1H-pyrazol-3-yl) propan-2-yl)-1,3,5-triazin-2-amine (150 mg, 464.21 μmol) and 3-methyl-6-(4,4,5,5-tetramethyl-1,3,2-dioxaborolan-2-yl) imidazo[1,5-a]pyridine (143.79 mg, 557.05 μmol) in THF (1.5 mL) and H₂O (1.5 mL) was added K₃PO₄ (197.07 mg, 928.42 μmol) and ditert-butyl(cyclopentyl)phosphane; dichloropalladium; iron (15.13 mg, 23.21 μmol). The mixture was stirred at 60° C. for 6 hr. The reaction was filtered and concentrated under reduced pressure to give a residue. The residue was purified by flash silica gel chromatography to give 4-chloro-N-(2-(1-(difluoromethyl)-1H-pyrazol-3-yl) propan-2-yl)-6-(3-methylimidazo[1,5-a]pyridin-6-yl)-1,3,5-triazin-2-amine (45 mg, 107.44 μmol, 23.15% yield) as a green solid.

MS(ESI) m/z=419.1 [M+H]⁺.

Step 3:4-((2-(1-(difluoromethyl)-1H-pyrazol-3-yl) propan-2-yl)amino)-6-(3-methylimidazo[1,5-a]pyridin-6-yl)-1,3,5-triazine-2-carbonitrile

A mixture of 4-chloro-N-[1-[1-(difluoromethyl) pyrazol-3-yl]-1-methyl-ethyl]-6-(3-methylimidazo[1,5-a]pyridin-6-yl)-1,3,5-triazin-2-amine (45 mg, 107.44 μmol), tetrabutylammonium; cyanide (31.73 mg, 118.19 μmol) and DABCO (14.46 mg, 128.93 μmol) in MeCN (1 mL) was degassed and purged with N2, and then the mixture was stirred at 15° C. for 16 h under N₂ atmosphere. The reaction was concentrated under reduced pressure to give a residue. The residue was purified by column chromatography to give 4-((2-(1-(difluoromethyl)-1H-pyrazol-3-yl) propan-2-yl)amino)-6-(3-methylimidazo[1,5-a]pyridin-6-yl)-1,3,5-triazine-2-carbonitrile (19.74 mg, 44.83% yield) as a yellow solid.

MS(ESI) m/z=410.3 [M+H]⁺.

1H NMR (400 MHZ, DMSO-d6) δ=9.48-9.25 (m, 1H), 9.04-8.80 (m, 1H), 8.14-8.03 (m, 1H), 7.93-7.57 (m, 3H), 7.18 (d, J=9.6 Hz, 1H), 6.48-8.41 (m, 1H), 2.88-2.78 (m, 3H), 1.73 (s, 6H).

### Example 13

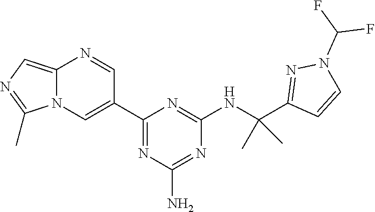 [SMILES: Cc1ncc2ncc(-c3nc(N)nc(NC(C)(C)c4ccn(C(F)F)n4)n3)cn12]

### N2-[1-[1-(difluoromethyl) pyrazol-3-yl]-1-methyl-ethyl]-6-(3-methylimidazo[1,5-a]pyrazin-6-yl)-1,3,5-triazine-2,4-diamine

Step 1: N-[(5-bromopyrazin-2-yl)methyl]acetamide

To a stirred solution of (5-bromopyrazin-2-yl) methanamine (20 g, 74.37 mmol) in DCM (600 mL) was added Et₃N (22.58 g, 223.10 mmol). Then acetyl chloride (11.68 g, 148.73 mmol) was added slowly at 0° C. and the reaction mixture stirred at 15° C. for 16 h. The reaction mixture was poured into water (300 mL) and extracted with DCM (500 mL×3). The combined organic layer was washed with brine (500 mL×2), dried over anhydrous Na₂SO₄ and concentrated under reduced pressure to give crude product N-[(5-bromopyrazin-2-yl)methyl]acetamide (15 g, 87.67% yield) as a brown solid.

MS(ESI) m/z 230.1 [M+H]⁺.

Step 2:6-bromo-3-methyl-imidazo[1,5-a]pyrazine

To a stirred solution of N-[(5-bromopyrazin-2-yl)methyl]acetamide (19 g, 82.59 mmol) and 2-methoxypyridine (18.02 g, 165.17 mmol) in DCM (370 mL) at 0° C., was added TFAA (34.69 g, 165.17 mmol), and the reaction mixture was stirred at 15° C. under N₂ for 12 h. The reaction mixture was concentrated under reduced pressure to give crude product. The crude product was purified by column chromatography to give 6-bromo-3-methyl-imidazo[1,5-a]pyrazine (3.3 g, 18.84% yield) as an off-white solid.

MS(ESI) m/z 211.9 [M+H]⁺.

Step 3: tributyl-(3-methylimidazo[1,5-a]pyrazin-6-yl) stannane

To a stirred solution of 6-bromo-3-methyl-imidazo[1,5-a]pyrazine (500 mg, 2.36 mmol) in dioxane (10 mL), were added Pd₂ (dba) 3 (215.92 mg, 235.80 μmol), dicyclohexyl-[2-(2,6-dimethoxyphenyl)phenyl]phosphane (193.60 mg, 471.59 μmol), Na₂CO₃ (749.76 mg, 7.07 mmol) and HEXABUTYLDITIN (2.74 g, 4.72 mmol, 2.36 mL) under N₂, and the reaction mixture was stirred at 110° C. under N₂ atmosphere for 12 h. The reaction mixture was concentrated under reduced pressure to give crude product. The crude product was purified by flash silica gel chromatography to give tributyl-(3-methylimidazo[1,5-a]pyrazin-6-yl) stannane (113 mg, 11.35% yield) as a yellow oil.

MS(ESI) m/z=424.0 [M+H]⁺.

Step 4: N2-(2-(1-(difluoromethyl)-1H-pyrazol-3-yl) propan-2-yl)-6-(3-methylimidazo[1,5-a]pyrazin-6-yl)-1,3,5-triazine-2,4-diamine

To a stirred solution of tributyl-(3-methylimidazo[1,5-a]pyrazin-6-yl) stannane (60 mg, 142.11 μmol), 6-chloro-N4-[1-[1-(difluoromethyl) pyrazol-3-yl]-1-methyl-ethyl]-1,3,5-triazine-2,4-diamine (Example 10-1, 43.16 mg, 142.11 μmol) and Pd(PPh3) 4 (16.42 mg, 14.21 μmol) were taken up into a microwave tube in DMSO (4 mL). The sealed tube was heated at 160° C. for 2 h under microwave. The crude product was purified by column chromatography to give N2-(2-(1-(difluoromethyl)-1H-pyrazol-3-yl) propan-2-yl)-6-(3-methylimidazo[1,5-a]pyrazin-6-yl)-1,3,5-triazine-2,4-diamine (1.31 mg, 2.30% yield) as a white solid.

MS(ESI) m/z=401.2 [M+H]⁺.

1H NMR (400 MHZ, Methanol-d4) δ=9.10 (d, J=3.6 Hz, 2H), 7.98 (d, J=2.4 Hz, 1H), 7.94 (s, 1H), 7.42 (t, J=59.6 Hz, 1H), 6.54 (d, J=2.4 Hz, 1H), 2.80 (s, 3H), 1.87 (s, 6H)

### Example 14

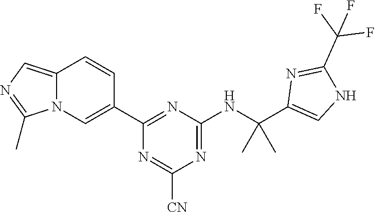 [SMILES: Cc1ncc2ccc(-c3nc(C#N)nc(NC(C)(C)c4c[nH]c(C(F)(F)F)n4)n3)cn12]

### 4-(3-methylimidazo[1,5-a]pyridin-6-yl)-6-[[1-methyl-1-[2-(trifluoromethyl)-1H-imidazol-4-yl]ethyl]amino]-1,3,5-triazine-2-carbonitrile (General Method for Target Compounds E)

MS(ESI) m/z=428.2 [M+H]⁺.

1H NMR (400 MHZ, CD3CN) 8=9.10-8.54 (m, 1H), 7.67-7.57 (m, 1H), 7.57-7.48 (m, 1H), 7.46-7.37 (m, 1H), 7.29-7.15 (m, 1H), 2.83-2.79 (m, 3H), 1.82-1.78 (m, 6H).

### Example 15

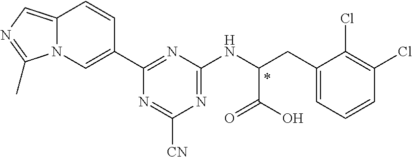 [SMILES: Cc1ncc2ccc(-c3nc(C#N)nc(NC(Cc4cccc(Cl)c4Cl)C(=O)O)n3)cn12]

### (2S)-2-[[4-cyano-6-(3-methylimidazo[1,5-a]pyridin-6-yl)-1,3,5-triazin-2-yl]amino]-3-(2,3-dichlorophenyl) propanoic acid (General Method for Target Compounds E)

MS(ESI) m/z=468.1 [M+H]⁺.

1H NMR (400 MHZ, DMSO-d6) δ=13.25 (br s, 1H), 9.26 (t, J=8.0 Hz, 1H), 8.94-8.82 (m, 1H), 7.62-7.42 (m, 1H), 7.54-7.17 (m, 5H), 5.10-4.75 (m, 1H), 3.58-3.46 (m, 1H), 3.20-3.16 (m, 1H), 2.75-2.65 (m, 3H).

### Example 16

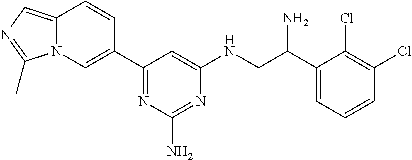 [SMILES: Cc1ncc2ccc(-c3cc(NCC(N)c4cccc(Cl)c4Cl)nc(N)n3)cn12]

### N4-[2-amino-2-(2,3-dichlorophenyl)ethyl]-6-(3-methylimidazo[1,5-a]pyridin-6-yl)pyrimidine-2,4-diamine (General Method for Target Compounds A)

MS(ESI) m/z=428.1 [M+H]⁺.

1H NMR (400 MHZ, Methanol-d4) δ=8.84 (s, 1H), 7.87-7.79 (m, 2H), 7.71-7.59 (m, 2H), 7.49 (t, J=8.0 Hz, 1H), 7.28 (dd, J=9.6, 0.8 Hz, 1H), 6.50 (s, 1H), 5.29 (t, J=6.4 Hz, 1H), 4.17-4.00 (m, 2H), 2.90 (s, 3H).

### Example 17

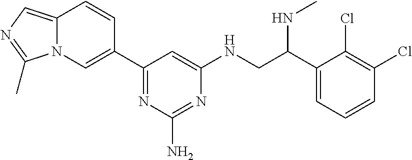 [SMILES: CNC(CNc1cc(-c2ccc3cnc(C)n3c2)nc(N)n1)c1cccc(Cl)c1Cl]

### N4-[2-(2,3-dichlorophenyl)-2-(methylamino)ethyl]-6-(3-methylimidazo[1,5-a]pyridin-6-yl)pyrimidine-2,4-diamine (General Method for Target Compounds A)

MS(ESI) m/z=442.1 [M+H]⁺.

1H NMR (400 MHz, DMSO-d6) δ=8.56 (s, 1H), 7.63 (d, J=7.6 Hz, 1H), 7.60-7.52 (m, 2H), 7.46-7.39 (m, 1H), 7.27 (s, 1H), 7.15 (d, J=8.0 Hz, 1H), 7.02 (br s, 1H), 6.31 (s, 1H), 6.10 (br s, 2H), 4.37 (br s, 1H), 3.80-3.36 (m, 3H), 2.64 (s, 3H), 2.24 (s, 3H).

### Example 18

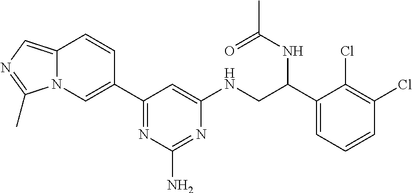 [SMILES: CC(=O)NC(CNc1cc(-c2ccc3cnc(C)n3c2)nc(N)n1)c1cccc(Cl)c1Cl]

### N-[2-[[2-amino-6-(3-methylimidazo[1,5-a]pyridin-6-yl)pyrimidin-4-yl]amino]-1-(2,3-dichlorophenyl)ethyl]acetamide

To a solution of N4-[2-amino-2-(2,3-dichlorophenyl)ethyl]-6-(3-methylimidazo[1,5-a]pyridin-6-yl)pyrimidine-2,4-diamine (Example 16, 26 mg, 60.70 μmol) in DCM (5 mL) was added DIEA (15.69 mg, 121.41 μmol) and (2,5-dioxopyrrolidin-1-yl)acetate (38.15 mg, 242.81 μmol). The mixture was stirred at 15° C. for 12 hr under N₂. The reaction was concentrated under reduced pressure to give a residue. The residue was purified by column chromatography to give product N-[2-[[2-amino-6-(3-methylimidazo[1,5-a]pyridin-6-yl)pyrimidin-4-yl]amino]-1-(2,3-dichlorophenyl)ethyl]acetamide (9.01 mg, 30.28% yield) as a yellow solid.

MS(ESI) m/z=470.1 [M+H]⁺.

*12191H NMR (400 MHZ, Methanol-d4) δ=8.86 (s, 1H), 7.87-7.74 (m, 2H), 7.50 (t, J=8.0 Hz, 2H), 7.35 (t, J=8.0 Hz, 1H), 7.26 (d, J=9.6 Hz, 1H), 6.46 (s, 1H), 5.73 (t, J=6.8 Hz, 1H), 3.89 (d, J=6.8 Hz, 2H), 2.91 (s, 3H), 2.01 (s, 3H).

### Example 19

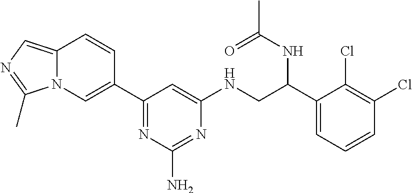 [SMILES: CC(=O)NC(CNc1cc(-c2ccc3cnc(C)n3c2)nc(N)n1)c1cccc(Cl)c1Cl]

### N-[2-[[2-amino-6-(3-methylimidazo[1,5-a]pyridin-6-yl)pyrimidin-4-yl]amino]-1-(2,3-dichlorophenyl)ethyl]-N-methyl-acetamide

To a stirred solution of N4-[2-(2,3-dichlorophenyl)-2-(methylamino)ethyl]-6-(3-methylimidazo[1,5-a]pyridin-6-yl)pyrimidine-2,4-diamine (Example 17, 100 mg, 226.07 μmol) in DCM (4 mL) were added (2,5-dioxopyrrolidin-1-yl)acetate (355.21 mg, 2.26 mmol) and DIEA (175.31 mg, 1.36 mmol), and the reaction mixture was stirred at 15° C. for 24 h under N₂. The reaction mixture was concentrated under reduced pressure to remove DCM. The crude product was purified by column chromatography to give N-[2-[[2-amino-6-(3-methylimidazo[1,5-a]pyridin-6-yl)pyrimidin-4-yl]amino]-1-(2,3-dichlorophenyl)ethyl]-N-methyl-acetamide (16.78 mg, 15.32% yield) as a white solid.

MS(ESI) m/z=484.2 [M+H]⁺.

1H NMR (400 MHz, Methanol-d4) δ=8.86-8.78 (m, 1H), 7.88-7.79 (m, 2H), 7.67-7.58 (m, 1H), 7.54 (d, J=7.6 Hz, 1H), 7.44-7.38 (m, 1H), 7.28 (d, J=10.0 Hz, 1H), 6.39 (s, 1H), 6.31-6.27 (m, 1H), 4.18-4.07 (m, 2H), 2.93-2.87 (m, 3H), 2.78-2.66 (m, 3H), 2.35-2.07 (m, 3H).

### Example 20

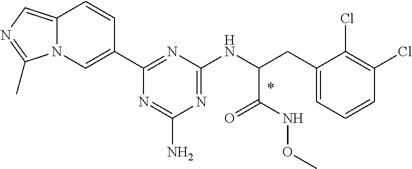 [SMILES: CONC(=O)C(Cc1cccc(Cl)c1Cl)Nc1nc(N)nc(-c2ccc3cnc(C)n3c2)n1]

### (2S or 2R)-2-[[4-amino-6-(3-methylimidazo[1,5-a]pyridin-6-yl)-1,3,5-triazin-2-yl]amino]-3-(2,3-dichlorophenyl)-N-methoxy-propanamide

Step 1: tert-butyl N-[(1S or 1R)-1-[(2,3-dichlorophenyl)methyl]-2-(methoxyamino)-2-oxo-ethyl]carbamate

To a stirred solution of (2S or 2R)-2-(tert-butoxycarbonylamino)-3-(2,3-dichlorophenyl) propanoic acid (140 mg, 418.92 μmol) in DCM (2 mL) were added O-methylhydroxylamine (69.97 mg, 837.84 μmol), DIPEA (162.42 mg, 1.26 mmol), EDCI (120.46 mg, 628.38 μmol) and HOBt (84.91 mg, 628.38 μmol) at 0° C. under N₂, and the reaction mixture was stirred at 15° C. for 12 h under N₂ atmosphere. The reaction mixture was concentrated under reduced pressure to give a crude product. The crude product was purified by column chromatography to give tert-butyl N-[(1S or 1R)-1-[(2,3-dichlorophenyl)methyl]-2-(methoxyamino)-2-oxo-ethyl]carbamate (140 mg, 92.00% yield) as a white solid.

MS(ESI) m/z=306.9 [M-tBu+H]⁺.

Step 2: (2S or 2R)-2-amino-3-(2,3-dichlorophenyl)-N-methoxy-propanamide

To a stirred solution of tert-butyl N-[(1S or 1R)-1-[(2,3-dichlorophenyl)methyl]-2-(methoxyamino)-2-oxo-ethyl]carbamate (180 mg, 495.55 μmol) in HCl/dioxane (2 mL, 4M) and dioxane (2 mL) and the reaction mixture was stirred at 15° C. for 2 h under N₂ atmosphere. The reaction mixture was concentrated under reduced pressure to give (2S or 2R)-2-amino-3-(2,3-dichlorophenyl)-N-methoxy-propanamide (140 mg, 94.30% yield) as an off-white solid.

MS(ESI) m/z=263.1 [M+H]⁺.

Step 3: (2S or 2R)-2-[(4-amino-6-chloro-1,3,5-triazin-2-yl)amino]-3-(2,3-dichlorophenyl)-N-methoxy-propanamide

To a stirred solution of (2S or 2R)-2-amino-3-(2,3-dichlorophenyl)-N-methoxy-propanamide (148 mg, 494.02 μmol) in i-PrOH (3 mL) were added DIEA (191.54 mg, 1.48 mmol) and 4,6-dichloro-1,3,5-triazin-2-amine (97.81 mg, 592.83 μmol), and the reaction mixture was stirred at 90° C. for 4 h under N₂. The reaction mixture was concentrated under reduced pressure to give a crude product. The crude product was purified by column chromatography to give (2S or 2R)-2-[(4-amino-6-chloro-1,3,5-triazin-2-yl)amino]-3-(2,3-dichlorophenyl)-N-methoxy-propanamide (149 mg, 77.01% yield) as an off-white solid.

MS(ESI) m/z=392.9 [M+2+H]⁺.

Step 4: (2S or 2R)-2-[[4-amino-6-(3-methylimidazo[1,5-a]pyridin-6-yl)-1,3,5-triazin-2-yl]amino]-3-(2,3-dichlorophenyl)-N-methoxy-propanamide

To a solution of (2S or 2R)-2-[(4-amino-6-chloro-1,3,5-triazin-2-yl)amino]-3-(2,3-dichlorophenyl)-N-methoxy-propanamide (20 mg, 51.07 μmol) and 3-methyl-6-(4,4,5,5-tetramethyl-1,3,2-dioxaborolan-2-yl) imidazo[1,5-a]pyridine (19.77 mg, 76.60 μmol) in THF (1 mL) and H₂O (0.1 mL) were added K₂CO₃ (14.12 mg, 102.13 μmol) and ditert-butyl(cyclopentyl)phosphane; dichloropalladium; iron (3.33 mg, 5.11 μmol). The reaction mixture was stirred at 75° C. for 4 h under N₂. The reaction mixture was concentrated under reduced pressure to give a crude product. The crude product was purified by column chromatography to give (2S or 2R)-2-[[4-amino-6-(3-methylimidazo[1,5-a]pyridin-6-yl)-1,3,5-triazin-2-yl]amino]-3-(2,3-dichlorophenyl)-N-methoxy-propanamide (1.84 mg, 7.39% yield) as a yellow solid.

MS(ESI) m/z=487.1 [M+H]⁺.

1H NMR (400 MHZ, Methanol-d4) δ=9.16-8.97 (m, 1H), 8.03-7.94 (m, 1H), 7.92 (s, 1H), 7.80 (dd, J=10.0, 1.2 Hz, 1H), 7.46-7.11 (m, 3H), 5.01-4.77 (m, 1H), 3.64-3.56 (m, 3H), 3.50-3.35 (m, 1H), 3.26-3.16 (m, 1H), 3.05-2.94 (m, 3H).

### Example 21

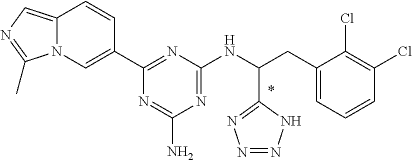 [SMILES: Cc1ncc2ccc(-c3nc(N)nc(NC(Cc4cccc(Cl)c4Cl)c4nnn[nH]4)n3)cn12]

### N4-[(1S or 1R)-2-(2,3-dichlorophenyl)-1-(1H-tetrazol-5-yl)ethyl]-6-(3-methylimidazo[1,5-a]pyridin-6-yl)-1,3,5-triazine-2,4-diamine

Step 1: (2S or 2R)-2-[(4-amino-6-chloro-1,3,5-triazin-2-yl)amino]-3-(2,3-dichlorophenyl) propanamide

To a solution of (2S or 2R)-2-amino-3-(2,3-dichlorophenyl) propanamide (700 mg, 3.00 mmol) in i-PrOH (14 mL) was added 4,6-dichloro-1,3,5-triazin-2-amine (495.45 mg, 3.00 mmol) and DIPEA (1.16 g, 9.01 mmol). The mixture was stirred at 90° C. for 6 hr under N₂ atmosphere. The reaction mixture was concentrated under reduced pressure to give a residue. The residue was purified by flash silica gel chromatography to give (2S or 2R)-2-[(4-amino-6-chloro-1,3,5-triazin-2-yl)amino]-3-(2,3-dichlorophenyl) propanamide (650 mg, 1.80 mmol, 59.86% yield) as a colorless oil.

MS(ESI) m/z=362.8 [M+H]⁺.

Step 2: (2S or 2R)-2-[[4-amino-6-(3-methylimidazo[1,5-a]pyridin-6-yl)-1,3,5-triazin-2-yl]amino]-3-(2,3-dichlorophenyl) propanamide

To a solution of (2S or 2R)-2-[(4-amino-6-chloro-1,3,5-triazin-2-yl)amino]-3-(2,3-dichlorophenyl) propanamide (650 mg, 1.80 mmol) in THF (6.5 mL) and H₂O (0.65 mL) was added 3-methyl-6-(4,4,5,5-tetramethyl-1,3,2-dioxaborolan-2-yl) imidazo[1,5-a]pyridine (556.77 mg, 2.16 mmol), K₂CO₃ (496.86 mg, 3.59 mmol) and ditert-butyl(cyclopentyl)phosphane; dichloropalladium; iron (117.15 mg, 179.75 μmol). The mixture was stirred at 75° C. for 3 hr under N₂. The reaction mixture was concentrated under reduced pressure to give a crude product. The residue was purified by column chromatography to give product (2S or 2R)-2-[[4-amino-6-(3-methylimidazo[1,5-a]pyridin-6-yl)-1,3,5-triazin-2-yl]amino]-3-(2,3-dichlorophenyl) propanamide (234 mg, 28.47% yield) as a yellow solid.

MS(ESI) m/z=457.0 [M+H]⁺.

Step 3: (2S or 2R)-2-[[4-amino-6-(3-methylimidazo[1,5-a]pyridin-6-yl)-1,3,5-triazin-2-yl]amino]-3-(2,3-dichlorophenyl) propanenitrile

To a solution of (2S or 2R)-2-[[4-amino-6-(3-methylimidazo[1,5-a]pyridin-6-yl)-1,3,5-triazin-2-yl]amino]-3-(2,3-dichlorophenyl) propanamide (100 mg, 218.67 μmol) in DCM (3 mL) was added methoxycarbonyl-(triethylammonio) sulfonyl-azanide (416.88 mg, 1.75 mmol). The mixture was stirred at 45° C. for 16 hr under N₂. The reaction was concentrated under reduced pressure to give a residue. The residue was purified by column chromatography to give product (2S or 2R)-2-[[4-amino-6-(3-methylimidazo[1,5-a]pyridin-6-yl)-1,3,5-triazin-2-yl]amino]-3-(2,3-dichlorophenyl) propanenitrile (120 mg, 62.46% yield) as a yellow solid.

MS(ESI) m/z=439.1 [M+H]⁺.

Step 4: N4-[(1S or 1R)-2-(2,3-dichlorophenyl)-1-(1H-tetrazol-5-yl)ethyl]-6-(3-methylimidazo[1,5-a]pyridin-6-yl)-1,3,5-triazine-2,4-diamine

To a solution of (2S or 2R)-2-[[4-amino-6-(3-methylimidazo[1,5-a]pyridin-6-yl)-1,3,5-triazin-2-yl]amino]-3-(2,3-dichlorophenyl) propanenitrile (30 mg, 68.29 μmol) in toluene (3 mL) was added AcOH (41.01 mg, 682.90 μmol), Et₃N (69.10 mg, 682.90 μmol) and NaN₃ (44.40 mg, 682.90 μmol). The mixture was stirred at 100° C. for 12 hr under N₂ atmosphere. The mixture was concentrated to give a crude product. The crude product was purified by column chromatography to give product N4-[(1S or 1R)-2-(2,3-dichlorophenyl)-1-(1H-tetrazol-5-yl)ethyl]-6-(3-methylimidazo[1,5-a]pyridin-6-yl)-1,3,5-triazine-2,4-diamine (6.6 mg, 20.04% yield) as a yellow solid.

MS(ESI) m/z=482.1 [M+H]⁺.

1H NMR (400 MHZ, Methanol-d4) δ=9.16-8.79 (m, 1H), 8.08-7.69 (m, 3H), 7.48-7.02 (m, 3H), 6.16-5.87 (m, 1H), 3.89-3.67 (m, 1H), 3.57-3.44 (m, 1H) 3.10-2.88 (m, 3H).

### Example 22

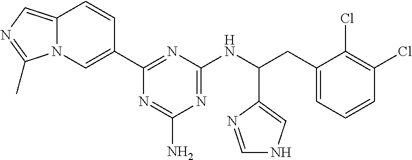 [SMILES: Cc1ncc2ccc(-c3nc(N)nc(NC(Cc4cccc(Cl)c4Cl)c4c[nH]cn4)n3)cn12]

### N4-[2-(2,3-dichlorophenyl)-1-(1-tritylimidazol-4-yl)ethyl]-6-(3-methylimidazo[1,5-a]pyridin-6-yl)-1,3,5-triazine-2,4-diamine (General Method for Target Compounds A)

MS(ESI) m/z=480.0 [M+H]⁺.

1H NMR (400 MHZ, DMSO-d6) δ=15.22 (br s, 1H), 14.74 (br s, 1H), 9.21-9.00 (m, 2H), 8.78 (br d, 1H), 8.10 (s, 1H), 7.93-7.86 (m, 1H), 7.78 (s, 1H), 7.76-7.64 (m, 1H), 7.63-7.40 (m, 2H), 7.35 (m, 1H), 7.31-7.16 (m, 1H), 7.22-7.15 (m, 1H), 6.06-6.03 (m, 0.5H), 5.60-5.57 (m, 0.5H), 3.57-3.35 (m, 2H), 3.06-2.90 (m, 3H).

### Example 23

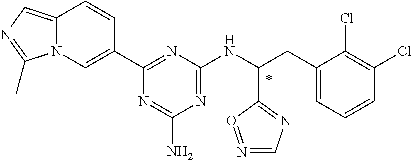

### N2-(2-(2,3-dichlorophenyl)-1-(1,2,4-oxadiazol-5-yl)ethyl)-6-(3-methylimidazo[1,5-a]pyridin-6-yl)-1,3,5-triazine-2,4-diamine (General Method for Target Compounds A)

MS(ESI) m/z=482.1 [M+H]⁺.

1H NMR (400 MHZ, DMSO-d6) δ=9.24 (br s, 2H), 8.91 (s, 1H), 7.96 (br s, 1H), 7.86 (br d, 2H), 7.80 (d, 1H), 7.63 (d, 1H), 7.32 (dd, 1H), 7.27-7.21 (m, 1H), 7.20-7.13 (m, 1H), 5.14 (t, 1H), 3.65-3.73 (m, 1H), 3.40-3.32 (m, 1H), 2.87 (s, 3H).

### Example 24

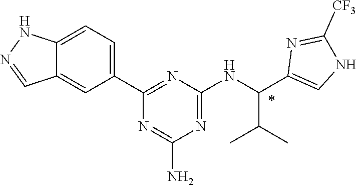 [SMILES: CC(C)C(Nc1nc(N)nc(-c2ccc3[nH]ncc3c2)n1)c1c[nH]c(C(F)(F)F)n1]

### 6-(1H-indazol-5-yl)-N4-[(1R or 1S)-2-methyl-1-[2-(trifluoromethyl)-1H-imidazol-4-yl]propyl]-1,3,5-triazine-2,4-diamine (General Method for Target Compounds C)

MS(ESI) m/z=418.2 [M+H]⁺.

1H NMR (400 MHZ, DMSO-d6) δ=13.43 (s, 1H), 8.78 (s, 1H), 8.63-8.38 (m, 1H), 8.37-8.31 (m, 1H), 8.31-8.02 (m, 2H), 7.67-8.00 (m, 2H), 7.66-7.39 (m, 1H), 7.35 (s, 1H), 5.22-5.00 (m, 1H), 2.27 (m, 1H), 1.01-0.93 (m, 3H), 0.90-0.83 (m, 3H).

### Example 25

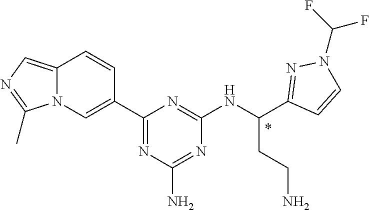 [SMILES: Cc1ncc2ccc(-c3nc(N)nc(NC(CCN)c4ccn(C(F)F)n4)n3)cn12]

### N2-[(1R or 1S)-3-amino-1-[1-(difluoromethyl) pyrazol-3-yl]propyl]-6-(3-methylimidazo[1,5-a]pyridin-6-yl)-1,3,5-triazine-2,4-diamine (General Method for Target Compounds A)

MS(ESI) m/z=415.1 [M+H]⁺.

1H NMR (400 MHZ, DMSO-d6) δ=9.15 (s, 1H), 8.38-8.04 (m, 5H), 7.96 (s, 1H), 7.90-7.58 (m, 3H), 7.30 (br s, 1H), 6.60 (d, 1H), 5.60-5.25 (m, 1H), 2.97 (s, 3H), 2.93 (d, 2H), 2.45-2.19 (m, 2H).

### Example 26

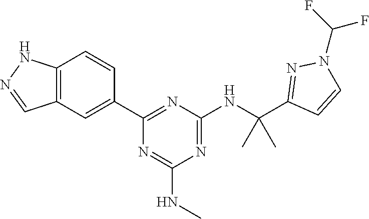 [SMILES: CNc1nc(NC(C)(C)c2ccn(C(F)F)n2)nc(-c2ccc3[nH]ncc3c2)n1]

### N4-[1-[1-(difluoromethyl) pyrazol-3-yl]-1-methyl-ethyl]-6-(1H-indazol-5-yl)-N2-methyl-1,3,5-triazine-2,4-diamine (General Method for Target Compounds D)

Step 1:4,6-dichloro-N-methyl-1,3,5-triazin-2-amine

To a solution of 2,4,6-trichloro-1,3,5-triazine (3 g, 16.27 mmol) in THF (20 mL) and cooled to −70° C. DIPEA (4.21 g, 32.54 mmol) and methanamine (1.10 g, 16.27 mmol) were added to the reaction mixture, which was stirred at −70° C. for 1 h and allowed to warm to 15° C. for 11 h under N₂ atmosphere. The reaction mixture was concentrated under reduced pressure to give a residue. The residue was purified by flash silica gel chromatography to give 4,6-dichloro-N-methyl-1,3,5-triazin-2-amine (5.03 g, 86.36% yield) as an off-white solid.

1H NMR (400 MHZ, DMSO-d6) δ=9.01 (br s, 1H), 2.83 (s, 3H).

Step 2:6-chloro-N4-[1-[1-(difluoromethyl) pyrazol-3-yl]-1-methyl-ethyl]-N2-methyl-1,3,5-triazine-2,4-diamine

To a solution of 4,6-dichloro-N-methyl-1,3,5-triazin-2-amine (169.16 mg, 945.00 μmol) and 2-[1-(difluoromethyl) pyrazol-3-yl]propan-2-amine (200 mg, 945.00 μmol) in i-PrOH (6 mL) was added DIPEA (366.40 mg, 2.84 mmol), then the mixture was stirred at 90° C. for 12 hr. The reaction mixture was concentrated under reduced pressure to give a residue. The residue was purified by flash silica gel chromatography to give 6-chloro-N4-[1-[1-(difluoromethyl) pyrazol-3-yl]-1-methyl-ethyl]-N2-methyl-1,3,5-triazine-2,4-diamine (205 mg, 68.28% yield) as a white solid.

1H NMR (400 MHZ, DMSO-d6) δ=8.11 (s, 1H), 8.02 (d, 1H), 7.86-7.55 (m, 2H), 6.32 (d, 1H), 2.41 (d, 3H), 1.64 (s, 6H).

Step 3: N4-[1-[1-(difluoromethyl) pyrazol-3-yl]-1-methyl-ethyl]-N2-methyl-6-(1-tetrahydropyran-2-ylindazol-5-yl)-1,3,5-triazine-2,4-diamine

To a stirred solution of 6-chloro-N4-[1-[1-(difluoromethyl) pyrazol-3-yl]-1-methyl-ethyl]-N2-methyl-1,3,5-triazine-2,4-diamine (205 mg, 645.21 μmol) in THF (2 mL) and H₂O (0.2 mL) were added 1-tetrahydropyran-2-yl-5-(4,4,5,5-tetramethyl-1,3,2-dioxaborolan-2-yl) indazole (317.65 mg, 967.82 μmol), ditert-butyl(cyclopentyl)phosphane; dichloropalladium; iron (42.05 mg, 64.52 μmol)ditert-butyl(cyclopentyl)phosphane; dichloropalladium; iron (42.05 mg, 64.52 μmol) and K₂CO₃ (178.34 mg, 1.29 mmol) and the reaction mixture was stirred at 75° C. for 12 h under N2 atmosphere. The reaction was filtered and concentrated under reduced pressure to give a residue. The residue was purified by flash silica gel chromatography to give the N4-[1-[1-(difluoromethyl) pyrazol-3-yl]-1-methyl-ethyl]-N2-methyl-6-(1-tetrahydropyran-2-ylindazol-5-yl)-1,3,5-triazine-2,4-diamine (170 mg, 54.49% yield) as a yellow solid.

MS(ESI) m/z=484.1 [M+H]⁺.

Step 4: N4-[1-[1-(difluoromethyl) pyrazol-3-yl]-1-methyl-ethyl]-6-(1H-indazol-5-yl)-N2-methyl-1,3,5-triazine-2,4-diamine

To a solution of N4-[1-[1-(difluoromethyl) pyrazol-3-yl]-1-methyl-ethyl]-N2-methyl-6-(1-tetrahydropyran-2-ylindazol-5-yl)-1,3,5-triazine-2,4-diamine (160 mg, 330.91 μmol) in DCM (5 mL) was added TFA (2.17 g, 19.01 mmol). The mixture was stirred at 15° C. for 12 h under N₂. The reaction mixture was concentrated under reduced pressure to give a crude product. It was purified by column chromatography to give N4-[1-[1-(difluoromethyl) pyrazol-3-yl]-1-methyl-ethyl]-6-(1H-indazol-5-yl)-N2-methyl-1,3,5-triazine-2,4-diamine (47.01 mg, 35.57% yield) as a white solid . . .

MS(ESI) m/z=399.7 [M+H]⁺.

1H NMR (400 MHZ, DMSO-d6) δ=8.59 (br s, 1H), 8.25-8.09 (m, 2H), 8.00 (d, 1H), 7.70 (t, 1H), 7.54 (d, 1H), 7.19 (br s, 2H), 6.45 (d, 1H), 2.81 (s, 3H), 1.79 (s, 6H).

### Example 27

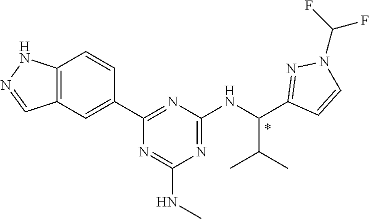 [SMILES: CNc1nc(NC(c2ccn(C(F)F)n2)C(C)C)nc(-c2ccc3[nH]ncc3c2)n1]

### N4-[(1R or 1S)-1-[1-(difluoromethyl) pyrazol-3-yl]-2-methyl-propyl]-6-(1H-indazol-5-yl)-N2-methyl-1,3,5-triazine-2,4-diamine (General Method for Target Compounds D)

MS(ESI) m/z=414.2 [M+H]⁺.

1H NMR (400 MHZ, METHANOL-d4) δ=8.79-8.61 (m, 1H), 8.27 (s, 1H), 8.16 (dd, 1H), 8.08-8.01 (m, 1H), 7.73 (d, 1H), 7.46 (t, 1H), 6.56 (d, 1H), 5.39-5.22 (m, 1H), 3.08 (s, 3H), 2.40-2.29 (m, 1H), 1.09 (d, 3H), 0.99 (d, 3H).

### Example 28

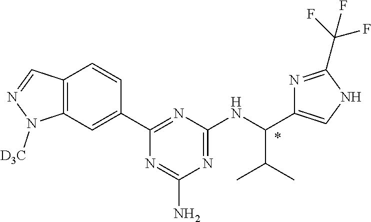 [SMILES: [2H]C([2H])([2H])n1ncc2ccc(-c3nc(N)nc(NC(c4c[nH]c(C(F)(F)F)n4)C(C)C)n3)cc21]

### N4-[(1R or 1S)-2-methyl-1-[2-(trifluoromethyl)-1H-imidazol-4-yl]propyl]-6-[1-(trideuteriomethyl) indazol-6-yl]-1, 3, 5-triazine-2, 4-diamine (General Method for Target Compounds A)

MS(ESI) m/z=435.2 [M+H]⁺.

1H NMR (400 MHZ, CD3OD) δ=8.55-8.49 (m, 1H), 8.14 (s, 1H), 8.05-7.85 (m, 2H), 7.35-7.23 (m, 1H), 5.39-5.08 (m, 1H), 2.48-2.22 (m, 1H), 1.11-1.04 (m, 3H), 1.01-0.94 (m, 3H).

### Example 29A & Example 29B

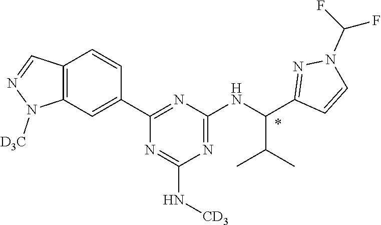 [SMILES: [2H]C([2H])([2H])Nc1nc(NC(c2ccn(C(F)F)n2)C(C)C)nc(-c2ccc3cnn(C([2H])([2H])[2H])c3c2)n1]

### N4-[(1S or 1R)-1-[1-(difluoromethyl) pyrazol-3-yl]-2-methyl-propyl]-N2-(trideuteriomethyl)-6-[1-(trideuteriomethyl) indazol-6-yl]-1,3,5-triazine-2,4-diamine (General Method for Target Compounds B)

29A

MS(ESI) m/z=434.2 [M+H]⁺.

1H NMR (400 MHZ, Methanol-d4) δ=8.44 (s, 1H), 8.13 (s, 1H), 8.04 (d, 1H), 7.98 (m, 2H), 7.46 (t, 1H), 6.56 (d, 1H), 5.40 (m, 1H), 2.40 (m, 1H), 1.09 (d, 3H), 0.98 (d, 3H).

29B

MS(ESI) m/z=434.3 [M+H]⁺.

1H NMR: (400 MHZ, DMSO-d6) δ=8.48 (s, 1H), 8.20 (m, 3H), 7.93 (m, 3H), 7.49 (br s, 1H), 6.60 (d, 1H), 5.20 (br s, 1H), 2.25 (m, 1H), 1.01 (d, 3H), 0.90 (d, 3H).

### Example 30

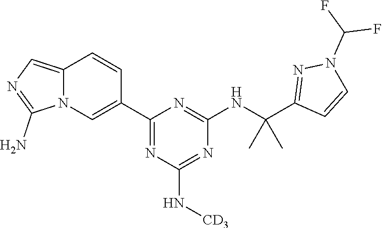 [SMILES: [2H]C([2H])([2H])Nc1nc(NC(C)(C)c2ccn(C(F)F)n2)nc(-c2ccc3cnc(N)n3c2)n1]

### 6-(3-aminoimidazo[1,5-a]pyridin-6-yl)-N2-[1-[1-(difluoromethyl) pyrazol-3-yl]-1-methyl-ethyl]-N4-(trideuteriomethyl)-1,3,5-triazine-2,4-diamine (General Method for Target Compounds D)

MS(ESI) m/z=418.2 [M+H]⁺.

1H NMR (400 MHZ, Methanol-d4) δ=8.72 (m, 1H), 8.01 (m, 1H), 7.55 (m, 2H), 7.22 (m, 2H), 6.48 (s, 1H), 1.83 (s, 6H).

### Example 31

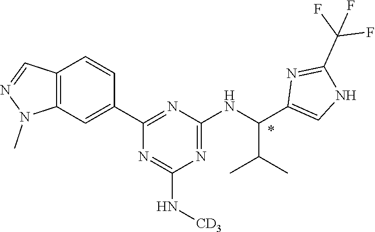 [SMILES: [2H]C([2H])([2H])Nc1nc(NC(c2c[nH]c(C(F)(F)F)n2)C(C)C)nc(-c2ccc3cnn(C)c3c2)n1]

6-(1-methylindazol-6-yl)-N4-[(1R)-2-methyl-1-[2-(trifluoromethyl)-1H-imidazol-4-yl]propyl]-N2-(trideuteriomethyl)-1,3,5-triazine-2,4-diamine (General Method for Target Compounds B)

MS(ESI) m/z=449.2 [M+H]⁺.

*13461H NMR (400 MHZ, Methanol-d4) δ=8.51 (m, 1H), 8.12 (s, 1H), 8.00 (m, 2H), 7.31 (s, 1H), 5.35 (m, 1H), 4.17 (s, 3H), 2.42 (m, 1H), 1.09 (d, 3H), 0.98 (d, 3H).

### Example 32

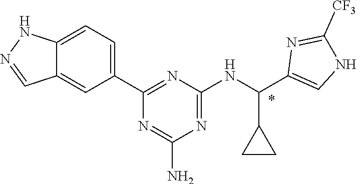 [SMILES: Nc1nc(NC(c2c[nH]c(C(F)(F)F)n2)C2CC2)nc(-c2ccc3[nH]ncc3c2)n1]

### N4-[(R or S)-cyclopropyl-[2-(trifluoromethyl)-1H-imidazol-4-yl]methyl]-6-(1H-indazol-5-yl)-1,3,5-triazine-2,4-diamine (General Method for Target Compounds B)

MS(ESI) m/z=416.1 [M+H]⁺.

1H NMR (400 MHZ, CD3OD) δ=8.78 (m, 1H), 8.12 (s, 1H), 8.16 (d, 1H), 7.81 (m, 1H), 7.41 (m, 1H), 4.79 (m, 1H), 1.58 (m, 1H), 0.76 (m, 2H), 0.62 (m, 1H), 0.49 (m, 1H).

### Example 33

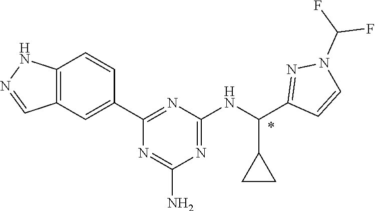 [SMILES: Nc1nc(NC(c2ccn(C(F)F)n2)C2CC2)nc(-c2ccc3[nH]ncc3c2)n1]

### N4-[(R)-cyclopropyl-[1-(difluoromethyl) pyrazol-3-yl]methyl]-6-(1H-indazol-5-yl)-1,3,5-triazine-2,4-diamine (General Method for Target Compounds B)

MS(ESI) m/z=398.2 [M+H]⁺.

1H NMR (400 MHZ, CD3OD) δ=8.69 (s, 1H), 8.27 (s, 1H), 8.21 (m, 1H), 8.02 (s, 1H), 7.76 (m, 1H), 7.45 (t, 1H), 6.62 (d, 1H), 4.81 (d, 1H), 1.52 (m, 1H), 0.76 (m, 2H), 0.62 (m, 2H).

### Example 34

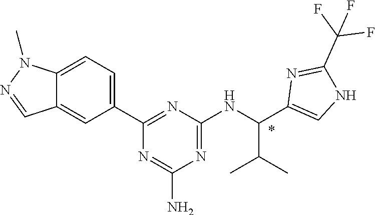 [SMILES: CC(C)C(Nc1nc(N)nc(-c2ccc3c(cnn3C)c2)n1)c1c[nH]c(C(F)(F)F)n1]

### 6-(1-methylindazol-5-yl)-N4-[(1R)-2-methyl-1-[2-(trifluoromethyl)-1H-imidazol-4-yl]propyl]-1,3,5-triazine-2,4-diamine (General Method for Target Compounds A)

MS(ESI) m/z=432.2 [M+H]⁺.

1H NMR (400 MHZ, DMSO-d6) δ=8.73 (m, 1H), 8.38 (m, 1H), 8.19 (d, 1H), 7.67 (d, J=9.2 Hz, 1H), 7.43 (m, 2H), 6.93 (m, 2H), 5.25 (m, 1H), 4.07 (s, 3H), 2.25 (m, 1H), 0.98 (m, 3H), 0.84 (m, 3H).

### Example 35

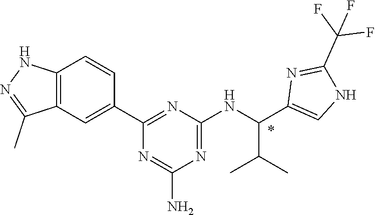 [SMILES: Cc1n[nH]c2ccc(-c3nc(N)nc(NC(c4c[nH]c(C(F)(F)F)n4)C(C)C)n3)cc12]

### 6-(3-methyl-1H-indazol-5-yl)-N4-[(1R)-2-methyl-1-[2-(trifluoromethyl)-1H-imidazol-4-yl]propyl]-1,3,5-triazine-2,4-diamine (General Method for Target Compounds A)

MS(ESI) m/z=432.2 [M+H]⁺.

1H NMR (400 MHZ, CD3OD) δ=8.68 (m, 1H), 8.30 (m, 1H), 7.62 (d, 1H), 7.28 (s, 1H), 5.34 (m, 1H), 2.64 (s, 3H), 2.39 (m, 1H), 1.07 (m, 3H), 0.97 (m, 3H).

### Example 36

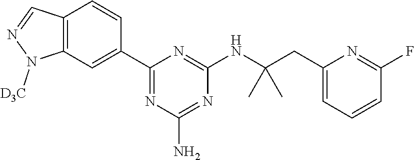 [SMILES: [2H]C([2H])([2H])n1ncc2ccc(-c3nc(N)nc(NC(C)(C)Cc4cccc(F)n4)n3)cc21]

### N2-[2-(6-fluoro-2-pyridyl)-1,1-dimethyl-ethyl]-6-[1-(trideuteriomethyl) indazol-6-yl]-1,3,5-triazine-2,4-diamine (General Method for Target Compounds A)

MS(ESI) m/z=396.2 [M+H]⁺.

1H NMR (400 MHZ, CD3OD) δ=8.68 (m, 1H), 8.30 (m, 2H), 7.87 (m, 2H), 7.14 (m, 1H), 6.87 (br d, 1H), 3.42 (s, 2H), 1.51 (s, 6H).

### Example 37

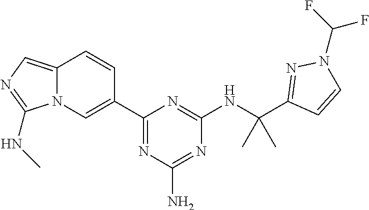 [SMILES: CNc1ncc2ccc(-c3nc(N)nc(NC(C)(C)c4ccn(C(F)F)n4)n3)cn12]

### N4-[1-[1-(difluoromethyl) pyrazol-3-yl]-1-methyl-ethyl]-6-[1-(methylamino) indazol-6-yl]-1,3,5-triazine-2,4-diamine (General Method for Target Compounds A)

MS(ESI) m/z=415.2 [M+H]⁺.

1H NMR (400 MHZ, Methanol-d4) δ=9.01 (m, 1H), 7.90 (s, 1H), 7.61 (m, 4H), 6.45 (s, 1H), 3.17 (s, 3H), 1.82 (s, 6H).

### Example 38

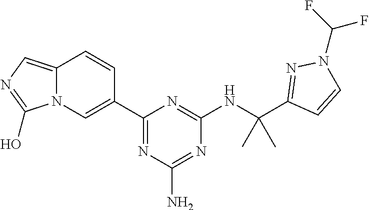 [SMILES: CC(C)(Nc1nc(N)nc(-c2ccc3cnc(O)n3c2)n1)c1ccn(C(F)F)n1]

### 6-[4-amino-6-[[1-[1-(difluoromethyl) pyrazol-3-yl]-1-methyl-ethyl]amino]-1,3,5-triazin-2-yl]imidazo[1,5-a]pyridin-3-ol (General Method for Target Compounds A)

MS(ESI) m/z=402.1 [M+H]⁺.

1H NMR (400 MHZ, Methanol-d4) δ=8.60 (m, 1H), 7.94 (s, 1H), 7.68 (m, 1H), 7.09 (m, 3H), 6.51 (s, 1H), 1.83 (s, 6H).

### Example 39

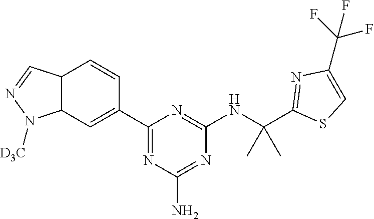 [SMILES: [2H]C([2H])([2H])N1N=CC2C=CC(c3nc(N)nc(NC(C)(C)c4nc(C(F)(F)F)cs4)n3)=CC21]

### N2-[1-methyl-1-[4-(trifluoromethyl) thiazol-2-yl]ethyl]-6-[1-(trideuteriomethyl) indazol-6-yl]-1,3,5-triazine-2,4-diamine (General Method for Target Compounds A)

MS(ESI) m/z=438.1 [M+H]⁺.

1H NMR (400 MHZ, Methanol-d4) δ=8.55 (m, 1H), 8.18 (m, 2H), 7.94 (br s, 1H), 7.81 (m, 1H), 1.95 (s, 6H).

### Example 40

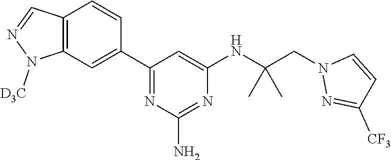 [SMILES: [2H]C([2H])([2H])n1ncc2ccc(-c3cc(NC(C)(C)Cn4ccc(C(F)(F)F)n4)nc(N)n3)cc21]

### N4-[1,1-dimethyl-2-[3-(trifluoromethyl) pyrazol-1-yl]ethyl]-6-[1-(trideuteriomethyl) indazol-6-yl]pyrimidine-2,4-diamine (General Method for Target Compounds A)

MS(ESI) m/z=434.2 [M+H]⁺.

1H NMR (400 MHZ, CD3OD) δ=8.13 (s, 1H), 8.01 (m, 2H), 7.67 (s, 1H), 7.44 (d, 1H), 6.59 (d, 1H), 6.40 (s, 1H), 4.84 (s, 2H), 1.52 (s, 6H).

### Example 41

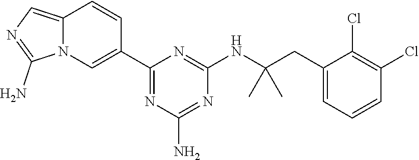 [SMILES: CC(C)(Cc1cccc(Cl)c1Cl)Nc1nc(N)nc(-c2ccc3cnc(N)n3c2)n1]

### 6-(3-aminoimidazo[1,5-a]pyridin-6-yl)-N2-[2-(2,3-dichlorophenyl)-1,1-dimethyl-ethyl]-1,3,5-triazine-2,4-diamine (General Method for Target Compounds B)

MS(ESI) m/z=443.1 [M+H]⁺.

1H NMR (400 MHZ, DMSO-d6) δ=8.94 (m, 1H), 8.33 (s, 2H), 7.50 (m, 3H), 7.38 (s, 1H), 7.26 (t, 1H), 7.13 (s, 1H), 7.05 (m, 2H), 6.87 (m, 1H), 3.52 (s, 2H), 1.39 (s, 6H).

### Example 42

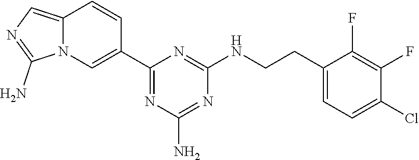 [SMILES: Nc1nc(NCCc2ccc(Cl)c(F)c2F)nc(-c2ccc3cnc(N)n3c2)n1]

### 6-(3-aminoimidazo[1,5-a]pyridin-6-yl)-N2-[2-(4-chloro-2,3-difluoro-phenyl)ethyl]-1,3,5-triazine-2,4-diamine (General Method for Target Compounds B)

MS(ESI) m/z=417.2 [M+H]⁺.

1H NMR (400 MHZ, CD3OD) δ=8.92 (s, 1H), 7.60 (m, 1H), 7.44 (m, 1H), 7.25 (d, 1H), 7.21 (m, 1H), 7.12 (m, 1H), 3.82 (t, 1H), 3.69 (t, 1H), 3.01 (t, 2H).

### Example 43

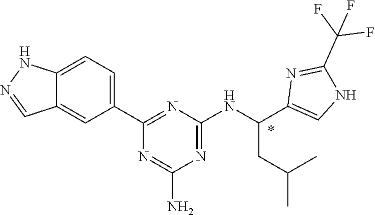 [SMILES: CC(C)CC(Nc1nc(N)nc(-c2ccc3[nH]ncc3c2)n1)c1c[nH]c(C(F)(F)F)n1]

### 6-(1H-indazol-5-yl)-N4-[(1S or 1R)-3-methyl-1-[2-(trifluoromethyl)-1H-imidazol-4-yl]butyl]-1,3,5-triazine-2,4-diamine (General Method for Target Compounds B)

MS(ESI) m/z=432.4 [M+H]⁺.

1H NMR (400 MHZ, DMSO-d6) δ=13.37 (m, 2H), 8.75 (d, 1H), 8.35 (m, 1H), 8.21 (s, 1H), 7.57 (d, 1H), 7.50 (m, 1H), 7.26 (m, 1H), 6.81 (br s, 1H), 6.68 (br s, 1H), 5.48 (m, 1H), 1.78 (br s, 1H), 1.69 (m, 1H), 0.96 (m, 6H).

### Example 44

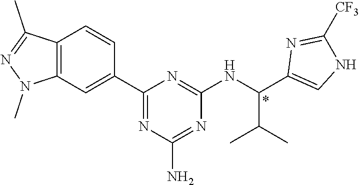 [SMILES: Cc1nn(C)c2cc(-c3nc(N)nc(NC(c4c[nH]c(C(F)(F)F)n4)C(C)C)n3)ccc12]

### 6-(1,3-dimethylindazol-6-yl)-N2-[(1R or 1S)-2-methyl-1-[2-(trifluoromethyl)-1H-imidazol-4-yl]propyl]-1,3,5-triazine-2,4-diamine (General Method for Target Compounds A)

MS(ESI) m/z=446.2 [M+H]⁺.

1H NMR (400 MHZ, Methanol-d4) δ=8.43 (m, 1H), 7.99 (m, 2H), 7.32 (m, 1H), 5.42 (m, 1H), 4.15 (m, 3H), 2.59 (s, 3H), 2.45 (m, 1H), 1.14 (m, 3H), 1.01 (m, 3H).

### Example 45

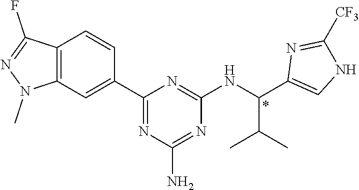 [SMILES: CC(C)C(Nc1nc(N)nc(-c2ccc3c(F)nn(C)c3c2)n1)c1c[nH]c(C(F)(F)F)n1]

### 6-(3-fluoro-1-methyl-indazol-6-yl)-N2-[(1R or 1S)-2-methyl-1-[2-(trifluoromethyl)-1H-imidazol-4-yl]propyl]-1,3,5-triazine-2,4-diamine (General Method for Target Compounds A)

MS(ESI) m/z=450.2 [M+H]⁺.

1H NMR (400 MHZ, DMSO-d6) δ=8.42 (br s, 1H), 8.11 (br d, 1H), 8.04-7.79 (m, 2H), 7.72-7.37 (m, 1H), 7.36-7.13 (m, 2H), 5.24-4.97 (m, 1H), 4.02-3.98 (m, 3H), 2.29-2.16 (m, 1H), 0.99-0.91 (m, 3H), 0.89-0.81 (m, 3H).

### * 1434Example 46

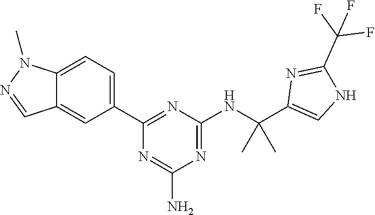 [SMILES: Cn1ncc2cc(-c3nc(N)nc(NC(C)(C)c4c[nH]c(C(F)(F)F)n4)n3)ccc21]

### 6-(1-methyl-1H-indazol-5-yl)-N2-(2-(2-(trifluoromethyl)-1H-imidazol-4-yl) propan-2-yl)-1,3,5-triazine-2,4-diamine

MS(ESI) m/z=399.9 [M+H]⁺.

1H NMR (400 MHZ, DMSO-d6): δ=8.52 (br s, 1H), 8.19-8.11 (m, 2H), 8.03 (d, 1H), 7.73 (t, 1H), 7.64 (br d, 1H), 7.58 (s, 1H), 7.05 (br s, 2H), 6.47 (s, 1H), 4.07 (s, 3H), 1.79 (s, 6H).

### Example 47A

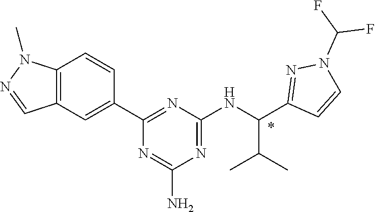 [SMILES: CC(C)C(Nc1nc(N)nc(-c2ccc3c(cnn3C)c2)n1)c1ccn(C(F)F)n1]

### (R or S)—N2-(1-(1-(difluoromethyl)-1H-pyrazol-3-yl)-2-methylpropyl)-6-(1-methyl-1H-indazol-5-yl)-1,3,5-triazine-2,4-diamine

MS(ESI) m/z=414.2 [M+H]⁺.

1H NMR (400 MHz, DMSO-d6): δ=8.71 (s, 1H), 8.29 (br d, 1H), 8.20 (s, 1H), 8.09 (br s, 1H), 7.79 (t, 1H), 7.76-7.62 (m, 2H), 7.02 (br s, 2H), 6.56 (d, 1H), 5.43-5.01 (m, 1H), 4.08 (s, 3H), 2.33-2.08 (m, 1H), 0.99 (d, 3H), 0.88 (d, 3H).

### Example 48

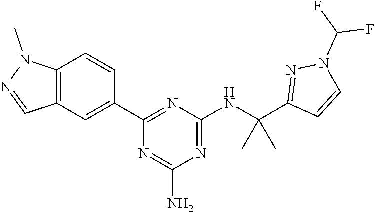 [SMILES: Cn1ncc2cc(-c3nc(N)nc(NC(C)(C)c4ccn(C(F)F)n4)n3)ccc21]

### N2-(2-(1-(difluoromethyl)-1H-pyrazol-3-yl) propan-2-yl)-6-(1-methyl-1H-indazol-5-yl)-1,3,5-triazine-2,4-diamine

MS(ESI) m/z=399.9 [M+H]⁺.

1H NMR (400 MHZ, DMSO-d6): δ=8.52 (br s, 1H), 8.19-8.11 (m, 2H), 8.03 (d, 1H), 7.73 (t, 1H), 7.64 (br d, 1H), 7.58 (s, 1H), 7.05 (br s, 2H), 6.47 (s, 1H), 4.07 (s, 3H), 1.79 (s, 6H).

### Example 135

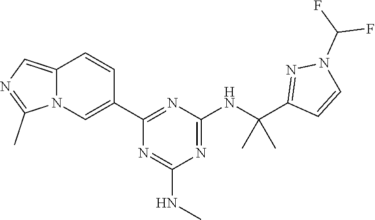 [SMILES: CNc1nc(NC(C)(C)c2ccn(C(F)F)n2)nc(-c2ccc3cnc(C)n3c2)n1]

### N4-[1-[1-(difluoromethyl) pyrazol-3-yl]-1-methyl-ethyl]-N2-methyl-6-(3-methylimidazo[1,5-a]pyridin-6-yl)-1,3,5-triazine-2,4-diamine (General Method for Target Compounds B)

MS(ESI) m/z 414.2 [M+H]⁺.

1H NMR (400 MHZ, DMSO-d6) δ=8.70 (s, 1H), 7.98 (d, 1H), 7.48 (t, 1H), 7.49-7.42 (m, 1H), 7.39 (br s, 1H), 7.26 (s, 1H), 7.22-6.88 (m, 2H), 6.42 (d, 1H), 3.05 (s, 3H), 2.63 (s, 3H), 1.77-1.75 (s, 6H).

### Example 136A & Example 136B

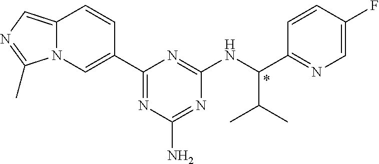 [SMILES: Cc1ncc2ccc(-c3nc(N)nc(NC(c4ccc(F)cn4)C(C)C)n3)cn12]

### N2-[(1R or 1S)-1-(5-fluoro-2-pyridyl)-2-methyl-propyl]-6-(3-methylimidazo[1,5-a]pyridin-6-yl)-1,3,5-triazine-2,4-diamine (General Method for Target Compounds A)

136A

MS(ESI) m/z 393.0 [M+H]⁺.

1H NMR (400 MHZ, Methanol-d4) δ=8.85 (d, 1H), 8.44-8.42 (m, 1H), 8.22 (s, 1H), 7.61-7.48 (m, 4H), 7.30 (s, 1H), 5.11-5.03 (m, 1H), 2.72 (d, 3H), 2.30-2.24 (m, 1H), 1.07-1.03 (m, 3H), 0.90-0.86 (m, 3H).

136B

MS(ESI) m/z=392.9 [M+H]⁺.

1H NMR (400 MHZ, Methanol-d4) δ=8.85 (d, 1H), 8.44-8.42 (m, 1H), 7.61-7.31 (m, 4H), 7.29 (s, 1H), 5.09 (dd, 1H), 2.71 (d, 3H), 2.30-2.24 (m, 1H), 1.07-1.03 (m, 3H), 0.90-0.86 (m, 3H).

### Example 137

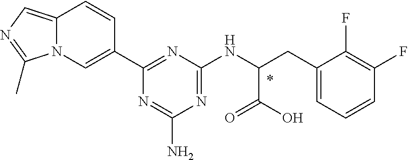 [SMILES: Cc1ncc2ccc(-c3nc(N)nc(NC(Cc4cccc(F)c4F)C(=O)O)n3)cn12]

### (2S or 2R)-2-[[4-amino-6-(3-methylimidazo[1,5-a]pyridin-6-yl)-1,3,5-triazin-2-yl]amino]-3-(2,3-difluorophenyl) propanoic acid (General Method for Target Compounds A)

MS(ESI) m/z 426.2 [M+H]⁺.

1H NMR (400 MHZ, DMSO-d6) δ=8.80 (s, 1H), 8.13 (s, 1H), 7.57-7.41 (m, 2H), 7.28 (s, 1H), 7.25-7.00 (m, 4H), 6.68 (br s, 2H), 4.82 (br d, 1H), 3.32-3.28 (m, 2H), 2.65 (s, 3H).

### Example 138

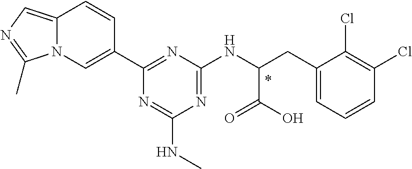 [SMILES: CNc1nc(NC(Cc2cccc(Cl)c2Cl)C(=O)O)nc(-c2ccc3cnc(C)n3c2)n1]

*1476

### (2S or 2R)-3-(2,3-dichlorophenyl)-2-[[4-(methylamino)-6-(3-methylimidazo[1,5-a]pyridin-6-yl)-1,3,5-triazin-2-yl]amino]propanoic acid (General Method for Target Compounds B)

MS(ESI) m/z 472.1 [M+H]⁺.

1H NMR (400 MHZ, DMSO-d6) δ=8.78 (s, 1H), 7.57-7.50 (m, 1H), 7.49-7.31 (m, 4H), 7.31-7.18 (m, 2H), 7.14 (br s, 1H), 4.91 (br s, 1H), 3.47-3.42 (m, 1H), 3.28-3.17 (m, 1H), 2.84 (br s, 3H), 2.66 (s, 3H).

### Example 139

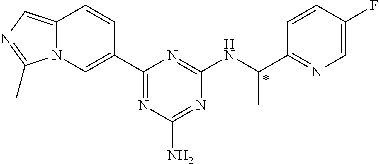 [SMILES: Cc1ncc2ccc(-c3nc(N)nc(NC(C)c4ccc(F)cn4)n3)cn12]

### N2-[(1R or 1S)-1-(5-fluoro-2-pyridyl)ethyl]-6-(3-methylimidazo[1,5-a]pyridin-6-yl)-1,3,5-triazine-2,4-diamine (General Method for Target Compounds A)

MS(ESI) m/z 364.9 [M+H]⁺.

1H NMR (400 MHZ, Methanol-d4) δ=8.85 (m, 1H), 8.41 (s, 1H), 8.26 (s, 1H), 7.62-7.47 (m, 4H), 7.29 (s, 1H), 5.36-5.26 (m, 1H), 2.70 (s, 3H), 1.55 (d, 3H).

### Example 140

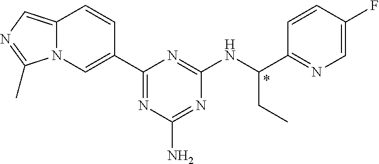 [SMILES: CCC(Nc1nc(N)nc(-c2ccc3cnc(C)n3c2)n1)c1ccc(F)cn1]

### N2-[(1R or 1S)-1-(5-fluoro-2-pyridyl) propyl]-6-(3-methylimidazo[1,5-a]pyridin-6-yl)-1,3,5-triazine-2,4-diamine (General Method for Target Compounds A)

MS(ESI) m/z 378.9 [M+H]⁺.

1H NMR (400 MHZ, Methanol-d4) δ=8.76 (m, 1H), 8.41 (d, J=7.6 Hz, 1H), 8.23 (s, 1H), 7.57-7.50 (m, 4H), 7.28 (s, 1H), 5.20-5.04 (m, 1H), 2.69 (s, 3H), 2.07-1.81 (m, 2H), 1.04-0.93 (m, 3H).

### Example 141A & Example 141B

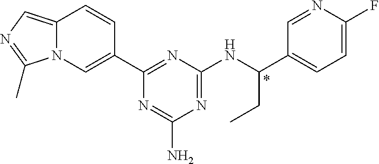 [SMILES: CCC(Nc1nc(N)nc(-c2ccc3cnc(C)n3c2)n1)c1ccc(F)nc1]

### N2-[(1R or 1S)-1-(6-fluoro-3-pyridyl) propyl]-6-(3-methylimidazo[1,5-a]pyridin-6-yl)-1,3,5-triazine-2,4-diamine (General Method for Target Compounds A)

141A

MS(ESI) m/z 379.2 [M+H]⁺.

1H NMR (400 MHZ, DMSO-d6) δ=8.77 (s, 1H), 8.30 (s, 1H), 8.02 (dt, 1H), 7.65 (s, 1H), 7.55-7.45 (m, 2H), 7.28 (s, 1H), 7.10 (dd, 1H), 6.64 (s, 2H), 5.04 (s, 1H), 2.66 (s, 3H), 2.01-1.76 (m, 2H), 0.93 (t, 3H).

141B

LCMS m/z (ESI+) 379.2 [M+H]⁺.

1H NMR (400 MHZ, DMSO-d6) δ=8.77 (s, 1H), 8.30 (s, 1H), 8.02 (dt, 1H), 7.65 (s, 1H), 7.55-7.45 (m, 2H), 7.28 (s, 1H), 7.10 (dd, 1H), 6.64 (s, 2H), 5.04 (s, 1H), 2.66 (s, 3H), 2.01-1.76 (m, 2H), 0.93 (t, 3H).

### Example 142A & Example 142B

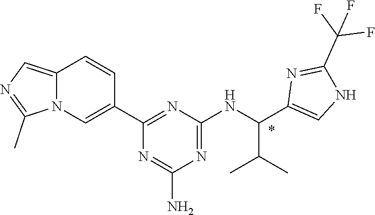 [SMILES: Cc1ncc2ccc(-c3nc(N)nc(NC(c4c[nH]c(C(F)(F)F)n4)C(C)C)n3)cn12]

### 6-(3-methylimidazo[1,5-a]pyridin-6-yl)-N2-[(1R or 1S)-2-methyl-1-[2-(trifluoromethyl)-1H-imidazol-4-yl]propyl]-1,3,5-triazine-2,4-diamine (General Method for Target Compounds A)

142A

LCMS(ESI) m/z=432.0 [M+H]⁺.

1H NMR (400 MHz, DMSO-d6) δ=8.81 (s, 1H), 8.19 (s, 1H), 7.52 (s, 2H), 7.28 (s, 1H), 7.21 (br s, 1H), 7.17-6.94 (m, 1H), 6.65 (br s, 2H), 5.09 (br s, 1H), 2.65 (s, 3H), 2.22 (qd, 1H), 0.96 (d, 3H), 0.87 (d, 3H).

142B

*1514LCMS m/z (ESI+) 432.3 [M+H]⁺.

1H NMR (400 MHZ, DMSO-d6) δ=8.81 (s, 1H), 8.16 (s, 1H), 7.52 (d, 2H), 7.28 (s, 1H), 7.21 (br s, 1H), 7.10 (br s, 1H), 6.64 (br s, 2H), 5.09 (br s, 1H), 2.65 (s, 3H), 2.22 (qd, 1H), 0.96 (d, 3H), 0.87 (d, 3H).

### Example 143A & Example 143B

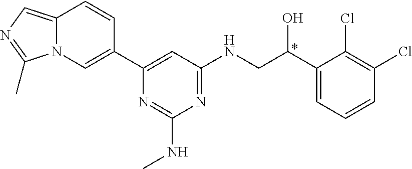 [SMILES: CNc1nc(NCC(O)c2cccc(Cl)c2Cl)cc(-c2ccc3cnc(C)n3c2)n1]

### (1R or 1S)-1-(2,3-dichlorophenyl)-2-[[2-(methylamino)-6-(3-methylimidazo[1,5-a]pyridin-6-yl)pyrimidin-4-yl]amino]ethanol (General Method for Target Compounds B)

143A

LCMS m/z (ESI+) 443.1 [M+H]⁺.

1H NMR (400 MHZ, Methanol-d4) δ=8.54 (s, 1H), 7.59 (d, 1H), 7.51 (d, 1H), 7.43 (d, 1H), 7.34-7.24 (m, 2H), 7.19 (d, 1H), 6.21 (s, 1H), 5.32 (m, 1H), 3.88-3.58 (m, 1H), 3.69 (s, 1H), 2.96 (s, 3H), 2.68 (s, 3H).

143B

LCMS m/z (ESI+) 443.1 [M+H]⁺.

1H NMR (400 MHZ, Methanol-d4): δ=8.57 (s, 1H), 7.62 (dd, 1H), 7.54 (d, 1H), 7.44 (dd, 1H), 7.33-7.29 (m, 2H), 7.19 (d, 1H), 6.24 (s, 1H), 5.35 (dd, 1H), 3.84-3.67 (m, 2H), 2.98 (s, 3H), 2.71 (s, 3H).

### Example 144

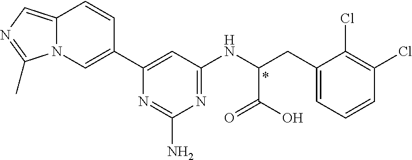 [SMILES: Cc1ncc2ccc(-c3cc(NC(Cc4cccc(Cl)c4Cl)C(=O)O)nc(N)n3)cn12]

### (2R or 2S)-2-[[2-amino-6-(3-methylimidazo[1,5-a]pyridin-6-yl)pyrimidin-4-yl]amino]-3-(2,3-difluorophenyl) propanoic acid (General Method for Target Compounds A)

LCMS m/z (ESI+) 425.0 [M+H]⁺.

1H NMR (400 MHZ, DMSO-d6) δ=8.53 (s, 1H), 8.14 (s, 1H), 7.53 (d, 1H), 7.26 (s, 1H), 7.24-7.17 (m, 2H), 7.17-7.04 (m, 3H), 6.97 (br s, 1H), 6.37 (s, 1H), 5.89 (s, 1H), 4.84 (br s, 1H), 3.30-3.12 (m, 2H), 2.64 (s, 3H).

### Example 145

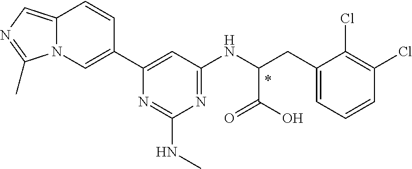 [SMILES: CNc1nc(NC(Cc2cccc(Cl)c2Cl)C(=O)O)cc(-c2ccc3cnc(C)n3c2)n1]

### (2R or 2S)-3-(2,3-dichlorophenyl)-2-[[2-(methylamino)-6-(3-methylimidazo[1,5-a]pyridin-6-yl)pyrimidin-4-yl]amino]propanoic acid (General Method for Target Compounds B)

LCMS m/z (ESI+) 471.0 [M+H]⁺.

1H NMR (400 MHZ, DMSO-d6): δ=8.52 (s, 1H), 7.52 (d, 1H), 7.43 (d, 1H), 7.37 (d, 1H), 7.29-7.21 (m, 2H), 7.16 (d, 1H), 6.93 (s, 1H), 6.27 (s, 1H), 6.17 (s, 1H), 4.88 (s, 1H), 3.35-3.31 (m, 2H), 2.81 (d, 3H), 2.63 (s, 3H).

### Example 146A & Example 146B

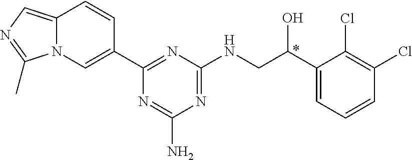 [SMILES: Cc1ncc2ccc(-c3nc(N)nc(NCC(O)c4cccc(Cl)c4Cl)n3)cn12]

### (1S or 1R)-[[4-amino-6-(3-methylimidazo[1,5-a]pyridin-6-yl)-1,3,5-triazin-2-yl]amino]-1-(2,3-dichlorophenyl) ethanol (General Method for Target Compounds A)

*1544

146A

LCMS m/z (ESI+) 429.9 [M+H]⁺.

1H NMR (400 MHZ, Methanol-d4) δ=8.87 (s, 1H), 7.71-7.41 (m, 3H), 7.34-7.26 (m, 2H), 7.23-7.13 (m, 1H), 5.33 (d, 1H), 3.82-3.70 (m, 1H), 3.66-3.55 (m, 1H), 2.85-2.66 (s, 3H).

146B

LCMS m/z (ESI+) 429.9 [M+H]⁺.

1H NMR (400 MHZ, Methanol-d4) δ=8.87 (s, 1H), 7.67-7.40 (m, 3H), 7.36-7.25 (m, 2H), 7.24-7.12 (m, 1H), 5.33 (d, 1H), 3.82-3.70 (m, 1H), 3.62 (dd, 1H), 2.73 (s, 3H).

### Example 147

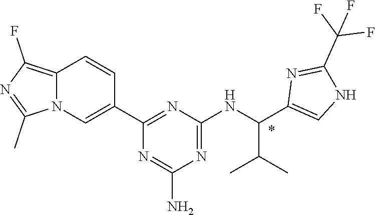 [SMILES: Cc1nc(F)c2ccc(-c3nc(N)nc(NC(c4c[nH]c(C(F)(F)F)n4)C(C)C)n3)cn12]

### 6-(1-fluoro-3-methyl-imidazo[1,5-a]pyridin-6-yl)-N2-[(1R)-2-methyl-1-[2-(trifluoromethyl)-1H-imidazol-4-yl]propyl]-1,3,5-triazine-2,4-diamine

LCMS m/z=450.2 [M+H]⁺.

1H NMR (400 MHZ, CD3OD) δ=8.77 (s, 1H), 7.48 (d, 1H), 7.38 (d, 1H), 7.24-7.03 (m, 1H), 5.23-5.10 (m, 1H), 2.63 (s, 3H), 2.40-2.05 (m, 1H), 1.08-0.97 (m, 3H), 0.96-0.87 (m, 3H).

### Example 148

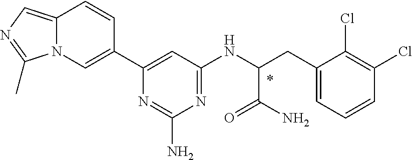 [SMILES: Cc1ncc2ccc(-c3cc(NC(Cc4cccc(Cl)c4Cl)C(N)=O)nc(N)n3)cn12]

### (2S or 2R)-2-[[2-amino-6-(3-methylimidazo[1,5-a]pyridin-6-yl)pyrimidin-4-yl]amino]-3-(2,3-dichlorophenyl) propanamide (General Method for Target Compounds A)

LCMS m/z (ESI+) 456.1 [M+H]⁺.

1H NMR (400 MHZ, Methanol-d4): δ=8.54 (s, 1H), 7.57 (d, 1H), 7.42-7.40 (m, 1H), 7.33-7.32 (m, 2H), 7.22-7.14 (m, 2H), 6.30 (br s, 1H), 3.50-3.45 (m, 1H), 3.25-3.21 (m, 1H), 2.71 (s, 3H).

### Example 149A & Example 149B

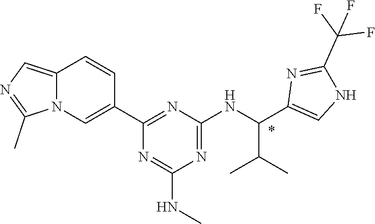 [SMILES: CNc1nc(NC(c2c[nH]c(C(F)(F)F)n2)C(C)C)nc(-c2ccc3cnc(C)n3c2)n1]

### N2-methyl-6-(3-methylimidazo[1,5-a]pyridin-6-yl)-N4-[(1S or 1R)-2-methyl-1-[2-(trifluoromethyl)-1H-imidazol-4-yl]propyl]-1,3,5-triazine-2,4-diamine (General Method for Target Compounds B)

149A

LCMS m/z (ESI+) 446.2 [M+H]⁺.

1H NMR (400 MHZ, Methanol-d4): δ=8.91 (br s, 1H), 7.62 (br s, 1H), 7.50 (br d, 1H), 7.30 (s, 1H), 7.21 (br s, 1H), 5.30-5.13 (m, 1H), 3.01 (br s, 3H), 2.71 (s, 3H), 2.25 (br s, 1H), 1.11-0.90 (m, 6H).

149B

LCMS m/z (ESI+) 446.2 [M+H]⁺.

1H NMR (400 MHZ, DMSO-d6) δ=8.82 (s, 1H), 7.53 (s, 2H), 7.31-6.91 (m, 4H), 5.11 (br s, 1H), 2.88 (br s, 3H), 2.66 (s, 3H), 2.25-2.19 (m, 1H), 0.97 (br d, J=6.8 Hz, 3H), 0.87 (d, 3H).

### Example 150

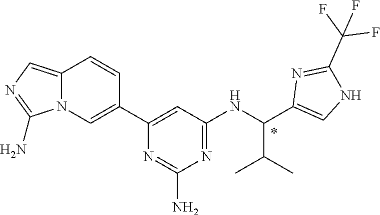 [SMILES: CC(C)C(Nc1cc(-c2ccc3cnc(N)n3c2)nc(N)n1)c1c[nH]c(C(F)(F)F)n1]

### 6-(3-methylimidazo[1,5-a]pyridin-6-yl)-N4-[(1S)-2-methyl-1-[2-(trifluoromethyl)-1H-imidazol-4-yl]propyl]pyrimidine-2,4-diamine (General Method for Target Compounds A)

LCMS m/z (ESI+) 431.1 [M+H]⁺.

1H NMR (400 MHZ, Methanol-d4) δ=9.04 (s, 1H), 8.00 (s, 1H), 7.93 (d, 1H), 7.44 (d, 1H), 7.29 (s, 1H), 6.67 (s, 1H), 5.24 (d, 1H), 3.04 (s, 3H), 2.46-2.24 (m, 1H), 1.05 (d, 3H), 0.95 (d, 3H).

### Example 151

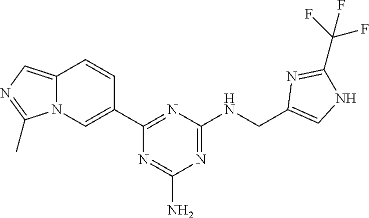 [SMILES: Cc1ncc2ccc(-c3nc(N)nc(NCc4c[nH]c(C(F)(F)F)n4)n3)cn12]

### 6-(3-methylimidazo[1,5-a]pyridin-6-yl)-N4-[[2-(trifluoromethyl)-1H-imidazol-4-yl]methyl]-1,3,5-triazine-2,4-diamine (General Method for Target Compounds A)

LCMS m/z (ESI+) 390.0 [M+H]⁺.

1H NMR (400 MHZ, DMSO-d6): δ=13.12 (br s, 1H), 8.81 (s, 1H), 7.52 (s, 2H), 7.36 (br s, 1H), 7.28 (s, 1H), 7.18 (br s, 1H), 6.70 (br s, 2H), 4.53 (br s, 2H), 2.65 (s, 3H).

### Example 152A & 152B

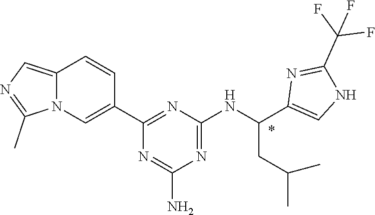 [SMILES: Cc1ncc2ccc(-c3nc(N)nc(NC(CC(C)C)c4c[nH]c(C(F)(F)F)n4)n3)cn12]

### 6-(3-methylimidazo[1,5-a]pyridin-6-yl)-N4-[(1S or 1R)-3-methyl-1-[2-(trifluoromethyl)-1H-imidazol-4-yl]butyl]-1,3,5-triazine-2,4-diamine (General Method for Target Compounds A)

152A

LCMS m/z (ESI+) 456.2 [M+H]⁺.

1H NMR (400 MHZ, DMSO-d6): δ=7.12 (s, 1H), 5.63 (d, 1H), 5.51-5.36 (m, 2H), 4.49 (q, 1H), 3.51 (d, 2H), 1.87-1.71 (m, 2H), 1.69-1.62 (m, 1H), 1.07 (s, 9H), 0.94-0.84 (m, 8H),−0.04 (m, 9H).

152B

LCMS m/z (ESI+) 446.2 [M+H]⁺.

1H NMR (400 MHZ, Methanol-d4): δ=8.90 (s, 1H), 7.68-7.56 (m, 1H), 7.49 (br d, 1H), 7.28 (s, 1H), 7.16 (m, 1H), 5.62-5.27 (m, 1H), 2.69 (s, 3H), 1.91-1.59 (m, 3H), 1.01 (d, 6H).

### Example 153A & 153B

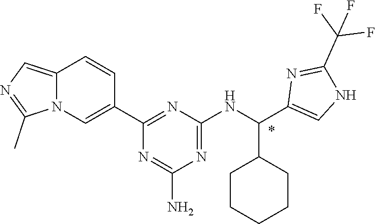 [SMILES: Cc1ncc2ccc(-c3nc(N)nc(NC(c4c[nH]c(C(F)(F)F)n4)C4CCCCC4)n3)cn12]

### N4-[(S or R)-cyclohexyl-[2-(trifluoromethyl)-1H-imidazol-4-yl]methyl]-6-(3-methylimidazo[1,5-a]pyridin-6-yl)-1,3,5-triazine-2,4-diamine (General Method for Target Compounds A)

153A

LCMS m/z (ESI+) 472.2 [M+H]⁺.

1H NMR (400 MHZ, DMSO-d6): δ=13.14 (br s, 1H), 8.81 (s, 1H), 8.13 (s, 1H), 7.52 (s, 2H), 7.28 (br s, 3H), 6.66 (br s, 2H), 5.08 (br s, 1H), 2.65 (s, 3H), 1.89-1.56 (m, 6H), 1.29-1.15 (m, 3H), 1.08-0.89 (m, 2H).

153B

LCMS m/z (ESI+) 472.2 [M+H]⁺.

1H NMR (400 MHZ, DMSO-d6): δ=13.08 (br s, 1H), 8.81 (s, 1H), 8.14 (s, 1H), 7.52 (s, 2H), 7.35-6.95 (m, 3H), 6.67 (br s, 2H), 5.09 (br s, 1H), 2.65 (s, 3H), 1.94-1.49 (m, 6H), 1.30-0.90 (m, 5H).

### Example 154

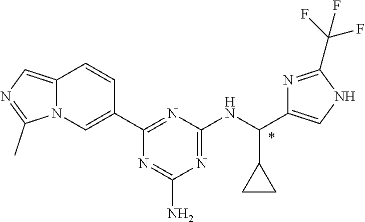 [SMILES: Cc1ncc2ccc(-c3nc(N)nc(NC(c4c[nH]c(C(F)(F)F)n4)C4CC4)n3)cn12]

### N4-[(S or R)-cyclopropyl-[2-(trifluoromethyl)-1H-imidazol-4-yl]methyl]-6-(3-methylimidazo[1,5-a]pyridin-6-yl)-1,3,5-triazine-2,4-diamine (General Method for Target Compounds A)

LCMS m/z (ESI+) 430.0 [M+H]⁺.

1H NMR (400 MHZ, Methanol-d4): δ=8.92 (br s, 1H), 8.15 (s, 1H), 7.69-7.58 (m, 1H), 7.55-7.46 (m, 1H), 7.32 (s, 1H), 7.23 (br s, 1H), 4.96 (br s, 1H), 2.72 (s, 3H), 1.38 (br s, 1H), 0.76-0.35 (m, 4H).

### Example 155A & Example 155B

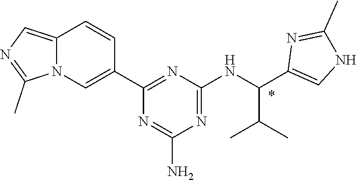 [SMILES: Cc1nc(C(Nc2nc(N)nc(-c3ccc4cnc(C)n4c3)n2)C(C)C)c[nH]1]

### 6-(3-methylimidazo[1,5-a]pyridin-6-yl)-N4-[(1S or 1R)-2-methyl-1-(2-methyl-1H-imidazol-4-yl) propyl]-1,3,5-triazine-2,4-diamine (General Method for Target Compounds A)

155A

LCMS m/z (ESI+) 378.2 [M+H]⁺.

1H NMR (400 MHZ, Methanol-d4): δ=9.11 (br s, 1H), 8.06-8.00 (m, 1H), 7.91 (s, 1H), 7.82 (d, 1H), 7.37 (br s, 1H), 5.36-4.93 (m, 1H), 3.02 (s, 3H), 2.63 (s, 3H), 2.34-2.24 (m, 1H), 1.11 (d, 3H), 1.03 (br d, 3H).

155B

LCMS m/z (ESI+) 378.2 [M+H]⁺.

1H NMR (400 MHZ, Methanol-d4): δ=9.10 (br s, 1H), 8.04-8.01 (m, 1H), 7.90 (s, 1H), 7.86-7.79 (m, 1H), 7.32-7.29 (m, 1H), 5.32-4.96 (m, 1H), 2.99 (s, 3H), 2.63 (s, 3H), 2.33-2.23 (m, 1H), 1.11 (br d, 3H), 1.03 (br d, J=6.4 Hz, 3H).

### Example 156

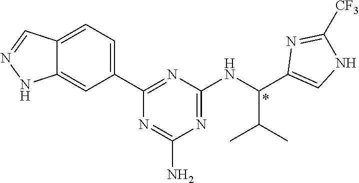 [SMILES: CC(C)C(Nc1nc(N)nc(-c2ccc3cn[nH]c3c2)n1)c1c[nH]c(C(F)(F)F)n1]

### 6-(1H-indazol-6-yl)-N4-[(1R or 1S)-2-methyl-1-[2-(trifluoromethyl)-1H-imidazol-4-yl]propyl]-1, 3, 5-triazine-2,4-diamine

LCMS m/z (ESI+) 418.1 [M+H]⁺.

1H NMR (400 MHZ, Methanol-d4): δ=8.50 (br s, 1H), 8.19 (br s, 1H), 7.97-7.94 (m, 2H), 7.32 (s, 1H), 5.41-5.12 (m, 1H), 2.37 (br s, 1H), 1.09 (br s, 3H), 0.99 (br s, 3H).

### Example 157

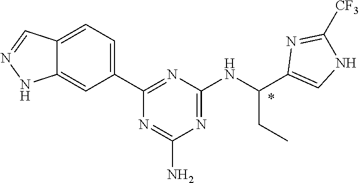 [SMILES: CCC(Nc1nc(N)nc(-c2ccc3cn[nH]c3c2)n1)c1c[nH]c(C(F)(F)F)n1]

### 6-(1H-indazol-6-yl)-N4-[(1S or 1R)-1-[2-(trifluoromethyl)-1H-imidazol-4-yl]propyl]-1,3,5-triazine-2,4-diamine (General Method for Target Compounds C)

LCMS m/z (ESI+) 404.2 [M+H]⁺.

1H NMR (400 MHZ, Methanol-d4): δ=8.35 (br s, 1H), 8.12-8.08 (m, 1H), 7.89 (d, 1H), 7.80 (d, 1H), 7.19 (s, 1H), 5.20-5.02 (m, 1H), 2.06-1.85 (m, 2H), 1.00-0.90 (m, 3H).

### Example 158

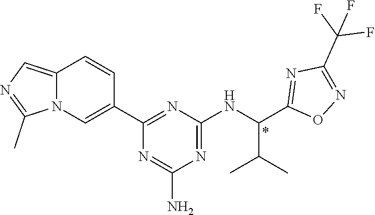 [SMILES: Cc1ncc2ccc(-c3nc(N)nc(NC(c4nc(C(F)(F)F)no4)C(C)C)n3)cn12]

### 6-(3-methylimidazo[1,5-a]pyridin-6-yl)-N4-[(1S or 1R)-2-methyl-1-[3-(trifluoromethyl)-1,2,4-oxadiazol-5-yl]propyl]-1,3,5-triazine-2,4-diamine (General Method for Target Compounds A)

LCMS m/z (ESI+) 434.1 [M+H]⁺.

1H NMR (400 MHZ, METHANOL-d4) δ=9.07-8.75 (m, 1H), 8.45 (s, 1H), 7.76-7.40 (m, 2H), 7.32 (s, 1H), 5.54-5.28 (m, 1H), 2.74 (d, 3H), 2.53-2.36 (m, 1H), 1.18 (dd, 3H), 1.05 (d, 3H).

### Example 159

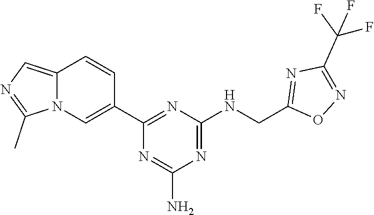 [SMILES: Cc1ncc2ccc(-c3nc(N)nc(NCc4nc(C(F)(F)F)no4)n3)cn12]

### 6-(3-methylimidazo[1,5-a]pyridin-6-yl)-N4-[[3-(trifluoromethyl)-1,2,4-oxadiazol-5-yl]methyl]-1,3,5-triazine-2,4-diamine (General Method for Target Compounds A)

LCMS m/z (ESI+) 392.0 [M+H]⁺.

1H NMR (400 MHZ, Methanol-d4) δ=9.02-8.78 (m, 1H), 8.20 (s, 1H), 7.71-7.41 (m, 2H), 7.30 (s, 1H), 4.97 (s, 2H), 2.71 (s, 3H).

### Example 160

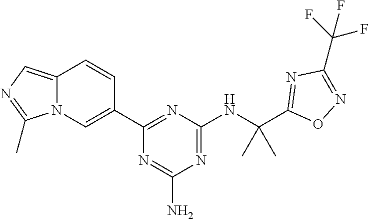 [SMILES: Cc1ncc2ccc(-c3nc(N)nc(NC(C)(C)c4nc(C(F)(F)F)no4)n3)cn12]

### 6-(3-methylimidazo[1,5-a]pyridin-6-yl)-N4-[1-methyl-1-[3-(trifluoromethyl)-1,2,4-oxadiazol-5-yl]ethyl]-1,3,5-triazine-2,4-diamine (General Method for Target Compounds A)

LCMS m/z (ESI+) 420.0 [M+H]⁺.

1H NMR (400 MHZ, METHANOL-d4) δ=8.72 (s, 1H), 7.42 (s, 1H), 7.29 (s, 1H), 7.20 (s, 1H), 2.71 (s, 3H), 1.89 (s, 6H).

### Example 161

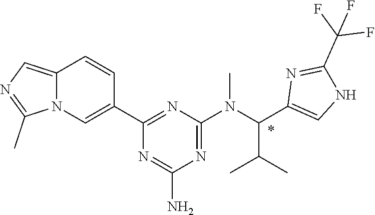 [SMILES: Cc1ncc2ccc(-c3nc(N)nc(N(C)C(c4c[nH]c(C(F)(F)F)n4)C(C)C)n3)cn12]

### N4-methyl-6-(3-methylimidazo[1,5-a]pyridin-6-yl)-N4-[(1S or 1R)-2-methyl-1-[2-(trifluoromethyl)-1H-imidazol-4-yl]propyl]-1,3,5-triazine-2,4-diamine

To a solution of 6-(3-methylimidazo[1,5-a]pyridin-6-yl)-N2-[(1R or 1S)-2-methyl-1-[2-(trifluoromethyl)-1H-imidazol-4-yl]propyl]-1,3,5-triazine-2,4-diamine (Example 144A & 144B, 170 mg, 394.04 μmol) in THF (2 mL) at 0° C. was added 60% NaH (18.91 mg, 788.08 μmol) and stirred for 1 h. Then the mixture was added dropwise Mel (104.87 mg, 738.82 μmol, 97.27 μL) at 0° C. The mixture was allowed to warm to 25° C. and stirred for 1 hr. The reaction mixture was diluted with H₂O (100 mL) and filtered to remove the in soluble. The filtrate was extracted with

EtOAc (100 mL×3). Then the organic layers were washed with brine (50 mL×3), dried over anhydrous Na₂SO₄, filtered, and concentrated under reduced pressure to give a residue. The residue was purified by flash silica gel chromatography to obtain N4-methyl-6-(3-methylimidazo[1,5-a]pyridin-6-yl)-N4-[(1S or 1R)-2-methyl-1-[2-(trifluoromethyl)-1H-imidazol-4-yl]propyl]-1,3,5-triazine-2,4-diamine (62.48 mg, 35.6% yield) as a yellow oil.

LCMS m/z (ESI+) 446.1 [M+H]⁺.

1H NMR (400 MHZ, DMSO-d6): δ=13.29 (s, 1H), 8.84 (s, 1H), 8.14 (s, 1H), 7.64-7.47 (m, 2H), 7.29 (br s, 2H), 6.67 (br s, 2H), 5.96-5.60 (m, 1H), 2.82 (br s, 3H), 2.66 (s, 3H), 2.59-2.53 (m, 1H), 0.93 (br d, 6H).

### Example 162

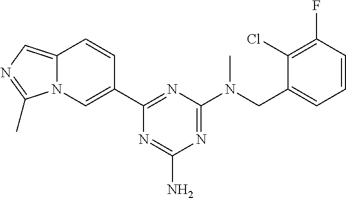 [SMILES: Cc1ncc2ccc(-c3nc(N)nc(N(C)Cc4cccc(F)c4Cl)n3)cn12]

### N4-[(2-chloro-3-fluoro-phenyl)methyl]-N4-methyl-6-(3-methylimidazo[1,5-a]pyridin-6-yl)-1,3,5-triazine-2,4-diamine (General Method for Target Compounds A) LCMS m/z (ESI+) 398.1 [M+H]⁺

1H NMR (400 MHZ, DMSO-d6) δ=8.81 (s, 1H), 7.53 (br s, 2H), 7.41-7.28 (m, 3H), 7.09 (d, 1H), 6.79 (s, 2H), 5.04 (s, 2H), 3.24 (s, 3H), 2.65 (s, 3H).

### Example 163

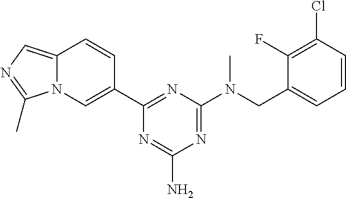 [SMILES: Cc1ncc2ccc(-c3nc(N)nc(N(C)Cc4cccc(Cl)c4F)n3)cn12]

### N4-[(3-chloro-2-fluoro-phenyl)methyl]-N4-methyl-6-(3-methylimidazo[1,5-a]pyridin-6-yl)-1,3,5-triazine-2,4-diamine (General Method for Target Compounds A)

LCMS m/z (ESI+) 398.1 [M+H]⁺.

1H NMR (400 MHZ, DMSO-d6) δ=8.81 (s, 1H), 8.16 (s, 1H), 7.51 (s, 2H), 7.46 (t, 1H), 7.33-7.23 (m, 2H), 7.21-7.13 (m, 1H), 6.75 (s, 2H), 4.99 (s, 2H), 3.20 (s, 3H), 2.64 (s, 3H).

### Example 164

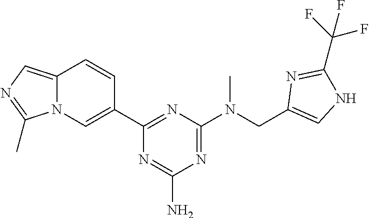 [SMILES: Cc1ncc2ccc(-c3nc(N)nc(N(C)Cc4c[nH]c(C(F)(F)F)n4)n3)cn12]

### N4-methyl-6-(3-methylimidazo[1,5-a]pyridin-6-yl)-N4-[[2-(trifluoromethyl)-1H-imidazol-4-yl]methyl]-1,3,5-triazine-2,4-diamine (General Method for Target Compounds A)

LCMS m/z (ESI+) 404.2 [M+H]⁺.

1H NMR (400 MHZ, DMSO-d6): δ=11.45 (s, 1H), 8.84 (d, 1H), 7.61-7.47 (m, 2H), 7.28 (s, 1H), 7.19 (s, 1H), 6.71 (s, 2H), 4.83 (br s, 2H), 3.18 (br s, 3H), 2.65 (m, 3H).

### Example 165

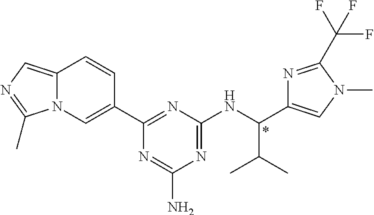 [SMILES: Cc1ncc2ccc(-c3nc(N)nc(NC(c4cn(C)c(C(F)(F)F)n4)C(C)C)n3)cn12]

### 6-(3-methylimidazo[1,5-a]pyridin-6-yl)-N4-[(1S or 1R)-2-methyl-1-[1-methyl-2-(trifluoromethyl) imidazol-4-yl]propyl]-1,3,5-triazine-2,4-diamine

To a solution of 6-(3-methylimidazo[1,5-a]pyridin-6-yl)-N2-[(1R or 1S)-2-methyl-1-[2-(trifluoromethyl)-1H-imidazol-4-yl]propyl]-1,3,5-triazine-2,4-diamine (Example 144A & 144B, 170 mg, 394.04 μmol) in THF (2 mL) at 0° C. was added 60% NaH (18.91 mg, 788.08 μmol) and stirred for 1 h. Then the mixture was added dropwise Mel (104.87 mg, 738.82 μmol, 97.27 μL) at 0° C. The mixture was allowed to warm to 25° C. and stirred for 1 hr. The reaction mixture was diluted with H₂O (100 mL) and filtered to remove the in soluble. The filtrate was extracted with EtOAc (100 mL×3). Then the organic layers were washed with brine (50 mL×3), dried over anhydrous Na₂SO₄, filtered, and concentrated under reduced pressure to give a residue. The residue was purified by flash silica gel chromatography to obtain 6-(3-methylimidazo[1,5-a]pyridin-6-yl)-N4-[(1S or 1R)-2-methyl-1-[1-methyl-2-(trifluoromethyl) imidazol-4-yl]propyl]-1,3,5-triazine-2,4-diamine (94.95 mg, 54.1% yield) as a yellow oil.

LCMS m/z (ESI+) 446.3 [M+H]⁺.

1H NMR (400 MHZ, Methanol-d4): δ=8.91 (br s, 1H), 8.37 (br s, 1H), 7.67-7.58 (m, 1H), 7.54-7.48 (m, 1H), 7.31 (s, 1H), 7.26-7.21 (m, 1H), 5.21-4.98 (m, 1H), 3.81 (s, 3H), 2.76-2.67 (m, 3H), 2.30-2.17 (m, 1H), 1.04-0.98 (m, 3H), 0.97-0.91 (m, 3H).

### Example 166

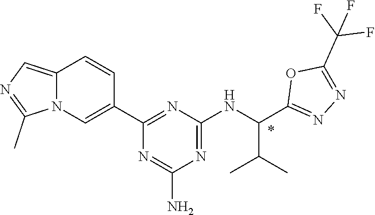 [SMILES: Cc1ncc2ccc(-c3nc(N)nc(NC(c4nnc(C(F)(F)F)o4)C(C)C)n3)cn12]

### 6-(3-methylimidazo[1,5-a]pyridin-6-yl)-N4-[(1S or 1R)-2-methyl-1-[5-(trifluoromethyl)-1,3,4-oxadiazol-2-yl]propyl]-1,3,5-triazine-2,4-diamine (General Method for Target Compounds A)

LCMS m/z (ESI+) 434.1 [M+H]⁺.

1H NMR (400 MHZ, DMSO-d6) δ=8.77 (s, 1H), 8.30 (s, 1H), 7.86 (s, 1H), 7.53-7.50 (m, 1H), 7.44 (s, 1H), 7.27 (s, 1H), 6.78 (s, 2H), 5.32 (t, J=7.2 Hz, 1H), 2.65 (s, 3H), 2.43-2.41 (m, 1H), 1.09 (d, 3H), 0.99 (d, 3H).

### Example 167

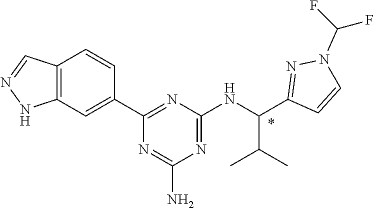 [SMILES: CC(C)C(Nc1nc(N)nc(-c2ccc3cn[nH]c3c2)n1)c1ccn(C(F)F)n1]

### N4-[(1R or 1S)-1-[1-(difluoromethyl) pyrazol-3-yl]-2-methyl-propyl]-6-(1H-indazol-6-yl)-1,3,5-triazine-2,4-diamine (General Method for Target Compounds C)

MS(ESI) m/z=400.1 [M+H]⁺.

1H NMR (400 MHZ, Methanol-d4): δ=8.50 (br s, 1H), 8.19 (br s, 1H), 8.05-7.91 (m, 3H), 7.47 (t, 1H), 6.57 (d, 1H), 5.39-4.92 (m, 1H), 2.32 (dd, 1H), 1.13-1.05 (m, 3H), 1.02-0.93 (m, 3H).

### Example 168

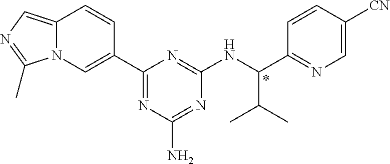 [SMILES: Cc1ncc2ccc(-c3nc(N)nc(NC(c4ccc(C#N)cn4)C(C)C)n3)cn12]

### 6-[(1R or 1S)-1-[[4-amino-6-(3-methylimidazo[1,5-a]pyridin-6-yl)-1,3,5-triazin-2-yl]amino]-2-methyl-propyl]pyridine-3-carbonitrile (General Method for Target Compounds A)

MS(ESI) m/z=400.2 [M+H]⁺.

1H NMR (400 MHz, Methanol-d4) δ=8.85 (d, 1H), 8.44-8.42 (m, 1H), 8.22 (s, 1H), 7.61-7.51 (m, 4H), 7.32 (s, 1H), 5.11-5.03 (m, 1H), 2.74 (d, 3H), 2.30-2.24 (m, 1H), 1.06-1.02 (m, 3H), 0.89-0.85 (m, 3H).

### Example 169

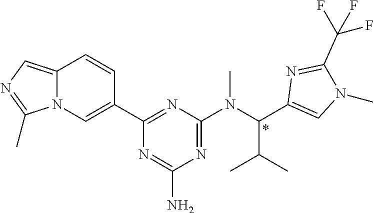 [SMILES: Cc1ncc2ccc(-c3nc(N)nc(N(C)C(c4cn(C)c(C(F)(F)F)n4)C(C)C)n3)cn12]

### N4-methyl-6-(3-methylimidazo[1,5-a]pyridin-6-yl)-N4-[(1S or 1R)-2-methyl-1-[1-methyl-2-(trifluoromethyl) imidazol-4-yl]propyl]-1,3,5-triazine-2,4-diamine

To a solution of 6-(3-methylimidazo[1,5-a]pyridin-6-yl)-N2-[(1R or 1S)-2-methyl-1-[2-(trifluoromethyl)-1H-imidazol-4-yl]propyl]-1,3,5-triazine-2,4-diamine (Example 144A & 144B, 200 mg, 463.57 μmol) in THF (2 mL) at 0° C. was added 60% NaH (22.25 mg, 927.75 μmol) and stirred for 1 h. Then the mixture was added dropwise Mel (123.37 mg, 869.20 μmol) at 0° C. The mixture was allowed to warm to 25° C. and stirred for 1 hr. The reaction mixture was diluted with H₂O (100 mL) and filtered to remove the in soluble. The filtrate was extracted with EtOAc (100 mL×3). Then the organic layers were washed with brine (50 mL×3), dried over anhydrous Na₂SO₄, filtered, and concentrated under reduced pressure to give a residue. The residue was purified by flash silica gel chromatography to obtain N4-methyl-6-(3-methylimidazo[1,5-a]pyridin-6-yl)-N4-[(1S or 1R)-2-methyl-1-[1-methyl-2-(trifluoromethyl) imidazol-4-yl]propyl]-1,3,5-triazine-2,4-diamine (59.64 mg, 28.1% yield) as a yellow oil.

MS(ESI) m/z=460.3 [M+H]⁺.

1H NMR (400 MHZ, Methanol-d4): δ=8.95 (br s, 1H), 8.35 (br s, 1H), 7.75-7.61 (m, 1H), 7.59-7.46 (m, 1H), 7.38-7.26 (m, 2H), 5.98-5.64 (m, 1H), 3.82 (s, 3H), 3.10 (br s, 3H), 2.72 (d, 3H), 2.64-2.51 (m, 1H), 1.02-0.89 (m, 6H).

### Example 170A & Example 170B

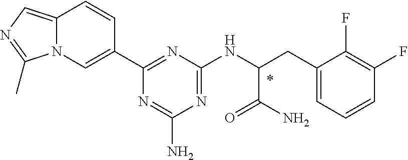 [SMILES: Cc1ncc2ccc(-c3nc(N)nc(NC(Cc4cccc(F)c4F)C(N)=O)n3)cn12]

### (2R or 2S)-2-[[4-amino-6-(3-methylimidazo[1,5-a]pyridin-6-yl)-1,3,5-triazin-2-yl]amino]-3-(2,3-dichlorophenyl) propanamide (General Method for Target Compounds A) 170A

MS(ESI) m/z=457.0 [M+H]⁺.

1H NMR (400 MHZ, METHANOL-d4) δ=8.93-8.84 (m, 1H), 7.60-7.46 (m, 2H), 7.42-7.31 (m, 1H), 7.29 (s, 1H), 7.24 (d, 1H), 7.20-7.06 (m, 1H), 5.21 (dd, 1H), 3.59-3.46 (m, 1H), 3.24-3.02 (m, 1H), 2.86-2.68 (m, 3H).

170B

MS(ESI) m/z=457.0 [M+H]⁺.

1H NMR (400 MHZ, DMSO-d6) δ=8.98-8.75 (m, 1H), 7.99 (s, 1H), 7.85-7.71 (m, 3H), 7.61-7.16 (m, 6H), 7.05 (br s, 1H), 5.01-4.68 (m, 1H), 3.45-3.01 (m, 2H), 2.92-2.82 (m, 3H).

### Example 171

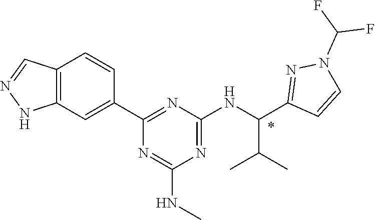 [SMILES: CNc1nc(NC(c2ccn(C(F)F)n2)C(C)C)nc(-c2ccc3cn[nH]c3c2)n1]

### N4-[(1R or 1S)-1-[1-(difluoromethyl) pyrazol-3-yl]-2-methyl-propyl]-6-(1H-indazol-6-yl)-N2-methyl-1,3,5-triazine-2,4-diamine (General Method for Target Compounds D)

MS(ESI) m/z=414.1 [M+H]⁺.

1H NMR (400 MHZ, DMSO-d6): δ=13.14 (s, 1H), 8.54 (s, 1H), 8.12-8.05 (m, 3H), 7.80 (d, 1H), 7.70 (t, 1H), 7.48-6.92 (m, 2H), 6.61 (d, 1H), 5.24-5.16 (m, 1H), 2.90 (br s, 3H), 2.35-2.18 (m, 1H), 1.01 (d, 3H), 0.89 (d, 3H).

### Example 172

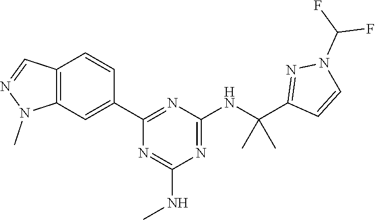 [SMILES: CNc1nc(NC(C)(C)c2ccn(C(F)F)n2)nc(-c2ccc3cnn(C)c3c2)n1]

### N4-[1-[1-(difluoromethyl) pyrazol-3-yl]-1-methyl-ethyl]-N2-methyl-6-(1-methylindazol-6-yl)-1,3,5-triazine-2,4-diamine (General Method for Target Compounds B)

MS(ESI) m/z=414.2 [M+H]⁺.

1H NMR (400 MHz DMSO-d6) δ=8.37 (s, 1H), 8.04 (s, 1H), 7.99 (d, 2H), 7.86-7.57 (m, 2H), 7.25-6.82 (m, 2H), 6.43 (d, 1H), 4.07 (s, 3H), 2.92-2.58 (m, 3H), 1.77 (s, 6H).

### Example 173

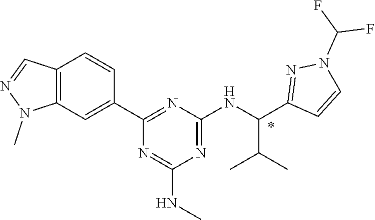 [SMILES: CNc1nc(NC(c2ccn(C(F)F)n2)C(C)C)nc(-c2ccc3cnn(C)c3c2)n1]

### N4-[(1R or 1S)-1-[1-(difluoromethyl) pyrazol-3-yl]-2-methyl-propyl]-N2-methyl-6-(1-methylindazol-6-yl)-1,3,5-triazine-2,4-diamine (General Method for Target Compounds B)

MS(ESI) m/z=428.2 [M+H]⁺.

1H NMR (400 MHZ, DMSO-d6) δ=8.52 (s, 1H), 8.14 (d, 1H), 8.10-8.02 (m, 2H), 7.87-7.79 (m, 1H), 7.75-7.53 (m, 1H), 7.28 (s, 1H), 7.03 (s, 1H), 6.62 (d, 1H), 5.22 (s, 1H), 4.13 (s, 3H), 2.93 (s, 3H), 2.25 (m, 1H), 1.03 (d, 3H), 0.91 (d, 3H).

### Example 174

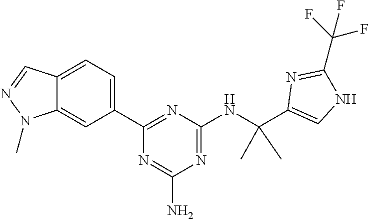 [SMILES: Cn1ncc2ccc(-c3nc(N)nc(NC(C)(C)c4c[nH]c(C(F)(F)F)n4)n3)cc21]

### 6-(1-methylindazol-6-yl)-N4-[1-methyl-1-[2-(trifluoromethyl)-1H-imidazol-4-yl]ethyl]-1,3,5-triazine-2,4-diamine (General Method for Target Compounds A)

MS(ESI) m/z=418.2 [M+H]⁺.

1H NMR (400 MHZ, METHANOL-d4) δ=8.66-8.22 (m, 1H), 8.02 (s, 2H), 7.75 (s, 1H), 7.25 (s, 1H), 4.12 (s, 3H), 1.83 (s, 6H).

### Example 175

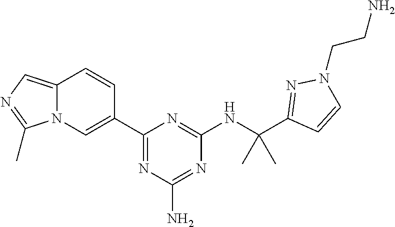 [SMILES: Cc1ncc2ccc(-c3nc(N)nc(NC(C)(C)c4ccn(CCN)n4)n3)cn12]

### N2-[1-[1-(2-aminoethyl) pyrazol-3-yl]-1-methyl-ethyl]-6-(3-methylimidazo[1,5-a]pyridin-6-yl)-1,3,5-triazine-2,4-diamine (General Method for Target Compounds A)

MS(ESI) m/z=393.2 [M+H]⁺.

1H NMR (Methanol-d4) δ=9.62 (br s, 0.5H), 9.14 (br s, 0.5H), 8.03 (d, 1H), 7.98-7.84 (m, 2H), 7.69-7.67 (m, 1H), 6.57-6.26 (m, 1H), 4.48-4.44 (m, 2H), 3.57-3.38 (m, 2H), 3.12-2.98 (m, 3H), 1.92 (s, 6H).

### Example 176

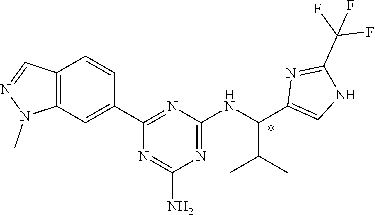 [SMILES: CC(C)C(Nc1nc(N)nc(-c2ccc3cnn(C)c3c2)n1)c1c[nH]c(C(F)(F)F)n1]

### 6-(1-methylindazol-6-yl)-N4-[(1R or 1S)-2-methyl-1-[2-(trifluoromethyl)-1H-imidazol-4-yl]propyl]-1,3,5-triazine-2,4-diamine (General Method for Target Compounds A)

MS(ESI) m/z=432.3 [M+H]⁺.

1H NMR (METHANOL-d4) δ=8.51 (s, 1H), 8.35 (s, 1H), 8.18-8.07 (m, 1H), 8.04 (s, 1H), 7.79 (d, 1H), 7.18 (br s, 1H), 5.35-5.04 (m, 1H), 4.14 (s, 3H), 2.30-2.22 (m, 1H), 1.07-0.93 (m, 6H).

### Example 177

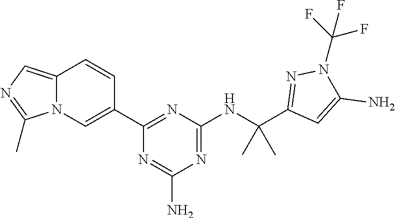 [SMILES: Cc1ncc2ccc(-c3nc(N)nc(NC(C)(C)c4cc(N)n(C(F)(F)F)n4)n3)cn12]

### N4-[1-[5-amino-1-(difluoromethyl) pyrazol-3-yl]-1-methyl-ethyl]-6-(3-methylimidazo[1,5-a]pyridin-6-yl)-1,3,5-triazine-2,4-diamine (General Method for Target Compounds A)

MS(ESI) m/z=415.2 [M+H]⁺.

1H NMR (400 MHZ, DMSO-d6): δ=8.75 (s, 1H), 7.58-7.32 (m, 3H), 7.28 (s, 1H), 6.89 (br s, 1H), 6.54 (br s, 2H), 5.51 (br s, 2H), 5.33 (s, 1H), 2.64 (s, 3H), 1.70 (s, 6H).

### Example 178

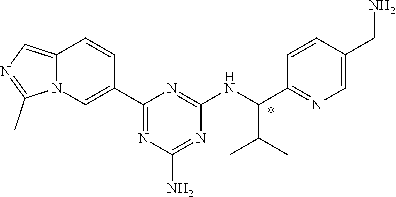 [SMILES: Cc1ncc2ccc(-c3nc(N)nc(NC(c4ccc(CN)cn4)C(C)C)n3)cn12]

### N2-[(1R or 1S)-1-[5-(aminomethyl)-2-pyridyl]-2-methyl-propyl]-6-(3-methylimidazo[1,5-a]pyridin-6-yl)-1,3,5-triazine-2,4-diamine (General Method for Target Compounds A)

MS(ESI) m/z=404.2 [M+H]⁺.

1H NMR (400 MHZ, METHANOL-d4) δ=8.97-8.81 (m, 1H), 8.57 (s, 1H), 7.85 (d, 1H), 7.69-7.57 (m, 1H), 7.56-7.47 (m, 2H), 7.31 (d, 1H), 5.04 (s, 1H), 3.98 (d, 2H), 2.74 (d, 3H), 2.39-2.08 (m, 1H), 1.09 (dd, 3H), 0.94-0.90 (m, 3H).

### Example 179

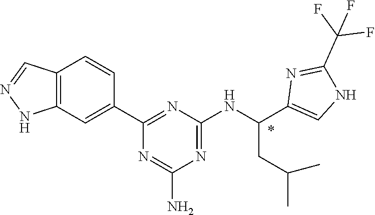 [SMILES: CC(C)CC(Nc1nc(N)nc(-c2ccc3cn[nH]c3c2)n1)c1c[nH]c(C(F)(F)F)n1]

### 6-(1H-indazol-6-yl)-N4-[(1R or 1S)-3-methyl-1-[2-(trifluoromethyl)-1H-imidazol-4-yl]butyl]-1,3,5-triazine-2,4-diamine (General Method for Target Compounds C)

MS(ESI) m/z=432.2 [M+H]⁺.

1H NMR (DMSO-d6) δ=13.32 (br s, 2H), 8.57-8.46 (m, 1H), 8.16-8.03 (m, 2H), 7.81 (dd, J=8.4, 3.6 Hz, 1H), 7.63-7.34 (m, 1H), 7.23 (br d, J=7.6 Hz, 1H), 6.73-7.00 (m, 2H), 5.60-5.15 (m, 1H) 1.89-1.76 (m, 1H), 1.73-1.58 (m, 2H), 0.97-0.92 (m, 6H).

### Example 180A & 180B

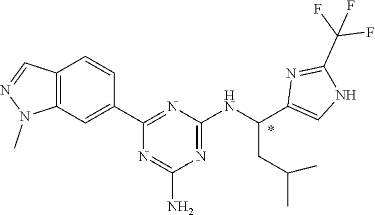 [SMILES: CC(C)CC(Nc1nc(N)nc(-c2ccc3cnn(C)c3c2)n1)c1c[nH]c(C(F)(F)F)n1]

### 6-(1-methylindazol-6-yl)-N4-[(1R or 1S)-3-methyl-1-[2-(trifluoromethyl)-1H-imidazol-4-yl]butyl]-1,3,5-triazine-2,4-diamine (General Method for Target Compounds A)

180A

MS(ESI) m/z=446.5 [M+H]⁺.

1H NMR (400 MHZ, Methanol-d4) δ=8.55-8.44 (m, 1H), 8.19-8.14 (m, 1H), 8.04-7.89 (m, 2H), 7.33-7.28 (m, 1H), 5.74-5.40 (m, 1H), 4.20 (s, 3H), 1.99-1.86 (m, 2H), 1.74 (m, 1H), 1.12-1.01 (m, 6H).

180B

MS(ESI) m/z=446.2 [M+H]⁺.

1H NMR (400 MHz, DMSO-d6) δ=13.46-13.29 (m, 1H), 8.48 (s, 1H), 8.12-8.07 (m, 2H), 7.81-7.79 (m, 1H), 7.64-7.45 (m, 1H), 7.40-7.19 (m, 1H), 6.91 (s, 1H), 6.71 (br s, 1H), 5.45-5.22 (m, 1H), 4.09 (s, 3H), 1.81-1.75 (m, 1H), 1.73-1.66 (m, 2H), 0.96-0.93 (m, 6H).

### Example 181

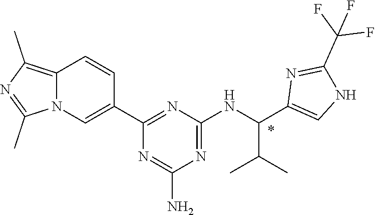 [SMILES: Cc1nc(C)n2cc(-c3nc(N)nc(NC(c4c[nH]c(C(F)(F)F)n4)C(C)C)n3)ccc12]

### 6-(1,3-dimethylimidazo[1,5-a]pyridin-6-yl)-N2-[(1S or 1R)-2-methyl-1-[2-(trifluoromethyl)-1H-imidazol-4-yl]propyl]-1,3,5-triazine-2,4-diamine (General Method for Target Compounds A)

MS(ESI) m/z=446.2 [M+H]⁺.

1H NMR (400 MHZ, DMSO-d6) δ=8.87-8.83 (m, 1H), 7.90-7.86 (m, 1H), 7.77-7.59 (m, 2H), 7.26 (s, 1H), 7.14 (s, 1H), 7.09 (br s, 1H), 5.18-4.99 (m, 1H), 2.90-2.84 (m, 3H), 2.59-2.58 (m, 3H), 2.28-2.12 (m, 1H), 0.96-0.93 (m, 3H) 0.86-0.83 (m, 3H).

### Biological Assays

### Materials and Methods

MASTL activity assay

Wild-type human active MASTL (154 pM) was incubated in assay buffer (50 mM HEPES, 100 mM NaCl, 0.1 mM EGTA, 10 mM MgCl₂, 0.01% Tween-20, 0.5 mM TCEP, pH 7.5) with biotin tagged 40-mer ENSA peptide (10 nM), test compound and ATP (18 μM) for 60 minutes at room temperature. Test compounds were assayed using a 12-point dose range consisting of a 0, DMSO control and 10 sequential doses consisting from 0.0005, 0.002, 0.005, 0.014, 0.04, 0.12, 0.37, 1.11, 3.33 and 10 μM. The DMSO concentration was the same (1%) in all samples. An equal volume of detection buffer (assay buffer+267.5 pM Ab-K, 1.25 nM SA-D2, 20 mM EDTA and 400 mM KF) was added to stop the reaction to make a final volume of 20 μl, RT for 60 mins. Activity was measured using a plate reader (PerkinElmer EnSight) by the FRET signal generated between SA-D2 (streptavidin-D2) and Ab-K (anti-phospho-Serine 67 ENSA antibody (rabbit polyclonal, using standard techniques by a commercial supplier) conjugated to Cryptate). HTRF reagents (CisBio) were prepared as per the manufacturer recommendations. The 40-mer biotin-tagged ENSA peptide (synthesised by Bionics) used was based around Serine 67 on ENSA (YPSLGQKPGGSDFLMKRLQKGQKYFDSGDYNMAKAKMKNK).

### Results

The results of the MASTL activity assay are shown below in Table 2:

TABLE 2

| Example | MASTL IC50 (μM) |
|---|---|
| 1 | *** |
| 2 | * |
| 3 | *** |
| 4 | *** |
| 5 | *** |
| 6 | *** |
| 7A | *** |
| 7B | *** |
| 8 | *** |
| 9 | *** |
| 10 | *** |
| 11 | *** |
| 12 | * |
| 13 | * |
| 14 | * |
| 15 | * |
| 16 | *** |
| 17 | *** |
| 18 | *** |
| 19 | *** |
| 20 | *** |
| 21 | *** |
| 22 | *** |
| 23 | * |
| 24 | *** |
| 25 | * |
| 26 | *** |
| 27 | *** |
| 28 | *** |
| 29A | *** |
| 29B | * |
| 30 | *** |
| 31 | *** |
| 32 | *** |
| 33 | *** |
| 34 | *** |
| 35 | *** |
| 36 | *** |
| 37 | *** |
| 38 | * |
| 39 | *** |
| 40 | *** |
| 41 | *** |
| 42 | *** |
| 43 | ** |
| 44 | *** |
| 45 | *** |
| 46 | *** |
| 47A | *** |
| 48 | *** |
| 135 | *** |
| 136A | *** |
| 136B | * |
| 137 | ** |
| 138 | *** |
| 139 | *** |
| 140 | *** |
| 141A | ** |
| 141B | ** |
| 142A | *** |
| 142B | *** |
| 143A | *** |
| 143B | * |
| 144 | *** |
| 145 | *** |
| 146A | *** |
| 146B | *** |
| 147 | *** |
| 148 | *** |
| 149A | *** |
| 149B | *** |
| 150 | *** |
| 151 | *** |
| 152A | *** |
| 152B | *** |
| 153A | *** |
| 153B | *** |
| 154 | *** |
| 155A | ** |
| 155B | *** |
| 156 | *** |
| 157 | *** |
| 158 | * |
| 159 | ** |
| 160 | ** |
| 161 | ** |
| 162 | ** |
| 163 | *** |
| 164 | *** |
| 165 | *** |
| 166 | * |
| 167 | *** |
| 168 | * |
| 169 | ** |
| 170A | * |
| 170B | * |
| 171 | *** |
| 172 | *** |
| 173 | * |
| 174 | *** |
| 175 | *** |
| 176 | *** |
| 177 | *** |
| 178 | *** |
| 179 | *** |
| 180A | * |
| 180B | *** |
| 181 | *** |

0.001 μM < *** < 0.5 μM, 0.5 μM < ** < 1 μM, * > 1 μM

1. A method of treating a disease or medical condition in a subject, wherein the disease or medical condition is mediated by microtubule associated serine/threonine-like kinase (MASTL), the method comprising:
administering, to a subject in need of such treating, an effective amount of a composition comprising a compound of the formula (I), or a pharmaceutically acceptable salt thereof: 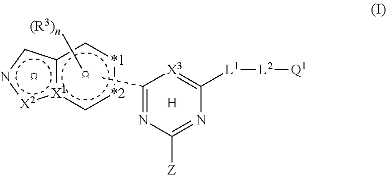
wherein the H ring in formula (I) is bonded to the carbon atom *1 or *2,
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from: C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{X1}R^{X2};wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

2. The method of claim 1,
wherein the H ring in formula (I) is bonded to the carbon atom *1 or *2,
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{X1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from: C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{X1}R^{X2}; wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

3. The method of claim 1,
wherein the H ring in formula (I) is bonded to the carbon atom *1 or *2,
Z is —NR¹R² or —CN;
R¹ and R² is independently selected from: H, D, and C₁₋₆ alkyl;
wherein said C₁₋₆ alkyl is optionally partially or fully deuterated;
R³ is each independently selected from: halo, C₁₋₆ alkyl and amino;
X₁ is N and X₂ is CR⁴, or X₁ is C and X₂ is NR⁵;
X₃ is CH or N;
R⁴ is selected from: H, NR^{x1}R^{X2}, —OH and C₁₋₆ alkyl;
R⁵ is selected from: H, and C₁₋₆ alkyl,
wherein said C₁₋₆ alkyl on R⁴ or R⁵ is optionally partially or fully deuterated;
L¹ is a bond or is selected from: NR⁶, O, and S;
R⁶ is selected from H, C₁₋₄ alkyl, and C₁₋₄ haloalkyl;
L² is a bond or —[CR⁷R⁸]p-,
wherein p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from: C₃₋₁₂ cycloalkyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2;
wherein said C₁₋₆ alkyl, C₂₋₆ alkenyl, and C₂₋₆ alkynyl is optionally substituted by one or more R¹¹; and
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl and NR^{X1}R^{X2};wherein R^{X1} and R^{X2} are independently selected from: H, C₁₋₄ alkyl optionally substituted by OH or 3- to 6-membered heterocyclyl;
n is an integer from 0 to 4; and
x is an integer from 0 to 3;
wherein when R⁴ or R⁵ is H or C₁₋₆ alkyl which is not deuterated, then
Z is —CN, or
—NR¹R² wherein at least one of R¹ and R² is D or partially or fully deuterated C₁₋₆ alkyl.

4. A method of treating a disease or medical condition in a subject, wherein the disease or medical condition is mediated by microtubule associated serine/threonine-like kinase (MASTL), the method comprising:
administering, to a subject in need of such treating, an effective amount of a composition comprising a compound of the formula (IV), or a pharmaceutically acceptable salt thereof: 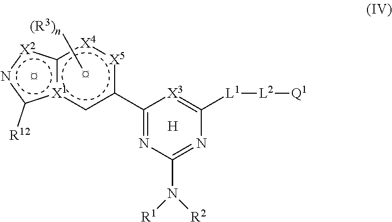
wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or is selected from: NR⁶, O, and S,
R⁶ is selected from H, C₁₋₄ alkyl, C₁₋₄ haloalkyl, and C₃₋₆ cycloalkyl,
wherein said C₃₋₆ cycloalkyl is optionally substituted by one or more substituents selected from: ═O, halo, C₁₋₄ alkyl and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, 3- to 6-membered heterocyclyl, 5- to 10-membered heteroaryl, or C₆₋₁₀ aryl optionally substituted by halogen or C₁-6 haloalkyl;
Q¹ is selected from C₃₋₁₂ cycloalkyl, C₃-12 cycloalkenyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —S(O)ₓR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, —OC(O)N(R¹⁰) 2, —NR¹⁰SO₂R¹⁰, —SO₂N(R¹⁰) 2 and —NR¹⁰C(O)N(R¹⁰) 2,
wherein said C₁₋₆ alkyl, C₂₋₆ alkenyl and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3.

5. The method of claim 4,
wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or NR⁶,
R⁶ is selected from H, C₁₋₄ alkyl, and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₃₋₁₂ cycloalkyl, 3- to 12-membered heterocyclyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{X1}R^{X2};
wherein R^{X1} and R^{X2} are independently selected from: H, C₁₋₄ alkyl optionally substituted by OH or 3- to 6-membered heterocyclyl, —OC₁₋₆ alkyl, —C(O)—C₁₋₆ alkyl, and 5- to 10-membered heteroaryl, or an R^{X1} and an R^{X2} attached to the same nitrogen atom together to form a 3- to 6-membered heterocyclyl;
n is an integer from 0 to 4; and
x is an integer from 0 to 3.

6. The method of claim 4,
wherein R¹ and R² are independently selected from: H, C₁₋₆ alkyl, C₂₋₆ alkenyl, C₂₋₆ alkynyl and C₁₋₆ haloalkyl;
R³ is each independently selected from: halo, C₁₋₆ alkyl and amino;
X₁ is N and X₂ is CR⁴, or X₁ is C and X₂ is NR⁵;
X₃ is CH or N;
R⁴, R⁵ and R¹² are independently selected from: H, halo, CN, C₁₋₆ alkyl and C₁₋₆ haloalkyl;
wherein when X₁ is N, then
X₄ is N and X₅ is CH, or X₄ is CH and X₅ is N; and
wherein when X₁ is C, then both X₄ and X₅ are CH
wherein said C₁₋₆ alkyl on R⁴ or R⁵ is optionally partially or fully deuterated;
L¹ is a bond or NR⁶,
R⁶ is selected from H, C₁₋₄ alkyl, and C₁₋₄ haloalkyl;
L² is a bond or —[CR⁷R⁸]p-,
where p is an integer from 1 to 4;
R⁷ and R⁸ are each independently selected from: H, C₁₋₄ alkyl, and C₁₋₄ haloalkyl, OH, COOH, C(O)NR^{x1}R^{X2}, and C₃₋₆ cycloalkyl, or an R⁷ and an R⁸ attached to the same carbon atom in L² together form a C₃₋₆ cycloalkyl or 3-6-membered heterocyclyl,
wherein said C₁₋₄ alkyl is optionally substituted by OH, O—C₁₋₄alkyl, or C₆₋₁₀ aryl optionally substituted by halogen or C₁₋₆ haloalkyl;
Q¹ is selected from C₃₋₁₂ cycloalkyl, C₆₋₁₀ aryl, and 5- to 10-membered heteroaryl;
wherein said C₆₋₁₀ aryl and 5- to 10-membered heteroaryl is optionally substituted by one or more R⁹;
each R⁹ is independently selected from: halo, —CN, —NO₂, C₁₋₆ alkyl, C₂₋₆ alkenyl, C₂₋₆ alkynyl, C₁₋₆ haloalkyl, —OR¹⁰, —N(R¹⁰) 2, —C(O)R¹⁰, —OC(O)R¹⁰, —C(O)OR¹⁰, —NR¹⁰C(O)R¹⁰, —NR¹⁰C(O)OR¹⁰, —C(O)N(R¹⁰) 2, and —OC(O)N(R¹⁰) 2,
wherein said C₁₋₆ alkyl, C₂₋₆ alkenyl and C₂₋₆ alkynyl is optionally substituted by one or more R¹¹,
wherein each R¹⁰ is independently selected from: H, C₁₋₆ alkyl and C₁₋₆ haloalkyl;
wherein each R¹¹ is independently selected from: halo, —CN, —NO₂, C₁₋₄ alkyl, C₁₋₄ haloalkyl, and NR^{x1}R^{X2};
wherein R^{X1} and R^{X2} are independently selected from: H, C₁₋₄ alkyl optionally substituted by OH or 3- to 6-membered heterocyclyl;
n is an integer from 0 to 4; and
x is an integer from 0 to 3.

7. The method of claim 1,
Wherein the group 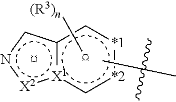
is selected from any of the following structures:

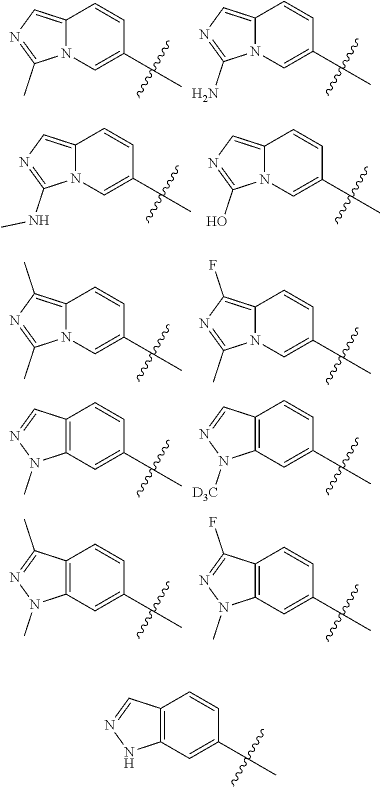

8. The method of claim 4,
Wherein the group 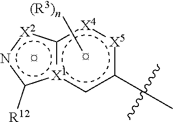
is selected from any of the following structures:

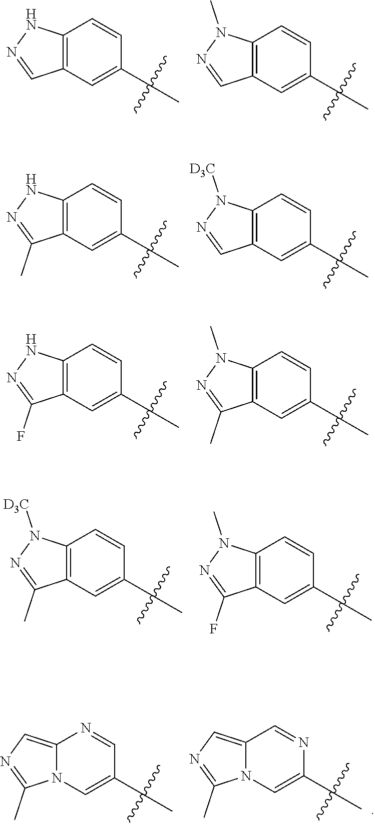

9. The method of claim 1,
wherein the group 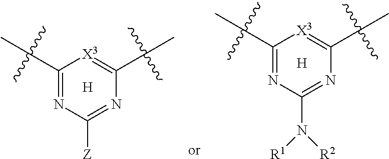
is selected from any of the following structures:

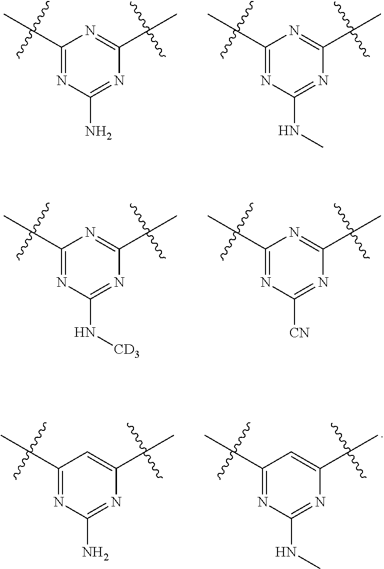

10. The method of claim 4,
wherein the group 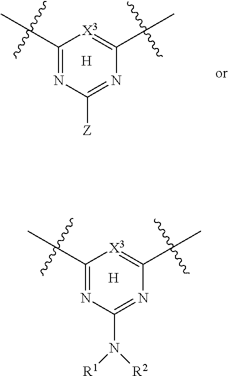
is selected from any of the following structures:

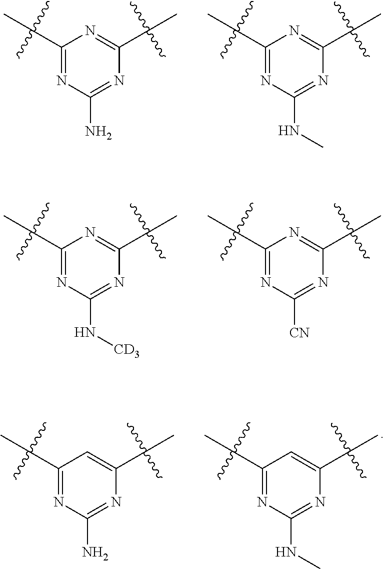

11. The method of claim 1, wherein the group-L¹-L²-Q¹ is selected from any of the following structures:

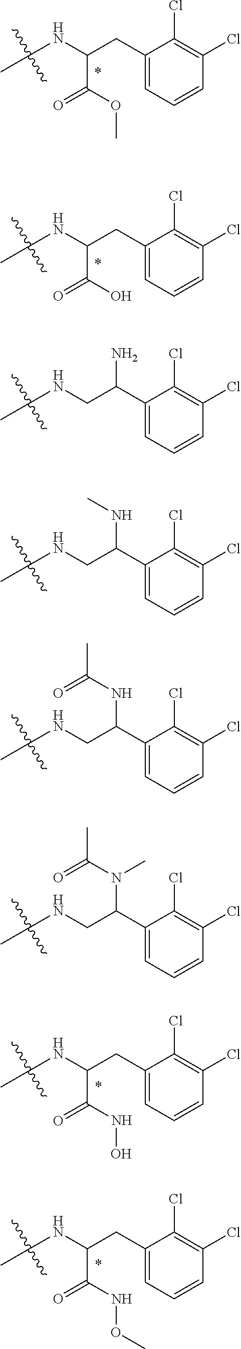

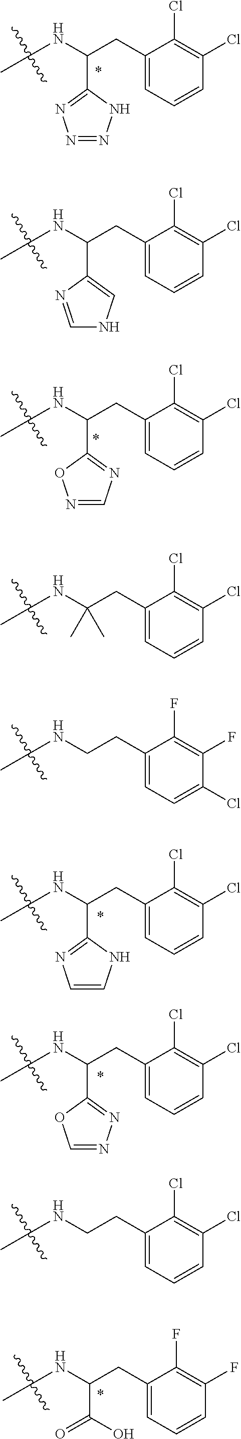

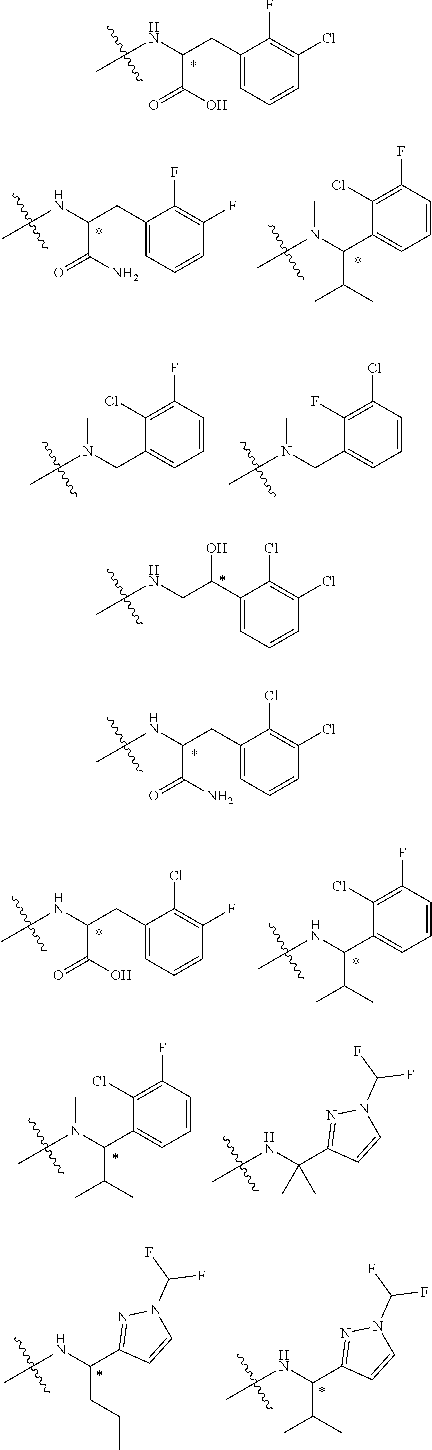

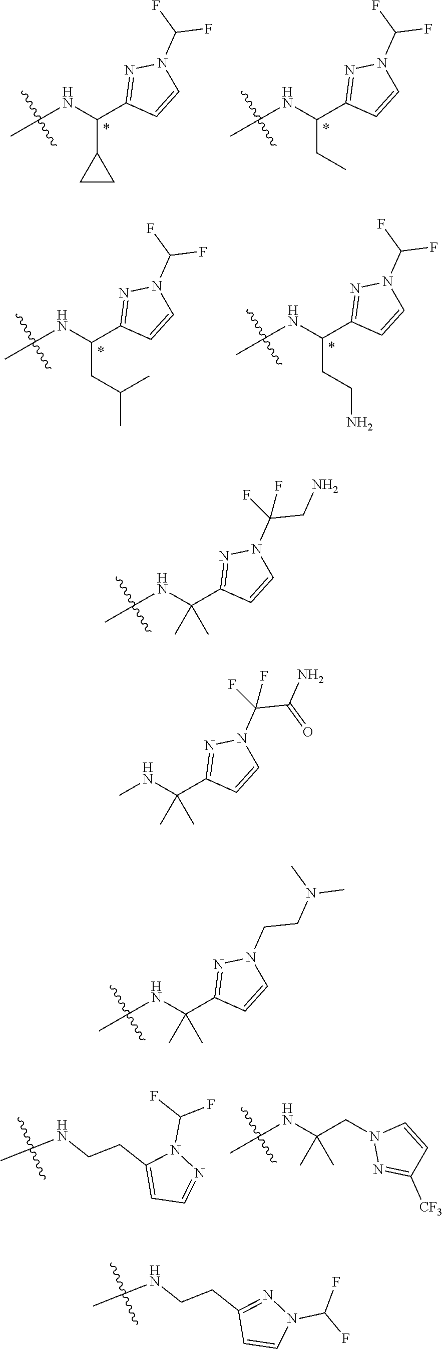

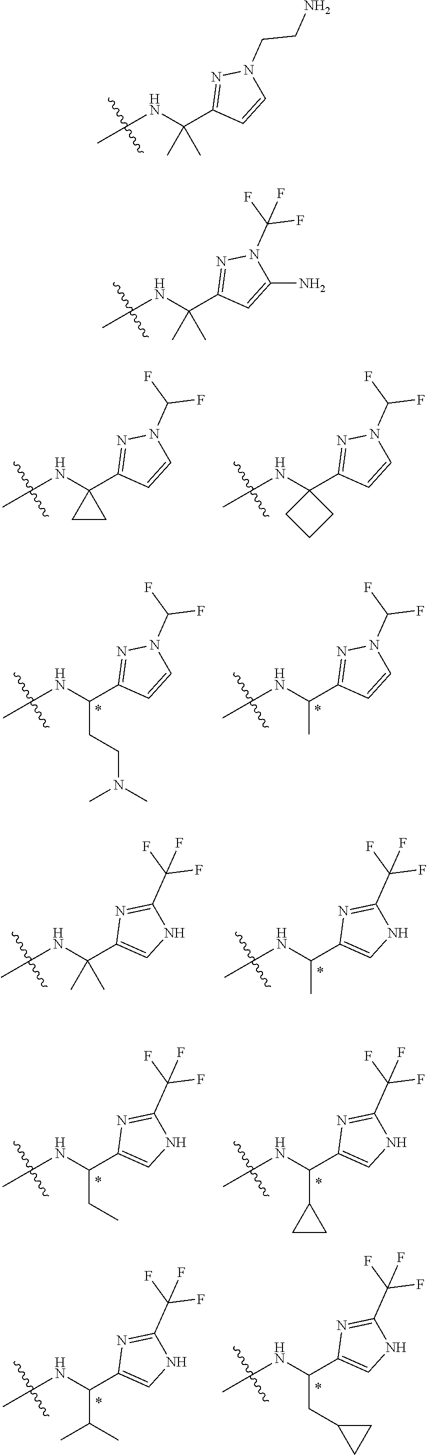

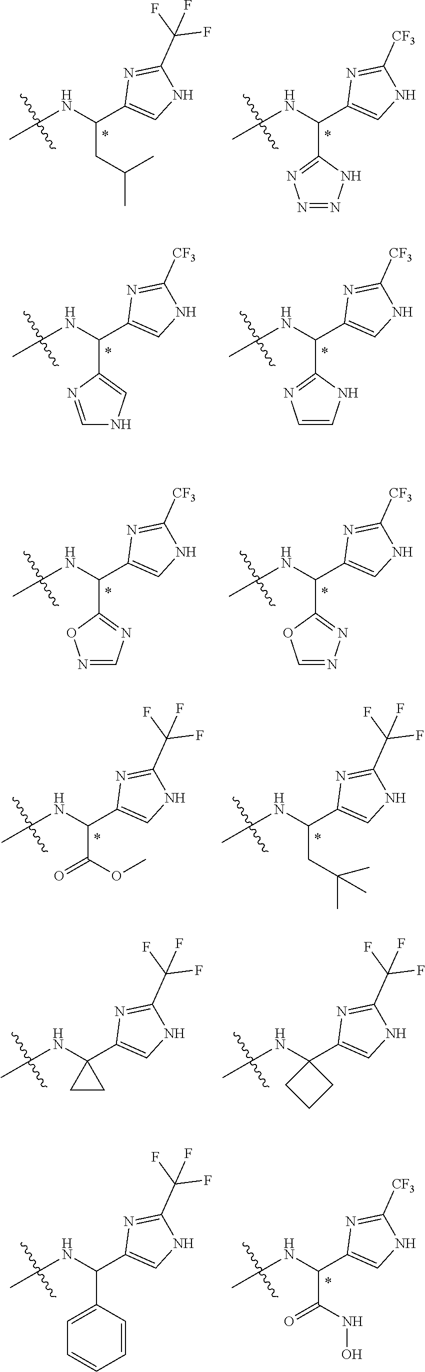

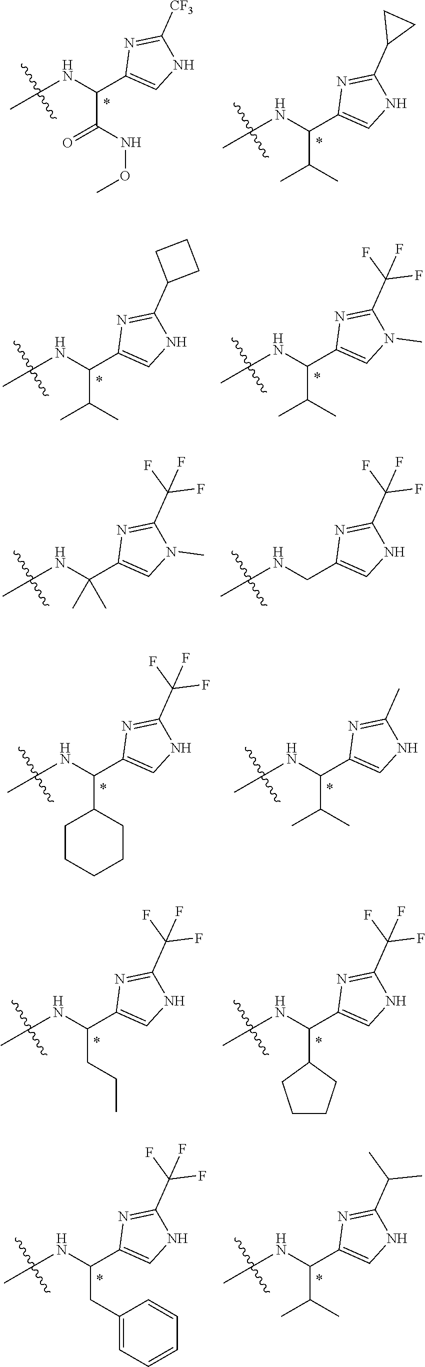

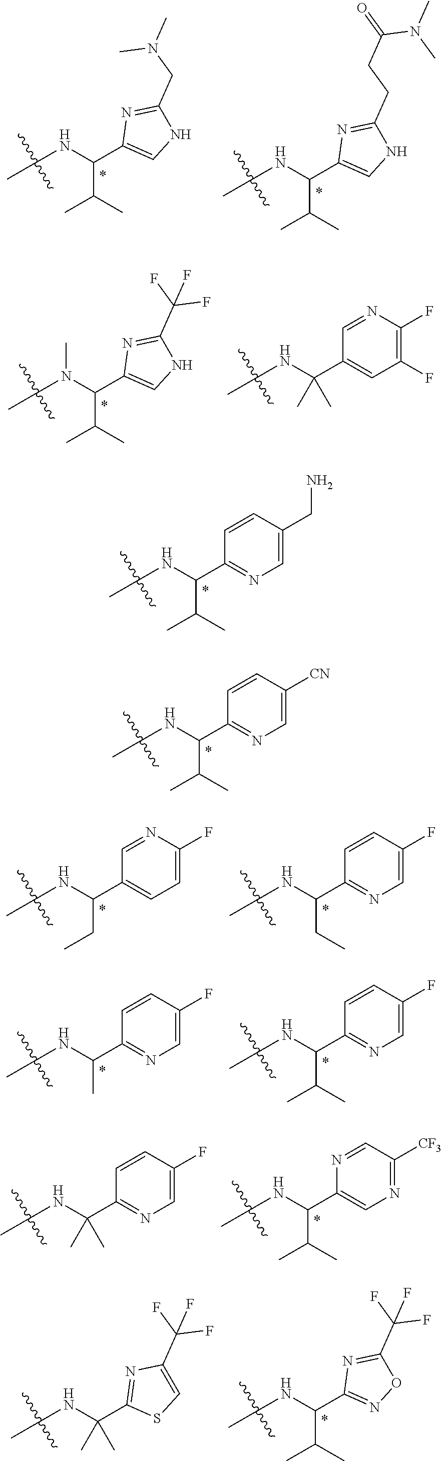

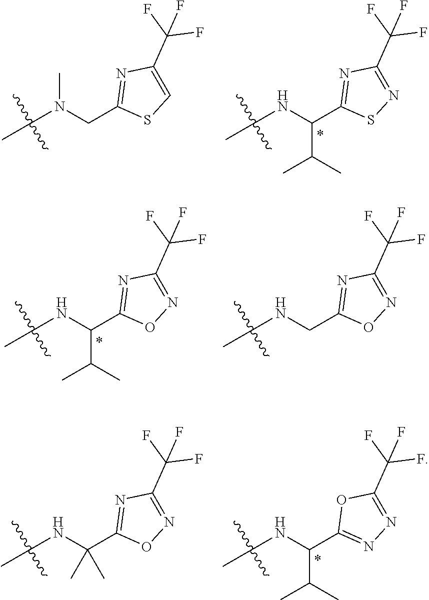 [SMILES: *N(C)Cc1nc(C(F)(F)F)cs1 ; *NC(c1nc(C(F)(F)F)ns1)C(C)C ; *NC(c1nc(C(F)(F)F)no1)C(C)C ; *NCc1nc(C(F)(F)F)no1 ; *NC(C)(C)c1nc(C(F)(F)F)no1 ; *NC(c1nnc(C(F)(F)F)o1)C(C)C]

12. The method of claim 4, wherein the group-L¹-L²-Q¹ is selected from any of the following structures:

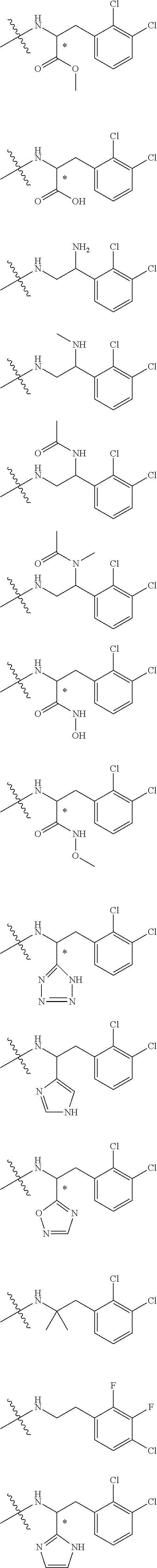

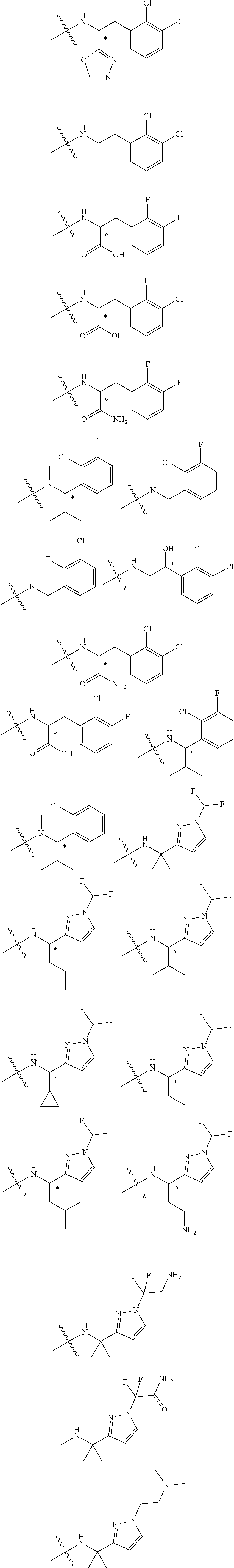

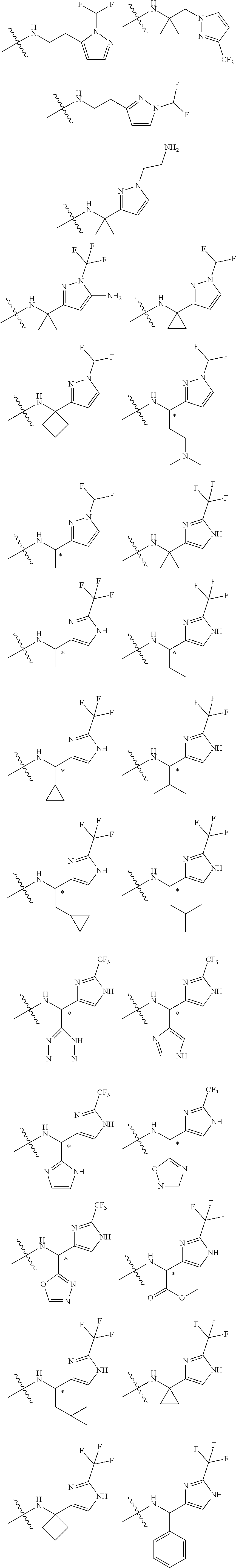

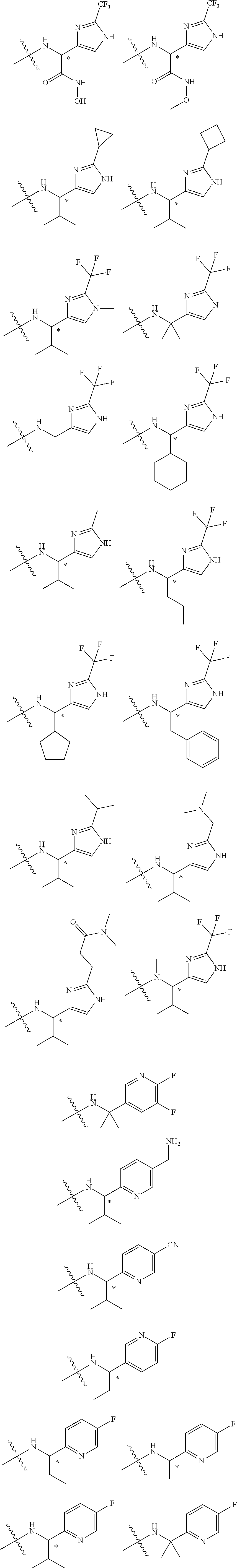

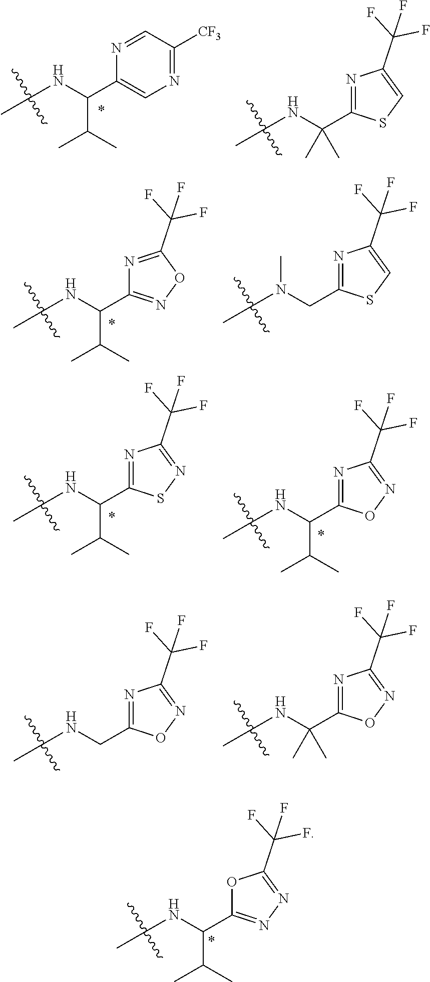

13. A method of treating a disease or medical condition in a subject, wherein the disease or medical condition is mediated by microtubule associated serine/threonine-like kinase (MASTL), the method comprising:
administering, to a subject in need of such treating, an effective amount of a composition comprising a compound or the pharmaceutically acceptable salt thereof, wherein the compound is any one selected from the group consisting of Compound Nos. 1 to 239 below:

| No | Structure |
|---|---|
| 1 | 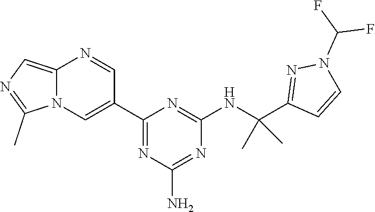 [SMILES: Cc1ncc2ncc(-c3nc(N)nc(NC(C)(C)c4ccn(C(F)F)n4)n3)cn12] |
| 2 | 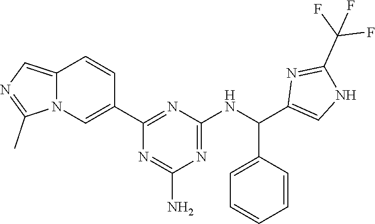 [SMILES: Cc1ncc2ccc(-c3nc(N)nc(NC(c4ccccc4)c4c[nH]c(C(F)(F)F)n4)n3)cn12] |
| 3 | 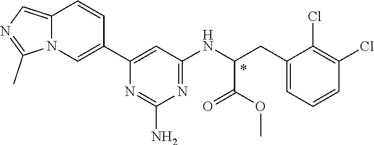 [SMILES: COC(=O)C(Cc1cccc(Cl)c1Cl)Nc1cc(-c2ccc3cnc(C)n3c2)nc(N)n1] |
| 4 | 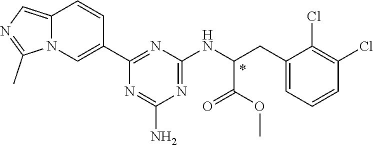 [SMILES: COC(=O)C(Cc1cccc(Cl)c1Cl)Nc1nc(N)nc(-c2ccc3cnc(C)n3c2)n1] |
| 5 | 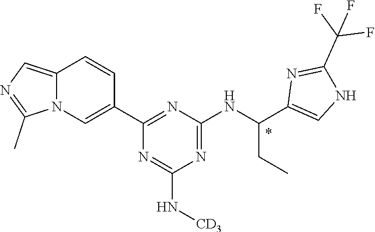 [SMILES: [2H]C([2H])([2H])Nc1nc(NC(CC)c2c[nH]c(C(F)(F)F)n2)nc(-c2ccc3cnc(C)n3c2)n1] |
| 6 | 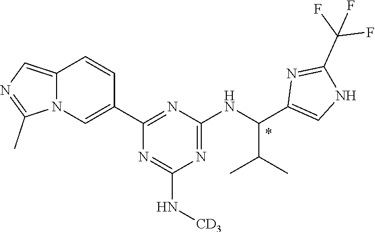 [SMILES: [2H]C([2H])([2H])Nc1nc(NC(c2c[nH]c(C(F)(F)F)n2)C(C)C)nc(-c2ccc3cnc(C)n3c2)n1] |
| 7 | 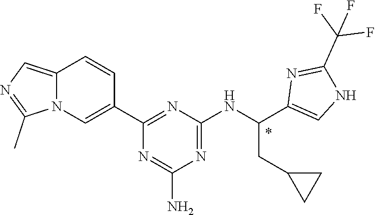 [SMILES: Cc1ncc2ccc(-c3nc(N)nc(NC(CC4CC4)c4c[nH]c(C(F)(F)F)n4)n3)cn12] |
| 8 | 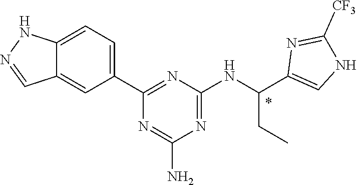 [SMILES: CCC(Nc1nc(N)nc(-c2ccc3[nH]ncc3c2)n1)c1c[nH]c(C(F)(F)F)n1] |
| 9 | 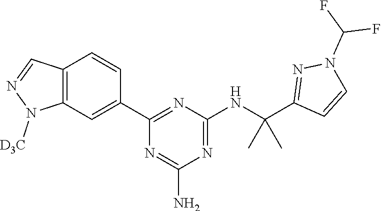 [SMILES: [2H]C([2H])([2H])n1ncc2ccc(-c3nc(N)nc(NC(C)(C)c4ccn(C(F)F)n4)n3)cc21] |
| 10 | 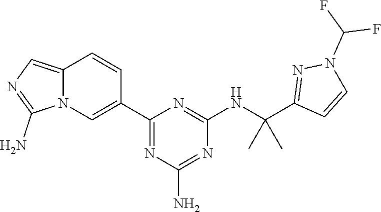 [SMILES: CC(C)(Nc1nc(N)nc(-c2ccc3cnc(N)n3c2)n1)c1ccn(C(F)F)n1] |
| 11 | 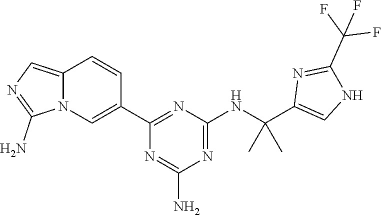 [SMILES: CC(C)(Nc1nc(N)nc(-c2ccc3cnc(N)n3c2)n1)c1c[nH]c(C(F)(F)F)n1] |
| 12 | 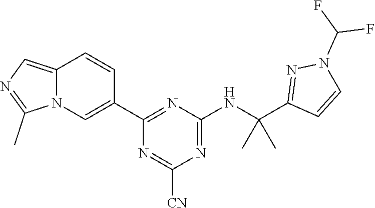 [SMILES: Cc1ncc2ccc(-c3nc(C#N)nc(NC(C)(C)c4ccn(C(F)F)n4)n3)cn12] |
| 13 | 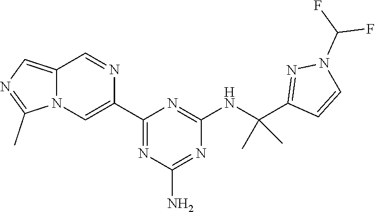 [SMILES: Cc1ncc2cnc(-c3nc(N)nc(NC(C)(C)c4ccn(C(F)F)n4)n3)cn12] |
| 14 | 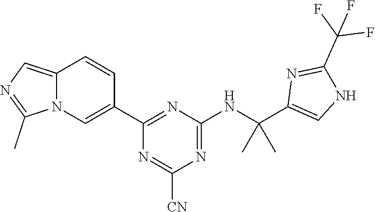 [SMILES: Cc1ncc2ccc(-c3nc(C#N)nc(NC(C)(C)c4c[nH]c(C(F)(F)F)n4)n3)cn12] |
| 15 | 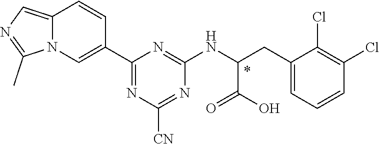 [SMILES: Cc1ncc2ccc(-c3nc(C#N)nc(NC(Cc4cccc(Cl)c4Cl)C(=O)O)n3)cn12] |
| 16 | 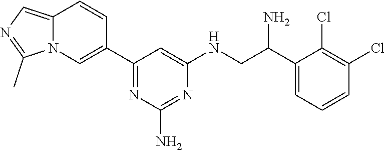 [SMILES: Cc1ncc2ccc(-c3cc(NCC(N)c4cccc(Cl)c4Cl)nc(N)n3)cn12] |
| 17 | 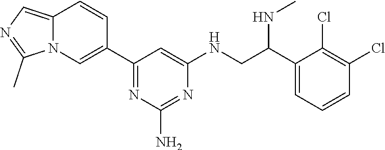 [SMILES: CNC(CNc1cc(-c2ccc3cnc(C)n3c2)nc(N)n1)c1cccc(Cl)c1Cl] |
| 18 | 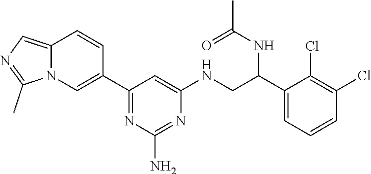 [SMILES: CC(=O)NC(CNc1cc(-c2ccc3cnc(C)n3c2)nc(N)n1)c1cccc(Cl)c1Cl] |
| 19 | 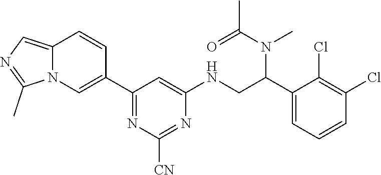 [SMILES: CC(=O)N(C)C(CNc1cc(-c2ccc3cnc(C)n3c2)nc(C#N)n1)c1cccc(Cl)c1Cl] |
| 20 | 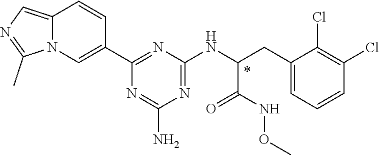 [SMILES: CONC(=O)C(Cc1cccc(Cl)c1Cl)Nc1nc(N)nc(-c2ccc3cnc(C)n3c2)n1] |
| 21 | 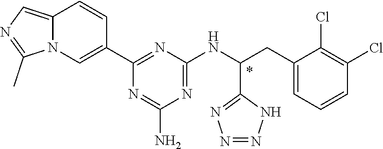 [SMILES: Cc1ncc2ccc(-c3nc(N)nc(NC(Cc4cccc(Cl)c4Cl)c4nnn[nH]4)n3)cn12] |
| 22 | 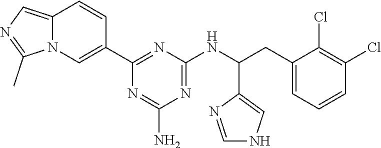 [SMILES: Cc1ncc2ccc(-c3nc(N)nc(NC(Cc4cccc(Cl)c4Cl)c4c[nH]cn4)n3)cn12] |
| 23 | 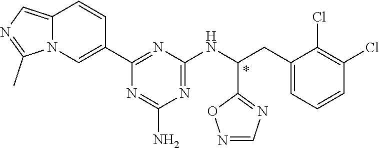 [SMILES: Cc1ncc2ccc(-c3nc(N)nc(NC(Cc4cccc(Cl)c4Cl)c4ncno4)n3)cn12] |
| 24 | 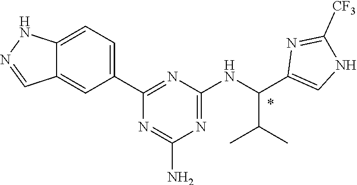 [SMILES: CC(C)C(Nc1nc(N)nc(-c2ccc3[nH]ncc3c2)n1)c1c[nH]c(C(F)(F)F)n1] |
| 25 | 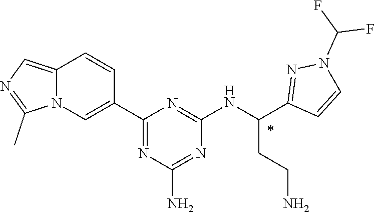 [SMILES: Cc1ncc2ccc(-c3nc(N)nc(NC(CCN)c4ccn(C(F)F)n4)n3)cn12] |
| 26 | 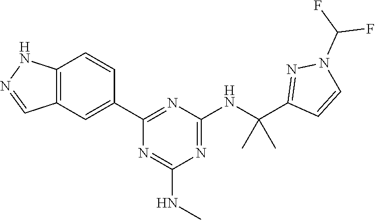 [SMILES: CNc1nc(NC(C)(C)c2ccn(C(F)F)n2)nc(-c2ccc3[nH]ncc3c2)n1] |
| 27 | 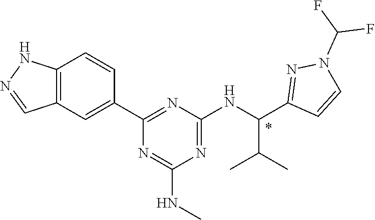 [SMILES: CNc1nc(NC(c2ccn(C(F)F)n2)C(C)C)nc(-c2ccc3[nH]ncc3c2)n1] |
| 28 | 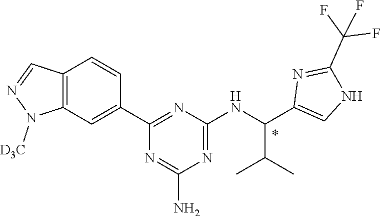 [SMILES: [2H]C([2H])([2H])n1ncc2ccc(-c3nc(N)nc(NC(c4c[nH]c(C(F)(F)F)n4)C(C)C)n3)cc21] |
| 29 | 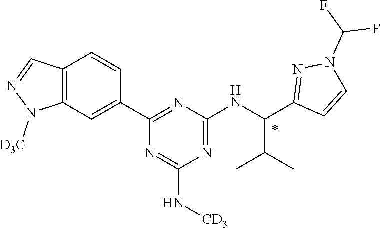 [SMILES: [2H]C([2H])([2H])Nc1nc(NC(c2ccn(C(F)F)n2)C(C)C)nc(-c2ccc3cnn(C([2H])([2H])[2H])c3c2)n1] |
| 30 | 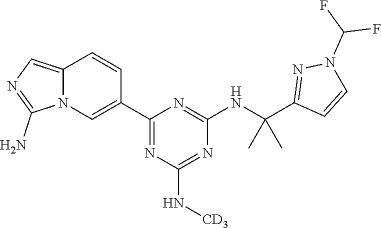 [SMILES: [2H]C([2H])([2H])Nc1nc(NC(C)(C)c2ccn(C(F)F)n2)nc(-c2ccc3cnc(N)n3c2)n1] |
| 31 | 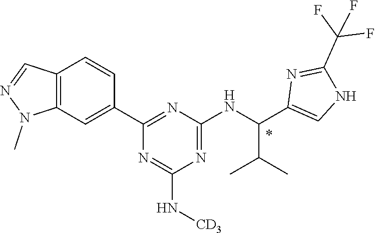 [SMILES: [2H]C([2H])([2H])Nc1nc(NC(c2c[nH]c(C(F)(F)F)n2)C(C)C)nc(-c2ccc3cnn(C)c3c2)n1] |
| 32 | 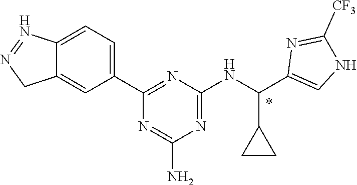 |
| 33 | 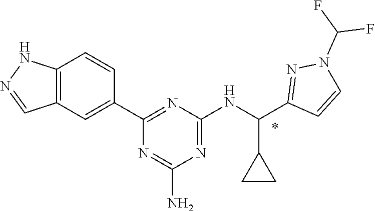 [SMILES: Nc1nc(NC(c2ccn(C(F)F)n2)C2CC2)nc(-c2ccc3[nH]ncc3c2)n1] |
| 34 | 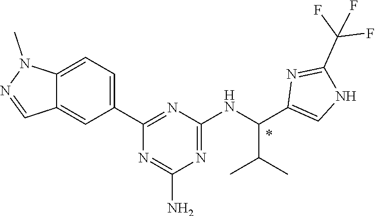 [SMILES: CC(C)C(Nc1nc(N)nc(-c2ccc3c(cnn3C)c2)n1)c1c[nH]c(C(F)(F)F)n1] |
| 35 | 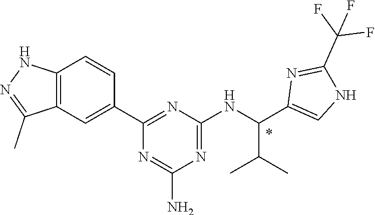 [SMILES: Cc1n[nH]c2ccc(-c3nc(N)nc(NC(c4c[nH]c(C(F)(F)F)n4)C(C)C)n3)cc12] |
| 36 | 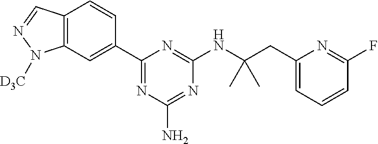 [SMILES: [2H]C([2H])([2H])n1ncc2ccc(-c3nc(N)nc(NC(C)(C)Cc4cccc(F)n4)n3)cc21] |
| 37 | 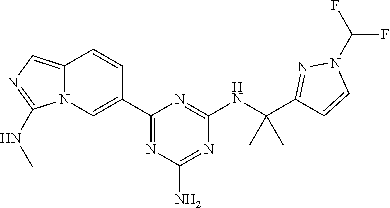 [SMILES: CNc1ncc2ccc(-c3nc(N)nc(NC(C)(C)c4ccn(C(F)F)n4)n3)cn12] |
| 38 | 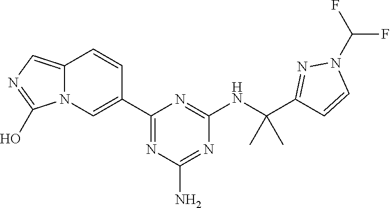 [SMILES: CC(C)(Nc1nc(N)nc(-c2ccc3cnc(O)n3c2)n1)c1ccn(C(F)F)n1] |
| 39 | 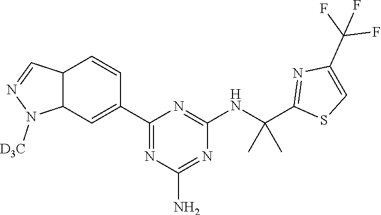 [SMILES: [2H]C([2H])([2H])N1N=CC2C=CC(c3nc(N)nc(NC(C)(C)c4nc(C(F)(F)F)cs4)n3)=CC21] |
| 40 | 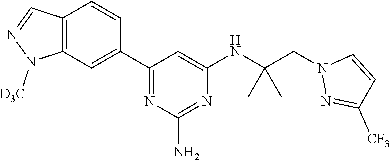 [SMILES: [2H]C([2H])([2H])n1ncc2ccc(-c3cc(NC(C)(C)Cn4ccc(C(F)(F)F)n4)nc(N)n3)cc21] |
| 41 | 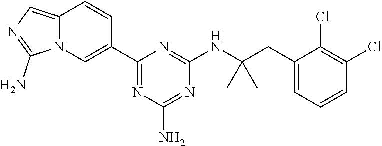 [SMILES: CC(C)(Cc1cccc(Cl)c1Cl)Nc1nc(N)nc(-c2ccc3cnc(N)n3c2)n1] |
| 42 | 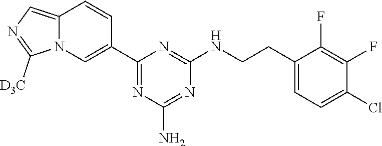 [SMILES: [2H]C([2H])([2H])c1ncc2ccc(-c3nc(N)nc(NCCc4ccc(Cl)c(F)c4F)n3)cn12] |
| 43 | 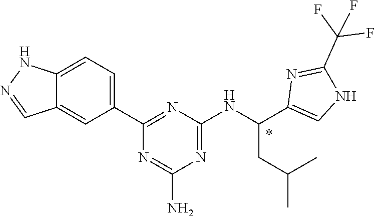 [SMILES: CC(C)CC(Nc1nc(N)nc(-c2ccc3[nH]ncc3c2)n1)c1c[nH]c(C(F)(F)F)n1] |
| 44 | 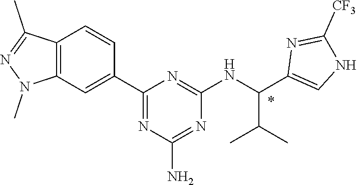 [SMILES: Cc1nn(C)c2cc(-c3nc(N)nc(NC(c4c[nH]c(C(F)(F)F)n4)C(C)C)n3)ccc12] |
| 45 | 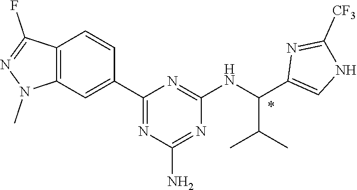 [SMILES: CC(C)C(Nc1nc(N)nc(-c2ccc3c(F)nn(C)c3c2)n1)c1c[nH]c(C(F)(F)F)n1] |
| 46 | 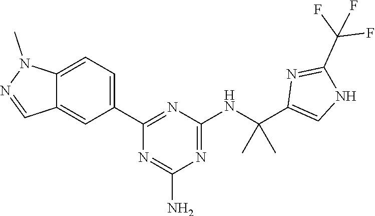 [SMILES: Cn1ncc2cc(-c3nc(N)nc(NC(C)(C)c4c[nH]c(C(F)(F)F)n4)n3)ccc21] |
| 47 | 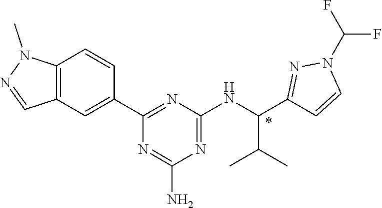 [SMILES: CC(C)C(Nc1nc(N)nc(-c2ccc3c(cnn3C)c2)n1)c1ccn(C(F)F)n1] |
| 48 | 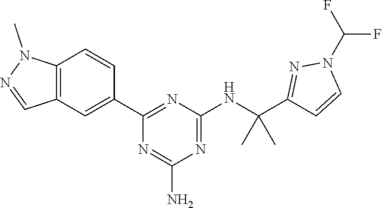 [SMILES: Cn1ncc2cc(-c3nc(N)nc(NC(C)(C)c4ccn(C(F)F)n4)n3)ccc21] |
| 49 | 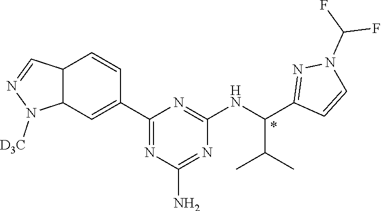 [SMILES: [2H]C([2H])([2H])N1N=CC2C=CC(c3nc(N)nc(NC(c4ccn(C(F)F)n4)C(C)C)n3)=CC21] |
| 50 | 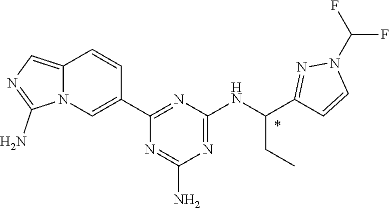 [SMILES: CCC(Nc1nc(N)nc(-c2ccc3cnc(N)n3c2)n1)c1ccn(C(F)F)n1] |
| 51 | 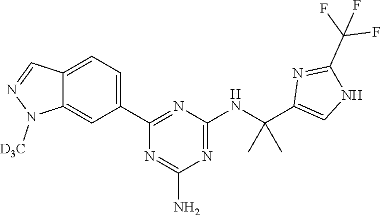 [SMILES: [2H]C([2H])([2H])n1ncc2ccc(-c3nc(N)nc(NC(C)(C)c4c[nH]c(C(F)(F)F)n4)n3)cc21] |
| 52 | 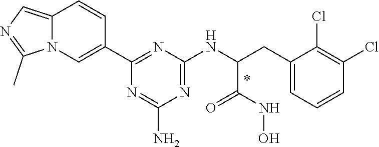 [SMILES: Cc1ncc2ccc(-c3nc(N)nc(NC(Cc4cccc(Cl)c4Cl)C(=O)NO)n3)cn12] |
| 53 | 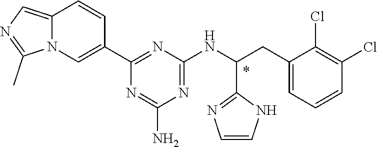 [SMILES: Cc1ncc2ccc(-c3nc(N)nc(NC(Cc4cccc(Cl)c4Cl)c4ncc[nH]4)n3)cn12] |
| 54 | 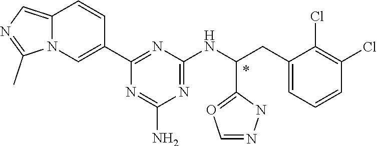 |
| 55 | 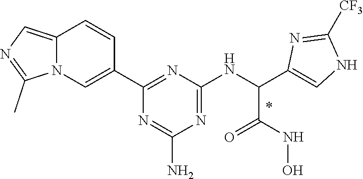 [SMILES: Cc1ncc2ccc(-c3nc(N)nc(NC(C(=O)NO)c4c[nH]c(C(F)(F)F)n4)n3)cn12] |
| 56 | 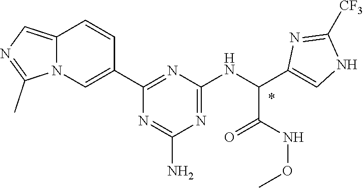 [SMILES: CONC(=O)C(Nc1nc(N)nc(-c2ccc3cnc(C)n3c2)n1)c1c[nH]c(C(F)(F)F)n1] |
| 57 | 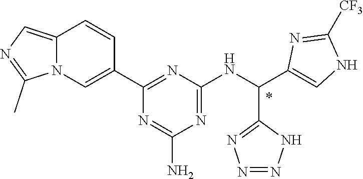 [SMILES: Cc1ncc2ccc(-c3nc(N)nc(NC(c4c[nH]c(C(F)(F)F)n4)c4nnn[nH]4)n3)cn12] |
| 58 | 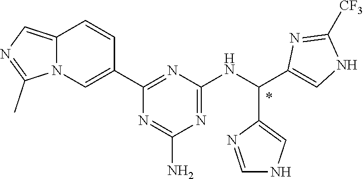 [SMILES: Cc1ncc2ccc(-c3nc(N)nc(NC(c4c[nH]cn4)c4c[nH]c(C(F)(F)F)n4)n3)cn12] |
| 59 | 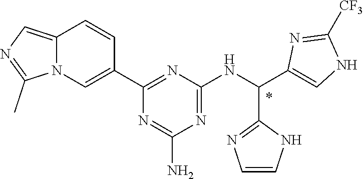 [SMILES: Cc1ncc2ccc(-c3nc(N)nc(NC(c4c[nH]c(C(F)(F)F)n4)c4ncc[nH]4)n3)cn12] |
| 60 | 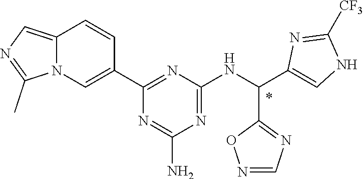 [SMILES: Cc1ncc2ccc(-c3nc(N)nc(NC(c4c[nH]c(C(F)(F)F)n4)c4ncno4)n3)cn12] |
| 61 | 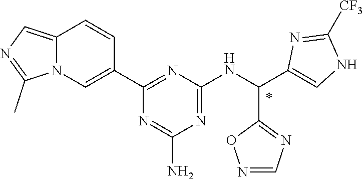 [SMILES: Cc1ncc2ccc(-c3nc(N)nc(NC(c4c[nH]c(C(F)(F)F)n4)c4ncno4)n3)cn12] |
| 62 | 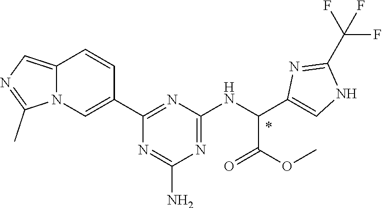 [SMILES: COC(=O)C(Nc1nc(N)nc(-c2ccc3cnc(C)n3c2)n1)c1c[nH]c(C(F)(F)F)n1] |
| 63 | 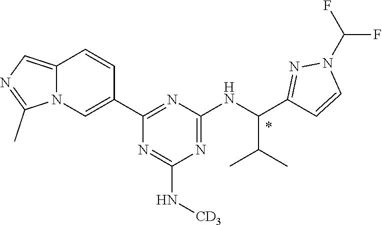 [SMILES: [2H]C([2H])([2H])Nc1nc(NC(c2ccn(C(F)F)n2)C(C)C)nc(-c2ccc3cnc(C)n3c2)n1] |
| 64 | 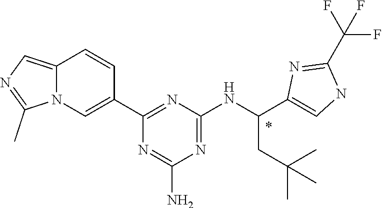 [SMILES: Cc1ncc2ccc(-c3nc(N)nc(NC(CC(C)(C)C)c4c[nH]c(C(F)(F)F)n4)n3)cn12] |
| 65 | 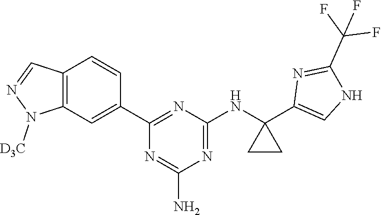 [SMILES: [2H]C([2H])([2H])n1ncc2ccc(-c3nc(N)nc(NC4(c5c[nH]c(C(F)(F)F)n5)CC4)n3)cc21] |
| 66 | 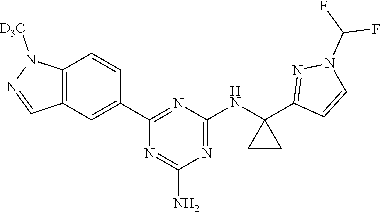 [SMILES: [2H]C([2H])([2H])n1ncc2cc(-c3nc(N)nc(NC4(c5ccn(C(F)F)n5)CC4)n3)ccc21] |
| 67 | 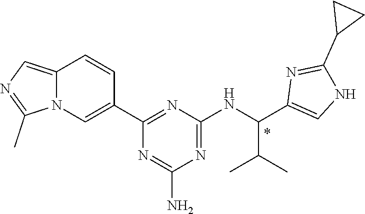 [SMILES: Cc1ncc2ccc(-c3nc(N)nc(NC(c4c[nH]c(C5CC5)n4)C(C)C)n3)cn12] |
| 68 | 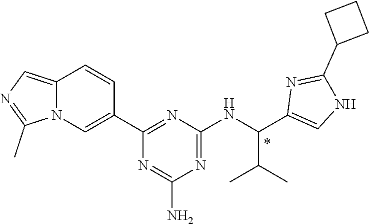 [SMILES: Cc1ncc2ccc(-c3nc(N)nc(NC(c4c[nH]c(C5CCC5)n4)C(C)C)n3)cn12] |
| 69 | 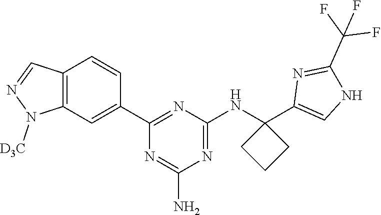 [SMILES: [2H]C([2H])([2H])n1ncc2ccc(-c3nc(N)nc(NC4(c5c[nH]c(C(F)(F)F)n5)CCC4)n3)cc21] |
| 70 | 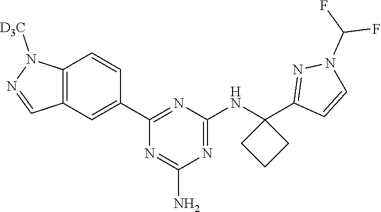 [SMILES: [2H]C([2H])([2H])n1ncc2cc(-c3nc(N)nc(NC4(c5ccn(C(F)F)n5)CCC4)n3)ccc21] |
| 71 | 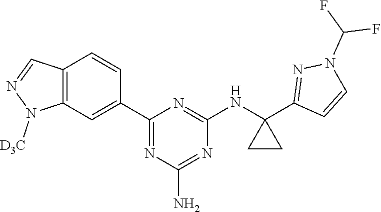 [SMILES: [2H]C([2H])([2H])n1ncc2ccc(-c3nc(N)nc(NC4(c5ccn(C(F)F)n5)CC4)n3)cc21] |
| 72 | 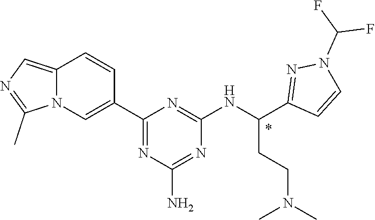 [SMILES: Cc1ncc2ccc(-c3nc(N)nc(NC(CCN(C)C)c4ccn(C(F)F)n4)n3)cn12] |
| 73 | 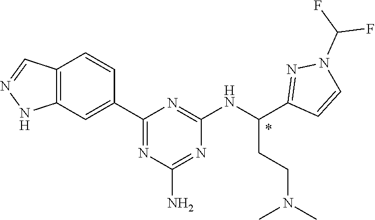 [SMILES: CN(C)CCC(Nc1nc(N)nc(-c2ccc3cn[nH]c3c2)n1)c1ccn(C(F)F)n1] |
| 74 | 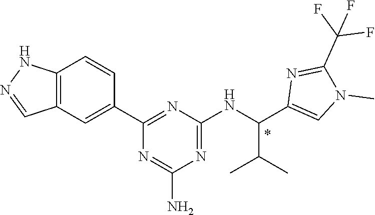 [SMILES: CC(C)C(Nc1nc(N)nc(-c2ccc3[nH]ncc3c2)n1)c1cn(C)c(C(F)(F)F)n1] |
| 75 | 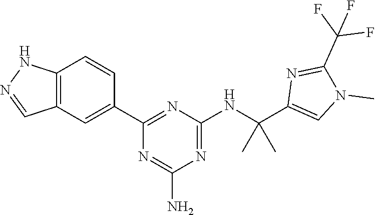 [SMILES: Cn1cc(C(C)(C)Nc2nc(N)nc(-c3ccc4[nH]ncc4c3)n2)nc1C(F)(F)F] |
| 76 | 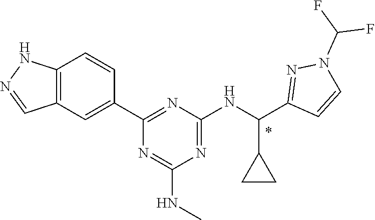 [SMILES: CNc1nc(NC(c2ccn(C(F)F)n2)C2CC2)nc(-c2ccc3[nH]ncc3c2)n1] |
| 77 | 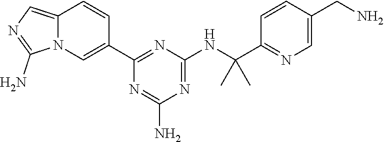 [SMILES: CC(C)(Nc1nc(N)nc(-c2ccc3cnc(N)n3c2)n1)c1ccc(CN)cn1] |
| 78 | 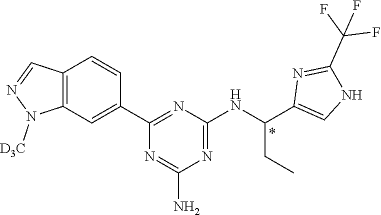 [SMILES: [2H]C([2H])([2H])n1ncc2ccc(-c3nc(N)nc(NC(CC)c4c[nH]c(C(F)(F)F)n4)n3)cc21] |
| 79 | 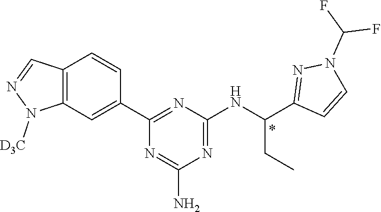 [SMILES: [2H]C([2H])([2H])n1ncc2ccc(-c3nc(N)nc(NC(CC)c4ccn(C(F)F)n4)n3)cc21] |
| 80 | 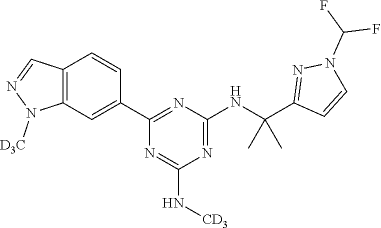 [SMILES: [2H]C([2H])([2H])Nc1nc(NC(C)(C)c2ccn(C(F)F)n2)nc(-c2ccc3cnn(C([2H])([2H])[2H])c3c2)n1] |
| 81 | 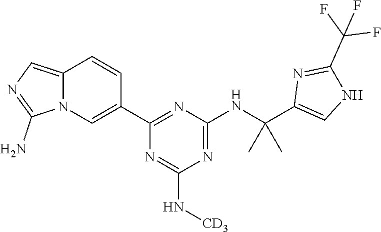 [SMILES: [2H]C([2H])([2H])Nc1nc(NC(C)(C)c2c[nH]c(C(F)(F)F)n2)nc(-c2ccc3cnc(N)n3c2)n1] |
| 82 | 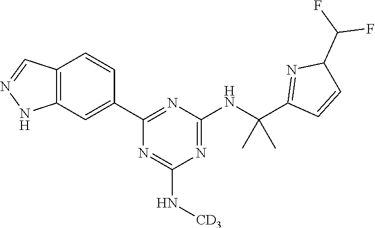 [SMILES: [2H]C([2H])([2H])Nc1nc(NC(C)(C)C2=NC(C(F)F)C=C2)nc(-c2ccc3cn[nH]c3c2)n1] |
| 83 | 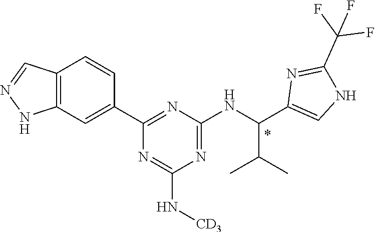 [SMILES: [2H]C([2H])([2H])Nc1nc(NC(c2c[nH]c(C(F)(F)F)n2)C(C)C)nc(-c2ccc3cn[nH]c3c2)n1] |
| 84 | 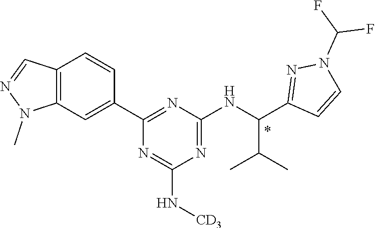 [SMILES: [2H]C([2H])([2H])Nc1nc(NC(c2ccn(C(F)F)n2)C(C)C)nc(-c2ccc3cnn(C)c3c2)n1] |
| 85 | 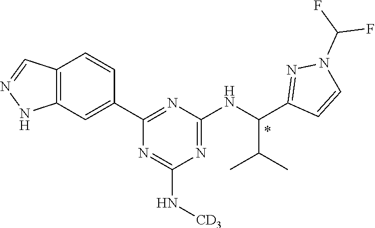 [SMILES: [2H]C([2H])([2H])Nc1nc(NC(c2ccn(C(F)F)n2)C(C)C)nc(-c2ccc3cn[nH]c3c2)n1] |
| 86 | 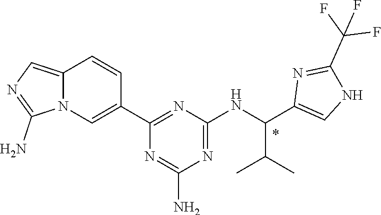 [SMILES: CC(C)C(Nc1nc(N)nc(-c2ccc3cnc(N)n3c2)n1)c1c[nH]c(C(F)(F)F)n1] |
| 87 | 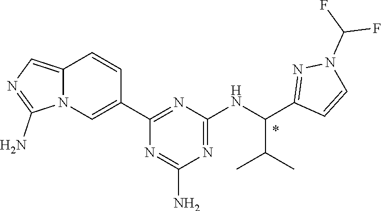 [SMILES: CC(C)C(Nc1nc(N)nc(-c2ccc3cnc(N)n3c2)n1)c1ccn(C(F)F)n1] |
| 88 | 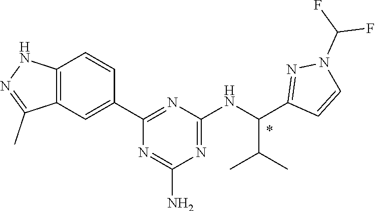 [SMILES: Cc1n[nH]c2ccc(-c3nc(N)nc(NC(c4ccn(C(F)F)n4)C(C)C)n3)cc12] |
| 89 | 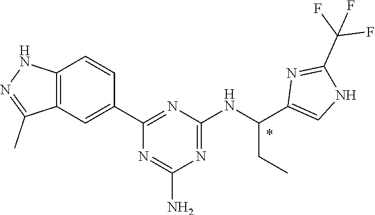 [SMILES: CCC(Nc1nc(N)nc(-c2ccc3[nH]nc(C)c3c2)n1)c1c[nH]c(C(F)(F)F)n1] |
| 90 | 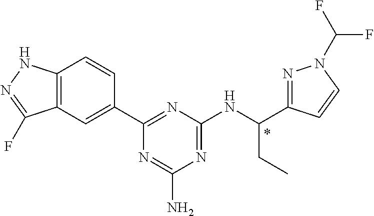 [SMILES: CCC(Nc1nc(N)nc(-c2ccc3[nH]nc(F)c3c2)n1)c1ccn(C(F)F)n1] |
| 91 | 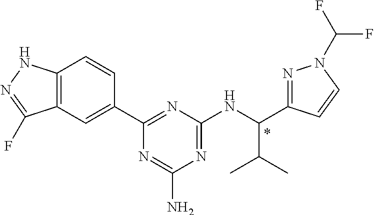 [SMILES: CC(C)C(Nc1nc(N)nc(-c2ccc3[nH]nc(F)c3c2)n1)c1ccn(C(F)F)n1] |
| 92 | 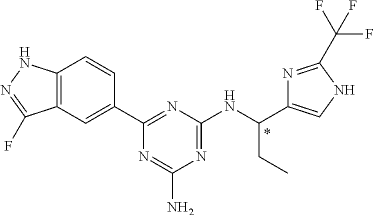 [SMILES: CCC(Nc1nc(N)nc(-c2ccc3[nH]nc(F)c3c2)n1)c1c[nH]c(C(F)(F)F)n1] |
| 93 | 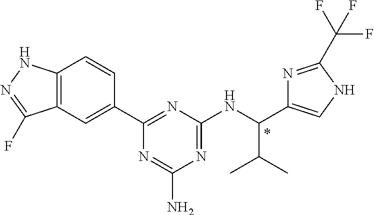 [SMILES: CC(C)C(Nc1nc(N)nc(-c2ccc3[nH]nc(F)c3c2)n1)c1c[nH]c(C(F)(F)F)n1] |
| 94 | 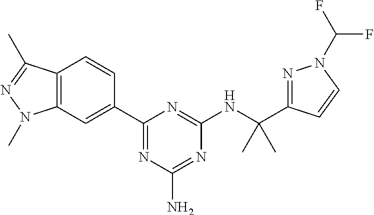 [SMILES: Cc1nn(C)c2cc(-c3nc(N)nc(NC(C)(C)c4ccn(C(F)F)n4)n3)ccc12] |
| 95 | 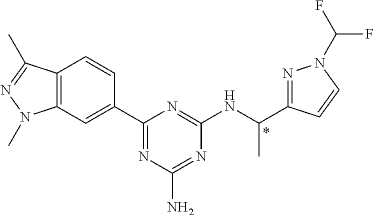 [SMILES: Cc1nn(C)c2cc(-c3nc(N)nc(NC(C)c4ccn(C(F)F)n4)n3)ccc12] |
| 96 | 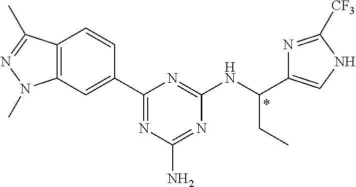 [SMILES: CCC(Nc1nc(N)nc(-c2ccc3c(C)nn(C)c3c2)n1)c1c[nH]c(C(F)(F)F)n1] |
| 97 | 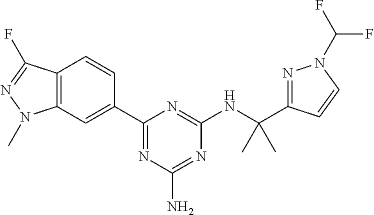 [SMILES: Cn1nc(F)c2ccc(-c3nc(N)nc(NC(C)(C)c4ccn(C(F)F)n4)n3)cc21] |
| 98 | 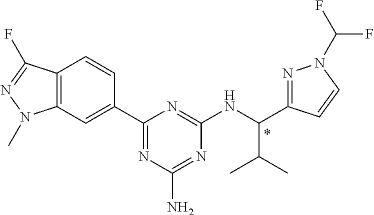 [SMILES: CC(C)C(Nc1nc(N)nc(-c2ccc3c(F)nn(C)c3c2)n1)c1ccn(C(F)F)n1] |
| 99 | 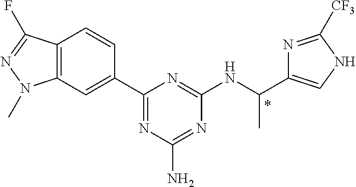 [SMILES: CC(Nc1nc(N)nc(-c2ccc3c(F)nn(C)c3c2)n1)c1c[nH]c(C(F)(F)F)n1] |
| 100 | 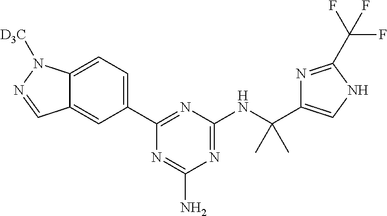 [SMILES: [2H]C([2H])([2H])n1ncc2cc(-c3nc(N)nc(NC(C)(C)c4c[nH]c(C(F)(F)F)n4)n3)ccc21] |
| 101 | 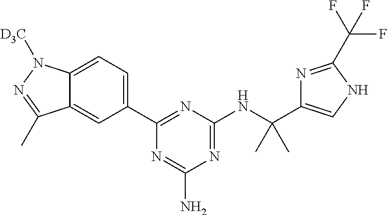 [SMILES: [2H]C([2H])([2H])n1nc(C)c2cc(-c3nc(N)nc(NC(C)(C)c4c[nH]c(C(F)(F)F)n4)n3)ccc21] |
| 102 | 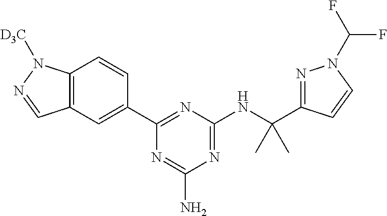 [SMILES: [2H]C([2H])([2H])n1ncc2cc(-c3nc(N)nc(NC(C)(C)c4ccn(C(F)F)n4)n3)ccc21] |
| 103 | 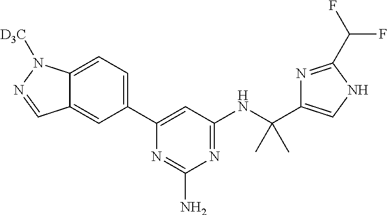 [SMILES: [2H]C([2H])([2H])n1ncc2cc(-c3cc(NC(C)(C)c4c[nH]c(C(F)F)n4)nc(N)n3)ccc21] |
| 104 | 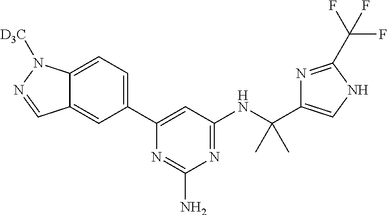 [SMILES: [2H]C([2H])([2H])n1ncc2cc(-c3cc(NC(C)(C)c4c[nH]c(C(F)(F)F)n4)nc(N)n3)ccc21] |
| 105 | 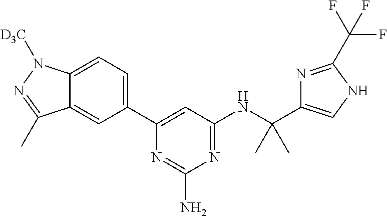 [SMILES: [2H]C([2H])([2H])n1nc(C)c2cc(-c3cc(NC(C)(C)c4c[nH]c(C(F)(F)F)n4)nc(N)n3)ccc21] |
| 106 | 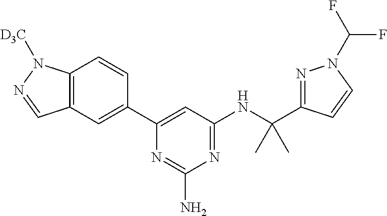 [SMILES: [2H]C([2H])([2H])n1ncc2cc(-c3cc(NC(C)(C)c4ccn(C(F)F)n4)nc(N)n3)ccc21] |
| 107 | 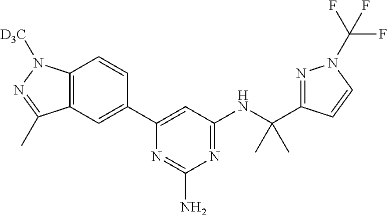 [SMILES: [2H]C([2H])([2H])n1nc(C)c2cc(-c3cc(NC(C)(C)c4ccn(C(F)(F)F)n4)nc(N)n3)ccc21] |
| 108 | 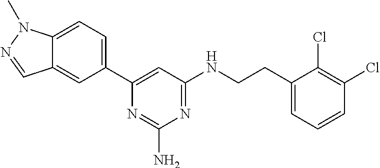 [SMILES: Cn1ncc2cc(-c3cc(NCCc4cccc(Cl)c4Cl)nc(N)n3)ccc21] |
| 109 | 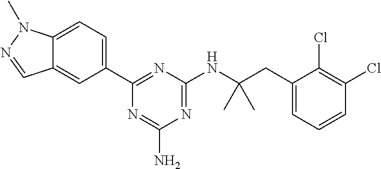 [SMILES: Cn1ncc2cc(-c3nc(N)nc(NC(C)(C)Cc4cccc(Cl)c4Cl)n3)ccc21] |
| 110 | 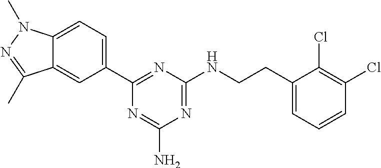 [SMILES: Cc1nn(C)c2ccc(-c3nc(N)nc(NCCc4cccc(Cl)c4Cl)n3)cc12] |
| 111 | 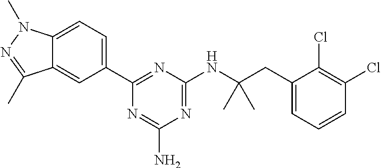 [SMILES: Cc1nn(C)c2ccc(-c3nc(N)nc(NC(C)(C)Cc4cccc(Cl)c4Cl)n3)cc12] |
| 112 | 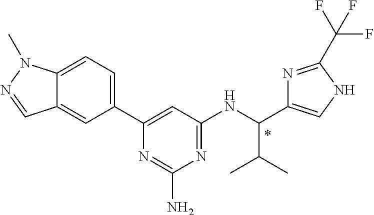 [SMILES: CC(C)C(Nc1cc(-c2ccc3c(cnn3C)c2)nc(N)n1)c1c[nH]c(C(F)(F)F)n1] |
| 113 | 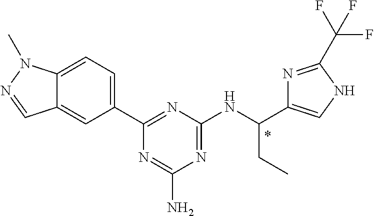 [SMILES: CCC(Nc1nc(N)nc(-c2ccc3c(cnn3C)c2)n1)c1c[nH]c(C(F)(F)F)n1] |
| 114 | 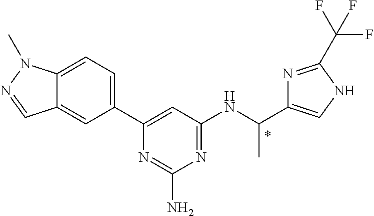 [SMILES: CC(Nc1cc(-c2ccc3c(cnn3C)c2)nc(N)n1)c1c[nH]c(C(F)(F)F)n1] |
| 115 | 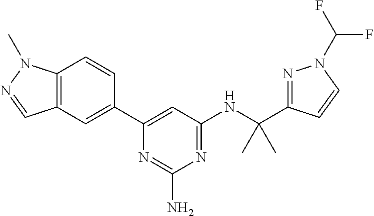 [SMILES: Cn1ncc2cc(-c3cc(NC(C)(C)c4ccn(C(F)F)n4)nc(N)n3)ccc21] |
| 116 | 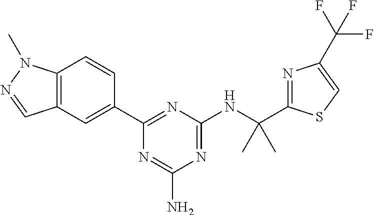 [SMILES: Cn1ncc2cc(-c3nc(N)nc(NC(C)(C)c4nc(C(F)(F)F)cs4)n3)ccc21] |
| 117 | 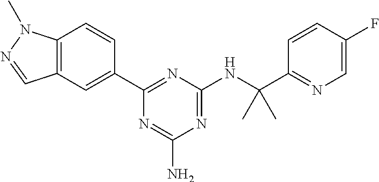 [SMILES: Cn1ncc2cc(-c3nc(N)nc(NC(C)(C)c4ccc(F)cn4)n3)ccc21] |
| 118 | 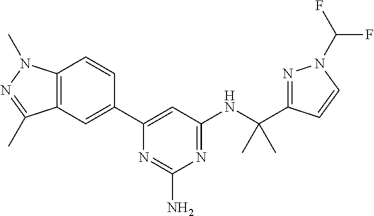 [SMILES: Cc1nn(C)c2ccc(-c3cc(NC(C)(C)c4ccn(C(F)F)n4)nc(N)n3)cc12] |
| 119 | 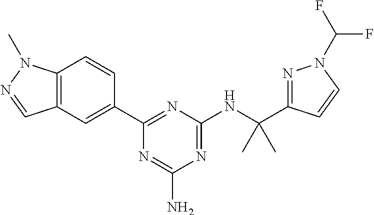 [SMILES: Cn1ncc2cc(-c3nc(N)nc(NC(C)(C)c4ccn(C(F)F)n4)n3)ccc21] |
| 120 | 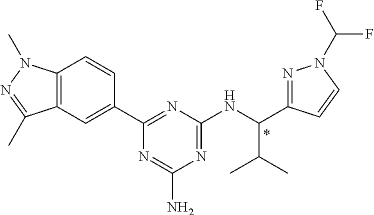 [SMILES: Cc1nn(C)c2ccc(-c3nc(N)nc(NC(c4ccn(C(F)F)n4)C(C)C)n3)cc12] |
| 121 | 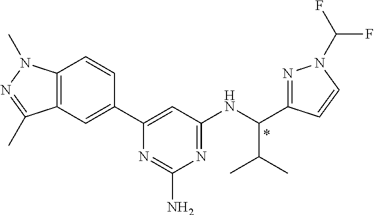 [SMILES: Cc1nn(C)c2ccc(-c3cc(NC(c4ccn(C(F)F)n4)C(C)C)nc(N)n3)cc12] |
| 122 | 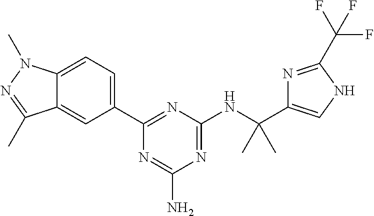 [SMILES: Cc1nn(C)c2ccc(-c3nc(N)nc(NC(C)(C)c4c[nH]c(C(F)(F)F)n4)n3)cc12] |
| 123 | 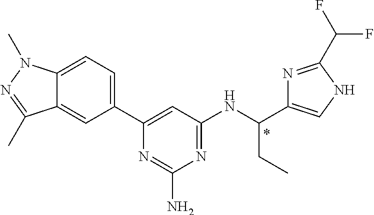 [SMILES: CCC(Nc1cc(-c2ccc3c(c2)c(C)nn3C)nc(N)n1)c1c[nH]c(C(F)F)n1] |
| 124 | 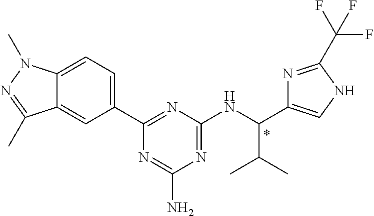 [SMILES: Cc1nn(C)c2ccc(-c3nc(N)nc(NC(c4c[nH]c(C(F)(F)F)n4)C(C)C)n3)cc12] |
| 125 | 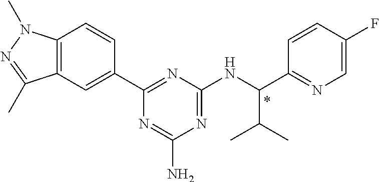 [SMILES: Cc1nn(C)c2ccc(-c3nc(N)nc(NC(c4ccc(F)cn4)C(C)C)n3)cc12] |
| 126 | 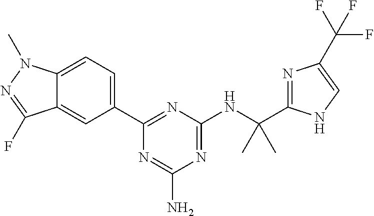 [SMILES: Cn1nc(F)c2cc(-c3nc(N)nc(NC(C)(C)c4nc(C(F)(F)F)c[nH]4)n3)ccc21] |
| 127 | 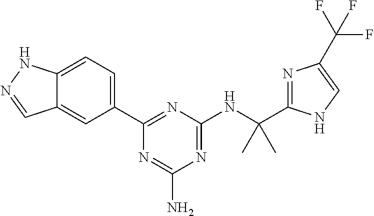 [SMILES: CC(C)(Nc1nc(N)nc(-c2ccc3[nH]ncc3c2)n1)c1nc(C(F)(F)F)c[nH]1] |
| 128 | 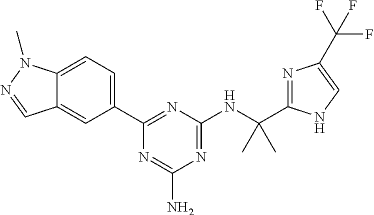 [SMILES: Cn1ncc2cc(-c3nc(N)nc(NC(C)(C)c4nc(C(F)(F)F)c[nH]4)n3)ccc21] |
| 129 | 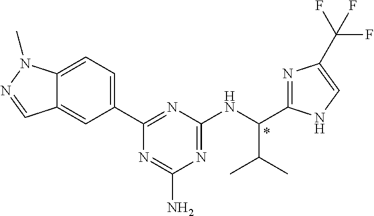 [SMILES: CC(C)C(Nc1nc(N)nc(-c2ccc3c(cnn3C)c2)n1)c1nc(C(F)(F)F)c[nH]1] |
| 130 | 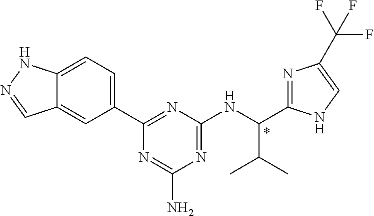 [SMILES: CC(C)C(Nc1nc(N)nc(-c2ccc3[nH]ncc3c2)n1)c1nc(C(F)(F)F)c[nH]1] |
| 131 | 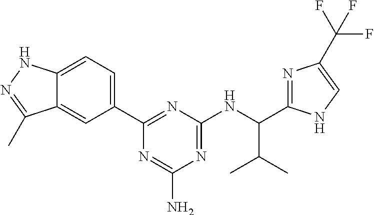 [SMILES: Cc1n[nH]c2ccc(-c3nc(N)nc(NC(c4nc(C(F)(F)F)c[nH]4)C(C)C)n3)cc12] |
| 132 | 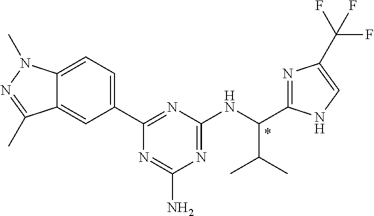 [SMILES: Cc1nn(C)c2ccc(-c3nc(N)nc(NC(c4nc(C(F)(F)F)c[nH]4)C(C)C)n3)cc12] |
| 133 | 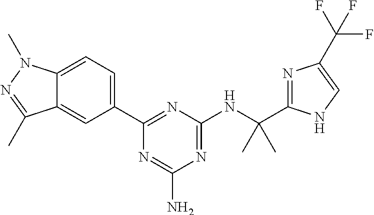 [SMILES: Cc1nn(C)c2ccc(-c3nc(N)nc(NC(C)(C)c4nc(C(F)(F)F)c[nH]4)n3)cc12] |
| 134 | 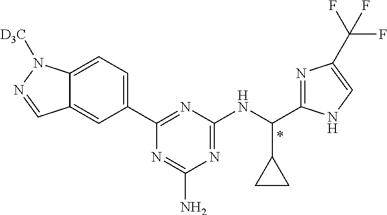 [SMILES: [2H]C([2H])([2H])n1ncc2cc(-c3nc(N)nc(NC(c4nc(C(F)(F)F)c[nH]4)C4CC4)n3)ccc21] |
| 135 | 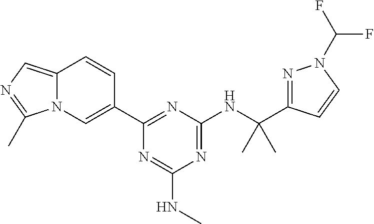 [SMILES: CNc1nc(NC(C)(C)c2ccn(C(F)F)n2)nc(-c2ccc3cnc(C)n3c2)n1] |
| 136 | 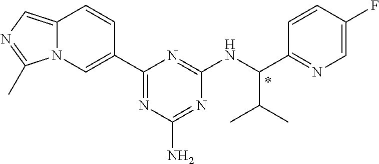 [SMILES: Cc1ncc2ccc(-c3nc(N)nc(NC(c4ccc(F)cn4)C(C)C)n3)cn12] |
| 137 | 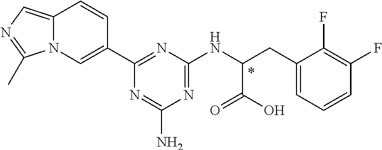 [SMILES: Cc1ncc2ccc(-c3nc(N)nc(NC(Cc4cccc(F)c4F)C(=O)O)n3)cn12] |
| 138 | 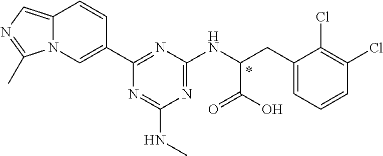 [SMILES: CNc1nc(NC(Cc2cccc(Cl)c2Cl)C(=O)O)nc(-c2ccc3cnc(C)n3c2)n1] |
| 139 | 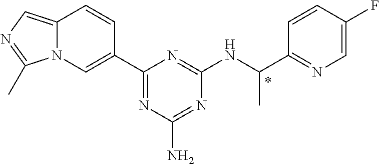 [SMILES: Cc1ncc2ccc(-c3nc(N)nc(NC(C)c4ccc(F)cn4)n3)cn12] |
| 140 | 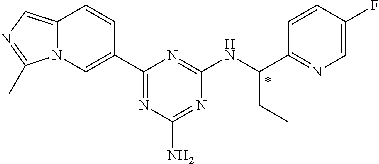 [SMILES: CCC(Nc1nc(N)nc(-c2ccc3cnc(C)n3c2)n1)c1ccc(F)cn1] |
| 141 | 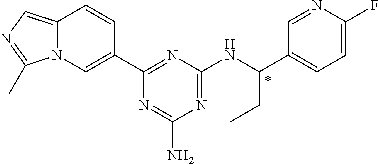 [SMILES: CCC(Nc1nc(N)nc(-c2ccc3cnc(C)n3c2)n1)c1ccc(F)nc1] |
| 142 | 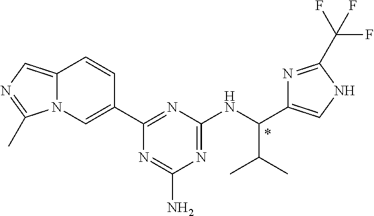 [SMILES: Cc1ncc2ccc(-c3nc(N)nc(NC(c4c[nH]c(C(F)(F)F)n4)C(C)C)n3)cn12] |
| 143 | 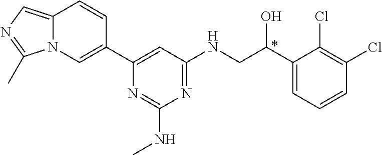 [SMILES: CNc1nc(NCC(O)c2cccc(Cl)c2Cl)cc(-c2ccc3cnc(C)n3c2)n1] |
| 144 | 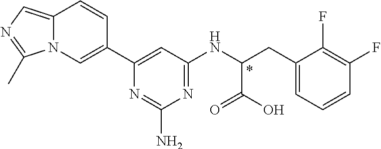 [SMILES: Cc1ncc2ccc(-c3cc(NC(Cc4cccc(F)c4F)C(=O)O)nc(N)n3)cn12] |
| 145 | 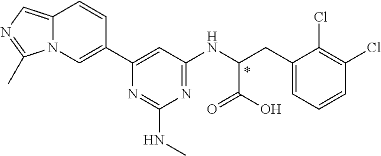 [SMILES: CNc1nc(NC(Cc2cccc(Cl)c2Cl)C(=O)O)cc(-c2ccc3cnc(C)n3c2)n1] |
| 146 | 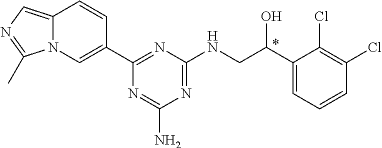 [SMILES: Cc1ncc2ccc(-c3nc(N)nc(NCC(O)c4cccc(Cl)c4Cl)n3)cn12] |
| 147 | 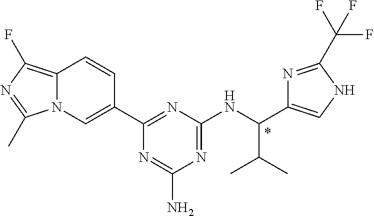 [SMILES: Cc1nc(F)c2ccc(-c3nc(N)nc(NC(c4c[nH]c(C(F)(F)F)n4)C(C)C)n3)cn12] |
| 148 | 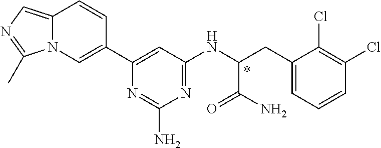 [SMILES: Cc1ncc2ccc(-c3cc(NC(Cc4cccc(Cl)c4Cl)C(N)=O)nc(N)n3)cn12] |
| 149 | 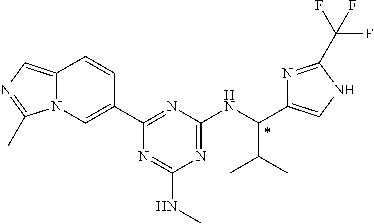 [SMILES: CNc1nc(NC(c2c[nH]c(C(F)(F)F)n2)C(C)C)nc(-c2ccc3cnc(C)n3c2)n1] |
| 150 | 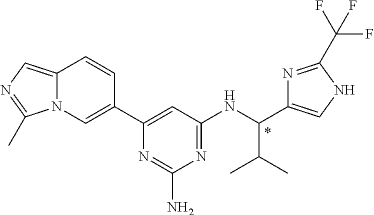 [SMILES: Cc1ncc2ccc(-c3cc(NC(c4c[nH]c(C(F)(F)F)n4)C(C)C)nc(N)n3)cn12] |
| 151 | 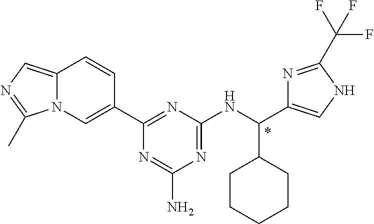 [SMILES: Cc1ncc2ccc(-c3nc(N)nc(NC(c4c[nH]c(C(F)(F)F)n4)C4CCCCC4)n3)cn12] |
| 152 | 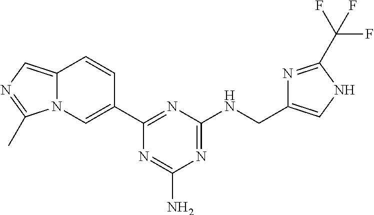 [SMILES: Cc1ncc2ccc(-c3nc(N)nc(NCc4c[nH]c(C(F)(F)F)n4)n3)cn12] |
| 153 | 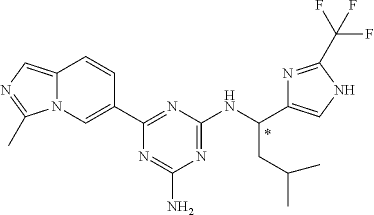 [SMILES: Cc1ncc2ccc(-c3nc(N)nc(NC(CC(C)C)c4c[nH]c(C(F)(F)F)n4)n3)cn12] |
| 154 | 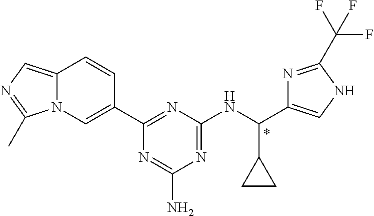 [SMILES: Cc1ncc2ccc(-c3nc(N)nc(NC(c4c[nH]c(C(F)(F)F)n4)C4CC4)n3)cn12] |
| 155 | 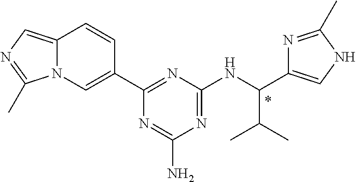 [SMILES: Cc1nc(C(Nc2nc(N)nc(-c3ccc4cnc(C)n4c3)n2)C(C)C)c[nH]1] |
| 156 | 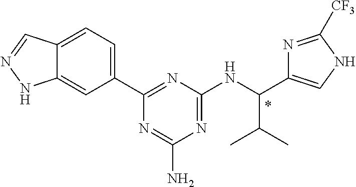 [SMILES: CC(C)C(Nc1nc(N)nc(-c2ccc3cn[nH]c3c2)n1)c1c[nH]c(C(F)(F)F)n1] |
| 157 | 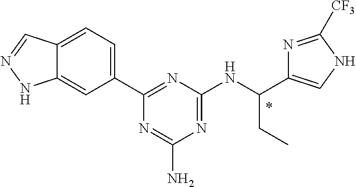 [SMILES: CCC(Nc1nc(N)nc(-c2ccc3cn[nH]c3c2)n1)c1c[nH]c(C(F)(F)F)n1] |
| 158 | 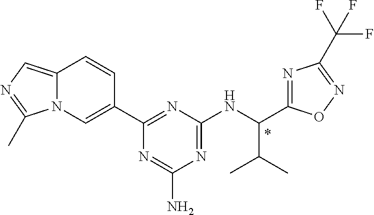 [SMILES: Cc1ncc2ccc(-c3nc(N)nc(NC(c4nc(C(F)(F)F)no4)C(C)C)n3)cn12] |
| 159 | 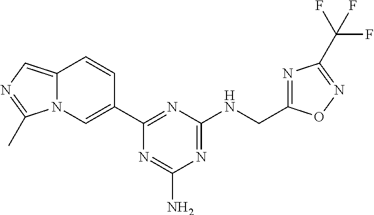 [SMILES: Cc1ncc2ccc(-c3nc(N)nc(NCc4nc(C(F)(F)F)no4)n3)cn12] |
| 160 | 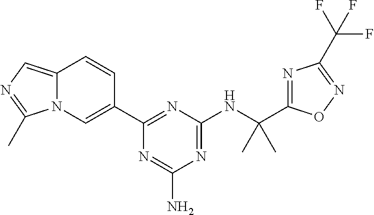 [SMILES: Cc1ncc2ccc(-c3nc(N)nc(NC(C)(C)c4nc(C(F)(F)F)no4)n3)cn12] |
| 161 | 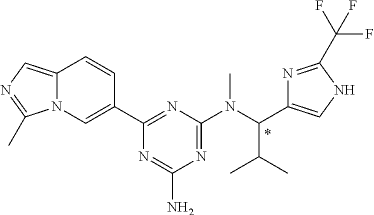 [SMILES: Cc1ncc2ccc(-c3nc(N)nc(N(C)C(c4c[nH]c(C(F)(F)F)n4)C(C)C)n3)cn12] |
| 162 | 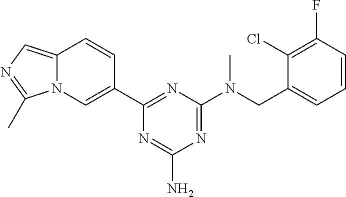 [SMILES: Cc1ncc2ccc(-c3nc(N)nc(N(C)Cc4cccc(F)c4Cl)n3)cn12] |
| 163 | 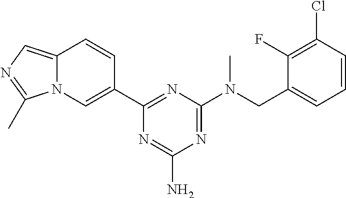 [SMILES: Cc1ncc2ccc(-c3nc(N)nc(N(C)Cc4cccc(Cl)c4F)n3)cn12] |
| 164 | 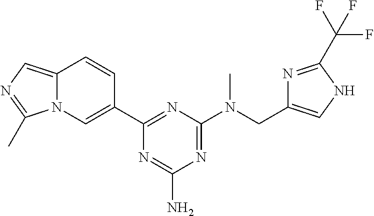 [SMILES: Cc1ncc2ccc(-c3nc(N)nc(N(C)Cc4c[nH]c(C(F)(F)F)n4)n3)cn12] |
| 165 | 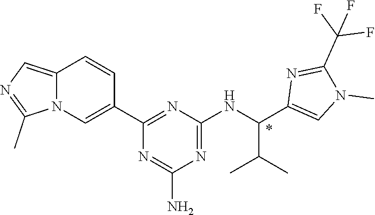 [SMILES: Cc1ncc2ccc(-c3nc(N)nc(NC(c4cn(C)c(C(F)(F)F)n4)C(C)C)n3)cn12] |
| 166 | 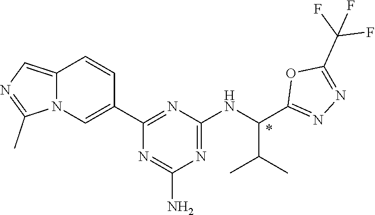 [SMILES: Cc1ncc2ccc(-c3nc(N)nc(NC(c4nnc(C(F)(F)F)o4)C(C)C)n3)cn12] |
| 167 | 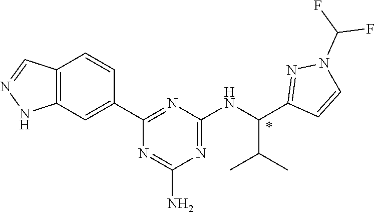 [SMILES: CC(C)C(Nc1nc(N)nc(-c2ccc3cn[nH]c3c2)n1)c1ccn(C(F)F)n1] |
| 168 | 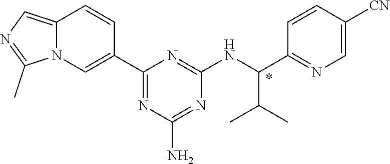 [SMILES: Cc1ncc2ccc(-c3nc(N)nc(NC(c4ccc(C#N)cn4)C(C)C)n3)cn12] |
| 169 | 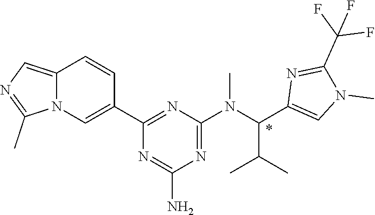 [SMILES: Cc1ncc2ccc(-c3nc(N)nc(N(C)C(c4cn(C)c(C(F)(F)F)n4)C(C)C)n3)cn12] |
| 170 | 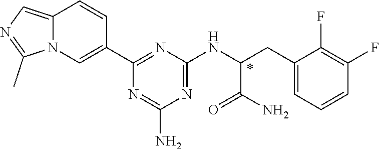 [SMILES: Cc1ncc2ccc(-c3nc(N)nc(NC(Cc4cccc(F)c4F)C(N)=O)n3)cn12] |
| 171 | 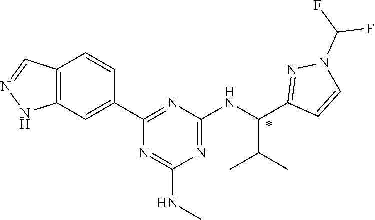 [SMILES: CNc1nc(NC(c2ccn(C(F)F)n2)C(C)C)nc(-c2ccc3cn[nH]c3c2)n1] |
| 172 | 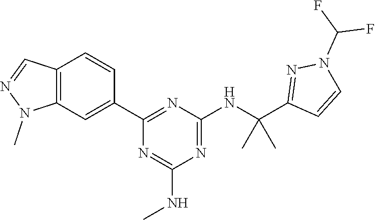 [SMILES: CNc1nc(NC(C)(C)c2ccn(C(F)F)n2)nc(-c2ccc3cnn(C)c3c2)n1] |
| 173 | 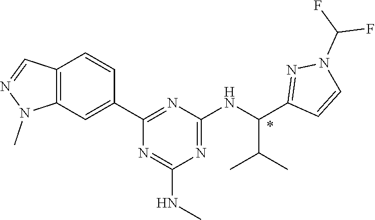 [SMILES: CNc1nc(NC(c2ccn(C(F)F)n2)C(C)C)nc(-c2ccc3cnn(C)c3c2)n1] |
| 174 | 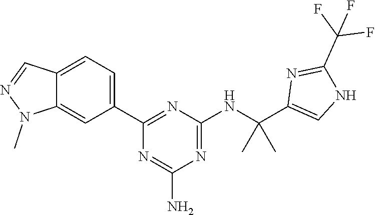 [SMILES: Cn1ncc2ccc(-c3nc(N)nc(NC(C)(C)c4c[nH]c(C(F)(F)F)n4)n3)cc21] |
| 175 | 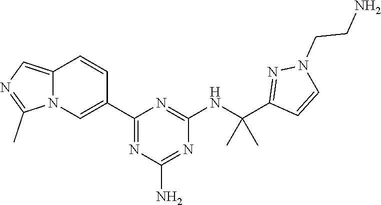 [SMILES: Cc1ncc2ccc(-c3nc(N)nc(NC(C)(C)c4ccn(CCN)n4)n3)cn12] |
| 176 | 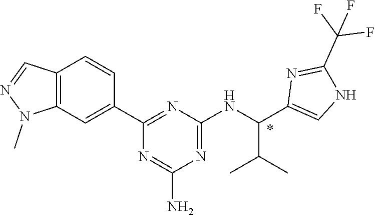 [SMILES: CC(C)C(Nc1nc(N)nc(-c2ccc3cnn(C)c3c2)n1)c1c[nH]c(C(F)(F)F)n1] |
| 177 | 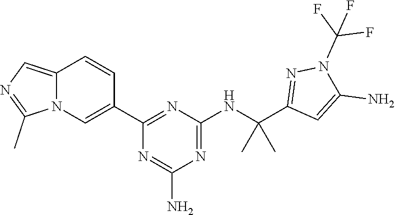 [SMILES: Cc1ncc2ccc(-c3nc(N)nc(NC(C)(C)c4cc(N)n(C(F)(F)F)n4)n3)cn12] |
| 178 | 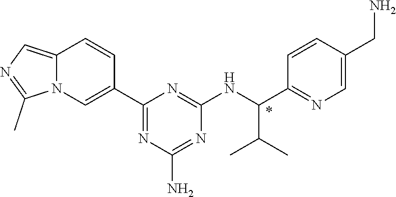 [SMILES: Cc1ncc2ccc(-c3nc(N)nc(NC(c4ccc(CN)cn4)C(C)C)n3)cn12] |
| 179 | 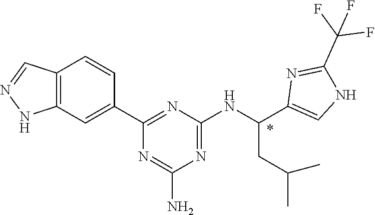 [SMILES: CC(C)CC(Nc1nc(N)nc(-c2ccc3cn[nH]c3c2)n1)c1c[nH]c(C(F)(F)F)n1] |
| 180 | 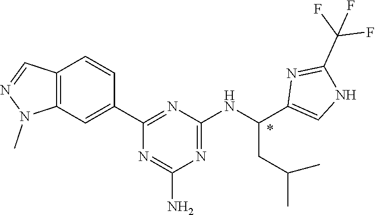 [SMILES: CC(C)CC(Nc1nc(N)nc(-c2ccc3cnn(C)c3c2)n1)c1c[nH]c(C(F)(F)F)n1] |
| 181 | 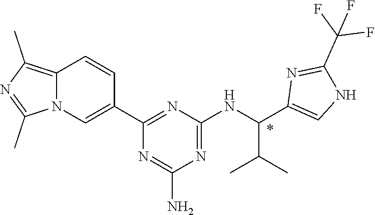 [SMILES: Cc1nc(C)n2cc(-c3nc(N)nc(NC(c4c[nH]c(C(F)(F)F)n4)C(C)C)n3)ccc12] |
| 182 | 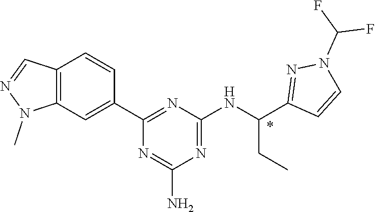 [SMILES: CCC(Nc1nc(N)nc(-c2ccc3cnn(C)c3c2)n1)c1ccn(C(F)F)n1] |
| 183 | 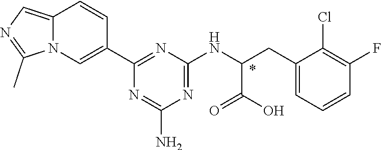 [SMILES: Cc1ncc2ccc(-c3nc(N)nc(NC(Cc4cccc(F)c4Cl)C(=O)O)n3)cn12] |
| 184 | 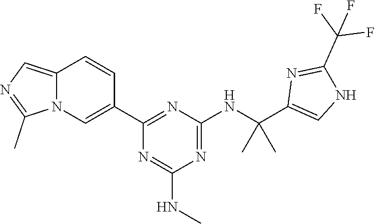 [SMILES: CNc1nc(NC(C)(C)c2c[nH]c(C(F)(F)F)n2)nc(-c2ccc3cnc(C)n3c2)n1] |
| 185 | 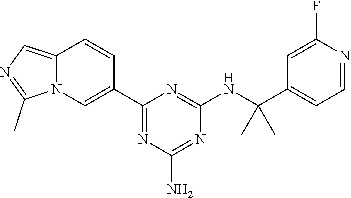 [SMILES: Cc1ncc2ccc(-c3nc(N)nc(NC(C)(C)c4ccnc(F)c4)n3)cn12] |
| 186 | 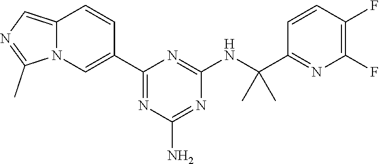 [SMILES: Cc1ncc2ccc(-c3nc(N)nc(NC(C)(C)c4ccc(F)c(F)n4)n3)cn12] |
| 187 | 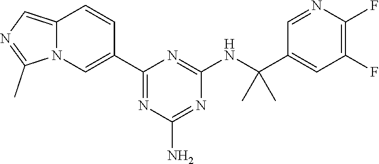 [SMILES: Cc1ncc2ccc(-c3nc(N)nc(NC(C)(C)c4cnc(F)c(F)c4)n3)cn12] |
| 188 | 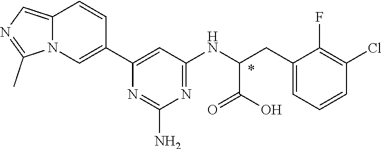 [SMILES: Cc1ncc2ccc(-c3cc(NC(Cc4cccc(Cl)c4F)C(=O)O)nc(N)n3)cn12] |
| 189 | 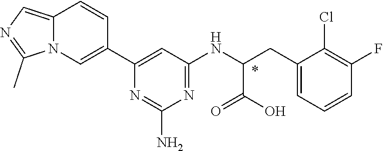 [SMILES: Cc1ncc2ccc(-c3cc(NC(Cc4cccc(F)c4Cl)C(=O)O)nc(N)n3)cn12] |
| 190 | 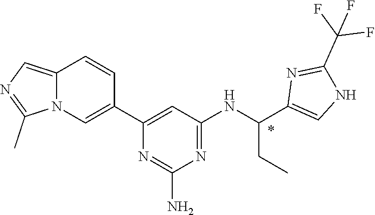 [SMILES: CCC(Nc1cc(-c2ccc3cnc(C)n3c2)nc(N)n1)c1c[nH]c(C(F)(F)F)n1] |
| 191 | 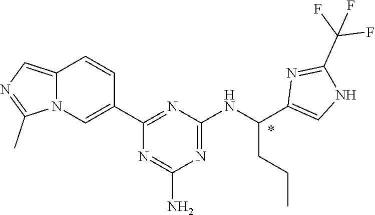 [SMILES: CCCC(Nc1nc(N)nc(-c2ccc3cnc(C)n3c2)n1)c1c[nH]c(C(F)(F)F)n1] |
| 192 | 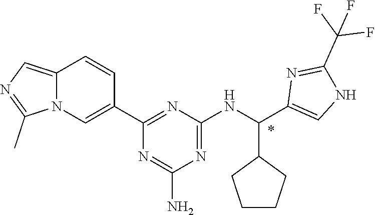 [SMILES: Cc1ncc2ccc(-c3nc(N)nc(NC(c4c[nH]c(C(F)(F)F)n4)C4CCCC4)n3)cn12] |
| 193 | 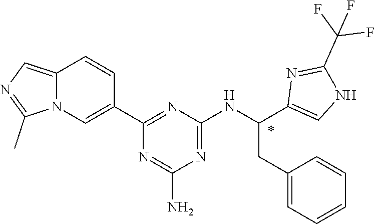 [SMILES: Cc1ncc2ccc(-c3nc(N)nc(NC(Cc4ccccc4)c4c[nH]c(C(F)(F)F)n4)n3)cn12] |
| 194 | 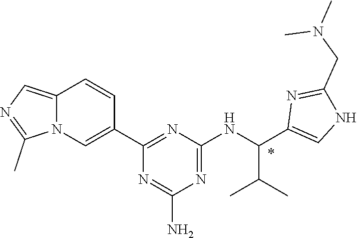 [SMILES: Cc1ncc2ccc(-c3nc(N)nc(NC(c4c[nH]c(CN(C)C)n4)C(C)C)n3)cn12] |
| 195 | 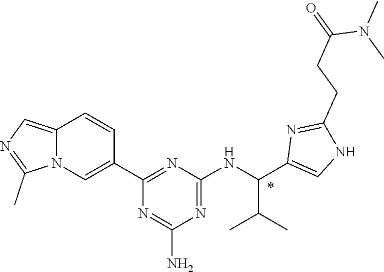 [SMILES: Cc1ncc2ccc(-c3nc(N)nc(NC(c4c[nH]c(CCC(=O)N(C)C)n4)C(C)C)n3)cn12] |
| 196 | 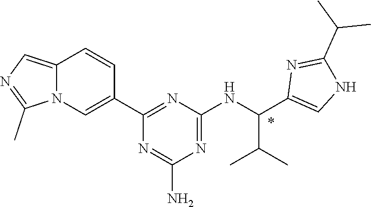 [SMILES: Cc1ncc2ccc(-c3nc(N)nc(NC(c4c[nH]c(C(C)C)n4)C(C)C)n3)cn12] |
| 197 | 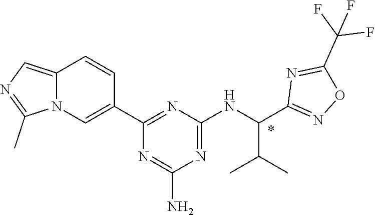 [SMILES: Cc1ncc2ccc(-c3nc(N)nc(NC(c4noc(C(F)(F)F)n4)C(C)C)n3)cn12] |
| 198 | 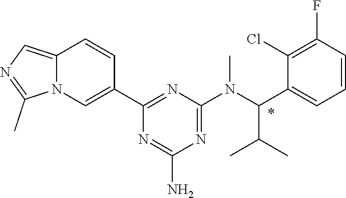 [SMILES: Cc1ncc2ccc(-c3nc(N)nc(N(C)C(c4cccc(F)c4Cl)C(C)C)n3)cn12] |
| 199 | 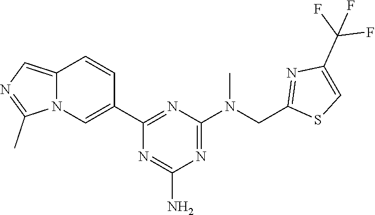 [SMILES: Cc1ncc2ccc(-c3nc(N)nc(N(C)Cc4nc(C(F)(F)F)cs4)n3)cn12] |
| 200 | 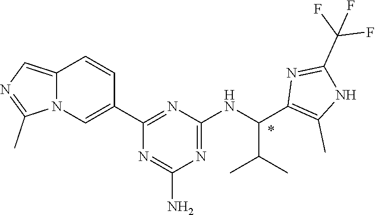 [SMILES: Cc1[nH]c(C(F)(F)F)nc1C(Nc1nc(N)nc(-c2ccc3cnc(C)n3c2)n1)C(C)C] |
| 201 | 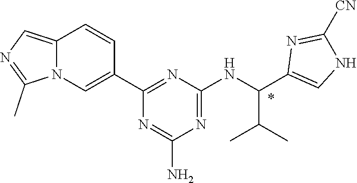 [SMILES: Cc1ncc2ccc(-c3nc(N)nc(NC(c4c[nH]c(C#N)n4)C(C)C)n3)cn12] |
| 202 | 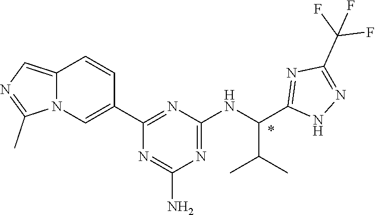 [SMILES: Cc1ncc2ccc(-c3nc(N)nc(NC(c4nc(C(F)(F)F)n[nH]4)C(C)C)n3)cn12] |
| 203 | 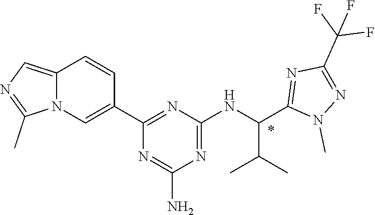 [SMILES: Cc1ncc2ccc(-c3nc(N)nc(NC(c4nc(C(F)(F)F)nn4C)C(C)C)n3)cn12] |
| 204 | 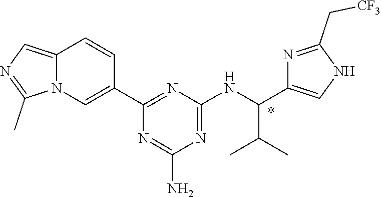 [SMILES: Cc1ncc2ccc(-c3nc(N)nc(NC(c4c[nH]c(CC(F)(F)F)n4)C(C)C)n3)cn12] |
| 205 | 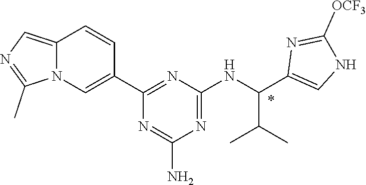 [SMILES: Cc1ncc2ccc(-c3nc(N)nc(NC(c4c[nH]c(OC(F)(F)F)n4)C(C)C)n3)cn12] |
| 206 | 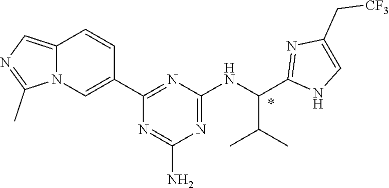 [SMILES: Cc1ncc2ccc(-c3nc(N)nc(NC(c4nc(CC(F)(F)F)c[nH]4)C(C)C)n3)cn12] |
| 207 | 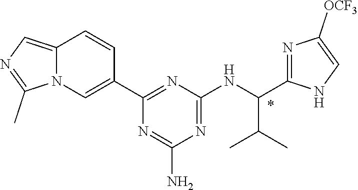 [SMILES: Cc1ncc2ccc(-c3nc(N)nc(NC(c4nc(OC(F)(F)F)c[nH]4)C(C)C)n3)cn12] |
| 208 | 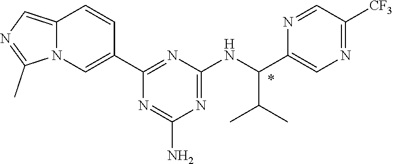 [SMILES: Cc1ncc2ccc(-c3nc(N)nc(NC(c4cnc(C(F)(F)F)cn4)C(C)C)n3)cn12] |
| 209 | 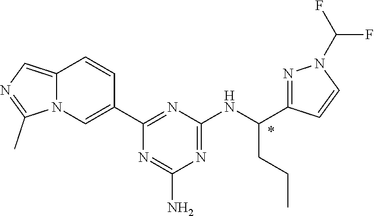 [SMILES: CCCC(Nc1nc(N)nc(-c2ccc3cnc(C)n3c2)n1)c1ccn(C(F)F)n1] |
| 210 | 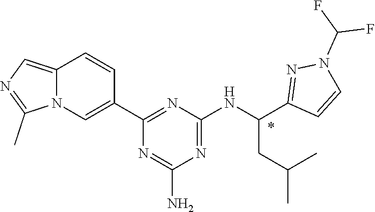 [SMILES: Cc1ncc2ccc(-c3nc(N)nc(NC(CC(C)C)c4ccn(C(F)F)n4)n3)cn12] |
| 211 | 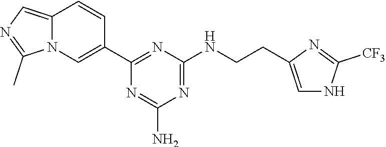 [SMILES: Cc1ncc2ccc(-c3nc(N)nc(NCCc4c[nH]c(C(F)(F)F)n4)n3)cn12] |
| 212 | 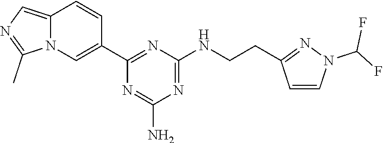 [SMILES: Cc1ncc2ccc(-c3nc(N)nc(NCCc4ccn(C(F)F)n4)n3)cn12] |
| 213 | 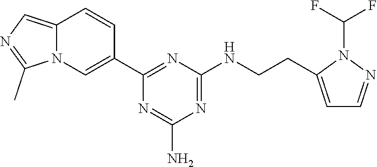 [SMILES: Cc1ncc2ccc(-c3nc(N)nc(NCCc4ccnn4C(F)F)n3)cn12] |
| 214 | 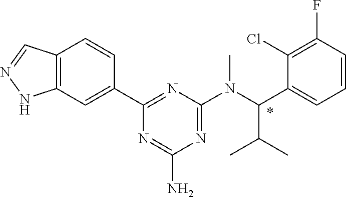 [SMILES: CC(C)C(c1cccc(F)c1Cl)N(C)c1nc(N)nc(-c2ccc3cn[nH]c3c2)n1] |
| 215 | 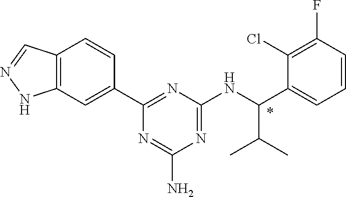 [SMILES: CC(C)C(Nc1nc(N)nc(-c2ccc3cn[nH]c3c2)n1)c1cccc(F)c1Cl] |
| 216 | 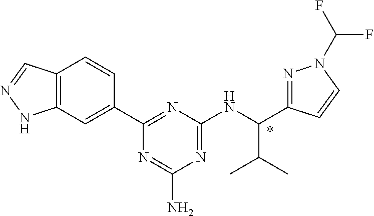 [SMILES: CC(C)C(Nc1nc(N)nc(-c2ccc3cn[nH]c3c2)n1)c1ccn(C(F)F)n1] |
| 217 | 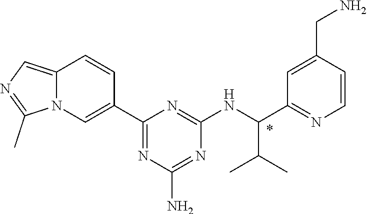 [SMILES: Cc1ncc2ccc(-c3nc(N)nc(NC(c4cc(CN)ccn4)C(C)C)n3)cn12] |
| 218 | 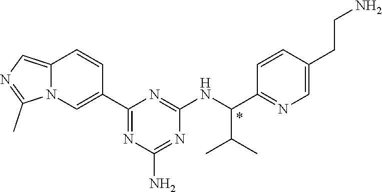 [SMILES: Cc1ncc2ccc(-c3nc(N)nc(NC(c4ccc(CCN)cn4)C(C)C)n3)cn12] |
| 219 | 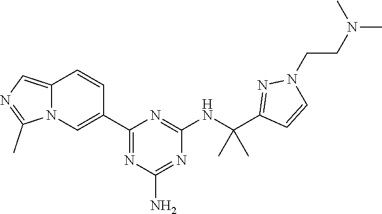 [SMILES: Cc1ncc2ccc(-c3nc(N)nc(NC(C)(C)c4ccn(CCN(C)C)n4)n3)cn12] |
| 220 | 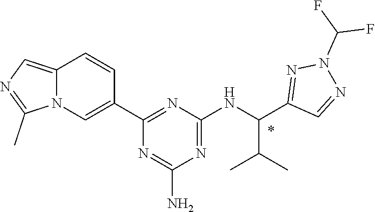 [SMILES: Cc1ncc2ccc(-c3nc(N)nc(NC(c4cnn(C(F)F)n4)C(C)C)n3)cn12] |
| 221 | 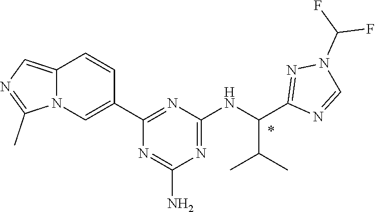 [SMILES: Cc1ncc2ccc(-c3nc(N)nc(NC(c4ncn(C(F)F)n4)C(C)C)n3)cn12] |
| 222 | 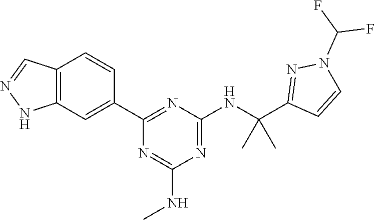 [SMILES: CNc1nc(NC(C)(C)c2ccn(C(F)F)n2)nc(-c2ccc3cn[nH]c3c2)n1] |
| 223 | 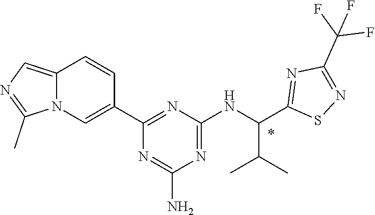 [SMILES: Cc1ncc2ccc(-c3nc(N)nc(NC(c4nc(C(F)(F)F)ns4)C(C)C)n3)cn12] |
| 224 | 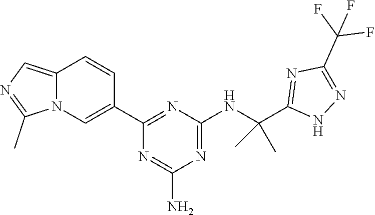 [SMILES: Cc1ncc2ccc(-c3nc(N)nc(NC(C)(C)c4nc(C(F)(F)F)n[nH]4)n3)cn12] |
| 225 | 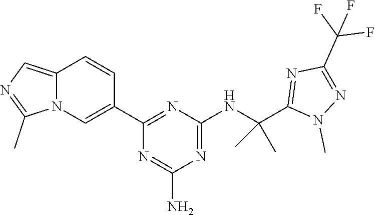 [SMILES: Cc1ncc2ccc(-c3nc(N)nc(NC(C)(C)c4nc(C(F)(F)F)nn4C)n3)cn12] |
| 226 | 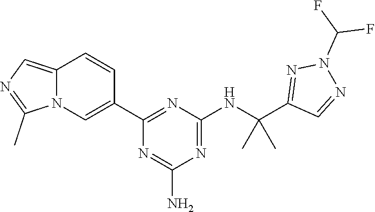 [SMILES: Cc1ncc2ccc(-c3nc(N)nc(NC(C)(C)c4cnn(C(F)F)n4)n3)cn12] |
| 227 | 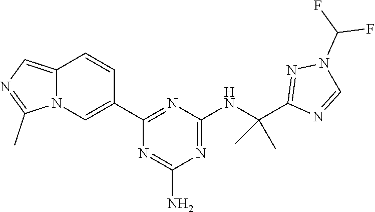 [SMILES: Cc1ncc2ccc(-c3nc(N)nc(NC(C)(C)c4ncn(C(F)F)n4)n3)cn12] |
| 228 | 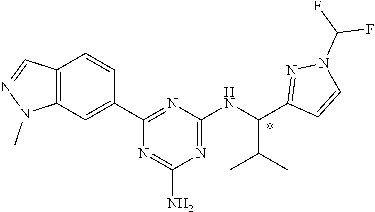 [SMILES: CC(C)C(Nc1nc(N)nc(-c2ccc3cnn(C)c3c2)n1)c1ccn(C(F)F)n1] |
| 229 | 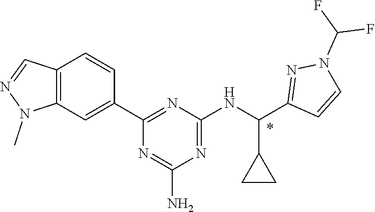 [SMILES: Cn1ncc2ccc(-c3nc(N)nc(NC(c4ccn(C(F)F)n4)C4CC4)n3)cc21] |
| 230 | 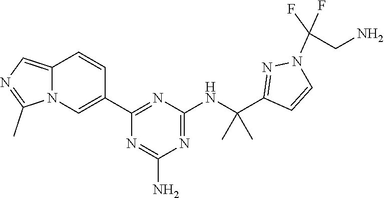 [SMILES: Cc1ncc2ccc(-c3nc(N)nc(NC(C)(C)c4ccn(C(F)(F)CN)n4)n3)cn12] |
| 231 | 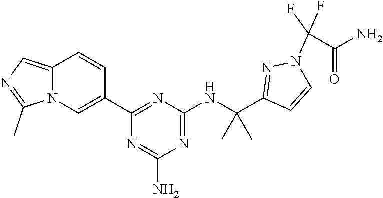 [SMILES: Cc1ncc2ccc(-c3nc(N)nc(NC(C)(C)c4ccn(C(F)(F)C(N)=O)n4)n3)cn12] |
| 232 | 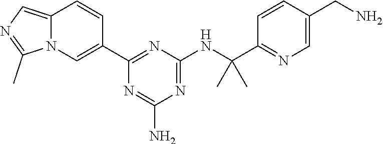 [SMILES: Cc1ncc2ccc(-c3nc(N)nc(NC(C)(C)c4ccc(CN)cn4)n3)cn12] |
| 233 | 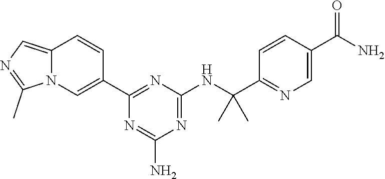 [SMILES: Cc1ncc2ccc(-c3nc(N)nc(NC(C)(C)c4ccc(C(N)=O)cn4)n3)cn12] |
| 234 | 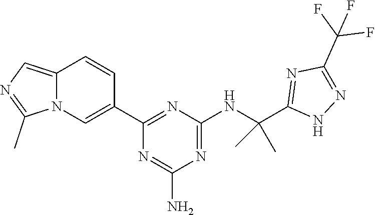 [SMILES: Cc1ncc2ccc(-c3nc(N)nc(NC(C)(C)c4nc(C(F)(F)F)n[nH]4)n3)cn12] |
| 235 | 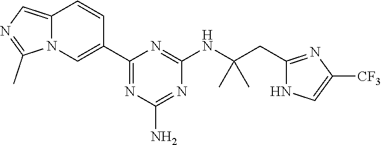 [SMILES: Cc1ncc2ccc(-c3nc(N)nc(NC(C)(C)Cc4nc(C(F)(F)F)c[nH]4)n3)cn12] |
| 236 | 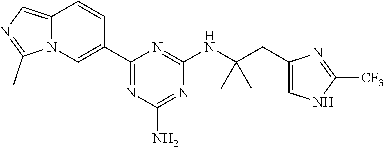 [SMILES: Cc1ncc2ccc(-c3nc(N)nc(NC(C)(C)Cc4c[nH]c(C(F)(F)F)n4)n3)cn12] |
| 237 | 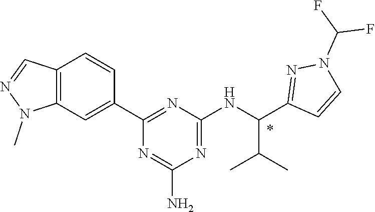 [SMILES: CC(C)C(Nc1nc(N)nc(-c2ccc3cnn(C)c3c2)n1)c1ccn(C(F)F)n1] |
| 238 | 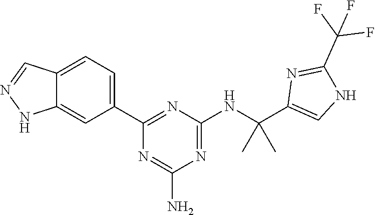 [SMILES: CC(C)(Nc1nc(N)nc(-c2ccc3cn[nH]c3c2)n1)c1c[nH]c(C(F)(F)F)n1] |
| 239 | 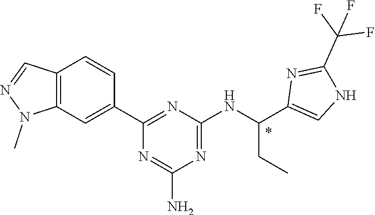 [SMILES: CCC(Nc1nc(N)nc(-c2ccc3cnn(C)c3c2)n1)c1c[nH]c(C(F)(F)F)n1] |

14. The method of claim 1, wherein the method is to treat a proliferative disease, a metabolic disorder or symptoms or conditions associated with a metabolic disease, or a platelet disorder, optionally wherein the platelet disorder is thrombocytopenia.

15. The method of claim 14, wherein the proliferative disease is a cancer, optionally wherein the cancer is selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and hematological cancer (e.g. leukaemia, lymphoma, myeloma and multiple myeloma).

16. The method of claim 14, wherein the metabolic disorder is selected from insulin resistance, diabetes and obesity, or wherein the symptoms and conditions associated with a metabolic disorder are selected from: increased blood sugar, increased cholesterol, increased triglyceride levels, heart disease, stroke, high blood pressure, and an increased risk of blood clots (e.g. deep vein thrombosis).

17. The method of claim 4, wherein the method is to treat a proliferative disease, a metabolic disorder or symptoms or conditions associated with a metabolic disease, or a platelet disorder, optionally wherein the platelet disorder is thrombocytopenia.

18. The method of claim 17, wherein the proliferative disease is a cancer, optionally wherein the cancer is selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and hematological cancer (e.g. leukaemia, lymphoma, myeloma and multiple myeloma).

19. The method of claim 17, wherein the metabolic disorder is selected from insulin resistance, diabetes and obesity, or wherein the symptoms and conditions associated with a metabolic disorder are selected from: increased blood sugar, increased cholesterol, increased triglyceride levels, heart disease, stroke, high blood pressure, and an increased risk of blood clots (e.g. deep vein thrombosis).

20. The method of claim 13, wherein the method is to treat a proliferative disease, a metabolic disorder or symptoms or conditions associated with a metabolic disease, or a platelet disorder, optionally wherein the platelet disorder is thrombocytopenia.

21. The method of claim 20, wherein the proliferative disease is a cancer, optionally wherein the cancer is selected from: breast, ovarian, lung, colorectal, prostate, oral, gastric, adrenocortical, pancreatic, kidney, sarcoma, liver, endometrial, thyroid, head or neck, brain (e.g. glioma), melanoma (e.g. ocular melanoma) and hematological cancer (e.g. leukaemia, lymphoma, myeloma and multiple myeloma).

22. The method of claim 20, wherein the metabolic disorder is selected from insulin resistance, diabetes and obesity, or wherein the symptoms and conditions associated with a metabolic disorder are selected from: increased blood sugar, increased cholesterol, increased triglyceride levels, heart disease, stroke, high blood pressure, and an increased risk of blood clots (e.g. deep vein thrombosis).

23. The method of claim 1, wherein the administration is combined with one or more additional anti-cancer agent and/or radiotherapy.

24. The method of claim 4, wherein the administration is combined with one or more additional anti-cancer agent and/or radiotherapy.

25. The method of claim 13, wherein the administration is combined with one or more additional anti-cancer agent and/or radiotherapy.

26. The method of claim 1, wherein the disease is one in which PD-L1 expression is dependent on interferon.

27. The method of claim 4, wherein the disease is one in which PD-L1 expression is dependent on interferon.

28. The method of claim 13, wherein the disease is one in which PD-L1 expression is dependent on interferon.
